SEQUENCE LISTING (191 sequences)

SEQ ID NO: 1 - protein, 607 aa, synthetic construct
MSSCINPSTLATSVNGFKCLPLATNRAAIRIMAKNKPVQCLVSTKYDNLTVDRRSANYQPSIWDHDFLQSLNSNYTDETYKRRAEELKGKVKTAIKDVTEPLDQLELIDNLQRLGLAYHFEPEIRNILRNIHNHNKDYNWRKENLYATSLEFRLLRQHGYPVSQEVFSGFKDDKVGFICDDFKGILSLHEASYYSLEGESIMEEAWQFTSKHLKEMMITSNSKEEDVFVAEQAKRALELPLHWKAPMLEARWFIHVYEKREDKNHLLLELAKLEFNTLQAIYQEELKDISGWWKDTGLGEKLSFARNRLVASFLWSMGIAFEPQFAYCRRVLTISIALITVIDDIYDVYGTLDELEIFTDAVARWDINYALKHLPGYMKMCFLALYNFVNEFAYYVLKQQDFDMLLSIKHAWLGLIQAYLVEAKWYHSKYTPKLEEYLENGLVSITGPLIITISYLSGTNPIIKKELEFLESNPDIVHWSSKIFRLQDDLGTSSDEIQRGDVPKSIQCYMHETGASEEVAREHIKDMMRQMWKKVNAYTADKDSPLTRTTAEFLLNLVRMSHFMYLHGDGHGVQNQETIDVGFTLLFQPIPLEDKDMAFTASPGTKG

SEQ ID NO: 2 - DNA, 1824 bp, synthetic construct
  source 1..1824: mol_type = other DNA
atgagcagctgcatcaatcccagcaccctggcaacatccgtgaatggcttcaaatgcctgcctctggcaacaaacagagctgctatccgcatcatggccaaaaacaagcccgtgcagtgcctggtgtccacaaaatacgataatctgacagtggaccggcggtctgccaactaccagccatctatctgggaccacgacttcctgcagtctctgaatagcaactataccgacgagacctacaagaggagggccgaagagctgaaaggcaaggtgaagaccgccatcaaggacgtgaccgagcccctggatcagctggagctgatcgataacctgcagcgcctgggactggcttaccattttgaacctgagattcgcaatattctgaggaacatccacaatcacaacaaggattataactggagaaaggagaacctgtacgctaccagcctcgagtttcgcctgctcaggcagcatgggtaccccgtgtcccaggaggtgttcagcggcttcaaagacgataaagtgggcttcatttgtgacgattttaagggcatcctgagtctgcacgaggcctcttactatagcctggagggagagagcatcatggaggaggcctggcagtttaccagcaaacatctcaaagagatgatgattacctccaattctaaggaggaggacgtgttcgtcgctgagcaggccaaaagagccctggagctgcccctgcactggaaagcccccatgctggaagctcggtggttcatccacgtgtatgagaaacgcgaggataaaaaccacctgctgctcgagctggccaaactcgagtttaacactctccaggccatctaccaggaggagctgaaggacatttccggctggtggaaggacaccggactgggcgaaaaactgagcttcgccaggaaccggctggtggcctccttcctgtggtccatgggtatcgccttcgagccacagtttgcctactgcaggagagtgctgactatcagcatcgctctgatcaccgtgattgacgacatttatgacgtgtacgggaccctggatgagctggagatctttactgacgccgtggcccggtgggatatcaactacgcccttaagcacctgcccggctacatgaagatgtgcttcctggccctgtacaactttgtgaatgaatttgcctactacgtgctgaagcagcaggactttgacatgctcctgtccattaagcacgcatggctgggactgatccaggcctatctggtggaggccaagtggtaccactccaagtacacacctaagctggaggagtacttggagaacggcctggtgagcatcaccggacccctgatcatcaccatctcctatctttctgggacaaaccctattatcaagaaggagctggaattcctggagtctaatcccgatatcgttcactggagctccaagattttcaggctgcaggacgacctggggaccagttcagatgagatccagagaggcgatgtgcctaagtccatccagtgttacatgcacgaaaccggcgcctccgaggaggtggcccgggaacacatcaaggacatgatgcgccagatgtggaagaaagtgaacgcctacaccgcagacaaggactcccccctgacccgcaccacagccgagttcctgctgaacctggtgagaatgagccacttcatgtacctgcacggagacggccacggcgtgcagaaccaggagacaatcgacgtgggcttcactctcctgttccagcccatccctctggaggataaagatatggccttcacagccagtcctggaaccaagggatga

SEQ ID NO: 3 - protein, 606 aa, synthetic construct
MSSSINPSTLVTSVNGFKCLPLATNKAAIRIMAKNKPVQCLVSAKYDNLTVDRRSANYQPSIWDHDFLQSLNSNYTDETYRRRAEELKGKVKTAIKDVTEPLDQLELIDNLQRLGLAYRFETEIRNILHNIYNNNKDYVWRKENLYATSLEFRLLRQHGYPVSQEVFNGFKDDQGGFICDDFKGILSLHEASYYRLEGESIMEEAWQFTSKHLKEVMISKSKEEDVFVAEQAKRALELPLHWKVPMLEARWFIHIYERREDKNHLLLELAKMEFNTLQAIYQEELKEISGWWKDTGLGEKLSFARNRLVASFLWSMGIAFEPQFAYCRRVLTISIALITVIDDIYDVYGTLDELEIFTDAVERWDINYALKHLPGYMKMCFLALYNFVNEFAYYVLKQQDFDMLLSIKNAWLGLIQAYLVEAKWYHSKYTPKLEEYLENGLVSITGPLIITISYLSGTNPIIKKELEFLESNPDIVHWSSKIFRLQDDLGTSSDEIQRGDVPKSIQCYMHETGASEEVAREHIKDMMRQMWKKVNAYTADKDSPLTRTTTEFLLNLVRMSHFMYLHGDGHGVQNQETIDVGFTLLFQPIPLEDKHMAFAASPGTKG

SEQ ID NO: 4 - DNA, 1821 bp, synthetic construct
  source 1..1821: mol_type = other DNA
atgagctccagcattaacccatccacccttgtgactagcgtgaatggcttcaagtgcctgcccctggcaactaacaaggccgccatccggatcatggccaagaacaagccagtgcagtgcctggtgtctgccaagtatgacaatctgacagtggacagacggagcgccaattaccagccaagcatctgggaccacgatttcctgcagagcctgaacagcaactacactgacgagacctacagacggcgcgctgaggagctgaaagggaaggtgaagaccgccatcaaggatgtgaccgagccactggaccagctggaactgattgataacctgcagagactgggcctggcctacagattcgaaaccgagatcaggaacattctgcacaacatttacaacaacaacaaggactacgtgtggagaaaagagaacctgtatgccaccagcctggagttcagactgctgcgccagcacggatacccagtgagccaggaggtgttcaatggcttcaaggacgaccagggcggattcatctgcgatgattttaaagggatcctgagcctgcacgaggcctcctactaccgcctggagggagaatctattatggaggaggcctggcagttcaccagcaagcacctgaaagaggtgatgatttccaagagcaaggaggaggacgtgtttgtcgccgaacaggccaagagagctctggaactgcctctgcactggaaggtgccaatgctggaagccaggtggtttatacacatttacgagagaagagaggacaagaatcacctgctgctggagctggctaaaatggagtttaataccttgcaggccatttatcaggaggagctgaaggaaatcagcggctggtggaaggatactggattgggcgagaagctcagctttgcccggaacagactggtggccagctttctgtggtctatgggcatcgccttcgagccccagtttgcctattgtcggagagtgctgacaattagcatcgccctgatcactgtgatcgacgacatctacgacgtgtacggcacactggacgagctggaaatcttcaccgatgccgtggagaggtgggacatcaactacgccctgaagcatctgccaggctacatgaagatgtgttttctggccctgtacaatttcgtgaatgagttcgcctattacgtgctcaagcagcaggactttgacatgctgctgtccatcaagaacgcttggctggggctgattcaggcttacctggtggaggccaaatggtaccactctaaatacactcctaaactggaagagtacctggaaaacggactggtgagcatcaccggcccactgatcattaccatcagctacctgtccgggactaaccccatcatcaaaaaggagctcgaatttctggaaagtaatcccgatatcgtgcactggagcagcaagattttcaggcttcaggatgatctggggacctcctccgatgagatccagagaggcgacgtgccaaaaagtattcagtgctacatgcacgagaccggggcctctgaggaggtggcccgggaacatattaaagatatgatgaggcagatgtggaaaaaggtgaatgcctatacagctgacaaggactcccccctgacaaggacaacaacagaattcttgctgaacctggtgagaatgagccatttcatgtacctgcacggcgacggccatggcgtgcagaatcaggagactattgacgtgggcttcacactgctgttccagcccatccccctggaggacaagcacatggcctttgcagccagccctggcactaaaggctaa

SEQ ID NO: 5 - protein, 606 aa, synthetic construct
MSSCINPSTLVTSANGFKCLPLATNKAAIRIMAKNKPVQCLVSAKYDNLIVDRRSANYQPSIWDHDFLQSLNSNYTDETYRRRAEELKGKVKIAIKDVTEPLDQLELIDNLQRLGLAYRFETEIRNILHNIYNNNKDYVWRKENLYATSLEFRLLRQHGYPVSQEVFNGFKDDQGGFIFDDFKGILSLHEASYYSLEGESIMEEAWQFTSKHLKEVMISKSMEEDVFVAEQAKRALELPLHWKVPMLEARWFIHVYEKREDKNHLLLELAKMEFNTLQAIYQEELKEISGWWKDTGLGEKLSFARNRLVASFLWSMGIAFEPQFAYCRRVLTISIALITVIDDIYDVYGTLDELEIFTDAVARWDINYALKHLPGYMKMCFLALYNFVNEFAYYVLKQQDFDMLLSIKNAWLGLIQAYLVEAKWYHSKYTPKLEEYLENGLVSITGPLIIAISYLSGTNPIIKKELEFLESNPDIVHWSSKIFRLQDDLGTSSDEIQRGDVPKSIQCYMHETGASEEVAREHIKDMMRQMWKKVNAYTADKDSPLTRTTTEFLLNLVRMSHFMYLHGDGHGVQNQETIDVGFTLLFQPIPLEDKDMAFTASPGTKG

SEQ ID NO: 6 - DNA, 1821 bp, synthetic construct
  source 1..1821: mol_type = other DNA
atgagctcctgtattaacccatccacccttgtgactagcgccaatggcttcaagtgcctgcccctggcaactaacaaggccgccatccggatcatggccaagaacaagccagtgcagtgcctggtgtctgccaagtatgacaatctgattgtggacagacggagcgccaattaccagccaagcatctgggaccacgatttcctgcagagcctgaacagcaactacactgacgagacctacagacggcgcgctgaggagctgaaagggaaggtgaagatcgccatcaaggatgtgaccgagccactggaccagctggaactgattgataacctgcagagactgggcctggcctacagattcgaaaccgagatcaggaacattctgcacaacatttacaacaacaacaaggactacgtgtggagaaaagagaacctgtatgccaccagcctggagttcagactgctgcgccagcacggatacccagtgagccaggaggtgttcaatggcttcaaggacgaccagggcggattcatcttcgatgattttaaagggatcctgagcctgcacgaggcctcctactactccctggagggagaatctattatggaggaggcctggcagttcaccagcaagcacctgaaagaggtgatgatttccaagagcatggaggaggacgtgtttgtcgccgaacaggccaagagagctctggaactgcctctgcactggaaggtgccaatgctggaagccaggtggtttatacacgtgtacgagaagagagaggacaagaatcacctgctgctggagctggctaaaatggagtttaataccttgcaggccatttatcaggaggagctgaaggaaatcagcggctggtggaaggatactggattgggcgagaagctcagctttgcccggaacagactggtggccagctttctgtggtctatgggcatcgccttcgagccccagtttgcctattgtcggagagtgctgacaattagcatcgccctgatcactgtgatcgacgacatctacgacgtgtacggcacactggacgagctggaaatcttcaccgatgccgtggcaaggtgggacatcaactacgccctgaagcatctgccaggctacatgaagatgtgttttctggccctgtacaatttcgtgaatgagttcgcctattacgtgctcaagcagcaggactttgacatgctgctgtccatcaagaacgcttggctggggctgattcaggcttacctggtggaggccaaatggtaccactctaaatacactcctaaactggaagagtacctggaaaacggactggtgagcatcaccggcccactgatcattgccatcagctacctgtccgggactaaccccatcatcaaaaaggagctcgaatttctggaaagtaatcccgatatcgtgcactggagcagcaagattttcaggcttcaggatgatctggggacctcctccgatgagatccagagaggcgacgtgccaaaaagtattcagtgctacatgcacgagaccggggcctctgaggaggtggcccgggaacatattaaagatatgatgaggcagatgtggaaaaaggtgaatgcctatacagctgacaaggactcccccctgacaaggacaacaacagaattcttgctgaacctggtgagaatgagccatttcatgtacctgcacggcgacggccatggcgtgcagaatcaggagactattgacgtgggcttcacactgctgttccagcccatccccctggaggacaaggacatggcctttacagccagccctggcactaaaggctaa

SEQ ID NO: 7 - protein, 606 aa, synthetic construct
MSSCINPSTLVTSVNAFKCLPLATNKAAIRIMAKYKPVQCLISAKYDNLTVDRRSANYQPSIWDHDFLQSLNSNYTDEAYKRRAEELRGKVKIAIKDVIEPLDQLELIDNLQRLGLAHRFETEIRNILNNIYNNNKDYNWRKENLYATSLEFRLLRQHGYPVSQEVFNGFKDDQGGFICDDFKGILSLHEASYYSLEGESIMEEAWQFTSKHLKEVMISKNMEEDVFVAEQAKRALELPLHWKVPMLEARWFIHIYERREDKNHLLLELAKMEFNTLQAIYQEELKEISGWWKDTGLGEKLSFARNRLVASFLWSMGIAFEPQFAYCRRVLTISIALITVIDDIYDVYGTLDELEIFTDAVERWDINYALKHLPGYMKMCFLALYNFVNEFAYYVLKQQDFDLLLSIKNAWLGLIQAYLVEAKWYHSKYTPKLEEYLENGLVSITGPLIITISYLSGTNPIIKKELEFLESNPDIVHWSSKIFRLQDDLGTSSDEIQRGDVPKSIQCYMHETGASEEVARQHIKDMMRQMWKKVNAYTADKDSPLTGTTTEFLLNLVRMSHFMYLHGDGHGVQNQETIDVGFTLLFQPIPLEDKHMAFTASPGTKG

SEQ ID NO: 8 - DNA, 1821 bp, synthetic construct
  source 1..1821: mol_type = other DNA
atgagctcctgtattaacccatccacccttgtgactagcgtgaatgccttcaagtgcctgcccctggcaactaacaaggccgccatccggatcatggccaagtacaagccagtgcagtgcctgatctctgccaagtatgacaatctgacagtggacagacggagcgccaattaccagccaagcatctgggaccacgatttcctgcagagcctgaacagcaactacactgacgaggcctacaagcggcgcgctgaggagctgcgcgggaaggtgaagatcgccatcaaggatgtgatcgagccactggaccagctggaactgattgataacctgcagagactgggcctggcccacagattcgaaaccgagatcaggaacattctgaataacatttacaacaacaacaaggactacaattggagaaaagagaacctgtatgccaccagcctggagttcagactgctgcgccagcacggatacccagtgagccaggaggtgttcaatggcttcaaggacgaccagggcggattcatctgcgatgattttaaagggatcctgagcctgcacgaggcctcctactactccctggagggagaatctattatggaggaggcctggcagttcaccagcaagcacctgaaagaggtgatgatttccaagaatatggaggaggacgtgtttgtcgccgaacaggccaagagagctctggaactgcctctgcactggaaggtgccaatgctggaagccaggtggtttatacacatttacgagagaagagaggacaagaatcacctgctgctggagctggctaaaatggagtttaataccttgcaggccatttatcaggaggagctgaaggaaatcagcggctggtggaaggatactggattgggcgagaagctcagctttgcccggaacagactggtggccagctttctgtggtctatgggcatcgccttcgagccccagtttgcctattgtcggagagtgctgacaattagcatcgccctgatcactgtgatcgacgacatctacgacgtgtacggcacactggacgagctggaaatcttcaccgatgccgtggagaggtgggacatcaactacgccctgaagcatctgccaggctacatgaagatgtgttttctggccctgtacaatttcgtgaatgagttcgcctattacgtgctcaagcagcaggactttgacctcctgctgtccatcaagaacgcttggctggggctgattcaggcttacctggtggaggccaaatggtaccactctaaatacactcctaaactggaagagtacctggaaaacggactggtgagcatcaccggcccactgatcattaccatcagctacctgtccgggactaaccccatcatcaaaaaggagctcgaatttctggaaagtaatcccgatatcgtgcactggagcagcaagattttcaggcttcaggatgatctggggacctcctccgatgagatccagagaggcgacgtgccaaaaagtattcagtgctacatgcacgagaccggggcctctgaggaggtggcccggcagcatattaaagatatgatgaggcagatgtggaaaaaggtgaatgcctatacagctgacaaggactcccccctgacagggacaacaacagaattcttgctgaacctggtgagaatgagccatttcatgtacctgcacggcgacggccatggcgtgcagaatcaggagactattgacgtgggcttcacactgctgttccagcccatccccctggaggacaagcacatggcctttacagccagccctggcactaaaggctaa

SEQ ID NO: 9 - protein, 607 aa, synthetic construct
MSSCINPSTLATSVNGFKYLPLATNRAAIRITAKNKPVQCLVSAKYDNLTVDRRSANYQPPIWDHDFLQSLNSDYTDETYRRRAEELKGKVKTAIEDVTEPLDQLELIDNLQRLGLAYHFETEIRNILHNIYNNNKDYIWRKENLYATSLEFRLLRQHGYPVSQEVSTGFKEDKGVFICDDFMGILSLHEASYYSLEGESIMEEAWQFTSKHLKEMMIISNSKEEDVFVAEQAKRALELPLHWKVPMLEARWFIHVYEKREDKNHLLLELAKLEFNVLQAIYQEELKDVSRWWKDIGLGEKLNFARDSLVASFVWSMGIVFEPQFAYCRRILTITFALISVIDDIYDVYGTLDELELFADAVERWDINYALNHLPDYMKICFLALYNLVNEFTYYVLKQQDFDILRSIKNAWLRNIQAYLVEAKWYHGKYTPTLGEFLENGLVSIGGPMVTMTAYLSGTNPIIEKELEFLESNQDIIHWSFKILRLQDDLGTSSDEIQRGDVPKSIQCYMHETGASEEVAREHIKDMMRQMWKKVNAYRADKDSPLSQTTVEFILNVVRVSHFMYLHGDGHGAQNQETMDVVFTLLFQPIPLDDKHIVATSSPVTKG

SEQ ID NO: 10 - DNA, 1824 bp, synthetic construct
  source 1..1824: mol_type = other DNA
atgtccagctgcattaacccttccacactggccacatccgtgaacggcttcaagtacctgcctctggccaccaatcgggccgccatcagaatcaccgccaaaaacaagccagtgcagtgtctggtgtccgccaagtacgacaatctgactgtggacagacgctccgccaattaccagccccctatctgggaccacgattttctgcagagcctgaattccgattataccgacgagacctacaggagaagggccgaagaactgaagggaaaagtcaagaccgccatcgaagacgtgaccgagccccttgatcagctggaactgatcgataatctgcagaggctggggctggcctaccactttgagacagagatcaggaacatcctgcacaatatttacaacaacaacaaggactatatttggcgcaaggagaacctgtacgccaccagcctggagttcaggctgctgaggcagcacggataccctgtgagccaggaggtgagcacaggctttaaggaggacaaaggcgtctttatctgtgacgatttcatgggaatcctgtccctgcatgaggcctcatactacagcctggagggcgagtccatcatggaagaggcttggcagttcacctccaaacacctgaaggagatgatgatcatctccaactctaaggaggaggacgtcttcgtggccgagcaggccaagagagctctggagctgccactgcactggaaggtgcccatgctggaggcccggtggttcatccacgtgtacgagaagcgcgaggataagaaccacctgctgctggaactcgccaaacttgagtttaatgtgctgcaggccatctaccaggaggagctgaaagatgtgagcagatggtggaaggatattggcctgggagagaaactgaatttcgcccgagacagcctggtcgcttccttcgtctggtctatgggcatcgtgttcgagccacagttcgcctattgcagacggatcctgactattacattcgccctgattagtgtgatcgacgacatctatgatgtgtacggtacactggacgagctggagctgttcgccgacgccgtggagaggtgggacatcaactacgccctgaaccacctgcccgactatatgaagatctgcttcctggctttgtacaacctggtgaacgagtttacctactacgtgctgaagcagcaggacttcgacatcctgaggagcatcaagaatgcctggctgcgaaatattcaggcctacctggtggaagctaagtggtaccacggcaaatatacaccgaccttgggcgagttcctggagaacggcctggtgtccatcggagggcctatggtgactatgaccgcctacttgagcggcaccaatcctatcattgagaaagagctggagtttctggagagcaatcaggacatcattcactggtctttcaagatcctgaggctgcaggatgatctgggcactagcagcgacgagatccagaggggcgacgttcctaaaagcatccagtgctacatgcatgagactggcgccagcgaagaggtggcccgcgagcatatcaaagacatgatgaggcagatgtggaaaaaggtgaacgcctacagagccgacaaagatagccctctgtcccagaccaccgtggagttcattctgaatgtggtgagagtgtctcacttcatgtacctccacggagacggacacggcgcccagaaccaggagaccatggatgtggtgtttaccctgctgttccagcctatcccactggatgacaagcacattgtggctacaagcagccccgtgaccaaaggctga

SEQ ID NO: 11 - protein, 608 aa, synthetic construct
MSSCINPSTLATSVNGFKCLPLATNRAAIRIMAKNKPVQCLVSTKYDNLTVDRRSANYQPSIWDHDFLQSLNSNYTDETYKRRAEELKGKVKTAIKDVTEPLDQLELIDNLQRLGLAYHFEPEIRNILRNIHNHNKDYNWRKENLYATSLEFRLLRQHGYPVSQEVFSGFKDDKVGFICDDFKGILSLHEASYYSLEGESIMEEAWQFTSKHLKEMMITSNSKEEDVFVAEQAKRALELPLHWKKVPMLEARWFIHVYEKREDKNHLLLELAKLEFNTLQAIYQEELKDISGWWKDTGLGEKLSFARNRLVASFLWSMGIAFEPQFAYCRRVLTISIALITVIDDIYDVYGTLDELEIFTDAVARWDINYALKHLPGYMKMCFLALYNFVNEFAYYVLKQQDFDMLLSIKHAWLGLIQAYLVEAKWYHSKYTPKLEEYLENGLVSITGPLIITISYLSGTNPIIKKELEFLESNPDIVHWSSKIFRLQDDLGTSSDEIQRGDVPKSIQCYMHETGASEEVAREHIKDMMRQMWKKVNAYTADKDSPLTRTTAEFLLNLVRMSHFMYLHGDGHGVQNQETIDVGFTLLFQPIPLEDKDMAFTASPGTKG

SEQ ID NO: 12 - DNA, 1827 bp, synthetic construct
  source 1..1827: mol_type = other DNA
atgtcctcctgcatcaatccgagcactctggcaacaagcgtgaacggcttcaagtgcctgccactggccaccaaccgcgccgccatcaggattatggccaagaataagcccgtgcagtgtctggtgtctactaaatatgacaatctgaccgtggacaggcggtccgccaactaccagccctccatctgggatcacgactttctgcagtccctcaactccaattacaccgacgagacctacaaaaggcgagccgaggagctgaagggcaaggtgaaaaccgccattaaggacgtgacagaacctctggaccagctggagctgatcgacaatctccagaggctgggcctggcttatcacttcgaacccgagatccgcaatatcctgcggaacattcacaatcataacaaggactacaattggaggaaggaaaacctgtatgccacctctctggagtttagactgctcagacagcacggctatcccgtcagccaggaggtgttctccggctttaaggatgacaaggtgggctttatttgcgatgacttcaaaggcatcctgtctctgcacgaggcctcctactacagtctggagggagagtccatcatggaagaggcatggcagttcacctcaaagcacctgaaggagatgatgatcaccagcaatagcaaggaggaggacgtgttcgtggctgagcaggctaagcgcgccctcgaactgccactgcactggaaaaaagtgccaatgctggaggctcgctggttcatccatgtgtacgagaagcgcgaagacaagaaccacctgctgttggaactcgccaagctggagttcaacacactgcaggccatctaccaggaagagctgaaggatattagtggctggtggaaagacaccggactgggggagaagctgagcttcgcccggaacagactggtggcctccttcctgtggagcatgggaatcgcctttgaacctcagtttgcctattgtcggagagtgctgacaatcagcatcgccctgatcaccgtgatcgacgacatttacgacgtctatggaaccctggacgagctggaaatctttacagacgccgtggctcgctgggatattaactacgccctgaagcacctgcctggctatatgaagatgtgcttcctcgccctgtacaactttgtgaacgagttcgcctattatgtgctgaagcagcaggattttgacatgctgctgagcattaagcacgcctggctgggcctgattcaggcctacctggtagaggccaagtggtaccacagcaagtacactcctaaactggaggagtatctggagaacggcctggtgtccatcactgggcccctgatcattaccatctcctacctgtccggcaccaacccgatcatcaagaaggagctggagttcctggagagcaatcctgacatcgtgcattggagttccaagattttcaggctgcaggatgacctgggcacaagctcagacgagattcagaggggcgatgtgcctaagtccatccagtgctatatgcacgagacaggagcatccgaagaagtggcccgcgagcacattaaggacatgatgcgccagatgtggaagaaagtgaatgcctacaccgccgacaaggactctcctctgacacgcaccaccgccgagttcctgctgaacctggtgagaatgtcccactttatgtatctgcacggcgacggccacggcgtgcagaaccaggagactatcgacgtgggatttaccctgctgttccagccaatccccctggaagacaaggacatggcattcactgcctctcccggcaccaagggctaa

SEQ ID NO: 13 - protein, 606 aa, synthetic construct
MSSSINPLTLVTSVNGFKCLPLATNKAAIRIMAKNKPVQCLVSAKYDNLTVDRRSANYQPSIWDHDFLQSLNSHSTDETYKRRAEELKGKVMTTIKDVTEPLDQLELIDNLQRLGLVYRFETEIRNILHNIYNNNKDYVWRKENLYATSLEFRLLRQHGYPVSQEVFNGFKDDQGGFICDDFKGILSLHEASHYSLEGESIMEEAWQFTSKHLKEVMISKSKEEDLFVAEQAKRALELPLHWKVPMLEARWFIHIYERREDKNHLLLELAKMEFNTLQAIYQEELKEISGWWKDTGLGEKLSFARNRLVASFLWSMGIAFEPQFAYCRRVLTISIALITVIDDIYDVYGTLDELELFTDAVERWDINYALKHLPGYMKMCFLALYNFVNEFAYYVLKQQDFDMLLSIKNAWLGLIQAYLVEAKWYHSKYTPKLEEYLENGLVSITGPLIITISYLSGTNPIIKKELEFLESNPDIVHWSSKIFRLQDDLGTSSDEIQRGDVPKSIQCYMHETGASEEVAREHIKDMMRQMWKKVNAYTADKDSPLTRTTTEFLLNLVRMSHFMYLHGDGHGVQNQQTIDVGFTLLFQPIPLGDKHMAFTASPGTKG

SEQ ID NO: 14 - DNA, 1821 bp, synthetic construct
  source 1..1821: mol_type = other DNA
atgtcctctagcatcaaccctctgaccctggtgacaagcgtgaacggctttaagtgtctgccactggccacaaacaaggccgccattcggatcatggccaaaaacaagcccgtgcagtgcctggtgtccgccaagtatgacaacctgacagtggatcggaggagcgcaaattaccagccctccatctgggaccacgattttctgcagtcactgaattctcattccaccgacgagacctacaagagacgggccgaggaactgaagggcaaggtcatgaccaccatcaaggacgtgactgagcctctggaccagctggaactgatcgacaatctgcagcggctcggcctggtgtacaggtttgagaccgagatcaggaacatcctgcacaatatttacaataacaacaaggactatgtgtggagaaaggagaatctgtacgccacaagcctggagttccgactgctgcgacagcatgggtatcctgtcagccaggaggtgtttaacggcttcaaagacgaccagggcggattcatctgcgacgatttcaagggcattctgagcctgcacgaggccagccactactcactcgaaggggaatccattatggaggaggcctggcagttcacaagcaagcaccttaaggaagttatgattagcaagagcaaagaggaagacctgtttgtggccgagcaggccaagagagccctggagcttcctctccactggaaggtgcccatgctggaggcccgatggttcattcacatctacgaaagaagagaggacaaaaaccacctgctgctggagctggccaaaatggaattcaataccctgcaggccatctaccaggaggagctgaaggagatcagcggctggtggaaggataccggcctgggcgagaagctgtccttcgcccggaataggctcgttgccagtttcctgtggtctatgggcatcgccttcgagccacagttcgcctactgtagaagagtgctgaccatcagcatcgcactgattaccgtgatcgacgacatctacgatgtgtacggcacactggacgaactggagctgtttacagacgccgtggagagatgggatatcaactacgccctgaagcacctgcccgggtatatgaagatgtgtttcctggccctctacaacttcgtcaacgagttcgcctactatgtgctgaagcagcaggacttcgacatgttgctgtccatcaagaacgcctggctgggcctgattcaggcatatctggtggaggccaagtggtaccactctaagtacactccaaagctggaggaatacttggagaacggactggtgagcatcactgggcctctgatcatcactattagctacctgagcggcaccaaccccattattaaaaaggagctggagttcctggagagtaatcccgatatcgtgcactggtcaagtaagattttcagactgcaggatgacctgggaacctcaagcgatgagatacagcgcggagacgtgccaaagtccattcagtgttatatgcacgagaccggcgcctcagaggaggtggcccgcgagcacattaaggacatgatgcggcagatgtggaagaaggtgaacgcctacaccgccgacaaggactcccccctgacaaggactacaaccgagtttctgctgaatctggtgagaatgtcccacttcatgtacctgcatggcgacggccacggcgtgcagaaccagcagaccatcgacgtgggattcaccctgctcttccagcccattccactgggcgacaagcacatggccttcaccgccagccctggcaccaagggctga

SEQ ID NO: 15 - protein, 557 aa, synthetic construct
MDRRSANYQPSIWDHDFLQSLNSNYTDETYKRRAEELKGKVKTAIKDVTEPLDQLELIDNLQRLGLAYHFEPEIRNILRNIHNHNKDYNWRKENLYATSLEFRLLRQHGYPVSQEVFSGFKDDKVGFICDDFKGILSLHEASYYSLEGESIMEEAWQFTSKHLKEMMITSNSKEEDVFVAEQAKRALELPLHWKAPMLEARWFIHVYEKREDKNHLLLELAKLEFNTLQAIYQEELKDISGWWKDTGLGEKLSFARNRLVASFLWSMGIAFEPQFAYCRRVLTISIALITVIDDIYDVYGTLDELEIFTDAVARWDINYALKHLPGYMKMCFLALYNFVNEFAYYVLKQQDFDMLLSIKHAWLGLIQAYLVEAKWYHSKYTPKLEEYLENGLVSITGPLIITISYLSGTNPIIKKELEFLESNPDIVHWSSKIFRLQDDLGTSSDEIQRGDVPKSIQCYMHETGASEEVAREHIKDMMRQMWKKVNAYTADKDSPLTRTTAEFLLNLVRMSHFMYLHGDGHGVQNQETIDVGFTLLFQPIPLEDKDMAFTASPGTKG

SEQ ID NO: 16 - DNA, 1674 bp, synthetic construct
  source 1..1674: mol_type = other DNA
atggatagacggtccgccaactaccagccctcaatctgggatcacgacttcctgcagagcctgaatagcaactacaccgacgagacttataagcggagggccgaagagctgaaagggaaggtgaagactgccataaaggatgtgactgagcccctcgatcagctggaactgattgacaacttgcagaggctgggcctggcctatcactttgagccagagatccgcaacatcctccgcaatatccacaaccataataaagattacaactggaggaaggaaaatctgtacgccacctccctggaattccggctgctgagacagcacgggtaccccgttagtcaggaagtgtttagcggcttcaaggacgacaaagtggggttcatctgcgatgatttcaagggcatcctgtccctgcacgaagccagctactactccctggagggggagagcatcatggaagaagcctggcagttcacctctaagcacctgaaggagatgatgattacatccaattccaaggaagaggatgtgttcgttgccgagcaggccaagagagccctggagctgcccctgcactggaaggcacccatgctggaggcccgctggttcatccacgtgtacgagaagagagaggacaagaaccacctgctgctggagctggccaagctggagtttaacacactgcaggccatataccaggaggagctgaaggatatctcaggatggtggaaagacaccggccttggcgagaagctgtccttcgccaggaatcggctcgtggcctcttttctgtggagcatgggcattgctttcgaaccccagttcgcttactgcagacgggtgctgaccatcagcatcgccctgatcaccgtgattgacgacatttacgacgtgtacggcaccctggacgagctggagattttcaccgacgctgtggccaggtgggatatcaactacgccctgaagcacctgcctggctatatgaagatgtgtttcctggccctgtacaatttcgtgaacgagttcgcatactacgtgctgaagcagcaggactttgacatgctgctgtccatcaagcatgcctggctgggactgatccaggcatacctggtggaggcaaagtggtaccacagcaaatatacacccaagctggaggagtatctggagaatggcctggtgagcatcaccggccccctgattattaccatttcctacctgagtggcacaaacccaatcatcaaaaaggagctggagttcctcgagagcaatccagatatcgtgcactggagcagcaaaattttccgcctgcaggacgacctcggcaccagcagcgacgaaattcagagaggcgacgtgccaaagagcatccagtgctatatgcacgagaccggcgcctccgaggaggtggccagggagcacatcaaggatatgatgcgccagatgtggaagaaggtgaatgcctacacagctgacaaggactccccactgaccagaaccaccgctgagttcctgctgaatctggtgcggatgagtcacttcatgtatctgcacggcgatggccatggggtgcagaatcaggagacaattgatgtggggttcacactgctctttcagcccatccccctggaggacaaggacatggcctttactgccagccccggcaccaagggctaa

SEQ ID NO: 17 - protein, 556 aa, synthetic construct
MDRRSANYQPSIWDHDFLQSLNSNYTDETYRRRAEELKGKVKTAIKDVTEPLDQLELIDNLQRLGLAYRFETEIRNILHNIYNNNKDYVWRKENLYATSLEFRLLRQHGYPVSQEVFNGFKDDQGGFICDDFKGILSLHEASYYRLEGESIMEEAWQFTSKHLKEVMISKSKEEDVFVAEQAKRALELPLHWKVPMLEARWFIHIYERREDKNHLLLELAKMEFNTLQAIYQEELKEISGWWKDTGLGEKLSFARNRLVASFLWSMGIAFEPQFAYCRRVLTISIALITVIDDIYDVYGTLDELEIFTDAVERWDINYALKHLPGYMKMCFLALYNFVNEFAYYVLKQQDFDMLLSIKNAWLGLIQAYLVEAKWYHSKYTPKLEEYLENGLVSITGPLIITISYLSGTNPIIKKELEFLESNPDIVHWSSKIFRLQDDLGTSSDEIQRGDVPKSIQCYMHETGASEEVAREHIKDMMRQMWKKVNAYTADKDSPLTRTTTEFLLNLVRMSHFMYLHGDGHGVQNQETIDVGFTLLFQPIPLEDKHMAFAASPGTKG

SEQ ID NO: 18 - DNA, 1671 bp, synthetic construct
  source 1..1671: mol_type = other DNA
atggaccggcggagcgccaattatcagccatccatctgggaccacgactttctgcagtccctgaactccaactacactgacgaaacctacagaagacgggccgaagagctgaagggcaaagtgaagacagccatcaaggatgtgaccgaacctctggaccagctggagctgatcgataacctgcagaggctgggcctggcttaccggttcgaaacagagatccggaacattctgcataacatttacaacaacaacaaagactacgtctggagaaaggaaaatctgtacgccacctccctggagttcagactgctgaggcagcacggctaccccgtgtcccaggaagttttcaacggcttcaaggatgaccaggggggattcatctgtgacgacttcaaaggcatcctgtctctgcacgaagcttcctactatagactggagggcgagtccatcatggaggaggcctggcagttcacatccaagcacctgaaggaggtgatgatctccaagtcaaaagaggaggacgtgtttgtggccgaacaggcaaagagagccctggagctgcccttgcattggaaggtgcccatgctggaggcacgctggtttattcacatttatgagcgcagagaggataaaaatcacctgctgctggagctggcgaaaatggagttcaataccctccaggccatctaccaggaggagctgaaagaaatcagcgggtggtggaaagacactggcctgggcgagaagctgtcatttgccaggaatcggctggtggcctccttcctgtggagcatgggcatcgccttcgagccccagttcgcttactgccggagagtgcttacaatctctattgccctcatcacagtgatcgatgatatctacgacgtgtacggcacgctggatgagctggagatttttaccgatgccgtggagaggtgggacatcaactacgccctgaaacacctgccaggatacatgaagatgtgtttcctggctctgtataacttcgtgaatgagtttgcctattatgtgctgaagcagcaggacttcgatatgctgctgtctatcaagaacgcctggctcggcctgattcaggcttacctggtggaagccaaatggtaccactctaagtacactcccaagctggaggagtacctggagaacgggttggtgagcatcaccggccctctgattatcaccatcagctacctgtccggcaccaacccaatcattaagaaggagctggagtttctggagtccaaccccgacattgtgcactggtcatctaagatcttccgcctgcaggatgacctgggcacctctagcgatgaaattcagagaggggacgtgcctaagtccatccaatgttacatgcacgagaccggagccagtgaggaggtggcccgcgaacacattaaggacatgatgaggcagatgtggaagaaggtgaacgcctacaccgccgataaggactcccccctgacacggaccaccacagagtttctgctgaatctggtgcggatgtcccacttcatgtacctgcatggggacggacacggagtgcagaatcaggaaacaatcgatgtgggctttacactgctgttccagcctatccccctggaggataagcacatggccttcgccgcctcccctggcacaaagggctga

SEQ ID NO: 19 - protein, 556 aa, synthetic construct
MDRRSANYQPSIWDHDFLQSLNSNYTDETYRRRAEELKGKVKIAIKDVTEPLDQLELIDNLQRLGLAYRFETEIRNILHNIYNNNKDYVWRKENLYATSLEFRLLRQHGYPVSQEVFNGFKDDQGGFIFDDFKGILSLHEASYYSLEGESIMEEAWQFTSKHLKEVMISKSMEEDVFVAEQAKRALELPLHWKVPMLEARWFIHVYEKREDKNHLLLELAKMEFNTLQAIYQEELKEISGWWKDTGLGEKLSFARNRLVASFLWSMGIAFEPQFAYCRRVLTISIALITVIDDIYDVYGTLDELEIFTDAVARWDINYALKHLPGYMKMCFLALYNFVNEFAYYVLKQQDFDMLLSIKNAWLGLIQAYLVEAKWYHSKYTPKLEEYLENGLVSITGPLIIAISYLSGTNPIIKKELEFLESNPDIVHWSSKIFRLQDDLGTSSDEIQRGDVPKSIQCYMHETGASEEVAREHIKDMMRQMWKKVNAYTADKDSPLTRTTTEFLLNLVRMSHFMYLHGDGHGVQNQETIDVGFTLLFQPIPLEDKDMAFTASPGTKG

SEQ ID NO: 20 - DNA, 1671 bp, synthetic construct
  source 1..1671: mol_type = other DNA
atggataggcggagtgctaattaccagccaagcatctgggatcacgatttcctgcagtccctgaactccaactataccgacgaaacataccggaggagagccgaggagctgaaggggaaagtgaagatcgccattaaggacgtgaccgagcccctggaccagctggagctgattgataacctgcagcgcctgggcctggcctatcggtttgagacggaaatccggaatatcctgcacaacatctataataataacaaggattacgtgtggagaaaggaaaatctgtacgccacctccctggagtttagactgctgaggcagcacggataccccgtgtcccaggaagtgttcaacggcttcaaggatgaccagggcggctttatcttcgatgacttcaagggaattctgtccctgcacgaggccagttactactctctggagggcgagtccatcatggaggaggcttggcagttcacctccaagcacctgaaagaggtgatgattagcaaatccatggaagaggacgtgtttgtggccgagcaggctaagagagccctggagctgcctctgcactggaaggtgccaatgctggaggcaaggtggtttatccacgtgtatgagaagcgcgaggataagaatcacctgctgctggagctggccaaaatggagttcaacactctgcaggcaatctaccaggaagagctgaaagagatcagcggctggtggaaagataccgggctgggggagaagctgagctttgcccgaaataggctggtggccagctttctgtggagcatggggattgctttcgagcctcagttcgcctactgccggagagtgctcaccatcagtatcgccctgatcaccgtgatcgacgacatctacgacgtgtacggcaccctggacgaactggagatcttcactgatgcagtggccaggtgggatatcaactatgcactgaaacacctgcccggatacatgaaaatgtgctttctggccctgtataacttcgtgaacgagttcgcttattacgtgctgaagcagcaggatttcgacatgctgctcagcatcaagaacgcctggctgggcctgatccaggcctacctggtggaggccaaatggtaccatagcaagtacacccccaagctggaagagtaccttgagaacggcctggtgtctattactggccctctgatcatcgccatcagctacctctctggcaccaacccaatcattaagaaggagctggagtttctggagtcaaacccagatatcgtgcattggtccagcaaaatcttccggctgcaggatgacctggggacctccagcgacgagatccaaagaggagacgtgccaaaatccatccagtgctatatgcacgaaaccggagccagcgaagaggtggccagagagcatatcaaggacatgatgaggcagatgtggaagaaagtaaacgcctacaccgcagataaggacagccccctcacccgcaccacaaccgaattcctgctgaatctggtgcggatgtcccatttcatgtacctgcatggcgatggccatggtgtccagaaccaggaaaccatcgatgtgggcttcaccctgctgtttcagcctatccctctggaggacaaggacatggcctttaccgcaagtcccggcacaaagggctga

SEQ ID NO: 21 - protein, 556 aa, synthetic construct
MDRRSANYQPSIWDHDFLQSLNSNYTDEAYKRRAEELRGKVKIAIKDVIEPLDQLELIDNLQRLGLAHRFETEIRNILNNIYNNNKDYNWRKENLYATSLEFRLLRQHGYPVSQEVFNGFKDDQGGFICDDFKGILSLHEASYYSLEGESIMEEAWQFTSKHLKEVMISKNMEEDVFVAEQAKRALELPLHWKVPMLEARWFIHIYERREDKNHLLLELAKMEFNTLQAIYQEELKEISGWWKDTGLGEKLSFARNRLVASFLWSMGIAFEPQFAYCRRVLTISIALITVIDDIYDVYGTLDELEIFTDAVERWDINYALKHLPGYMKMCFLALYNFVNEFAYYVLKQQDFDLLLSIKNAWLGLIQAYLVEAKWYHSKYTPKLEEYLENGLVSITGPLIITISYLSGTNPIIKKELEFLESNPDIVHWSSKIFRLQDDLGTSSDEIQRGDVPKSIQCYMHETGASEEVARQHIKDMMRQMWKKVNAYTADKDSPLTGTTTEFLLNLVRMSHFMYLHGDGHGVQNQETIDVGFTLLFQPIPLEDKHMAFTASPGTKG

SEQ ID NO: 22 - DNA, 1671 bp, synthetic construct
  source 1..1671: mol_type = other DNA
atggaccggcggagcgccaattatcagccatccatctgggaccacgactttctgcagtccctgaactccaactacactgacgaagcctacaagagacgggccgaagagctgcggggcaaagtgaagattgccatcaaggatgtgatcgaacctctggaccagctggagctgatcgataacctgcagaggctgggcctggctcaccggttcgaaacagagatccggaacattctgaataacatttacaacaacaacaaagactacaactggagaaaggaaaatctgtacgccacctccctggagttcagactgctgaggcagcacggctaccccgtgtcccaggaagttttcaacggcttcaaggatgaccaggggggattcatctgtgacgacttcaaaggcatcctgtctctgcacgaagcttcctactattcactggagggcgagtccatcatggaggaggcctggcagttcacatccaagcacctgaaggaggtgatgatctccaagaacatggaggaggacgtgtttgtggccgaacaggcaaagagagccctggagctgcccttgcattggaaggtgcccatgctggaggcacgctggtttattcacatttatgagcgcagagaggataaaaatcacctgctgctggagctggcgaaaatggagttcaataccctccaggccatctaccaggaggagctgaaagaaatcagcgggtggtggaaagacactggcctgggcgagaagctgtcatttgccaggaatcggctggtggcctccttcctgtggagcatgggcatcgccttcgagccccagttcgcttactgccggagagtgcttacaatctctattgccctcatcacagtgatcgatgatatctacgacgtgtacggcacgctggatgagctggagatttttaccgatgccgtggagaggtgggacatcaactacgccctgaaacacctgccaggatacatgaagatgtgtttcctggctctgtataacttcgtgaatgagtttgcctattatgtgctgaagcagcaggacttcgatctgctgctgtctatcaagaacgcctggctcggcctgattcaggcttacctggtggaagccaaatggtaccactctaagtacactcccaagctggaggagtacctggagaacgggttggtgagcatcaccggccctctgattatcaccatcagctacctgtccggcaccaacccaatcattaagaaggagctggagtttctggagtccaaccccgacattgtgcactggtcatctaagatcttccgcctgcaggatgacctgggcacctctagcgatgaaattcagagaggggacgtgcctaagtccatccaatgttacatgcacgagaccggagccagtgaggaggtggcccgccagcacattaaggacatgatgaggcagatgtggaagaaggtgaacgcctacaccgccgataaggactcccccctgacaggcaccaccacagagtttctgctgaatctggtgcggatgtcccacttcatgtacctgcatggggacggacacggagtgcagaatcaggaaacaatcgatgtgggctttacactgctgttccagcctatccccctggaggataagcacatggccttcaccgcctcccctggcacaaagggctga

SEQ ID NO: 23 - protein, 557 aa, synthetic construct
MDRRSANYQPPIWDHDFLQSLNSDYTDETYRRRAEELKGKVKTAIEDVTEPLDQLELIDNLQRLGLAYHFETEIRNILHNIYNNNKDYIWRKENLYATSLEFRLLRQHGYPVSQEVSTGFKEDKGVFICDDFMGILSLHEASYYSLEGESIMEEAWQFTSKHLKEMMIISNSKEEDVFVAEQAKRALELPLHWKVPMLEARWFIHVYEKREDKNHLLLELAKLEFNVLQAIYQEELKDVSRWWKDIGLGEKLNFARDSLVASFVWSMGIVFEPQFAYCRRILTITFALISVIDDIYDVYGTLDELELFADAVERWDINYALNHLPDYMKICFLALYNLVNEFTYYVLKQQDFDILRSIKNAWLRNIQAYLVEAKWYHGKYTPTLGEFLENGLVSIGGPMVTMTAYLSGTNPIIEKELEFLESNQDIIHWSFKILRLQDDLGTSSDEIQRGDVPKSIQCYMHETGASEEVAREHIKDMMRQMWKKVNAYRADKDSPLSQTTVEFILNVVRVSHFMYLHGDGHGAQNQETMDVVFTLLFQPIPLDDKHIVATSSPVTKG

SEQ ID NO: 24 - DNA, 1674 bp, synthetic construct
  source 1..1674: mol_type = other DNA
atggatagacggtccgccaactaccagccccctatctgggatcacgacttcctgcagagcctgaatagcgactacaccgacgagacttatagacggagggccgaagagctgaaagggaaggtgaagactgccatagaggatgtgactgagcccctcgatcagctggaactgattgacaacttgcagaggctgggcctggcctatcactttgagacagagatccgcaacatcctccacaatatctacaacaataataaagattacatctggaggaaggaaaatctgtacgccacctccctggaattccggctgctgagacagcacgggtaccccgttagtcaggaagtgagtacaggcttcaaggaggacaaaggagtgttcatctgcgatgatttcatgggcatcctgtccctgcacgaagccagctactactccctggagggggagagcatcatggaagaagcctggcagttcacctctaagcacctgaaggagatgatgattatttccaattccaaggaagaggatgtgttcgttgccgagcaggccaagagagccctggagctgcccctgcactggaaggtgcccatgctggaggcccgctggttcatccacgtgtacgagaagagagaggacaagaaccacctgctgctggagctggccaagctggagtttaacgtgctgcaggccatataccaggaggagctgaaggatgtctcaagatggtggaaagacatcggccttggcgagaagctgaacttcgccagggattccctcgtggcctcttttgtgtggagcatgggcattgtgttcgaaccccagttcgcttactgcagacggatcctgaccatcacattcgccctgatctccgtgattgacgacatttacgacgtgtacggcaccctggacgagctggagctgttcgccgacgctgtggagaggtgggatatcaactacgccctgaaccacctgcctgactatatgaagatctgtttcctggccctgtacaatctggtgaacgagttcacatactacgtgctgaagcagcaggactttgacatcctgagatccatcaagaatgcctggctgaggaatatccaggcatacctggtggaggcaaagtggtaccacggaaaatatacacccacactgggggagtttctggagaatggcctggtgagcatcggcggccccatggtgactatgactgcctacctgagtggcacaaacccaatcatcgagaaggagctggagttcctcgagagcaatcaggatatcattcactggagctttaaaattctgcgcctgcaggacgacctcggcaccagcagcgacgaaattcagagaggcgacgtgccaaagagcatccagtgctatatgcacgagaccggcgcctccgaggaggtggccagggagcacatcaaggatatgatgcgccagatgtggaagaaggtgaatgcctacagggctgacaaggactccccactgtcccagaccaccgtggagttcatcctgaatgtggtgcgggtgagtcacttcatgtatctgcacggcgatggccatggggcccagaatcaggagacaatggatgtggtgttcacactgctctttcagcccatccccctggacgacaagcacatcgtggccacttctagccccgtgaccaagggctaa

SEQ ID NO: 25 - protein, 558 aa, synthetic construct
MDRRSANYQPSIWDHDFLQSLNSNYTDETYKRRAEELKGKVKTAIKDVTEPLDQLELIDNLQRLGLAYHFEPEIRNILRNIHNHNKDYNWRKENLYATSLEFRLLRQHGYPVSQEVFSGFKDDKVGFICDDFKGILSLHEASYYSLEGESIMEEAWQFTSKHLKEMMITSNSKEEDVFVAEQAKRALELPLHWKKVPMLEARWFIHVYEKREDKNHLLLELAKLEFNTLQAIYQEELKDISGWWKDTGLGEKLSFARNRLVASFLWSMGIAFEPQFAYCRRVLTISIALITVIDDIYDVYGTLDELEIFTDAVARWDINYALKHLPGYMKMCFLALYNFVNEFAYYVLKQQDFDMLLSIKHAWLGLIQAYLVEAKWYHSKYTPKLEEYLENGLVSITGPLIITISYLSGTNPIIKKELEFLESNPDIVHWSSKIFRLQDDLGTSSDEIQRGDVPKSIQCYMHETGASEEVAREHIKDMMRQMWKKVNAYTADKDSPLTRTTAEFLLNLVRMSHFMYLHGDGHGVQNQETIDVGFTLLFQPIPLEDKDMAFTASPGTKG

SEQ ID NO: 26 - DNA, 1677 bp, synthetic construct
  source 1..1677: mol_type = other DNA
atggatcgcagatctgccaattatcagccttccatttgggaccatgatttcctgcagtccctgaatagcaactacacagacgagacctacaagcgtcgggccgaggagcttaagggaaaggtgaagaccgcgatcaaggacgtgactgagccactggaccagctggagctgattgacaacctgcagaggctgggactggcctaccacttcgagccagaaatccgcaatatcctgcgcaatattcataaccataacaaggactacaactggaggaaggagaatctgtacgccacatccctggaattcaggcttctgagacagcacggatacccagtgagccaggaggtgttcagcggcttcaaggacgacaaagtgggcttcatttgcgatgacttcaagggaatcctgagtctgcacgaagctagctattactcactggaaggcgagagcatcatggaagaggcctggcagtttaccagcaagcacctgaaggagatgatgatcacttctaattctaaggaggaagacgtgttcgtggccgagcaggccaaacgcgcccttgagctgcccctgcactggaaaaaggtccctatgctggaagccagatggtttatccatgtgtatgagaaaagggaggacaagaaccacctgctgctggagctggccaagctggagttcaacactctgcaggccatttaccaggaggagctgaaggatatcagcggctggtggaaggacaccggcctgggcgaaaaactgtctttcgccagaaacagactggtggcatcctttctgtggagcatgggaatcgcctttgaacctcagttcgcctactgcaggagagtgctgaccatttccatcgccctgattacagtgatcgatgatatctacgacgtctacggcaccctggacgagctggagatttttacagacgccgtggctaggtgggatattaattacgccctgaagcacctgcctggatatatgaagatgtgcttcctggccctgtacaactttgtgaacgagtttgcctactacgtgctgaaacagcaggacttcgacatgctgctgtctatcaagcatgcttggctgggactgatccaggcctacctggtggaagccaagtggtatcacagcaagtatacacccaagctggaggagtacctggagaacggcctggtgagcattacaggccccctgatcatcacaatctcatatctctccgggaccaacccaatcattaaaaaggaactggaattcctggaatccaaccctgacattgtgcactggtctagcaagatctttaggctgcaggacgacctgggaaccagctctgatgagattcagcgcggcgatgtgcccaagtccatccagtgttacatgcacgagaccggcgcctctgaggaagtggccagggagcacatcaaggatatgatgaggcagatgtggaaaaaagttaatgcctacaccgccgacaaggactcacctctgactaggacaaccgcagaattcctgctgaatctggtgcggatgtctcactttatgtacctgcatggggacgggcacggcgtgcagaaccaggagacaatcgatgtgggcttcaccctgctgtttcagcccattcccctggaggacaaagacatggccttcacagcctctcccggcacaaaaggctga

SEQ ID NO: 27 - protein, 556 aa, synthetic construct
MDRRSANYQPSIWDHDFLQSLNSHSTDETYKRRAEELKGKVMTTIKDVTEPLDQLELIDNLQRLGLVYRFETEIRNILHNIYNNNKDYVWRKENLYATSLEFRLLRQHGYPVSQEVFNGFKDDQGGFICDDFKGILSLHEASHYSLEGESIMEEAWQFTSKHLKEVMISKSKEEDLFVAEQAKRALELPLHWKVPMLEARWFIHIYERREDKNHLLLELAKMEFNTLQAIYQEELKEISGWWKDTGLGEKLSFARNRLVASFLWSMGIAFEPQFAYCRRVLTISIALITVIDDIYDVYGTLDELELFTDAVERWDINYALKHLPGYMKMCFLALYNFVNEFAYYVLKQQDFDMLLSIKNAWLGLIQAYLVEAKWYHSKYTPKLEEYLENGLVSITGPLIITISYLSGTNPIIKKELEFLESNPDIVHWSSKIFRLQDDLGTSSDEIQRGDVPKSIQCYMHETGASEEVAREHIKDMMRQMWKKVNAYTADKDSPLTRTTTEFLLNLVRMSHFMYLHGDGHGVQNQQTIDVGFTLLFQPIPLGDKHMAFTASPGTKG

SEQ ID NO: 28 - DNA, 1671 bp, synthetic construct
  source 1..1671: mol_type = other DNA
atggaccggcggagcgccaattatcagccatccatctgggaccacgactttctgcagtccctgaactcccactccactgacgaaacctacaagagacgggccgaagagctgaagggcaaagtgatgacaaccatcaaggatgtgaccgaacctctggaccagctggagctgatcgataacctgcagaggctgggcctggtgtaccggttcgaaacagagatccggaacattctgcataacatttacaacaacaacaaagactacgtctggagaaaggaaaatctgtacgccacctccctggagttcagactgctgaggcagcacggctaccccgtgtcccaggaagttttcaacggcttcaaggatgaccaggggggattcatctgtgacgacttcaaaggcatcctgtctctgcacgaagcttcccactattcactggagggcgagtccatcatggaggaggcctggcagttcacatccaagcacctgaaggaggtgatgatctccaagtcaaaagaggaggacctgtttgtggccgaacaggcaaagagagccctggagctgcccttgcattggaaggtgcccatgctggaggcacgctggtttattcacatttatgagcgcagagaggataaaaatcacctgctgctggagctggcgaaaatggagttcaataccctccaggccatctaccaggaggagctgaaagaaatcagcgggtggtggaaagacactggcctgggcgagaagctgtcatttgccaggaatcggctggtggcctccttcctgtggagcatgggcatcgccttcgagccccagttcgcttactgccggagagtgcttacaatctctattgccctcatcacagtgatcgatgatatctacgacgtgtacggcacgctggatgagctggagctgtttaccgatgccgtggagaggtgggacatcaactacgccctgaaacacctgccaggatacatgaagatgtgtttcctggctctgtataacttcgtgaatgagtttgcctattatgtgctgaagcagcaggacttcgatatgctgctgtctatcaagaacgcctggctcggcctgattcaggcttacctggtggaagccaaatggtaccactctaagtacactcccaagctggaggagtacctggagaacgggttggtgagcatcaccggccctctgattatcaccatcagctacctgtccggcaccaacccaatcattaagaaggagctggagtttctggagtccaaccccgacattgtgcactggtcatctaagatcttccgcctgcaggatgacctgggcacctctagcgatgaaattcagagaggggacgtgcctaagtccatccaatgttacatgcacgagaccggagccagtgaggaggtggcccgcgaacacattaaggacatgatgaggcagatgtggaagaaggtgaacgcctacaccgccgataaggactcccccctgacacggaccaccacagagtttctgctgaatctggtgcggatgtcccacttcatgtacctgcatggggacggacacggagtgcagaatcagcagacaatcgatgtgggctttacactgctgttccagcctatccccctgggcgataagcacatggccttcaccgcctcccctggcacaaagggctga

SEQ ID NO: 29 - protein, 562 aa, synthetic construct
MHHHHHHDRRSANYQPSIWDHDFLQSLNSNYTDEAYKRRAEELRGKVKIAIKDVIEPLDQLELIDNLQRLGLAHRFETEIRNILNNIYNNNKDYNWRKENLYATSLEFRLLRQHGYPVSQEVFNGFKDDQGGFICDDFKGILSLHEASYYSLEGESIMEEAWQFTSKHLKEVMISKNMEEDVFVAEQAKRALELPLHWKVPMLEARWFIHIYERREDKNHLLLELAKMEFNTLQAIYQEELKEISGWWKDTGLGEKLSFARNRLVASFLWSMGIAFEPQFAYCRRVLTISIALITVIDDIYDVYGTLDELEIFTDAVERWDINYALKHLPGYMKMCFLALYNFVNEFAYYVLKQQDFDLLLSIKNAWLGLIQAYLVEAKWYHSKYTPKLEEYLENGLVSITGPLIITISYLSGTNPIIKKELEFLESNPDIVHWSSKIFRLQDDLGTSSDEIQRGDVPKSIQCYMHETGASEEVARQHIKDMMRQMWKKVNAYTADKDSPLTGTTTEFLLNLVRMSHFMYLHGDGHGVQNQETIDVGFTLLFQPIPLEDKHMAFTASPGTKG

SEQ ID NO: 30 - DNA, 1688 bp, synthetic construct
  source 1..1688: mol_type = other DNA
tgcatcaccatcatcaccacgacagaagaagtgctaactaccagccatccatttgggaccacgatttcctgcagagcctgaacagcaattacacagatgaggcctataagaggagagcagaggagctgcgcggcaaggtgaagatcgccatcaaggacgtgatcgagcccctggatcagctggagctgatcgacaacctccagcggctgggcctggcccaccgcttcgagacagagatccggaacatcctgaacaacatctacaacaacaacaaggactacaactggcggaaggagaacctgtacgccaccagcctggagtttcggctgctgagacagcacggctaccccgtgagccaggaggtgttcaatggctttaaggacgatcagggcggcttcatctgcgacgacttcaagggcatcctgtctctgcacgaggcctcctactattctctggagggcgagagcatcatggaggaggcctggcagttcacctccaagcacctgaaggaagtgatgatcagcaagaacatggaggaggacgtgtttgtggccgagcaggccaagagagccctggagctgcccctgcactggaaggtgcctatgctggaggccaggtggttcatccacatctatgagaggcgcgaggataagaatcacctgctgctggagctggccaagatggagtttaacacactccaggccatctaccaggaggagctgaaggagatcagcggatggtggaaggacaccggcctgggagagaagctgtctttcgccaggaatcgcctggtggcctcttttctgtggagcatgggcatcgccttcgagcctcagtttgcctattgccggagagtgctgacaatcagcatcgccctgatcaccgtgatcgacgacatctacgacgtgtacggcacactggacgagctggagattttcaccgatgccgtggagcggtgggacatcaactacgccctgaagcacctgccaggctatatgaagatgtgcttcctggccctgtacaatttcgtgaacgagtttgcctactatgtgctgaagcagcaggactttgatctgctgctgagcatcaagaatgcctggctgggcctgatccaggcctacctggtggaggccaagtggtatcactctaagtatacacccaagctggaggagtatctggagaacggcctggtgagcatcacaggcccactgatcatcaccatcagctacctgtccggcaccaatcccatcatcaagaaggagctggagttcctggagtccaaccctgacatcgtgcactggagcagcaagattttccggctccaggacgatctgggcacatctagcgatgagatccagcggggcgacgtgccaaagagcatccagtgttacatgcacgagacaggagcctccgaggaggtggcaagacagcacatcaaggacatgatgaggcagatgtggaagaaggtgaacgcctatacagccgacaaggattcccccctgaccggcaccacaaccgagttcctgctgaatctggtgagaatgtctcactttatgtacctgcacggcgatggccacggcgtgcagaaccaggagacaatcgacgtgggcttcaccctgctgtttcagcctatccccctggaggacaagcacatggcattcaccgcaagccctggcactaaaggatga

SEQ ID NO: 31 - protein, 606 aa, synthetic construct
MSSCINPSTLVTSVNGFKCLPLATNKAAIRIMAKNKPVQCLVSAKYDNLTVDRRSANYQPSIWDHDFLQSLNSNYTDETYKRRAEELKGKVKTAIKDVTEPLDQLELIDNLQRLGLAYRFETEIRNILHNIYNNNKDYNWRKENLYATSLEFRLLRQHGYPVSQEVFNGFKDDQGGFICDDFKGILSLHEASYYSLEGESIMEEAWQFTSKHLKEVMISKNKEEDVFVAEQAKRALELPLHWKVPMLEARWFIHVYEKREDKNHLLLELAKMEFNTLQAIYQEELKEISGWWKDTGLGEKLSFARNRLVASFLWSMGIAFEPQFAYCRRVLTISIALITVIDDIYDVYGTLDELEIFTDAVERWDINYALKHLPGYMKMCFLALYNFVNEFAYYVLKQQDFDMLLSIKNAWLGLIQAYLVEAKWYHSKYTPKLEEYLENGLVSITGPLIITISYLSGTNPIIKKELEFLESNPDIVHWSSKIFRLQDDLGTSSDEIQRGDVPKSIQCYMHETGASEEVAREHIKDMMRQMWKKVNAYTADKDSPLTRTTTEFLLNLVRMSHFMYLHGDGHGVQNQETIDVGFTLLFQPIPLEDKHMAFTASPGTKG

SEQ ID NO: 32 - DNA, 1821 bp, synthetic construct
  source 1..1821: mol_type = other DNA
atgagctcctgtattaacccatccacccttgtgactagcgtgaatggcttcaagtgcctgcccctggcaactaacaaggccgccatccggatcatggccaagaacaagccagtgcagtgcctggtgtctgccaagtatgacaatctgacagtggacagacggagcgccaattaccagccaagcatctgggaccacgatttcctgcagagcctgaacagcaactacactgacgagacctacaagcggcgcgctgaggagctgaaagggaaggtgaagaccgccatcaaggatgtgaccgagccactggaccagctggaactgattgataacctgcagagactgggcctggcctacagattcgaaaccgagatcaggaacattctgcacaacatttacaacaacaacaaggactacaattggagaaaagagaacctgtatgccaccagcctggagttcagactgctgcgccagcacggatacccagtgagccaggaggtgttcaatggcttcaaggacgaccagggcggattcatctgcgatgattttaaagggatcctgagcctgcacgaggcctcctactactccctggagggagaatctattatggaggaggcctggcagttcaccagcaagcacctgaaagaggtgatgatttccaagaataaggaggaggacgtgtttgtcgccgaacaggccaagagagctctggaactgcctctgcactggaaggtgccaatgctggaagccaggtggtttatacacgtgtacgagaagagagaggacaagaatcacctgctgctggagctggctaaaatggagtttaataccttgcaggccatttatcaggaggagctgaaggaaatcagcggctggtggaaggatactggattgggcgagaagctcagctttgcccggaacagactggtggccagctttctgtggtctatgggcatcgccttcgagccccagtttgcctattgtcggagagtgctgacaattagcatcgccctgatcactgtgatcgacgacatctacgacgtgtacggcacactggacgagctggaaatcttcaccgatgccgtggagaggtgggacatcaactacgccctgaagcatctgccaggctacatgaagatgtgttttctggccctgtacaatttcgtgaatgagttcgcctattacgtgctcaagcagcaggactttgacatgctgctgtccatcaagaacgcttggctggggctgattcaggcttacctggtggaggccaaatggtaccactctaaatacactcctaaactggaagagtacctggaaaacggactggtgagcatcaccggcccactgatcattaccatcagctacctgtccgggactaaccccatcatcaaaaaggagctcgaatttctggaaagtaatcccgatatcgtgcactggagcagcaagattttcaggcttcaggatgatctggggacctcctccgatgagatccagagaggcgacgtgccaaaaagtattcagtgctacatgcacgagaccggggcctctgaggaggtggcccgggaacatattaaagatatgatgaggcagatgtggaaaaaggtgaatgcctatacagctgacaaggactcccccctgacaaggacaacaacagaattcttgctgaacctggtgagaatgagccatttcatgtacctgcacggcgacggccatggcgtgcagaatcaggagactattgacgtgggcttcacactgctgttccagcccatccccctggaggacaagcacatggcctttacagccagccctggcactaaaggctaa

SEQ ID NO: 33 - protein, 556 aa, synthetic construct
MDRRSANYQPSIWDHDFLQSLNSNYTDETYKRRAEELKGKVKTAIKDVTEPLDQLELIDNLQRLGLAYRFETEIRNILHNIYNNNKDYNWRKENLYATSLEFRLLRQHGYPVSQEVFNGFKDDQGGFICDDFKGILSLHEASYYSLEGESIMEEAWQFTSKHLKEVMISKNKEEDVFVAEQAKRALELPLHWKVPMLEARWFIHVYEKREDKNHLLLELAKMEFNTLQAIYQEELKEISGWWKDTGLGEKLSFARNRLVASFLWSMGIAFEPQFAYCRRVLTISIALITVIDDIYDVYGTLDELEIFTDAVERWDINYALKHLPGYMKMCFLALYNFVNEFAYYVLKQQDFDMLLSIKNAWLGLIQAYLVEAKWYHSKYTPKLEEYLENGLVSITGPLIITISYLSGTNPIIKKELEFLESNPDIVHWSSKIFRLQDDLGTSSDEIQRGDVPKSIQCYMHETGASEEVAREHIKDMMRQMWKKVNAYTADKDSPLTRTTTEFLLNLVRMSHFMYLHGDGHGVQNQETIDVGFTLLFQPIPLEDKHMAFTASPGTKG

SEQ ID NO: 34 - DNA, 1671 bp, synthetic construct
  source 1..1671: mol_type = other DNA
atggaccggcggagcgccaattatcagccatccatctgggaccacgactttctgcagtccctgaactccaactacactgacgaaacctacaagagacgggccgaagagctgaagggcaaagtgaagacagccatcaaggatgtgaccgaacctctggaccagctggagctgatcgataacctgcagaggctgggcctggcttaccggttcgaaacagagatccggaacattctgcataacatttacaacaacaacaaagactacaactggagaaaggaaaatctgtacgccacctccctggagttcagactgctgaggcagcacggctaccccgtgtcccaggaagttttcaacggcttcaaggatgaccaggggggattcatctgtgacgacttcaaaggcatcctgtctctgcacgaagcttcctactattcactggagggcgagtccatcatggaggaggcctggcagttcacatccaagcacctgaaggaggtgatgatctccaagaacaaagaggaggacgtgtttgtggccgaacaggcaaagagagccctggagctgcccttgcattggaaggtgcccatgctggaggcacgctggtttattcacgtgtatgagaaaagagaggataaaaatcacctgctgctggagctggcgaaaatggagttcaataccctccaggccatctaccaggaggagctgaaagaaatcagcgggtggtggaaagacactggcctgggcgagaagctgtcatttgccaggaatcggctggtggcctccttcctgtggagcatgggcatcgccttcgagccccagttcgcttactgccggagagtgcttacaatctctattgccctcatcacagtgatcgatgatatctacgacgtgtacggcacgctggatgagctggagatttttaccgatgccgtggagaggtgggacatcaactacgccctgaaacacctgccaggatacatgaagatgtgtttcctggctctgtataacttcgtgaatgagtttgcctattatgtgctgaagcagcaggacttcgatatgctgctgtctatcaagaacgcctggctcggcctgattcaggcttacctggtggaagccaaatggtaccactctaagtacactcccaagctggaggagtacctggagaacgggttggtgagcatcaccggccctctgattatcaccatcagctacctgtccggcaccaacccaatcattaagaaggagctggagtttctggagtccaaccccgacattgtgcactggtcatctaagatcttccgcctgcaggatgacctgggcacctctagcgatgaaattcagagaggggacgtgcctaagtccatccaatgttacatgcacgagaccggagccagtgaggaggtggcccgcgaacacattaaggacatgatgaggcagatgtggaagaaggtgaacgcctacaccgccgataaggactcccccctgacacggaccaccacagagtttctgctgaatctggtgcggatgtcccacttcatgtacctgcatggggacggacacggagtgcagaatcaggaaacaatcgatgtgggctttacactgctgttccagcctatccccctggaggataagcacatggccttcaccgcctcccctggcacaaagggctga

SEQ ID NO: 35 - protein, 607 aa, synthetic construct
MSSXINPSTLXTSVNGFKCLPLATNXAAIRIMAKNKPVQCLVSXKYDNLTVDRRSANYQPSIWDHDFLQSLNSNYTDETYXRRAEELKGKVKXAIKDVTEPLDQLELIDNLQRLGLAYXFEXEIRNILXNIXNXNKDYXWRKENLYATSLEFRLLRQHGYPVSQEVFXGFKDDXXGFICDDFKGILSLHEASYYSLEGESIMEEAWQFTSKHLKEXMIXXXXXEEDVFVAEQAKRALELPLHWKVPMLEARWFIHXYEXREDKNHLLLELAKXEFNTLQAIYQEELKXISGWWKDTGLGEKLSFARNRLVASFLWSMGIAFEPQFAYCRRVLTISIALITVIDDIYDVYGTLDELEXFTDAVXRWDINYALKHLPGYMKMCFLALYNFVNEFAYYVLKQQDFDMLLSIKXAWLGLIQAYLVEAKWYHSKYTPKLEEYLENGLVSITGPLIITISYLSGTNPIIKKELEFLESNPDIVHWSSKIFRLQDDLGTSSDEIQRGDVPKSIQCYMHETGASEEVAREHIKDMMRQMWKKVNAYTADKDSPLTRTTXEFLLNLVRMSHFMYLHGDGHGVQNQETIDVGFTLLFQPIPLEDKXMAFTASPGTKG

SEQ ID NO: 36 - protein, 557 aa, synthetic construct
MDRRSANYQPSIWDHDFLQSLNSNYTDETYXRRAEELKGKVKXAIKDVTEPLDQLELIDNLQRLGLAYXFEXEIRNILXNIXNXNKDYXWRKENLYATSLEFRLLRQHGYPVSQEVFXGFKDDXXGFICDDFKGILSLHEASYYSLEGESIMEEAWQFTSKHLKEXMIXXXXXEEDVFVAEQAKRALELPLHWKVPMLEARWFIHXYEXREDKNHLLLELAKXEFNTLQAIYQEELKXISGWWKDTGLGEKLSFARNRLVASFLWSMGIAFEPQFAYCRRVLTISIALITVIDDIYDVYGTLDELEXFTDAVXRWDINYALKHLPGYMKMCFLALYNFVNEFAYYVLKQQDFDMLLSIKXAWLGLIQAYLVEAKWYHSKYTPKLEEYLENGLVSITGPLIITISYLSGTNPIIKKELEFLESNPDIVHWSSKIFRLQDDLGTSSDEIQRGDVPKSIQCYMHETGASEEVAREHIKDMMRQMWKKVNAYTADKDSPLTRTTXEFLLNLVRMSHFMYLHGDGHGVQNQETIDVGFTLLFQPIPLEDKXMAFTASPGTKG

SEQ ID NO: 37 - protein, 608 aa, synthetic construct
MSSXINPXTLXTSXNXFKXLPLATNXAAIRIXAKXKPVQCLXSXKYDNLXVDRRSANYQPXIWDHDFLQSLNSXXTDEXYXRRAEELXGKVXXXIXDVXEPLDQLELIDNLQRLGLXXXFEXEIRNILXNIXNXNKDYXWRKENLYATSLEFRLLRQHGYPVSQEVXXGFKXDXXXFIXDDFXGILSLHEASXYXLEGESIMEEAWQFTSKHLKEXMIXXXXXEEDXFVAEQAKRALELPLHWKXXPMLEARWFIHXYEXREDKNHLLLELAKXEFNXLQAIYQEELKXXSXWWKDXGLGEKLXFARXXLVASFXWSMGIXFEPQFAYCRRXLTIXXALIXVIDDIYDVYGTLDELEXFXDAVXRWDINYALXHLPXYMKXCFLALYNXVNEFXYYVLKQQDFDXLXSIKXAWLXXIQAYLVEAKWYHXKYTPXLXEXLENGLVSIXGPXXXXXXYLSGTNPIIXKELEFLESNXDIXHWSXKIXRLQDDLGTSSDEIQRGDVPKSIQCYMHETGASEEVARXHIKDMMRQMWKKVNAYXADKDSPLXXTTXEFXLNXVRXSHFMYLHGDGHGXQNQXTXDVXFTLLFQPIPLXDKXXXXXXSPXTKG

SEQ ID NO: 38 - protein, 558 aa, synthetic construct
MDRRSANYQPXIWDHDFLQSLNSXXTDEXYXRRAEELXGKVXXXIXDVXEPLDQLELIDNLQRLGLXXXFEXEIRNILXNIXNXNKDYXWRKENLYATSLEFRLLRQHGYPVSQEVXXGFKXDXXXFIXDDFXGILSLHEASXYXLEGESIMEEAWQFTSKHLKEXMIXXXXXEEDXFVAEQAKRALELPLHWKXXPMLEARWFIHXYEXREDKNHLLLELAKXEFNXLQAIYQEELKXXSXWWKDXGLGEKLXFARXXLVASFXWSMGIXFEPQFAYCRRXLTIXXALIXVIDDIYDVYGTLDELEXFXDAVXRWDINYALXHLPXYMKXCFLALYNXVNEFXYYVLKQQDFDXLXSIKXAWLXXIQAYLVEAKWYHXKYTPXLXEXLENGLVSIXGPXXXXXXYLSGTNPIIXKELEFLESNXDIXHWSXKIXRLQDDLGTSSDEIQRGDVPKSIQCYMHETGASEEVARXHIKDMMRQMWKKVNAYXADKDSPLXXTTXEFXLNXVRXSHFMYLHGDGHGXQNQXTXDVXFTLLFQPIPLXDKXXXXXXSPXTKG

SEQ ID NO: 39 - DNA, 4589 bp, synthetic construct
  source 1..4589: mol_type = other DNA
ctcttattggtcgaaggctcgtccagctccgagcgtgcgtaaggtgagggctccttccgctccgcgactgcgttaactggagccaggctgagcgtcggcgccggggttcggtggcctctagtgagatctggaggatccaaggattctgtagctacaatgttgtcaagactttttcgaatgcatggcctctttgtggcctcccatccctgggaagtcatagtggggacagtgacactgaccatctgcatgatgtccatgaacatgtttactggtaacaataagatctgtggttggaattatgaatgtccaaagtttgaagaggatgttttgagcagtgacattataattctgacaataacacgatgcatagccatcctgtatatttacttccagttccagaatttacgtcaacttggatcaaaatatattttgggtattgctggccttttcacaattttctcaagttttgtattcagtacagttgtcattcacttcttagacaaagaattgacaggcttgaatgaagctttgccctttttcctacttttgattgacctttccagagcaagcacattagcaaagtttgccctcagttccaactcacaggatgaagtaagggaaaatattgctcgtggaatggcaattttaggtcctacgtttaccctcgatgctcttgttgaatgtcttgtgattggagttggtaccatgtcaggggtacgtcagcttgaaattatgtgctgctttggctgcatgtcagttcttgccaactacttcgtgttcatgactttcttcccagcttgtgtgtccttggtattagagctttctcgggaaagccgcgagggtcgtccaatttggcagctcagccattttgcccgagttttagaagaagaagaaaataagccgaatcctgtaactcagagggtcaagatgattatgtctctaggcttggttcttgttcatgctcacagtcgctggatagctgatccttctcctcaaaacagtacagcagatacttctaaggtttcattaggactggatgaaaatgtgtccaagagaattgaaccaagtgtttccctctggcagttttatctctctaaaatgatcagcatggatattgaacaagttattaccctaagtttagctctccttctggctgtcaagtacatcttctttgaacaaacagagacagaatctacactctcattaaaaaaccctatcacatctcctgtagtgacacaaaagaaagtcccagacaattgttgtagacgtgaacctatgctggtcagaaataaccagaaatgtgattcagtagaggaagagacagggataaaccgagaaagaaaagttgaggttataaaacccttagtggctgaaacagataccccaaacagagctacatttgtggttggtaactcctccttactcgatacttcatcagtactggtgacacaggaacctgaaattgaacttcccagggaacctcggcctaatgaagaatgtctacagatacttgggaatgcagagaaaggtgcaaaattccttagtgatgctgagatcatccagttagtcaatgctaagcatatcccagcctacaagttggaaactctgatggaaactcatgagcgtggtgtatctattcgccgacagttactttccaagaagctttcagaaccttcttctctccagtacctaccttacagggattataattactccttggtgatgggagcttgttgtgagaatgttattggatatatgcccatccctgttggagtggcaggacccctttgcttagatgaaaaagaatttcaggttccaatggcaacaacagaaggttgtcttgtggccagcaccaatagaggctgcagagcaataggtcttggtggaggtgccagcagccgagtccttgcagatgggatgactcgtggcccagttgtgcgtcttccacgtgcttgtgactctgcagaagtgaaagcctggctcgaaacatctgaagggttcgcagtgataaaggaggcatttgacagcactagcagatttgcacgtctacagaaacttcatacaagtatagctggacgcaacctttatatccgtttccagtccaggtcaggggatgccatggggatgaacatgatttcaaagggtacagagaaagcactttcaaaacttcacgagtatttccctgaaatgcagattctagccgttagtggtaactattgtactgacaagaaacctgctgctataaattggatagagggaagaggaaaatctgttgtttgtgaagctgtcattccagccaaggttgtcagagaagtattaaagactaccacagaggctatgattgaggtcaacattaacaagaatttagtgggctctgccatggctgggagcataggaggctacaacgcccatgcagcaaacattgtcaccgccatctacattgcctgtggacaggatgcagcacagaatgttggtagttcaaactgtattactttaatggaagcaagtggtcccacaaatgaagatttatatatcagctgcaccatgccatctatagagataggaacggtgggtggtgggaccaacctactacctcagcaagcctgtttgcagatgctaggtgttcaaggagcatgcaaagataatcctggggaaaatgcccggcagcttgcccgaattgtgtgtgggaccgtaatggctggggaattgtcacttatggcagcattggcagcaggacatcttgtcaaaagtcacatgattcacaacaggtcgaagatcaatttacaagacctccaaggagcttgcaccaagaagacagcctgaatagcccgacagttctgaactggaacatgggcattgggttctaaaggactaacataaaatctgtgaattaaaaaagctcaatgcattgtcttgtggaggatgaatagatgtgatcactgagacagccacttggtttttggctctttcagagaggtctcaggttctttccatgcagactcctcagatctgaacacagtttagtgctttacatgctgtgctctttgaagagatttcaacaagaatattgtatgttaaagcatcagagatggtaatctacagctcacctctgaaggcaaatataagctgggaaaaaagttttgatgaaattcttgaagttcatggtgatcagtgcaattgaccttctccctcactcctgccagttgaaaatggatttttaaattatactgtagctgatgaaactcctgattttgtagttaatttattaagtctgggatgtagaacttcaagaagtaagagctaagttctaagttcatgtttgtaaattaatacttcatttggtgctggtctattttgattttggggggtaatcagcattattcttcagaaggggacctgttttcttcaagggaagaaacactcttattcccaaactacagaataatgtgttaaacatgctaaatagttctatcaggaaaacaaatcactgtatttatctccgcaggctatttgttcagagaggccttttgtttaaatataaatgtttaaatataaatgtttgtctggattggctataacatgtctttcagcattaggcttttaagaaacacagggttttgtattctttactaaagatatcagagctcttaatgttgcttagatgagggtgactgtcaagtacaagcaagactgggaccttagaaatcattgtagaaacacagttttgaaagaaaaataccatgtctctaagccaactttaattgcttaaaagacatttttatttagttgaaaaatctagttttttttgtaaactgtatcaaatctgtatatgttgtaataaaacttatgctagtttattggaagtgttcaagaaataaaaatcaacttgtgtactgataaaatactctagcctgggccagagaagataatgttctttaatgttgtccaggaaaccctggcttgcttgccgagcctaatgaaagggaaagtcagctttcagagccagtgaaggagccacgtgaatggccctagaactgtgcctagttcctgtggccaggaggttggtgactgaaacattcacacagggctctttgatggacccacgaacgctcttagctttctcagggggtcagcagagttattgaatcttaattttttttaatgtacaagttttgtataaataataaagaactccttattttgtattacatctaatgcttcaagtgttgctcttggaaagctgatgatgtctcttgtagaagatggactctgaaaaacattccaggaaaccatggcagcatggagagcctcttagtgattgtgtctgcattgttattgtggaagatttaccttttctgttgtacgtaaagcttaaattgcttttgttgtgactttttagccagtgactttttctgagcttttcatggaagtggcagtgaaaaatatgttgagtgttcattttagtgactgtaattaatatcttgctggattaatgttttgtacaattactaaattgtatacattttgttatagaatacttttttctagtttcagtaaataatgaaaaggaagttaataccaaaaaaaaa

SEQ ID NO: 40 - protein, 464 aa, synthetic construct
MSSVLVTQEPEIELPREPRPNEECLQILGNAEKGAKFLSDAEIIQLVNAKHIPAYKLETLMETHERGVSIRRQLLSKKLSEPSSLQYLPYRDYNYSLVMGACCENVIGYMPIPVGVAGPLCLDEKEFQVPMATTEGCLVASTNRGCRAIGLGGGASSRVLADGMTRGPVVRLPRACDSAEVKAWLETSEGFAVIKEAFDSTSRFARLQKLHTSIAGRNLYIRFQSRSGDAMGMNMISKGTEKALSKLHEYFPEMQILAVSGNYCTDKKPAAINWIEGRGKSVVCEAVIPAKVVREVLKTTTEAMIEVNINKNLVGSAMAGSIGGYNAHAANIVTAIYIACGQDAAQNVGSSNCITLMEASGPTNEDLYISCTMPSIEIGTVGGGTNLLPQQACLQMLGVQGACKDNPGENARQLARIVCGTVMAGELSLMAALAAGHLVKSHMIHNRSKINLQDLQGACTKKTA

SEQ ID NO: 41 - DNA, 1392 bp, synthetic construct
  source 1..1392: mol_type = other DNA
atgtcatcagtactggtgacacaggaacctgaaattgaacttcccagggaacctcggcctaatgaagaatgtctacagatacttgggaatgcagagaaaggtgcaaaattccttagtgatgctgagatcatccagttagtcaatgctaagcatatcccagcctacaagttggaaactctgatggaaactcatgagcgtggtgtatctattcgccgacagttactttccaagaagctttcagaaccttcttctctccagtacctaccttacagggattataattactccttggtgatgggagcttgttgtgagaatgttattggatatatgcccatccctgttggagtggcaggacccctttgcttagatgaaaaagaatttcaggttccaatggcaacaacagaaggttgtcttgtggccagcaccaatagaggctgcagagcaataggtcttggtggaggtgccagcagccgagtccttgcagatgggatgactcgtggcccagttgtgcgtcttccacgtgcttgtgactctgcagaagtgaaagcctggctcgaaacatctgaagggttcgcagtgataaaggaggcatttgacagcactagcagatttgcacgtctacagaaacttcatacaagtatagctggacgcaacctttatatccgtttccagtccaggtcaggggatgccatggggatgaacatgatttcaaagggtacagagaaagcactttcaaaacttcacgagtatttccctgaaatgcagattctagccgttagtggtaactattgtactgacaagaaacctgctgctataaattggatagagggaagaggaaaatctgttgtttgtgaagctgtcattccagccaaggttgtcagagaagtattaaagactaccacagaggctatgattgaggtcaacattaacaagaatttagtgggctctgccatggctgggagcataggaggctacaacgcccatgcagcaaacattgtcaccgccatctacattgcctgtggacaggatgcagcacagaatgttggtagttcaaactgtattactttaatggaagcaagtggtcccacaaatgaagatttatatatcagctgcaccatgccatctatagagataggaacggtgggtggtgggaccaacctactacctcagcaagcctgtttgcagatgctaggtgttcaaggagcatgcaaagataatcctggggaaaatgcccggcagcttgcccgaattgtgtgtgggaccgtaatggctggggaattgtcacttatggcagcattggcagcaggacatcttgtcaaaagtcacatgattcacaacaggtcgaagatcaatttacaagacctccaaggagcttgcaccaagaagacagcc

SEQ ID NO: 42 - protein, 51 aa, synthetic construct
SSCINPSTLVTSVNGFKCLPLATNKAAIRIMAKNKPVQCLVSAKYDNLTVD

SEQ ID NO: 43 - protein, 51 aa, synthetic construct
SSXINPSTLXTSVNGFKCLPLATNXAAIRIMAKNKPVQCLVSXKYDNLTVD

SEQ ID NO: 44 - protein, 51 aa, synthetic construct
SSXINPXTLXTSXNXFKXLPLATNXAAIRIXAKXKPVQCLXSXKYDNLXVD

SEQ ID NO: 45 - DNA, 9638 bp, synthetic construct
  source 1..9638: mol_type = other DNA
ttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatgtcatcagtactggtgacacaggaacctgaaattgaacttcccagggaacctcggcctaatgaagaatgtctacagatacttgggaatgcagagaaaggtgcaaaattccttagtgatgctgagatcatccagttagtcaatgctaagcatatcccagcctacaagttggaaactctgatggaaactcatgagcgtggtgtatctattcgccgacagttactttccaagaagctttcagaaccttcttctctccagtacctaccttacagggattataattactccttggtgatgggagcttgttgtgagaatgttattggatatatgcccatccctgttggagtggcaggacccctttgcttagatgaaaaagaatttcaggttccaatggcaacaacagaaggttgtcttgtggccagcaccaatagaggctgcagagcaataggtcttggtggaggtgccagcagccgagtccttgcagatgggatgactcgtggcccagttgtgcgtcttccacgtgcttgtgactctgcagaagtgaaagcctggctcgaaacatctgaagggttcgcagtgataaaggaggcatttgacagcactagcagatttgcacgtctacagaaacttcatacaagtatagctggacgcaacctttatatccgtttccagtccaggtcaggggatgccatggggatgaacatgatttcaaagggtacagagaaagcactttcaaaacttcacgagtatttccctgaaatgcagattctagccgttagtggtaactattgtactgacaagaaacctgctgctataaattggatagagggaagaggaaaatctgttgtttgtgaagctgtcattccagccaaggttgtcagagaagtattaaagactaccacagaggctatgattgaggtcaacattaacaagaatttagtgggctctgccatggctgggagcataggaggctacaacgcccatgcagcaaacattgtcaccgccatctacattgcctgtggacaggatgcagcacagaatgttggtagttcaaactgtattactttaatggaagcaagtggtcccacaaatgaagatttatatatcagctgcaccatgccatctatagagataggaacggtgggtggtgggaccaacctactacctcagcaagcctgtttgcagatgctaggtgttcaaggagcatgcaaagataatcctggggaaaatgcccggcagcttgcccgaattgtgtgtgggaccgtaatggctggggaattgtcacttatggcagcattggcagcaggacatcttgtcaaaagtcacatgattcacaacaggtcgaagatcaatttacaagacctccaaggagcttgcaccaagaagacagccggaagcggagccacgaacttctctctgttaaagcaagcaggagatgttgaagaaaaccccgggcctatgagcgagctgatcaaggagaacatgcacatgaagctgtacatggagggcaccgtgaacaaccaccacttcaagtgcacatccgagggcgaaggcaagccctacgagggcacccagaccatgaagatcaaggtggtcgagggcggccctctccccttcgccttcgacatcctggctaccagcttcatgtacggcagcaaagccttcatcaaccacacccagggcatccccgacttctttaagcagtccttccctgagggcttcacatgggagagaatcaccacatacgaagacgggggcgtgctgaccgctacccaggacaccagcttccagaacggctgcatcatctacaacgtcaagatcaacggggtgaacttcccatccaacggccctgtgatgcagaagaaaacacgcggctgggaggccaacaccgagatgctgtaccccgctgacggcggcctgagaggccacagccagatggccctgaagctcgtgggcgggggctacctgcactgctccttcaagaccacatacagatccaagaaacccgctaagaacctcaagatgcccggcttccacttcgtggaccacagactggaaagaatcaaggaggccgacaaagagacctacgtcgagcagcacgagatggctgtggccaagtactgcgacctccctagcaaactggggcacagatgaacccagctttcttgtacaaagtggtgatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcgccaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaatttagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctcgagttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatctttt

SEQ ID NO: 46 - DNA, 39310 bp, synthetic construct
  source 1..39310: mol_type = other DNA
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtcgaagcttggatccggtacctctagaattctcgagcggccgctagcgacatcgatcaactttgtatagaaaagttgccagacccccgggtccgcccggagcagctgcgctgtcggggccaggccgggctcccagtggattcgcgggcacagacgcccaggaccgcgcttcccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcgcaagtttgtacaaaaaagcaggctgccaccatgcatcaccatcatcaccacgacagaagaagtgctaactaccagccatccatttgggaccacgatttcctgcagagcctgaacagcaattacacagatgaggcctataagaggagagcagaggagctgcgcggcaaggtgaagatcgccatcaaggacgtgatcgagcccctggatcagctggagctgatcgacaacctccagcggctgggcctggcccaccgcttcgagacagagatccggaacatcctgaacaacatctacaacaacaacaaggactacaactggcggaaggagaacctgtacgccaccagcctggagtttcggctgctgagacagcacggctaccccgtgagccaggaggtgttcaatggctttaaggacgatcagggcggcttcatctgcgacgacttcaagggcatcctgtctctgcacgaggcctcctactattctctggagggcgagagcatcatggaggaggcctggcagttcacctccaagcacctgaaggaagtgatgatcagcaagaacatggaggaggacgtgtttgtggccgagcaggccaagagagccctggagctgcccctgcactggaaggtgcctatgctggaggccaggtggttcatccacatctatgagaggcgcgaggataagaatcacctgctgctggagctggccaagatggagtttaacacactccaggccatctaccaggaggagctgaaggagatcagcggatggtggaaggacaccggcctgggagagaagctgtctttcgccaggaatcgcctggtggcctcttttctgtggagcatgggcatcgccttcgagcctcagtttgcctattgccggagagtgctgacaatcagcatcgccctgatcaccgtgatcgacgacatctacgacgtgtacggcacactggacgagctggagattttcaccgatgccgtggagcggtgggacatcaactacgccctgaagcacctgccaggctatatgaagatgtgcttcctggccctgtacaatttcgtgaacgagtttgcctactatgtgctgaagcagcaggactttgatctgctgctgagcatcaagaatgcctggctgggcctgatccaggcctacctggtggaggccaagtggtatcactctaagtatacacccaagctggaggagtatctggagaacggcctggtgagcatcacaggcccactgatcatcaccatcagctacctgtccggcaccaatcccatcatcaagaaggagctggagttcctggagtccaaccctgacatcgtgcactggagcagcaagattttccggctccaggacgatctgggcacatctagcgatgagatccagcggggcgacgtgccaaagagcatccagtgttacatgcacgagacaggagcctccgaggaggtggcaagacagcacatcaaggacatgatgaggcagatgtggaagaaggtgaacgcctatacagccgacaaggattcccccctgaccggcaccacaaccgagttcctgctgaatctggtgagaatgtctcactttatgtacctgcacggcgatggccacggcgtgcagaaccaggagacaatcgacgtgggcttcaccctgctgtttcagcctatccccctggaggacaagcacatggcattcaccgcaagccctggcactaaaggaggaagcggagagggcaggggaagtcttctaacatgcggggacgtggaggaaaatcccggccccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgggaagcggagccacgaacttctctctgttaaagcaagcaggagatgttgaagaaaaccccgggcctatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaacccagctttcttgtacaaagtggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacaactttattatacatagttgatggccggccgcttcgaggggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcgatcgattcgacagatcactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagcttttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggagttcttactttaaaatgtttaaccccactaacaaccacaggcggatctctacagctaaaagtgggagggggacttacagtggatgacactgatggtaccttacaagaaaacatacgtgctacagcacccattactaaaaataatcactctgtagaactatccattggaaatggattagaaactcaaaacaataaactatgtgccaaattgggaaatgggttaaaatttaacaacggtgacatttgtataaaggatagtattaacaccttatggactggaataaaccctccacctaactgtcaaattgtggaaaacactaatacaaatgatggcaaacttactttagtattagtaaaaaatggagggcttgttaatggctacgtgtctctagttggtgtatcagacactgtgaaccaaatgttcacacaaaagacagcaaacatccaattaagattatattttgactcttctggaaatctattaactgaggaatcagacttaaaaattccacttaaaaataaatcttctacagcgaccagtgaaactgtagccagcagcaaagcctttatgccaagtactacagcttatcccttcaacaccactactagggatagtgaaaactacattcatggaatatgttactacatgactagttatgatagaagtctatttcccttgaacatttctataatgctaaacagccgtatgatttcttccaatgttgcctatgccatacaatttgaatggaatctaaatgcaagtgaatctccagaaagcaacatagctacgctgaccacatccccctttttcttttcttacattacagaagacgacaactaaaatgcccaagaataaagaatcgtttgtgttatgtttcaacgtgtttatttttcaattgcagaaaatttcgaatcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaatttaaatccgcatgcgatatcgagctctcccgggaattcggatctgcgacgcgaggctggatggccttccccattatgattcttctcgcttccggcggcatcgggatgcccgcgttgcaggccatgctgtccaggcaggtagatgacgaccatcagggacagcttcaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggatccgaattcccgggagagctcgatatcgcatgcggatttaaattaattaa

SEQ ID NO: 47 - DNA, 39960 bp, synthetic construct
  source 1..39960: mol_type = other DNA
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtcgaagcttggatccggtacctctagaattctcgagcggccgctagcgacatcgatcaactttgtatagaaaagttggccatagaaccagagaagtgagtggatgtgatgcccagctccagaagtgactccagaacaccctgttccaaagcagaggacacactgattttttttttaataggctgcaggacttactgttggtgggacgccctgctttgcgaagggaaaggaggagtttgccctgagcacaggcccccaccctccactgggctttccccagctcccttgtcttcttatcacggtagtggcccagtccctggcccctgactccagaaggtggccctcctggaaacccaggtcgtgcagtcaacgatgtactcgccgggacagcgatgtctgctgcactccatccctcccctgttcatttgtccttcatgcccgtctggagtagatgctttttgcagaggtggcaccctgtaaagctctcctgtctgactttttttttttttttagactgagttttgctcttgttgcctaggctggagtgcaatggcacaatctcagctcactgcaccctctgcctcccgggttcaagcgattctcctgcctcagcctcccgagtagttgggattacaggcatgcaccaccacgcccagctaatttttgtatttttagtagagacaaggtttcaccgtgatggccaggctggtcttgaactccaggactcaagtgatgctcctgcctaggcctctcaaagtgttgggattacaggcgtgagccactgcacccggcctgcacgcgttctttgaaagcagtcgagggggcgctaggtgtgggcagggacgagctggcgcggcgtcgctgggtgcaccgcgaccacgggcagagccacgcggcgggaggactacaactcccggcacaccccgcgccgccccgcctctactcccagaaggccgcggggggtggaccgcctaagagggcgtgcgctcccgacatgccccgcggcgcgccattaaccgccagatttgaatcgcgggacccgttggcagaggtggcggcggcggccaagtttgtacaaaaaagcaggctgccaccatgcatcaccatcatcaccacgacagaagaagtgctaactaccagccatccatttgggaccacgatttcctgcagagcctgaacagcaattacacagatgaggcctataagaggagagcagaggagctgcgcggcaaggtgaagatcgccatcaaggacgtgatcgagcccctggatcagctggagctgatcgacaacctccagcggctgggcctggcccaccgcttcgagacagagatccggaacatcctgaacaacatctacaacaacaacaaggactacaactggcggaaggagaacctgtacgccaccagcctggagtttcggctgctgagacagcacggctaccccgtgagccaggaggtgttcaatggctttaaggacgatcagggcggcttcatctgcgacgacttcaagggcatcctgtctctgcacgaggcctcctactattctctggagggcgagagcatcatggaggaggcctggcagttcacctccaagcacctgaaggaagtgatgatcagcaagaacatggaggaggacgtgtttgtggccgagcaggccaagagagccctggagctgcccctgcactggaaggtgcctatgctggaggccaggtggttcatccacatctatgagaggcgcgaggataagaatcacctgctgctggagctggccaagatggagtttaacacactccaggccatctaccaggaggagctgaaggagatcagcggatggtggaaggacaccggcctgggagagaagctgtctttcgccaggaatcgcctggtggcctcttttctgtggagcatgggcatcgccttcgagcctcagtttgcctattgccggagagtgctgacaatcagcatcgccctgatcaccgtgatcgacgacatctacgacgtgtacggcacactggacgagctggagattttcaccgatgccgtggagcggtgggacatcaactacgccctgaagcacctgccaggctatatgaagatgtgcttcctggccctgtacaatttcgtgaacgagtttgcctactatgtgctgaagcagcaggactttgatctgctgctgagcatcaagaatgcctggctgggcctgatccaggcctacctggtggaggccaagtggtatcactctaagtatacacccaagctggaggagtatctggagaacggcctggtgagcatcacaggcccactgatcatcaccatcagctacctgtccggcaccaatcccatcatcaagaaggagctggagttcctggagtccaaccctgacatcgtgcactggagcagcaagattttccggctccaggacgatctgggcacatctagcgatgagatccagcggggcgacgtgccaaagagcatccagtgttacatgcacgagacaggagcctccgaggaggtggcaagacagcacatcaaggacatgatgaggcagatgtggaagaaggtgaacgcctatacagccgacaaggattcccccctgaccggcaccacaaccgagttcctgctgaatctggtgagaatgtctcactttatgtacctgcacggcgatggccacggcgtgcagaaccaggagacaatcgacgtgggcttcaccctgctgtttcagcctatccccctggaggacaagcacatggcattcaccgcaagccctggcactaaaggaggaagcggagagggcaggggaagtcttctaacatgcggggacgtggaggaaaatcccggccccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgggaagcggagccacgaacttctctctgttaaagcaagcaggagatgttgaagaaaaccccgggcctatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaacccagctttcttgtacaaagtggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacaactttattatacatagttgatggccggccgcttcgaggggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcgatcgattcgacagatcactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagcttttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggagttcttactttaaaatgtttaaccccactaacaaccacaggcggatctctacagctaaaagtgggagggggacttacagtggatgacactgatggtaccttacaagaaaacatacgtgctacagcacccattactaaaaataatcactctgtagaactatccattggaaatggattagaaactcaaaacaataaactatgtgccaaattgggaaatgggttaaaatttaacaacggtgacatttgtataaaggatagtattaacaccttatggactggaataaaccctccacctaactgtcaaattgtggaaaacactaatacaaatgatggcaaacttactttagtattagtaaaaaatggagggcttgttaatggctacgtgtctctagttggtgtatcagacactgtgaaccaaatgttcacacaaaagacagcaaacatccaattaagattatattttgactcttctggaaatctattaactgaggaatcagacttaaaaattccacttaaaaataaatcttctacagcgaccagtgaaactgtagccagcagcaaagcctttatgccaagtactacagcttatcccttcaacaccactactagggatagtgaaaactacattcatggaatatgttactacatgactagttatgatagaagtctatttcccttgaacatttctataatgctaaacagccgtatgatttcttccaatgttgcctatgccatacaatttgaatggaatctaaatgcaagtgaatctccagaaagcaacatagctacgctgaccacatccccctttttcttttcttacattacagaagacgacaactaaaatgcccaagaataaagaatcgtttgtgttatgtttcaacgtgtttatttttcaattgcagaaaatttcgaatcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaatttaaatccgcatgcgatatcgagctctcccgggaattcggatctgcgacgcgaggctggatggccttccccattatgattcttctcgcttccggcggcatcgggatgcccgcgttgcaggccatgctgtccaggcaggtagatgacgaccatcagggacagcttcaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggatccgaattcccgggagagctcgatatcgcatgcggatttaaattaattaa

SEQ ID NO: 48 - DNA, 40081 bp, synthetic construct
  source 1..40081: mol_type = other DNA
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtcgaagcttggatccggtacctctagaattctcgagcggccgctagcgacatcgatcaactttgtatagaaaagttggccatagaaccagagaagtgagtggatgtgatgcccagctccagaagtgactccagaacaccctgttccaaagcagaggacacactgattttttttttaataggctgcaggacttactgttggtgggacgccctgctttgcgaagggaaaggaggagtttgccctgagcacaggcccccaccctccactgggctttccccagctcccttgtcttcttatcacggtagtggcccagtccctggcccctgactccagaaggtggccctcctggaaacccaggtcgtgcagtcaacgatgtactcgccgggacagcgatgtctgctgcactccatccctcccctgttcatttgtccttcatgcccgtctggagtagatgctttttgcagaggtggcaccctgtaaagctctcctgtctgactttttttttttttttagactgagttttgctcttgttgcctaggctggagtgcaatggcacaatctcagctcactgcaccctctgcctcccgggttcaagcgattctcctgcctcagcctcccgagtagttgggattacaggcatgcaccaccacgcccagctaatttttgtatttttagtagagacaaggtttcaccgtgatggccaggctggtcttgaactccaggactcaagtgatgctcctgcctaggcctctcaaagtgttgggattacaggcgtgagccactgcacccggcctgcacgcgttctttgaaagcagtcgagggggcgctaggtgtgggcagggacgagctggcgcggcgtcgctgggtgcaccgcgaccacgggcagagccacgcggcgggaggactacaactcccggcacaccccgcgccgccccgcctctactcccagaaggccgcggggggtggaccgcctaagagggcgtgcgctcccgacatgccccgcggcgcgccattaaccgccagatttgaatcgcgggacccgttggcagaggtggcggcggcggccaagtttgtacaaaaaagcaggctgccaccatgcatcaccatcatcaccacgacagaagaagtgctaactaccagccatccatttgggaccacgatttcctgcagagcctgaacagcaattacacagatgaggcctataagaggagagcagaggagctgcgcggcaaggtgaagatcgccatcaaggacgtgatcgagcccctggatcagctggagctgatcgacaacctccagcggctgggcctggcccaccgcttcgagacagagatccggaacatcctgaacaacatctacaacaacaacaaggactacaactggcggaaggagaacctgtacgccaccagcctggagtttcggctgctgagacagcacggctaccccgtgagccaggaggtgttcaatggctttaaggacgatcagggcggcttcatctgcgacgacttcaagggcatcctgtctctgcacgaggcctcctactattctctggagggcgagagcatcatggaggaggcctggcagttcacctccaagcacctgaaggaagtgatgatcagcaagaacatggaggaggacgtgtttgtggccgagcaggccaagagagccctggagctgcccctgcactggaaggtgcctatgctggaggccaggtggttcatccacatctatgagaggcgcgaggataagaatcacctgctgctggagctggccaagatggagtttaacacactccaggccatctaccaggaggagctgaaggagatcagcggatggtggaaggacaccggcctgggagagaagctgtctttcgccaggaatcgcctggtggcctcttttctgtggagcatgggcatcgccttcgagcctcagtttgcctattgccggagagtgctgacaatcagcatcgccctgatcaccgtgatcgacgacatctacgacgtgtacggcacactggacgagctggagattttcaccgatgccgtggagcggtgggacatcaactacgccctgaagcacctgccaggctatatgaagatgtgcttcctggccctgtacaatttcgtgaacgagtttgcctactatgtgctgaagcagcaggactttgatctgctgctgagcatcaagaatgcctggctgggcctgatccaggcctacctggtggaggccaagtggtatcactctaagtatacacccaagctggaggagtatctggagaacggcctggtgagcatcacaggcccactgatcatcaccatcagctacctgtccggcaccaatcccatcatcaagaaggagctggagttcctggagtccaaccctgacatcgtgcactggagcagcaagattttccggctccaggacgatctgggcacatctagcgatgagatccagcggggcgacgtgccaaagagcatccagtgttacatgcacgagacaggagcctccgaggaggtggcaagacagcacatcaaggacatgatgaggcagatgtggaagaaggtgaacgcctatacagccgacaaggattcccccctgaccggcaccacaaccgagttcctgctgaatctggtgagaatgtctcactttatgtacctgcacggcgatggccacggcgtgcagaaccaggagacaatcgacgtgggcttcaccctgctgtttcagcctatccccctggaggacaagcacatggcattcaccgcaagccctggcactaaaggaggaagcggagagggcaggggaagtcttctaacatgcggggacgtggaggaaaatcccggccccatgtcatcagtactggtgacacaggaacctgaaattgaacttcccagggaacctcggcctaatgaagaatgtctacagatacttgggaatgcagagaaaggtgcaaaattccttagtgatgctgagatcatccagttagtcaatgctaagcatatcccagcctacaagttggaaactctgatggaaactcatgagcgtggtgtatctattcgccgacagttactttccaagaagctttcagaaccttcttctctccagtacctaccttacagggattataattactccttggtgatgggagcttgttgtgagaatgttattggatatatgcccatccctgttggagtggcaggacccctttgcttagatgaaaaagaatttcaggttccaatggcaacaacagaaggttgtcttgtggccagcaccaatagaggctgcagagcaataggtcttggtggaggtgccagcagccgagtccttgcagatgggatgactcgtggcccagttgtgcgtcttccacgtgcttgtgactctgcagaagtgaaagcctggctcgaaacatctgaagggttcgcagtgataaaggaggcatttgacagcactagcagatttgcacgtctacagaaacttcatacaagtatagctggacgcaacctttatatccgtttccagtccaggtcaggggatgccatggggatgaacatgatttcaaagggtacagagaaagcactttcaaaacttcacgagtatttccctgaaatgcagattctagccgttagtggtaactattgtactgacaagaaacctgctgctataaattggatagagggaagaggaaaatctgttgtttgtgaagctgtcattccagccaaggttgtcagagaagtattaaagactaccacagaggctatgattgaggtcaacattaacaagaatttagtgggctctgccatggctgggagcataggaggctacaacgcccatgcagcaaacattgtcaccgccatctacattgcctgtggacaggatgcagcacagaatgttggtagttcaaactgtattactttaatggaagcaagtggtcccacaaatgaagatttatatatcagctgcaccatgccatctatagagataggaacggtgggtggtgggaccaacctactacctcagcaagcctgtttgcagatgctaggtgttcaaggagcatgcaaagataatcctggggaaaatgcccggcagcttgcccgaattgtgtgtgggaccgtaatggctggggaattgtcacttatggcagcattggcagcaggacatcttgtcaaaagtcacatgattcacaacaggtcgaagatcaatttacaagacctccaaggagcttgcaccaagaagacagccggaagcggagccacgaacttctctctgttaaagcaagcaggagatgttgaagaaaaccccgggcctatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaaacccagctttcttgtacaaagtggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaacaactttattatacatagttgatggccggccgcttcgaggggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcgatcgattcgacagatcactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagcttttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggagttcttactttaaaatgtttaaccccactaacaaccacaggcggatctctacagctaaaagtgggagggggacttacagtggatgacactgatggtaccttacaagaaaacatacgtgctacagcacccattactaaaaataatcactctgtagaactatccattggaaatggattagaaactcaaaacaataaactatgtgccaaattgggaaatgggttaaaatttaacaacggtgacatttgtataaaggatagtattaacaccttatggactggaataaaccctccacctaactgtcaaattgtggaaaacactaatacaaatgatggcaaacttactttagtattagtaaaaaatggagggcttgttaatggctacgtgtctctagttggtgtatcagacactgtgaaccaaatgttcacacaaaagacagcaaacatccaattaagattatattttgactcttctggaaatctattaactgaggaatcagacttaaaaattccacttaaaaataaatcttctacagcgaccagtgaaactgtagccagcagcaaagcctttatgccaagtactacagcttatcccttcaacaccactactagggatagtgaaaactacattcatggaatatgttactacatgactagttatgatagaagtctatttcccttgaacatttctataatgctaaacagccgtatgatttcttccaatgttgcctatgccatacaatttgaatggaatctaaatgcaagtgaatctccagaaagcaacatagctacgctgaccacatccccctttttcttttcttacattacagaagacgacaactaaaatgcccaagaataaagaatcgtttgtgttatgtttcaacgtgtttatttttcaattgcagaaaatttcgaatcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaatttaaatccgcatgcgatatcgagctctcccgggaattcggatctgcgacgcgaggctggatggccttccccattatgattcttctcgcttccggcggcatcgggatgcccgcgttgcaggccatgctgtccaggcaggtagatgacgaccatcagggacagcttcaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggatccgaattcccgggagagctcgatatcgcatgcggatttaaattaattaa

SEQ ID NO: 49 - DNA, 39431 bp, synthetic construct
  source 1..39431: mol_type = other DNA
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtcgaagcttggatccggtacctctagaattctcgagcggccgctagcgacatcgatcaactttgtatagaaaagttgccagacccccgggtccgcccggagcagctgcgctgtcggggccaggccgggctcccagtggattcgcgggcacagacgcccaggaccgcgcttcccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcgcaagtttgtacaaaaaagcaggctgccaccatgcatcaccatcatcaccacgacagaagaagtgctaactaccagccatccatttgggaccacgatttcctgcagagcctgaacagcaattacacagatgaggcctataagaggagagcagaggagctgcgcggcaaggtgaagatcgccatcaaggacgtgatcgagcccctggatcagctggagctgatcgacaacctccagcggctgggcctggcccaccgcttcgagacagagatccggaacatcctgaacaacatctacaacaacaacaaggactacaactggcggaaggagaacctgtacgccaccagcctggagtttcggctgctgagacagcacggctaccccgtgagccaggaggtgttcaatggctttaaggacgatcagggcggcttcatctgcgacgacttcaagggcatcctgtctctgcacgaggcctcctactattctctggagggcgagagcatcatggaggaggcctggcagttcacctccaagcacctgaaggaagtgatgatcagcaagaacatggaggaggacgtgtttgtggccgagcaggccaagagagccctggagctgcccctgcactggaaggtgcctatgctggaggccaggtggttcatccacatctatgagaggcgcgaggataagaatcacctgctgctggagctggccaagatggagtttaacacactccaggccatctaccaggaggagctgaaggagatcagcggatggtggaaggacaccggcctgggagagaagctgtctttcgccaggaatcgcctggtggcctcttttctgtggagcatgggcatcgccttcgagcctcagtttgcctattgccggagagtgctgacaatcagcatcgccctgatcaccgtgatcgacgacatctacgacgtgtacggcacactggacgagctggagattttcaccgatgccgtggagcggtgggacatcaactacgccctgaagcacctgccaggctatatgaagatgtgcttcctggccctgtacaatttcgtgaacgagtttgcctactatgtgctgaagcagcaggactttgatctgctgctgagcatcaagaatgcctggctgggcctgatccaggcctacctggtggaggccaagtggtatcactctaagtatacacccaagctggaggagtatctggagaacggcctggtgagcatcacaggcccactgatcatcaccatcagctacctgtccggcaccaatcccatcatcaagaaggagctggagttcctggagtccaaccctgacatcgtgcactggagcagcaagattttccggctccaggacgatctgggcacatctagcgatgagatccagcggggcgacgtgccaaagagcatccagtgttacatgcacgagacaggagcctccgaggaggtggcaagacagcacatcaaggacatgatgaggcagatgtggaagaaggtgaacgcctatacagccgacaaggattcccccctgaccggcaccacaaccgagttcctgctgaatctggtgagaatgtctcactttatgtacctgcacggcgatggccacggcgtgcagaaccaggagacaatcgacgtgggcttcaccctgctgtttcagcctatccccctggaggacaagcacatggcattcaccgcaagccctggcactaaaggaggaagcggagagggcaggggaagtcttctaacatgcggggacgtggaggaaaatcccggccccatgtcatcagtactggtgacacaggaacctgaaattgaacttcccagggaacctcggcctaatgaagaatgtctacagatacttgggaatgcagagaaaggtgcaaaattccttagtgatgctgagatcatccagttagtcaatgctaagcatatcccagcctacaagttggaaactctgatggaaactcatgagcgtggtgtatctattcgccgacagttactttccaagaagctttcagaaccttcttctctccagtacctaccttacagggattataattactccttggtgatgggagcttgttgtgagaatgttattggatatatgcccatccctgttggagtggcaggacccctttgcttagatgaaaaagaatttcaggttccaatggcaacaacagaaggttgtcttgtggccagcaccaatagaggctgcagagcaataggtcttggtggaggtgccagcagccgagtccttgcagatgggatgactcgtggcccagttgtgcgtcttccacgtgcttgtgactctgcagaagtgaaagcctggctcgaaacatctgaagggttcgcagtgataaaggaggcatttgacagcactagcagatttgcacgtctacagaaacttcatacaagtatagctggacgcaacctttatatccgtttccagtccaggtcaggggatgccatggggatgaacatgatttcaaagggtacagagaaagcactttcaaaacttcacgagtatttccctgaaatgcagattctagccgttagtggtaactattgtactgacaagaaacctgctgctataaattggatagagggaagaggaaaatctgttgtttgtgaagctgtcattccagccaaggttgtcagagaagtattaaagactaccacagaggctatgattgaggtcaacattaacaagaatttagtgggctctgccatggctgggagcataggaggctacaacgcccatgcagcaaacattgtcaccgccatctacattgcctgtggacaggatgcagcacagaatgttggtagttcaaactgtattactttaatggaagcaagtggtcccacaaatgaagatttatatatcagctgcaccatgccatctatagagataggaacggtgggtggtgggaccaacctactacctcagcaagcctgtttgcagatgctaggtgttcaaggagcatgcaaagataatcctggggaaaatgcccggcagcttgcccgaattgtgtgtgggaccgtaatggctggggaattgtcacttatggcagcattggcagcaggacatcttgtcaaaagtcacatgattcacaacaggtcgaagatcaatttacaagacctccaaggagcttgcaccaagaagacagccggaagcggagccacgaacttctctctgttaaagcaagcaggagatgttgaagaaaaccccgggcctatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaaacccagctttcttgtacaaagtggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaacaactttattatacatagttgatggccggccgcttcgaggggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcgatcgattcgacagatcactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagcttttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggagttcttactttaaaatgtttaaccccactaacaaccacaggcggatctctacagctaaaagtgggagggggacttacagtggatgacactgatggtaccttacaagaaaacatacgtgctacagcacccattactaaaaataatcactctgtagaactatccattggaaatggattagaaactcaaaacaataaactatgtgccaaattgggaaatgggttaaaatttaacaacggtgacatttgtataaaggatagtattaacaccttatggactggaataaaccctccacctaactgtcaaattgtggaaaacactaatacaaatgatggcaaacttactttagtattagtaaaaaatggagggcttgttaatggctacgtgtctctagttggtgtatcagacactgtgaaccaaatgttcacacaaaagacagcaaacatccaattaagattatattttgactcttctggaaatctattaactgaggaatcagacttaaaaattccacttaaaaataaatcttctacagcgaccagtgaaactgtagccagcagcaaagcctttatgccaagtactacagcttatcccttcaacaccactactagggatagtgaaaactacattcatggaatatgttactacatgactagttatgatagaagtctatttcccttgaacatttctataatgctaaacagccgtatgatttcttccaatgttgcctatgccatacaatttgaatggaatctaaatgcaagtgaatctccagaaagcaacatagctacgctgaccacatccccctttttcttttcttacattacagaagacgacaactaaaatgcccaagaataaagaatcgtttgtgttatgtttcaacgtgtttatttttcaattgcagaaaatttcgaatcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaatttaaatccgcatgcgatatcgagctctcccgggaattcggatctgcgacgcgaggctggatggccttccccattatgattcttctcgcttccggcggcatcgggatgcccgcgttgcaggccatgctgtccaggcaggtagatgacgaccatcagggacagcttcaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggatccgaattcccgggagagctcgatatcgcatgcggatttaaattaattaa

SEQ ID NO: 50 - DNA, 9431 bp, synthetic construct
  source 1..9431: mol_type = other DNA
ttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatgcatcaccatcatcaccacgacagaagaagtgctaactaccagccatccatttgggaccacgatttcctgcagagcctgaacagcaattacacagatgaggcctataagaggagagcagaggagctgcgcggcaaggtgaagatcgccatcaaggacgtgatcgagcccctggatcagctggagctgatcgacaacctccagcggctgggcctggcccaccgcttcgagacagagatccggaacatcctgaacaacatctacaacaacaacaaggactacaactggcggaaggagaacctgtacgccaccagcctggagtttcggctgctgagacagcacggctaccccgtgagccaggaggtgttcaatggctttaaggacgatcagggcggcttcatctgcgacgacttcaagggcatcctgtctctgcacgaggcctcctactattctctggagggcgagagcatcatggaggaggcctggcagttcacctccaagcacctgaaggaagtgatgatcagcaagaacatggaggaggacgtgtttgtggccgagcaggccaagagagccctggagctgcccctgcactggaaggtgcctatgctggaggccaggtggttcatccacatctatgagaggcgcgaggataagaatcacctgctgctggagctggccaagatggagtttaacacactccaggccatctaccaggaggagctgaaggagatcagcggatggtggaaggacaccggcctgggagagaagctgtctttcgccaggaatcgcctggtggcctcttttctgtggagcatgggcatcgccttcgagcctcagtttgcctattgccggagagtgctgacaatcagcatcgccctgatcaccgtgatcgacgacatctacgacgtgtacggcacactggacgagctggagattttcaccgatgccgtggagcggtgggacatcaactacgccctgaagcacctgccaggctatatgaagatgtgcttcctggccctgtacaatttcgtgaacgagtttgcctactatgtgctgaagcagcaggactttgatctgctgctgagcatcaagaatgcctggctgggcctgatccaggcctacctggtggaggccaagtggtatcactctaagtatacacccaagctggaggagtatctggagaacggcctggtgagcatcacaggcccactgatcatcaccatcagctacctgtccggcaccaatcccatcatcaagaaggagctggagttcctggagtccaaccctgacatcgtgcactggagcagcaagattttccggctccaggacgatctgggcacatctagcgatgagatccagcggggcgacgtgccaaagagcatccagtgttacatgcacgagacaggagcctccgaggaggtggcaagacagcacatcaaggacatgatgaggcagatgtggaagaaggtgaacgcctatacagccgacaaggattcccccctgaccggcaccacaaccgagttcctgctgaatctggtgagaatgtctcactttatgtacctgcacggcgatggccacggcgtgcagaaccaggagacaatcgacgtgggcttcaccctgctgtttcagcctatccccctggaggacaagcacatggcattcaccgcaagccctggcactaaaggaggaagcggagccacgaacttctctctgttaaagcaagcaggagatgttgaagaaaaccccgggcctatgagcgagctgatcaaggagaacatgcacatgaagctgtacatggagggcaccgtgaacaaccaccacttcaagtgcacatccgagggcgaaggcaagccctacgagggcacccagaccatgaagatcaaggtggtcgagggcggccctctccccttcgccttcgacatcctggctaccagcttcatgtacggcagcaaagccttcatcaaccacacccagggcatccccgacttctttaagcagtccttccctgagggcttcacatgggagagaatcaccacatacgaagacgggggcgtgctgaccgctacccaggacaccagcttccagaacggctgcatcatctacaacgtcaagatcaacggggtgaacttcccatccaacggccctgtgatgcagaagaaaacacgcggctgggaggccaacaccgagatgctgtaccccgctgacggcggcctgagaggccacagccagatggccctgaagctcgtgggcgggggctacctgcactgctccttcaagaccacatacagatccaagaaacccgctaagaacctcaagatgcccggcttccacttcgtggaccacagactggaaagaatcaaggaggccgacaaagagacctacgtcgagcagcacgagatggctgtggccaagtactgcgacctccctagcaaactggggcacagaggaagcggagagggcaggggaagtcttctaacatgcggggacgtggaggaaaatcccggccccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacccagctttcttgtacaaagtggtgatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggatccctcgagttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatctttt

SEQ ID NO: 51 - protein, 11 aa, synthetic construct
RRXXXXXXXAW

SEQ ID NO: 52 - protein, 11 aa, synthetic construct
RRXXXXXXXRW

SEQ ID NO: 53 - protein, 11 aa, synthetic construct
RRXXXXXXXNW

SEQ ID NO: 54 - protein, 11 aa, synthetic construct
RRXXXXXXXDW

SEQ ID NO: 55 - protein, 11 aa, synthetic construct
RRXXXXXXXCW

SEQ ID NO: 56 - protein, 11 aa, synthetic construct
RRXXXXXXXQW

SEQ ID NO: 57 - protein, 11 aa, synthetic construct
RRXXXXXXXEW

SEQ ID NO: 58 - protein, 11 aa, synthetic construct
RRXXXXXXXGW

SEQ ID NO: 59 - protein, 11 aa, synthetic construct
RRXXXXXXXHW

SEQ ID NO: 60 - protein, 11 aa, synthetic construct
RRXXXXXXXIW

SEQ ID NO: 61 - protein, 11 aa, synthetic construct
RRXXXXXXXLW

SEQ ID NO: 62 - protein, 11 aa, synthetic construct
RRXXXXXXXKW

SEQ ID NO: 63 - protein, 11 aa, synthetic construct
RRXXXXXXXMW

SEQ ID NO: 64 - protein, 11 aa, synthetic construct
RRXXXXXXXFW

SEQ ID NO: 65 - protein, 11 aa, synthetic construct
RRXXXXXXXPW

SEQ ID NO: 66 - protein, 11 aa, synthetic construct
RRXXXXXXXSW

SEQ ID NO: 67 - protein, 11 aa, synthetic construct
RRXXXXXXXTW

SEQ ID NO: 68 - protein, 11 aa, synthetic construct
RRXXXXXXXWW

SEQ ID NO: 69 - protein, 11 aa, synthetic construct
RRXXXXXXXYW

SEQ ID NO: 70 - protein, 11 aa, synthetic construct
RRXXXXXXXVW

SEQ ID NO: 71 - protein, 5 aa, synthetic construct
DDXAD

SEQ ID NO: 72 - protein, 5 aa, synthetic construct
DDXRD

SEQ ID NO: 73 - protein, 5 aa, synthetic construct
DDXND

SEQ ID NO: 74 - protein, 5 aa, synthetic construct
DDXDD

SEQ ID NO: 75 - protein, 5 aa, synthetic construct
DDXCD

SEQ ID NO: 76 - protein, 5 aa, synthetic construct
DDXQD

SEQ ID NO: 77 - protein, 5 aa, synthetic construct
DDXED

SEQ ID NO: 78 - protein, 5 aa, synthetic construct
DDXGD

SEQ ID NO: 79 - protein, 5 aa, synthetic construct
DDXHD

SEQ ID NO: 80 - protein, 5 aa, synthetic construct
DDXID

SEQ ID NO: 81 - protein, 5 aa, synthetic construct
DDXLD

SEQ ID NO: 82 - protein, 5 aa, synthetic construct
DDXKD

SEQ ID NO: 83 - protein, 5 aa, synthetic construct
DDXMD

SEQ ID NO: 84 - protein, 5 aa, synthetic construct
DDXFD

SEQ ID NO: 85 - protein, 5 aa, synthetic construct
DDXPD

SEQ ID NO: 86 - protein, 5 aa, synthetic construct
DDXSD

SEQ ID NO: 87 - protein, 5 aa, synthetic construct
DDXTD

SEQ ID NO: 88 - protein, 5 aa, synthetic construct
DDXWD

SEQ ID NO: 89 - protein, 5 aa, synthetic construct
DDXYD

SEQ ID NO: 90 - protein, 5 aa, synthetic construct
DDXVD

SEQ ID NO: 91 - protein, 5 aa, synthetic construct
NDXAD

SEQ ID NO: 92 - protein, 5 aa, synthetic construct
NDXRD

SEQ ID NO: 93 - protein, 5 aa, synthetic construct
NDXND

SEQ ID NO: 94 - protein, 5 aa, synthetic construct
NDXDD

SEQ ID NO: 95 - protein, 5 aa, synthetic construct
NDXCD

SEQ ID NO: 96 - protein, 5 aa, synthetic construct
NDXQD

SEQ ID NO: 97 - protein, 5 aa, synthetic construct
NDXED

SEQ ID NO: 98 - protein, 5 aa, synthetic construct
NDXGD

SEQ ID NO: 99 - protein, 5 aa, synthetic construct
NDXHD

SEQ ID NO: 100 - protein, 5 aa, synthetic construct
NDXID

SEQ ID NO: 101 - protein, 5 aa, synthetic construct
NDXLD

SEQ ID NO: 102 - protein, 5 aa, synthetic construct
NDXKD

SEQ ID NO: 103 - protein, 5 aa, synthetic construct
NDXMD

SEQ ID NO: 104 - protein, 5 aa, synthetic construct
NDXFD

SEQ ID NO: 105 - protein, 5 aa, synthetic construct
NDXPD

SEQ ID NO: 106 - protein, 5 aa, synthetic construct
NDXSD

SEQ ID NO: 107 - protein, 5 aa, synthetic construct
NDXTD

SEQ ID NO: 108 - protein, 5 aa, synthetic construct
NDXWD

SEQ ID NO: 109 - protein, 5 aa, synthetic construct
NDXYD

SEQ ID NO: 110 - protein, 5 aa, synthetic construct
NDXVD

SEQ ID NO: 111 - protein, 5 aa, synthetic construct
DDXAE

SEQ ID NO: 112 - protein, 5 aa, synthetic construct
DDXRE

SEQ ID NO: 113 - protein, 5 aa, synthetic construct
DDXNE

SEQ ID NO: 114 - protein, 5 aa, synthetic construct
DDXDE

SEQ ID NO: 115 - protein, 5 aa, synthetic construct
DDXCE

SEQ ID NO: 116 - protein, 5 aa, synthetic construct
DDXQE

SEQ ID NO: 117 - protein, 5 aa, synthetic construct
DDXEE

SEQ ID NO: 118 - protein, 5 aa, synthetic construct
DDXGE

SEQ ID NO: 119 - protein, 5 aa, synthetic construct
DDXHE

SEQ ID NO: 120 - protein, 5 aa, synthetic construct
DDXIE

SEQ ID NO: 121 - protein, 5 aa, synthetic construct
DDXLE

SEQ ID NO: 122 - protein, 5 aa, synthetic construct
DDXKE

SEQ ID NO: 123 - protein, 5 aa, synthetic construct
DDXME

SEQ ID NO: 124 - protein, 5 aa, synthetic construct
DDXFE

SEQ ID NO: 125 - protein, 5 aa, synthetic construct
DDXPE

SEQ ID NO: 126 - protein, 5 aa, synthetic construct
DDXSE

SEQ ID NO: 127 - protein, 5 aa, synthetic construct
DDXTE

SEQ ID NO: 128 - protein, 5 aa, synthetic construct
DDXWE

SEQ ID NO: 129 - protein, 5 aa, synthetic construct
DDXYE

SEQ ID NO: 130 - protein, 5 aa, synthetic construct
DDXVE

SEQ ID NO: 131 - protein, 4 aa, synthetic construct
DADD

SEQ ID NO: 132 - protein, 4 aa, synthetic construct
DRDD

SEQ ID NO: 133 - protein, 4 aa, synthetic construct
DNDD

SEQ ID NO: 134 - protein, 4 aa, synthetic construct
DDDD

SEQ ID NO: 135 - protein, 4 aa, synthetic construct
DCDD

SEQ ID NO: 136 - protein, 4 aa, synthetic construct
DQDD

SEQ ID NO: 137 - protein, 4 aa, synthetic construct
DEDD

SEQ ID NO: 138 - protein, 4 aa, synthetic construct
DGDD

SEQ ID NO: 139 - protein, 4 aa, synthetic construct
DHDD

SEQ ID NO: 140 - protein, 4 aa, synthetic construct
DIDD

SEQ ID NO: 141 - protein, 4 aa, synthetic construct
DLDD

SEQ ID NO: 142 - protein, 4 aa, synthetic construct
DKDD

SEQ ID NO: 143 - protein, 4 aa, synthetic construct
DMDD

SEQ ID NO: 144 - protein, 4 aa, synthetic construct
DFDD

SEQ ID NO: 145 - protein, 4 aa, synthetic construct
DPDD

SEQ ID NO: 146 - protein, 4 aa, synthetic construct
DSDD

SEQ ID NO: 147 - protein, 4 aa, synthetic construct
DTDD

SEQ ID NO: 148 - protein, 4 aa, synthetic construct
DWDD

SEQ ID NO: 149 - protein, 4 aa, synthetic construct
DYDD

SEQ ID NO: 150 - protein, 4 aa, synthetic construct
DVDD

SEQ ID NO: 151 - protein, 5 aa, synthetic construct
DDIYD

SEQ ID NO: 152 - protein, 7 aa, synthetic construct
VXDDXXD

SEQ ID NO: 153 - protein, 7 aa, synthetic construct
VXDDXXE

SEQ ID NO: 154 - protein, 6 aa, synthetic construct
IXDDXD

SEQ ID NO: 155 - protein, 6 aa, synthetic construct
LXDDXD

SEQ ID NO: 156 - protein, 6 aa, synthetic construct
VXDDXD

SEQ ID NO: 157 - protein, 6 aa, synthetic construct
IXDDXE

SEQ ID NO: 158 - protein, 6 aa, synthetic construct
LXDDXE

SEQ ID NO: 159 - protein, 6 aa, synthetic construct
VXDDXE

SEQ ID NO: 160 - protein, 9 aa, synthetic construct
NDLXSXXXE

SEQ ID NO: 161 - protein, 9 aa, synthetic construct
NDIXSXXXE

SEQ ID NO: 162 - protein, 9 aa, synthetic construct
NDVXSXXXE

SEQ ID NO: 163 - protein, 9 aa, synthetic construct
NDLXTXXXE

SEQ ID NO: 164 - protein, 9 aa, synthetic construct
NDIXTXXXE

SEQ ID NO: 165 - protein, 9 aa, synthetic construct
NDVXTXXXE

SEQ ID NO: 166 - protein, 9 aa, synthetic construct
DDLXSXXXE

SEQ ID NO: 167 - protein, 9 aa, synthetic construct
DDIXSXXXE

SEQ ID NO: 168 - protein, 9 aa, synthetic construct
DDVXSXXXE

SEQ ID NO: 169 - protein, 9 aa, synthetic construct
DDLXTXXXE

SEQ ID NO: 170 - protein, 9 aa, synthetic construct
DDIXTXXXE

SEQ ID NO: 171 - protein, 9 aa, synthetic construct
DDVXTXXXE

SEQ ID NO: 172 - protein, 9 aa, synthetic construct
NDXXSXXXE

SEQ ID NO: 173 - protein, 9 aa, synthetic construct
NDXXTXXXE

SEQ ID NO: 174 - protein, 9 aa, synthetic construct
DDXXSXXXE

SEQ ID NO: 175 - protein, 9 aa, synthetic construct
DDXXTXXXE

SEQ ID NO: 176 - DNA, 987 bp, Homo sapiens
  source 1..987: mol_type = other DNA
gccatagaaccagagaagtgagtggatgtgatgcccagctccagaagtgactccagaacaccctgttccaaagcagaggacacactgattttttttttaataggctgcaggacttactgttggtgggacgccctgctttgcgaagggaaaggaggagtttgccctgagcacaggcccccaccctccactgggctttccccagctcccttgtcttcttatcacggtagtggcccagtccctggcccctgactccagaaggtggccctcctggaaacccaggtcgtgcagtcaacgatgtactcgccgggacagcgatgtctgctgcactccatccctcccctgttcatttgtccttcatgcccgtctggagtagatgctttttgcagaggtggcaccctgtaaagctctcctgtctgactttttttttttttttagactgagttttgctcttgttgcctaggctggagtgcaatggcacaatctcagctcactgcaccctctgcctcccgggttcaagcgattctcctgcctcagcctcccgagtagttgggattacaggcatgcaccaccacgcccagctaatttttgtatttttagtagagacaaggtttcaccgtgatggccaggctggtcttgaactccaggactcaagtgatgctcctgcctaggcctctcaaagtgttgggattacaggcgtgagccactgcacccggcctgcacgcgttctttgaaagcagtcgagggggcgctaggtgtgggcagggacgagctggcgcggcgtcgctgggtgcaccgcgaccacgggcagagccacgcggcgggaggactacaactcccggcacaccccgcgccgccccgcctctactcccagaaggccgcggggggtggaccgcctaagagggcgtgcgctcccgacatgccccgcggcgcgccattaaccgccagatttgaatcgcgggacccgttggcagaggtggcggcggcggc

SEQ ID NO: 177 - DNA, 337 bp, Homo sapiens
  source 1..337: mol_type = other DNA
ccagacccccgggtccgcccggagcagctgcgctgtcggggccaggccgggctcccagtggattcgcgggcacagacgcccaggaccgcgcttcccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcg

SEQ ID NO: 178 - DNA, 1156 bp, Homo sapiens
  source 1..1156: mol_type = other DNA
aaacgtctgacccccacccccactccgccccgcccagttcttcaacctaatttctgattcgtgccaaagcttgtcctctgctcaaaatcgtggaagacgccgagtatggggaccgaagacctgggttcaagcccggcttggaatccctgcccatccctggcatttcatctctccgggcttatttgctggtttctccgaatgcgggccttgtctggttcacgctggatccccaacgcctagaacagtgcgtggcacgcagttcgtccttctataaatatcggactaaatgcatctctgtgatggtaatacccacacggtgttgtgagaatgaatgagtgattctgtgcaagttcctagtgatctgttacaaaaagtactggtcgctaaattactcttataataaagcatacttttaggataataaagcactattcgcgaattggttaccgctattatgaaattactgagcaatacatatctacatctgatcagtctccagaattatgccaaatcgtaccttcttctgaaagtatgtcctaattatctgcacctgaccctagtgatgctgtgaatgtgcaagtatagatacatcctccgaaggaaggatctttactccttttacctcctgaatgggctgcgtctgctgaaagcgcggggaatggcgttggaagcttggccctacttccagcattgccgcctactggttgggttactccagcaagtcactccccttccctgggcctcagtgtctctactgtagcattcccaggtctggaattccatccactttagcaaggatggacgcgccacagagagacgcgttcctagcccgcgcttcccacctgtcttcaggcgcatcccgcttccctcaaacttaggaaatgcctctgggaggtcctgtccggctccggactcactaccgaccacccgcaaacagcagggtcccctgggcttcccaagccgcgcacctctccgccccgcccctgcgccctccttcctcgcgtctgcccctctcccccaccccgccttctccctccccgccccagcggcgcatgcgccgcgctcggagcgtgtttttataaaagtccggccgcggccagaaacttcagtttgttggctgcggcagcaggtagcaaagtgacgccgagggcctgag

SEQ ID NO: 179 - DNA, 4421 bp, Homo sapiens
  source 1..4421: mol_type = other DNA
gatcacttgaggttaggagtttgagaccagcctggccaacatgtcaaaaccctgtctctactaaaaatataaaaattagctgggcatggtggtgagtgcctataatttcagctatttgggaggctgaggcaggagaatcgcttgaacccaggaggcggaggtggcagtgagccgagattgtgccactgcaccccagcctgggcgactagagcaagaccctatctaaaaaaaacaaaaaacaaacaacaaacaaacaaagaatctttgttaaatatctaagtctatatatttatgggtgtctatatctgaagagggaaagcccagttatgaggctgttccagtcaggtgagagataactgggcatatgatctagggtagacagagaaatgggaaaagatttgggaaataatatataaaactataaactctatgtgtgtgtgtattgttacacaacatgtgaacagtagtcatctctaagattcttctatgaattcattcaataaacgtttattgcatgtctgccatgcgtcaggcaccattttaggcactgcaaacttgaagggatgacagacacagaccctgctgtcttttagcttatcatctattgaggagagggagaacatagcgaaaataaataggaatcaactagggccaagtgatagtgacttggggaactatttgagataaactggtcaaggaaagcctgatgaggtagaaggtggggacttgactctggaggtgggggctaagactcgggaccagactctagattagagttccagatttaacacctagaagtcactgcccctttccatggcaatgactcaacaacccgttaccaacctttttctagaaatttctgtataatctgccccttaatttgcatgttaactaaaagtgggtagaaatatgagtgcagagctgcctctgagctgctactctgggcacacggccttatggggtagccctgctctgcaaagaccagtgcctctgctcctgatgtacactgccacttcaatataagctgctgtctaatgccacctgcttgcccttgaattttttttttttttttgaaatggagtctctttctgttgcccaggctggagtcagtggcgcgatctcggctcactgcagctccgcctcccgggttcacgccattctcctgcctcagcctcccgagtagctgggactacaggagcccgccaccacgcctaatttttttgtattttttttttttttgtagagatggggtttcaccgtgttagctaggatggtctcgatctcctgacctcgtgatccgtccacctcagcctcccaaagtgctgggattacaggtgtgagccaccgcgcccggcatcccttgaattctttactgggtgaagccaagaatcttcccaggctaagtccaaattttggggcctgcctgccctgcatcatgaggaggtatctgagtggaacgtcaatgaggaggaagaatgagttggagacagccctggagaagaatattctagatagaaggaaaaggaagagcaaagacccttgggtgagaaagagtttgtatttttgaggaaagcatgctagtgtgaatgccaagcagtattctgtgggaagatctcaggaggtgtctaagggcatggagataagtggtcagatgcacggtctgttttataggtggaattaactgcttgctgatggattgactggctgtgagggtgagtggcaagaaggaatcgaagatgagttagggtggtggcgatgccatttgctgagacaactgggaaagaaaaagatttgggaaaaaagttgagttcagctttggacatgttaagtgtgatatgctagtcacttcagtggagatgacaaatggcaagctggagaataagcctgaactccagggaggacctcctgtagatttactatggtgagtcatcagcatgcatatgatataacagtcatgggctagaagttagtttctcctcagggagtttgaaactgtaactagttcagagaagagggtggagggcagccccgataccccagcatttaccaatagagcaaacagggactcaggagcctggggagtgaggttagccggaaaccctcagagtggagcactggtgctcttactgagagaggaaggtgtgtccagatggaggatgtgattaactgtcctcaacatccctgagagaaggagtaagacaagggcagggaagagaagagaatgcaagatttggcaacatgtaggtcattatgatgactatgacaaaagcagtttgagctcaattctgtgtggagtatagggaaggagggttgaggacgtgcatttagaagggtacatagttctcaagaagttttgctgagcacatctgtaatcccagctatttgggacgctgaagtgggaggactgcttgagcccaggagttcaagaccagcctgggcaacatatcgagtccctgcttaaaaaaaaaaaaaaggaagttttgctgagaggctagatggattatgatttttgtttatttttcctgtttatccatatattatttttcaacaatgagtattgattacttatataataattttaaggctgtacacattgcagacagcaccccactgtttgaaaaactcctcctcagtagaacatggcagaccttcatcttccttccctgaaccttttccaaccttaggcttgccattctccaccagtgctaatgtcatgtctcttgaaatctgtattgaagtcagtatttcattcttgccagtttccactgtgtgtttaaatttggagtctggtgtctagcattagctggggttggggcttccactcctctcagcattggtaagcctcctcacccaccccatcccatgtccaagatcacccagttacacacttaccatctacccagttcattcacatcatcagtcccagagctgcagagatgctctttttctacctcctacttctctggctcttagagaggcagcatgggataatggggcaagcgaatagggccttaaagtagagggacaagggttctcttccctatctgccacttattagctatgtgacctcgtgtaagtctcttttctttttgagacagggtctccctctgtcacctaggctggagtacagtggtatgatcatagctcactgcagcctcgaactcctgggctcaagctatccttccaccttagccttctgagcagcagggactacaggcacatgccaccatgtccggctgatttatttatttttatttgggaagatgggggtctcactatgtcgcccaggctggtcatgaactcctggtctcaagcaaccctccaaccttggactcccaaagtgctgggattacaggtgtgagccctggccttgccacaatttcctcatctgtaaaacggggttagtgaaactcacatcctatcagtggttttgaggatgggccgactcttgtattgcctgctctagtacaatcagcagctaaggcggctcactttccggccgtgctacaataggtaagaactaggatgctttagacgtgtgactgggcagtgggagcccctcacatgatcccgagatgccagacagtgtctctccgcacagggcgtgtgctggtccagaggcccgtttttccagtcgccccacaccccgggtccgcgatcacgctccccccacccatagccgagcctgacgcggcggtggctcatgcgcctttccgtcccagcctttagccacggaccacacgtcccatctcaggcgccccgcccctcccccgccccccgcccccggcgcgcctccccaggctgccggctccggtgtctgagcggccgcgcccgcgagccgtgagcgatgattggctgcgccacggcggcgggcggtccgtgggcgcacacaccctccccgcgcagccaatgggcgtgcgcacgtcactgatccggagcccgcgggccggcagcccctcaataagccacattgttgcatgaaactccggcgcaggagtcccgggctgccgctggcaacatcgtgtcacccagctaagaaaatccgcgggcccgagccacgcgcctgtgaatcggagaggtcccactgcccgagtggagccgggctgagattcttctcaagttgagcctcagtgatcctgtggccgaagttagcgccttgacgtgggacaaccggacacgtcgccaggagagaactgaggcgccttctagcagttgtgacgccaaaatcacgtctccggagacccgcgccctccgccagccgggcgcaccctcgccggtagccttctttgtgcgccgtccggactcccagctcccggcccggcagccgagccccagcacaaagcagtcggaccgcgccgcccgcctcccctctcgcgtctccgcctcggtttcccaactctgcgccgtcgggccgcggcagg

SEQ ID NO: 180 - DNA, 3834 bp, synthetic construct
  source 1..3834: mol_type = other DNA
ggcggccgctgagggtggtgggtgcgcaagaggcggggctgggcggctgcaggaagcaagaggagagaaacaaggttaatgctccgggaataaacccctctactccagggtccagtgggaccctcgtttagactcacgcttctgcgtccccgtcctcccaccccaccccccagccaacatggcgcagcagactccaaggtcattctgcggacgcccttgggagagcacccacgtttccctcacccgaccccacggggtccctgtcgctctctctctcacctgaccggcctggcagccgcactgcggcttccccgaggcatgactgcggtgggatcaagttggagctgagtaagaagcgtggaccgtagcagccgctcgctcagtgccgggcaactaacacggcagcgtccttagagtcaggtgaaatgggcgggatctggggcggggcctctgatccacgccctccaaatgggaggggccaaagctcgcccttccattaacctcctggatttagggctccggagctatcccagtgcagggcggagctacgcgagtcctggggacaccggtcagctttggaaagcccaaggcttagttaggcacggggcagcgagggcaggtctttctgtcagaactcaagcaatgcaataggggtttgccacgagcccaggaggaaagaaagagacacatagaccgccagcggagaagcgaatggagactgcaggccaggttgtgttctctgagacccatcacaagacagagtttgaaaataaccatcccaggatcaccaaaggcccttccctgtcctctggaactcggttttcacagacttttcctcagagaccttggctgggatcatgccataacctctggagagagaaaaaaaaaaaaaaggttaaaagagcacacacctgtaaccccagaacttgggggggggcaggtataggtaggtgcatcttgtgtgttcgaggccagcctggtctacagactgagttccagggctacacagaactgtctcccacaaaacaaaacaaaataaaacaaataataataacctttggccagagtagaaagggcacagggggccttgagttctttctcctcttctttcagtgtttattttactgtgacaacagagatcacttggcacaaacaattcaaatgcctcagcaacaggggtcaaaactattcaggaccatgcattgcccattcaggtgtcccaaacctggtttctttaagcagcctctgtagcaggcctctttcattaagacttcagctttcctcccaagtggaatcaccacccatctgcaggatagactttctggtgaggtgagtaggtaaaagacaagccactctttcctttaaaaaaatgccagactcaggctagagaggtggcttaactgttaagagcactgactggggctggagagagatggttcagtggttatgagagctgtctgctcttccagaggtcctgagttcaattcccagcaactacatggtggctcacaactgtctataatgagatctgatgccctcttctagtgtgtctgaaggcagcgacaatgtacccacatacacgaaataaataaatacatcttttaaaaaagggggggcagggggctggcgagatggctcagtggttaagagtgccgactgctcttctgaaggtcccgagttcaaatcccagcaaccacatggtggctcacaaccatccataacgaaatctgatgccctcttcttgagtgtctgaagacagctacagtgtacttacatataataaataaataaatcttttaaaaaaatgtgctgttgtagaaaattaaaaaaaaaaaggggggggcaggcttgagcagaccccactggcttatctatttggcctctgcttaccttgtatccagtaggcaagtggtaacattcttccagcttcaaccccttctgtgggcctccgtggctagcccaccttccagatcctctactaagtgtggtaatgtggggtaatggggcaggttggggggtaagggggtgggaagtggagggtggggggggtggggggtctggctgataagctgcaagttcctcagaaaatagtcgtgcatccctggcaaacactgaaggctgtttaggttgcacaaataaatgttttagggtttgggggttcttttgttgagacaggatcttcatatagcctggctcactctgtagagcaggttggtctcaaacccacagaaatccacttgcttctgtctcccaaatgtcaggattaaaggcatgcatccaatgaagagtttatttttaaaatgctatgcatggtggtgcatgcctttaatagaggcagatctctgagttcaaggccagcctactctacagagtcccaggattgccagggctacacagagaaacccactcttgggggtggagggtaggactatgaatgcctcatccattttatgattctttgaccaacactgctgagatgagtctgaaccagacctggaaattctagctatgatgatacatgcttgcagtcctatcagtcagaataggcaaagacaggaatcttgagttggaggcctgcctgggctacatgtacacatactagactctgtcaaaaaaaagagagagagagagagagagagagagagagagagagagagagagagactctgtcataaaaaaaagaaaagaggggtgggtgggaagggaggaaggacgacgggaaggaatggcagcctttaaaaggtgaggctttttaaaagattgcagatggccaagtaaaacttaccactctctgcccctactttgcagcagctcagggccccactggcccaccagaattcagaagagagctgaaggcctggtggaagaggcctgcagtgccttgtagagccattgtcatccaagagggaacactgcacagttggacactcgctgcagagattagagtagttgaactgttttcagcacgtagacctccctctcagatgtgattctgtccctgtctcagatggctgagcctgactggtcaggaaaagcctgttggctagtgtgccccccggccagggaacaacctgagattccagtgtccaactcaaacatccctgaccctttcctcccagccaactgacccagttgcctgctagtagagaaaaaatctggtccctccctccaagatcctcgctgactgcctctggtctgaaattgtttaagtgtgcgcatttgcatcagccatttgcatcagcgtgtgccaagtgtcagtagaggtcagaagaaggcatcagttcctagatggccaccctgtgggtgctaggaacgggagccaggtttctctgcaggagcaacaagtgctcctaaccactgatccatctttccagacccgtctctgttttgttttaaggtgtggggactggccaacttccgccatatgcctcagtttcccctgaggtcacatttcaatagtccgctccttgcaagagctattgtaccactttcctgttagctagggctgtttagattggggatctgaccacctgccacaggctgaaacaagtcaagccaccatggagagacctggcgaaggatgagacttctgaagtggggctggagagcagaattgagctctccccggctctcactagttctaagggacccagcccctccgggcactcctccctaactcagaccgctgctgcaggccggttgagtttagaacaaaaggcagggggaggcggggcggtggggggtgcggtcccggcgccggcgggggcggggcgaatgctataaggggcggcggcccggcctggcccagcaggcccaacagccccggggcggatg

SEQ ID NO: 181 - DNA, 2311 bp, Homo sapiens
  source 1..2311: mol_type = other DNA
tagactgttgagtacaacacgtgtaggccagaggagacagtggcctatacttgggacaaataaagaggtctgtcctatttaagaaaatcaaccctgtaaaggaaattaataggactaagtacattttagtaaggcctctaagcaggctctaaagattatgaaaaatacacgggacagcagacacaaaagcccttaaagagcatgaagactttctaagttatttcactggaagcctgatagtggggcaagtgtaaggcaaaattcttaattaaattgaaaatgataagttgaattctgtcttcgagaacatagaaaagaattatgaaatgccaacatgtggttacaagtaatgcagacccaaggctccccagggacaagaagtcttgtgttaactctttgtggctctgaaagaaagagagagagaaaagattaagcctccttgtggagatcatgtgatgacttcctgattccagccagagcgagcatttccatggaaacttctcttcctcttcacccacacactgctccatgtacctgcaaagcctgttctgtctcaaaaaagttgtttggatgagccgtgacttttttttttcttaaataatgagacaaactccagaaaaagagaaaaaagcagagcagtctgacattccggcatcatcgaaatagtgatggcttttcctagaatgcttcagctaaggacccaaaatactaatgatctcctcaaagcttcagaggggcaactttgatttgactactctttttgtcactcttcagctcacaaaagagctcactttagttcaaaacacaaagctttaagcccctccatagattggtccaggtttaattttctatgatgagtggaggcctcagtttaatgctccaacttgatagatgaaacacagttccctcctctacacatttcccctgactcaggagtttgtatatattctcagttgtctgtccaacttatgcccactctttgagatattaatcaaggcactcccttgataacacttgcatattattatcaaaattatgcaattctttctaatatcagcccacaaatacatctcttccattaaaagtttgactaattatctatactactcatttgaaaactaacatagttaagttgtatttttagccatgaatttcagtttccctagctcactatacacagagaaggaaacttttgaaataattgagatgatcaaaaatatttgctgaagtaaatatatttctccttttcattcactcactaattgagaatgtctttgcacaaaacacattgcaaaaacattttcaaaaaaattcctaatttctagaattgataggaaaaacaatatggctacagcattggagagagagagaaaggagagaggagaaaggagagagagagaaaggagagaggagagagacagaggagagagagagaggatagagggggagagagagagaggagagagacagaggagagagagagaggatagaggggagagagagggagagggagagagagggagagagagggagagagagagagagagagggagagagagagagaaagagagagagagggagagagagagagagagctctttaacgtgagatatcccacaatgaacaaatctgcccagttatcaaagtgcagctatccttaggagttgtcagaaaatgcatcaggattatcagagaaaagtatcagaaagatttttttttctgatacgttgtataaaataaacaaactgaaattcaataacatataaggaattctgtctgggctctgaagacaatctctctctgcatattgagttcttcaaacattgtagcctctttatggtctctgagaaataactaccttaaacccataatctttaatacttcctaaactttcttaataagagaagctctattcctgacactacctctcatttgcaaggtcaaatcatcattagttttgtagtctattaactgggtttgcttaggtcaggcattattattactaaccttattgttaatattctaaccataagaattaaactattaatggtgaatagagtttttcactttaacataggcctatcccactggtgggatacgagccaattcgaaagaaaaagtcagtcatgtgcttttcagaggatgaaagcttaagataaagactaaaagtgtttgatgctggaggtgggagtggtattatataggtctcagccaagacatgtgataatcactgtagtagtagctggaaagagaaatctgtgactccaattagccagttcctgcagaccttgtgaggactagaggaagaatg

SEQ ID NO: 182 - DNA, 1327 bp, Homo sapiens
  source 1..1327: mol_type = other DNA
gatccccagagactttccagatatctgaagaagtcctgatgtcactgccccggtccttccccaggtagagcaacactcctcgtcgcaacccaactggctccccttaccttctacacacacacacacacacacacacacacacacacacacacacacaaatccaagacaacactactaaggcttctttgggagggggaagtagggataggtaagaggaaagtaagggacctcctatccagcctccatggaatcctgacttcttttccttgttatttcaacttcttccaccccatcttttaaactttagactccagccacagaagcttacaactaaaagaaactctaaggccaatttaatccaaggtttcattctatgtgctggagatggtgtacagtagggtgaggaaaccaaattctcagttggcactggtgtacccttgtacaggtgatgtaacatctctgtgcctcagtttgctcactataaaatagagacggtaggggtcatggtgagcactacctgactagcatataagaagctttcagcaagtgcagactactcttacccacttcccccaagcacagttggggtgggggacagctgaagaggtggaaacatgtgcctgagaatcctaatgaaatcggggtaaaggagcctggaacacatcctgtgaccccgcctgtcctgtaggaagccagtctctggaaagtaaaatggaagggctgcttgggaactttgaggatatttagcccaccccctcatttttacttggggaaactaaggcccagagacctaaggtgactgcctaagttagcaaggagaagtcttgggtattcatcccaggttggggggacccaattatttctcaatcccattgtattctggaatgggcaatttgtccacgtcactgtgacctaggaacacgcgaatgagaacccacagctgagggcctctgcgcacagaacagctgttctccccaggaaatcaactttttttaattgagaagctaaaaaattattctaagagaggtagcccatcctaaaaatagctgtaatgcagaagttcatgttcaaccaatcatttttgcttacgatgcaaaaattgaaaactaagtttattagagaggttagagaaggaggagctctaagcagaaaaaatcctgtgccgggaaaccttgattgtggctttttaatgaatgaagaggcctccctgagcttacaatataaaagggggacagagaggtgaaggtctacacatcaggggcttgctcttgcaaaaccaaaccacaagacagacttgcaaaagaaggcatgcacagctcagcactgc

SEQ ID NO: 183 - DNA, 300 bp, Homo sapiens
  source 1..300: mol_type = other DNA
ctcctcctcccgccctgcctcccgcgcctcggcccgcgcgagctagacgttcgggcagcccccggcgcagcgcggccgcagcgcctccgccccccgcacggtgtgagcgcccgcccgccgaggcggccggagtcccgagctagccccggcggcgccgccgcccagaccggacgacaggccacctcgtcgcgtccgcccgagtccccgcctcgccgccaacgccacaaccaccgcgcacggccccctgactccgtccagtattgatcgggagagccggagcgagctcttcggggagcagcg

SEQ ID NO: 184 - DNA, 2941 bp, Homo sapiens
  source 1..2941: mol_type = other DNA
gaattcagaattttagaccctttggccttggggtccatcctggagaccctgaggtctaagctacagcccctcagccaaccacagacccttctctggctcccaaaaggagttcagtcccagagggtggtcacccacccttcagggatgagaagttttcaaggggtattactcaggcactaaccccaggaaagatgacagcacattgccataaagttttggttgttttctaagccagtgcaactgcttattttagggattttccgggatagggtggggaagtggaaggaatcggcgagtagaagagaaagcctgggagggtggaagttagggatctaggggaagtttggctgatttggggatgcgggtgggggaggtgctggatggagttaagtgaaggatagggtgcctgagggaggatgcccgaagtcctcccagacccacttactcacggtggcagcggcgacactccagtctatcaaagatccgccgggatggagagccaggaggcgggggctgcccctgaggtagcggggaggccggggggccggggggcggacgggacgagtgcaatattggcgggggaaaaaacaacactgcaccgcgtcccgtccctcccgcccgcccgggcccggatcccgctccccaccgcctgaagccggcccgacccggaacccgggccgctggggagttgggttcaccttggaggccagagagacttggcgcccggaagcaaagggaatggcaagggggaggggggagggagaacgggagtttgcggagtccagaaggccgctttccgacgcccgggcgttgcgcgcgcttgctctttaagtactcagactgcgcggcgcgagccgtccgcatggtgacgcgtgtcccagcaaccgaactgaatggctgttgcttggcaatgccgggagttgaggtttggggccgcccacctagctactcgtgttttctccggcctgcgagttggggggctcccgcctccccggcccgctcctgggcgcgctgacgtcagatgtccccaccccgcccagcgcctgccccaagggtctcgccgcacacaaagctcggcctcgggcgccggcgcgcgggcgagagcggtggtctctcgcctgctgatctgatgcgctccaatcccgtgcctcgccgaagtgtttttaaagtgttctttccaacctgtgtctttggggctgagaactgttttctgaatacaggcggaactgcttccgtcggcctagaggcacgctgcgactgcgggacccaagttccacgtgctgccgcggcctgggatagcttcctcccctcgtgcactgctgccgcacacacctcttggctgtcgcgcattacgcacctcacgtgtgcttttgccccccgctacgtgcctacctgtccccaataccactctgctccccaaaggatagttctgtgtccgtaaatcccattctgtcaccccacctactctctgccccccccttttttgttttgagacggagctttgctctgtcgcccaggctggagtgcaatggcgcgatctcggctcactgcaacctccgcctcccgggttcaagcgattctcctgcctcagcctcctgagtagctggggttacagcgcccgccaccacgctcggctaatttttgtagtttttagtagagacgaggtttcaccatcttggccaggctggtcttgaacccctgaccttgtgatccactcgcctcggccttccaaagtgttgggattacgggcgtgacgaccgtgccacgcatctgcctcttaagtacataacggcccacacagaacgtgtccaactcccccgcccacgttccaacgtcctctcccacatacctcggtgccccttccacatacctcaggaccccacccgcttagctccatttcctccagacgccaccaccacgcgtcccggagtgccccctcctaaagctcccagccgtccaccatgctgtgcgttcctccctccctggccacggcagtgacccttctctcccgggccctgcttccctctcgcgggctctgctgcctcacttaggcagcgctgcccttactcctctccgcccggtccgagcggcccctcagcttcggcgcccagccccgcaaggctcccggtgaccactagagggcgggaggagctcctggccagtggtggagagtggcaaggaaggaccctagggttcatcggagcccaggtttactcccttaagtggaaatttcttcccccactcctccttggctttctccaaggagggaacccaggctgctggaaagtccggctggggcggggactgtgggttcaggggagaacggggtgtggaacgggacagggagcggttagaagggtggggctattccgggaagtggtggggggagggagcccaaaactagcacctagtccactcattatccagccctcttatttctcggccgctctgcttcagtggacccggggagggcggggaagtggagtgggagacctaggggtgggcttcccgaccttgctgtacaggacctcgacctagctggctttgttccccatccccacgttagttgttgccctgaggctaaaactagagcccaggggccccaagttccagactgcccctcccccctcccccggagccagggagtggttggtgaaagggggaggccagctggagaacaaacgggtagtcagggggttgagcgattagagcccttgtaccctacccaggaatggttggggaggaggaggaagaggtaggaggtaggggagggggcggggttttgtcacctgtcacctgctcgctgtgcctagggcgggcgggcggggagtggggggaccggtataaagcggtaggcgcctgtgcccgctccacctctcaagcagccagcgcctgcctgaatctgttctgccccctccccacccatttcaccaccaccatg

SEQ ID NO: 185 - DNA, 3831 bp, Homo sapiens
  source 1..3831: mol_type = other DNA
caacgggtacccttgcctgagtaagggggctgtgggtagagtgtgctggaacggacgtgtcctcgcagcctcatgcccgtgtgcgtggcgtgtgccctttagcccgagatttcaggtagctgcgacgggtgacaacttctctcccagccccctacaaaagagacctggcgcgaggggagcgaggccgtgagatgccagctggggctcctgcgggagcgcacccggagatccgagcctgccagaggcaggcggcgggcgcagagcggagaaagaggggcttctctccctagacgctgaacgatctaggatccgtccccgtcccccacctcgggacagaaaggacagtttgtctaggtttggagagaaaaaaccactgcataggccgtgcccaaaagccgctggccaagtcccccaagcgactgtcttctgcgccccgatgtctctgtcctcagcgccccccccccacacccggcacccctgctgtgcgtttcgatactgggcgtgctggcgccacaatctccgctcttgcctcgtcttcctggaaatggcacagagtcctttgggaaacccttgctctgaggatcagcgagttggatggccaggaggaggactttctgtgccagccgggagcaaccggctccgcggtcctgacactcgcccctccatttctcaaccccgtaggccagcaccgccccggcttttcccaggcgctcacgcgccgcggtggccctcaggggcttttgtcaccctgccagtgggggctctcgctctagccgcacagagaccaagccgggttctgcaggccctgagggaggtggggggtgggaagtgaatgcgggaaacatgatggggagaggagaaactgaagctgagtaggatttaggacctcccctgatgtcgggtcgccatcccaacactcatttcttgggctggtaatcacagcccctatgtaaaaggggggcggggggggcaggtgcgtaagaccattctcaccctcctctctacagagcctggacatggttcagaggaaaccgaccactagccatttccagcatctaacaattcttgggctggaaaaacaaagaatgcagaaaacgaaacttccttgtacatttaatttaaccacaattcatctagaattgtctgcctggcattggaatattctttctctgaaacaaaaatgaaacagaagtctctggaagaccttaagcggctgacttctttgttaaataagactccccatgatttaagctcatttcttgcttagaggagccttcccactctcagccggctccccagcctcccacctccaccaccttcaccaagactctgaaccctgtctgttgctaccattaagcaattctgtcctgttgactcaaactccagttaaaatgaccgagttagggctggaaagcaacactcaaccctctctcatactccctgcaccatcatcgttcctagcccaaaagctcttagacaggggctctgccaacccagggggattccgtgttactcagacattggagtgtgaccattcatgttatatagatgggcccctggaaatccccatgataaggtacactctgattgcaggcagcttgaataggattctggctctgtagaattaaaccaactgaccagatggttagaagtgataacgaaactacccaagttaatccagggatactaaccacagtttctgtacagcttctgttttaattgctgccagtctatgcttttttacgcaatgcagacatgaaattccaggtgcctcaaatacttcacaaaatggtcagccacaaagcccagatctcacttcacagacagttgtgtggtagggaaatgagcacagaaggaacgagcaatgcacctggcagttcagaatcaatcagaagcaaaggtgagcaaggatcctcaagtacttgttgctggccaagtctcctttaactgatctgcagtctttccaaggattaagaagtaatcttccatctacacccaggcaccaggaaaaggacctagctcaggggaaatgtgtcagccaagtgaattagtcccactctgctgaacacaccaccctttgaacatctcgcctcttcctagattggcctctttgctgtcctcctgcttcactcttcatatacccaagacccagctcaaacaattctctttggaagcctcctctgagtcccccaggaaaggaaggcattcttaagtccttcatttatctctcgtgcaatgcccaccctatatgagctggcttcctttcctatctccccttttaaattatcacctcctagagggcactggccaagtttgttcatttctacatccctgctgtcagcacaaagaagcctcctctccaggcccccaacccccgtgatattttttgaatggctgtatatcaatcatttaattatgggatgaactattgttttagatcttaagccaagccaatagtgctccaattattttctcagcaaggaagtaacacaggagtcagttgcttcaaaccaaagcccagttatcagccgttcggtctctaggccactgaggagcagaggggatgccttgagacgtgcaaaagacttggggccaggtggcctgtgttcacatcccagctccaccaattatgtgcaagagaatggggtgagctccttaaactctcttaagcctcagtttccacatctctaaaatgggggtaattatccctaccacctaggacagttggggagatcaagggactcgtgaatgtgaatgaattatatcagtactggaagccttctgcttacttctgtgaaagagcttgtgtcccacacctgcttcccgtttttgtccgtaattagaaaacggcaggcaaattctctggagtgttacagcacttgggagcagcatccccttagggactttgggaaagagctcttgaggaagtcaagcattaggtattggaaaacaaaaatagaagaaaaacaaaaaataaactgaagcctacatttcaaaaatgaaagcaaaccagacttttatttttaatactgaagactataaattgtttcaccacgtaggtagatttcaataaatcagggataatgagatggtagaggaaaacatggggggaaacaacttacgaggttcccattatgagcccaacgcaaggctaggcattttcacatatattccatcatttaaccttcatgacgcccccatgtgaagaaataagagtcagaaccattaaggaccaggcatgtggtcacacgggctcagcagtggaacccggtttgttctgcctctagagtctgggttttttccactatggcattttcagaatggaaagactccaaggcagtcagcaagtcagcatagatttcctggtagggaagaggccaggaatgtcagtgtcagacccttctgaggtcaggcgctgaacttctccaagctctgcctttctgcagtttagatcagtcaacttcttaggggtcaaagtatgtgctttttgaagccacagccctccccgacatgtgcgtcagcagatgatggctgaacccaaacccttccctactattggaaaaacaactcaaaaagtctgcacactgatgaggaactctagagcttaatgttgatgtggaaagataatacatttttcaatttaagagtatgtctgagaggctaaaccagaaatgtgtaaatttggtgagactttaaacagcctgtgaccgacgggccaatcttcctcttttccttccagatgtcacactggatccttggcctccagggtccattaaggtgagaataagatctctgggctggctggaactagcctaagactgaaaagcagcc

SEQ ID NO: 186 - DNA, 539 bp, Homo sapiens
  source 1..539: mol_type = genomic DNA
cccgggggtcctggaagccacaaggtaaacacaacacatccccctccttgactatcaattttactagaggatgtggtgggaaaaccattatttgatattaaaacaaataggcttgggatggagtaggatgcaagctcccaggaaagtttaagataaaacctgagacttaaaagggtgttaagagtggcagcctagggaatttatcccggactccgggggagggggcagagtcaccagcctctgcatttagggattctccgaggaaaagtgtgagaacggctgcaggcaacccagcttcccggcgctaggagggacgcacccaggcctgcgcgaagagagggagaaagtgaagctgggagttgccactcccagacttgttggaatgcagttggagggggcgagctgggagcgcgcttgctcccaatcacaggagaaggaggaggtggaggaggagggctgcttgaggaagtataagaatgaagttgtgaagctgagattcccctccattgggaccggagaaaccagggagcccccccggg

SEQ ID NO: 187 - DNA, 1305 bp, Homo sapiens
  source 1..1305: mol_type = other DNA
ggatccgtcccgggactagcagggctttgggcagcaacccgcaggagcccgaccgcctctggccaggtccgggcagctggtgggggaggttccagaggtccacgccattcgtggacgcagtctctagtgtcctctccgcgtcccacttcactgccccatcccctttcctgcgagagcctggacttggaaggcacctgggagggtgtaagcgccttggtgtgtgcccatctgggtccccagaagagcggcgggaactgcggccgcccggacggtgcggccagactccagtgtggaaggggaggcagctgttctcccaggcggccgtggggggcagcagaggggacggcgacaggtgcgggagcccctcccggggtagaagtggaaaggcgggctccggggtctgttcccaggctggaaaccacccccgccccccatccaaatccccgggagaggcccggccggcgccgggtctggaggaggaagcggccagagacagtgcaatttcacgcggtctctgtggctcgggttcctgggctgggtggatgaattatggggtttcgagtctgggagaaactgaggtggcctggacgtgaggcaaaaaacaccctccccctcaaaaacacacagagagaaatattcacattctgagagaaaatccaccaagtgaaccaaccggctaggggagttgagtgatttggttaatgggcgaggccaactttcagggggcagggctttggagagctttccactccctcattcattacccttccctggatctgggggctttcggaatctcgacctccccttggcctatctcctgcagaaaaattagggtgagccccatcctcgatctgctccgccaagttgcgggaccgcggggcgtggcacgctcaggggcaggcggtccgaggctccgcaatccccactccagcctcgcgcgggagggggcgcggcccgtgtgactcacccccttccctctgcgttcctccctccctctctctctctctctcacacacacacacccctcccctgccatccctccccggactccggctccggctccgattgcaatttgcaacctccgctgccgtcgccgcagcagccaccaattcgccagcggttcaggtggctcttgcctcgatgtcctagcctaggggcccccgggccggacttggctgggctcccttcaccctctgcggagtcatgagggcgaacgacgctctgcaggtgctgggcttgcttttcagcctggcccggggctccgaggtgggcaactctcaggcaggtaagtgcccagagagcacc

SEQ ID NO: 188 - DNA, 1343 bp, Homo sapiens
  source 1..1343: mol_type = other DNA
ggatccagcaatatggtggcaggctggactaaaggagagatgactgggaagcaatttcctgtggtgcatgacagctgatggatggatgtcagaaacagtggtgtctgatgatccatttgaagccatttcctcctctatattgctattactgtccatctccccctaaattttcagtaagcacctattatataaagcaccttagtattaaaaaatgaaggagatgaaagagaaggttgtgcagttgtattttgggccaagaagagtgggagaggtggcagggccagcgatgaagagcctgccagagtgatggaggcctgagcaaggagcaagttggtgaagaaagattaggacattgccatgtggagtcgctgtggaagcctgtttgttctcacgagctcagtggagaagaggtaaaagtagggaccagtagctgagtcattatgagaaagagggtttcatggtggtggaagtgacacattgcctcgattctcttgaagctttctgctttgttgcttgagtggagagaagcacctctgctattgcgtatggagggaagctctttgcatggattttgaaggcggcctctgcatttcggactactgggtgctcccccacaggctcctaacaccttgctgcttctccaggtggggtctgacgtggagtcagctcacagacctgccattcctctctcatagtactcctcattccagtgatatcttggcctgcttcatgaaccctgagcccagagttcctaaagcaccaaacccagtgaagcagagacacttctggcatgggtctgtgggttgcttctcaggggccaggccagcaagaatgattcagcacacaggccaacctgtgcaagctttatgcatgcattttagggcaatgggaagagtggtgagtgaggtttatggtaaatctttaaccacattcaattttttctaagacttttctgctttagaacatgtagaaatggagaaatgaccaggggctgcacaatgctgtgcttattatattgctgtagagagaaggatgctgccagctctccatagcctggggtgaacttggcctatgtaatgaggtagcagggagtcaggcaggtgagttcttcctcttgtattgccttttccagtaaatgccaatacactccccagctcacctttacctaacatctaggtcttaatccaagttgtcctcccactccccagggtgaattgatcttcctgccacggcacctcctgagcatctgttggctgagtctccatccccacccgtgaaaacagccttgtgatgtgctgtttaatatcacagaatggaaacagtgttttgattcaccaggatcc

SEQ ID NO: 189 - DNA, 223 bp, Homo sapiens
  source 1..223: mol_type = other DNA
caaagagctctgtgtccttgaacataaaatacaaataaccgctatgctgttaattattggcaaatgtcccattttcaacctaaggaaataccataaagtaacagatataccaacaaaaggttactagttaacaggcattgcctgaaaagagtataaaagaatttcagcatgattttccatattgtgcttccaccactgccaataacaaaataactagcaacca

SEQ ID NO: 190 - DNA, 1893 bp, Homo sapiens
  source 1..1893: mol_type = other DNA
agtgaatgctgttgctgctcgtctggaagccagacgttgagaaccccttctagagtgagctctcccgcagcaaattctactggcccccaaagtatgtgttttgtgtgtcttaaaaatttgttgagaaccattagcaaaaaaacaaacaaaaaaacttaattcctagaattccagagaaatcccatggagctttttgccagtcacgtcaagagaggccacaaacgtgccacttaaccagagcttcggaaaggcggcggctgggccggccacgtgcaccgagactcggggccaggtgcagccgccccagggccgaggcctcggaactggcccccggtcccggccccaagcggtccagcgattcccccaagccgtccgcccctccagatttatttacgttttcctgacttccccctgcccgctgtgggacaaacagcctccccacttgcatctgcgaggggagtagcgcgcacttccgccaagttccgcccccacccagcccgaggcccggctgccgccatcttgcggggggcgcacctcacaggtcgggagctgggcgggaaggggcgtggtcccgggacccgccccgccggggcttttgggagcgcgggcagcgagcgcactcggcggacgcaagggcggcggggagcacacggagcactgcaggcgccgggtgaggcgtgcggcggccggggtcgggacgggggttctgggcggggggttcctggtggagggcccgggcgggcggcggggttcggcggcaggtgcggcgggcagcctagggggcgcggcgcggggttctcgcccggcacccccggggcaggtggagctgagccggcccgcggccccgcgaccttcccctcggcgccgggtcccctcaggtctctcccgaaggaaacgcggagcctgggtgcctgggcgccgtccctcggcggctcccgagcggttgcagtttttgaaagagtttctcaaaggcttgacggttgtgactgcagccgcggggcaacggttgctacacaaagtgaaacttgccgagtgctcggcttctcacgggcttcctggcagccccgggaagttcctcggcggaccccgagcccgcgccccctctccacggatccctccccagcgagtgcccccccgcccgccctgtgccccctctcccctgacccctccctgtcgggtgccccgcgggctcgcgctggctgtcctgggactccttcctcctaggtgttcctcctgcccctcgccctctctctcccaggcgcgcgctccctctccccgggcctttccccgccgggtatccctgggcccgcgccccgtcttctccgcctctctccgctgggtgcacctcgagtgtcccccagacccctccccgcccggccggcgctctctcccctgaccctcctggccgagtgttccccggggcccgcgccccctccccccgatcctccccactgagtgttccccctgccctctctctcccgggcctgcgccccccaccagccccttcatgctgggggtcccctgggtgcgcaccccctctcctcggacccacccccaactggggggcacctccagtgcccgccggctgccccttgggcgcgcgcccccgctctcgggcgcctcctcgccgggggcccggcccggccccgccccgcccgtgccccctccccatgcccgcagtgctgggcggggcgctgactcacccgggcccgggctggccggttcttaagcggcagcgcgctgcgggcgccgagtgtcgggcgcggcaggaggacgaggcagggcgggcgggcgctctaagggttctgctctgactccaggttgggacagcgtcttcgctgctgctggatagtcgtgttttcggggatcgaggatactcaccagaaaccgaaa

SEQ ID NO: 191 - DNA, 284 bp, synthetic construct
  source 1..284: mol_type = other DNA
ttaaactcttatgtaaaatttgataagatgttttacacaactttaatacattgacaaggtcttgtggagaaaacagttccagatggtaaatatacacaagggatttagtcaaacaattttttggcaagaatattatgaattttgtaatcggttggcagccaatgaaatacaaagatgagtctagttaacacgtatattaatctacaattattggttaaagaatagtgctaatttccctccgtttgtcctagcttttctcttctgtcaaccccacacgcctttgg

(19) United States
(12) Patent Application Publication
(10) Pub. No.: US 2024/0319166 A1
(43) Pub. Date: Sep. 26, 2024

(54) Genetically-encoded volatile synthetic biomarkers for breath-based cancer detection

(71) Applicant: The Board of Trustees of the Leland Stanford Junior University, Standford, CA (US)

(72) Inventors: Ophir Vermesh, Los Angeles, CA (US); Aloma L. D'Souza, Pacifica, CA (US); Israt Shamima Alam, Mountain View, CA (US); Sanjiv Sam Gambhir, Portola Valley, CA (US)

(21) Appl. No.: 18/579,619

(22) PCT Filed: Aug. 1, 2022
(86) PCT No.: PCT/US22/74400

§ 371 Date: Jan. 16, 2024

Related U.S. Application Data
(60) Provisional application No. 63/227,587, filed on Jul. 30, 2021.

(51) Int. Cl. G01N 33/497; C12N 9/04; C12N 9/88; C12N 15/85

(52) U.S. Cl. CPC G01N 33/4975; C12N 9/0006; C12N 9/88; C12N 15/85; C12N 2800/22; C12Y 101/01088; C12Y 402/0302; G01N 2333/902; G01N 2333/988

(57) ABSTRACT

Genetically-encoded volatile synthetic biomarkers and methods for detection of various cancers in a subject are provided. In various aspects, embodiments provide compositions for breath-based cancer detection comprising at least one nucleic acid molecule encoding a synthase that catalyzes production of said volatile organic biomarker. The invention also provides devices, such as an electronic nose device, portable electronic nose device, and/or breath analyzer, for breath-based cancer detection comprising said compositions and at least one analyzer.

### SEQUENCE LISTING

This application includes a sequence listing submitted in written form and in computer readable form. The sequence listing is incorporated to this application in its entirety.

### FIELD OF THE INVENTION

This invention relates to genetically-encoded limonene for breath-based cancer detection methods and compositions.

### BACKGROUND OF THE INVENTION

Breath analysis provides rapid and non-invasive biomolecule detection, with great promise for early cancer detection and surveillance. The human body emits hundreds of volatile organic compounds (VOCs)—organic molecules that readily vaporize at room temperature—in the breath.

Breath, a less complex matrix than blood and other bodily fluids, can be sampled easily, painlessly, and inexpensively. Moreover, breath can be directly analyzed using real-time mass spectrometry, reducing the need for sample storage and processing. While no single VOC can reliably signal cancer presence on its own, VOC signatures or “breathprints” have been reported that can distinguish a number of cancers—including lung, colon, breast, and prostate cancers—from benign disease and healthy controls in relatively small study populations. However, as with liquid biopsies, clinical implementation of breath VOCs for early cancer detection is limited by low signal from cancer cells and high background signal from nonmalignant tissues. Furthermore, identification of reliable cancer-specific VOC signatures has been impeded by a lack of standardized breath sampling and analysis protocols, high inter-individual variability, a multitude of confounding variables, and false correlations due to statistical overfitting of high-dimensional datasets—a common pitfall in early stage 'omics approaches due to typically small study populations relative to the numerous endogenous parameters analyzed—limiting their generalizability. Thus, there is a need in the art for biomarkers and methods that can effectively and selectively detect various cancers. The present invention satisfies this unmet need.

### SUMMARY OF THE INVENTION

In one embodiment, the genetically-encoded biomarkers (e.g., volatile organic compounds, such as limonene) represent a strategy that overcomes the limitations of endogenous biomarkers.

Herein in an exemplary embodiment, the inventors provide a novel strategy for breath-based cancer detection which uses limonene, a plant VOC found in citrus fruits, as a sensitive and specific volatile reporter of cancer.

In a clinical strategy, a person undergoing screening or surveillance for cancer can be administered (intravenously, intranasally, orally, or by another route) a DNA vector containing a gene coding for the enzyme limonene synthase, driven by a tumor-specific promoter. Selectively expressed in cancer cells, the enzyme catalyzes production of the VOC limonene, which diffuses into the bloodstream and is transported to the lungs, where it is exhaled in the breath and detected by a breath analyzer, uniquely signaling the presence of early cancer and subsequently the extent of disease.

Applications of the embodiments are for example in screening and surveillance tests for cancer with likely customers being patients, outpatient clinics, hospitals, and the general population.

The present invention is based, in part, on the results that administering delivery vectors encoding the enzyme limonene synthase to cancer cells in culture resulted in limonene production by those cancer cells. Furthermore, the present invention is also based, in part, on the results that in vivo administration of delivery vectors encoding the enzyme limonene synthase, driven by a tumor-specific promoter, resulted in selective production of limonene in cancer cells. Thus, in various embodiments, the present invention relates, in part, to genetically-encoded biomarkers (e.g., volatile organic compounds, such as limonene) and methods of use thereof for detection of various cancers in a subject in need thereof.

In some aspects, the present invention provides compositions for breath-based cancer detection comprising at least one nucleic acid molecule encoding a synthase that catalyzes production of said biomarker of interest (e.g., volatile organic compounds, such as limonene). In other aspects, the present invention provides compositions for breath-based cancer detection comprising at least one synthase that catalyzes production of said biomarker of interest (e.g., volatile organic compounds, such as limonene).

In some aspects, the present invention also provides devices, such as electronic nose device, portable electronic nose device, breath analyzer, and/or breathalyzer, for breath-based cancer detection comprising said compositions and at least one analyzer.

In various aspects, the present invention provides a composition comprising a nucleic acid molecule encoding an exogenous synthase that expresses preferentially in cancer cells compared to noncancerous cells and catalyzes production of a volatile organic compound that is not endogenously produced.

In some embodiments, the volatile organic compound is a terpene. In some embodiments, the volatile organic compound is limonene.

In some embodiments, the exogenous synthase is an enzyme limonene synthase. In some embodiments, the enzyme limonene synthase comprises at least one amino acid sequence that is at least about 70% identical to the amino acid sequence selected from SEQ ID NOs: 1, 3, 5, 7, 9, 11, 13, 15, 17, 19, 21, 23, 25, 27, 29, 31, 33, or 35-38, or a fragment thereof.

In some embodiments, the nucleic acid molecule encoding an exogenous synthase comprises at least one vector. In some embodiments, the vector comprises at least one selected from adenovirus, retrovirus, adeno-associated virus, herpes virus, poxvirus, vaccinia virus, lentivirus, or any combination thereof. In some embodiments, the composition comprises at least one nucleotide sequence that is at least about 70% identical to the nucleotide sequence selected from SEQ ID NOs: 2, 4, 6, 8, 10, 12, 14, 16, 18, 20, 22, 24, 26, 28, 30, 32, 34, or 45-50, or a fragment thereof.

In some embodiments, the exogenous synthase contains at least one of the conserved amino acid motifs in the enzyme limonene synthase or its enzyme class (SEQ ID NOs: 51-175).

In some embodiments, the composition comprises at least one selected from a genetic delivery vector, minicircle, liposome, plasmid, viral vector, or any combination thereof.

In some embodiments, the composition further comprises at least one gene delivery vector containing at least one nucleotide sequence encoding 3-hydroxy-3-methylglutaryl coenzyme-A (HMG-CoA) reductase (HMGR). In some embodiment, the composition comprises at least one gene delivery vector containing at least one nucleotide sequence encoding a truncated form of HMGR. In a preferred embodiment, the composition comprises at least one gene delivery vector containing at least one nucleotide sequence encoding a truncated form of HMGR in which the N-terminal regulatory domain has been deleted. In a preferred embodiment, the composition comprises at least one gene delivery vector containing at least one gene encoding only the catalytic portion of HMGR. In some embodiments, the gene delivery vector comprises at least one nucleotide sequence that is at least about 70% identical to the nucleotide sequence selected from SEQ ID NO: 39 or a fragment thereof or SEQ ID NO: 41 or a fragment thereof. In some embodiments, the truncated HMGR comprises at least one amino acid sequence that is at least about 70% identical to the amino acid sequence selected from SEQ ID NO: 40 or a fragment thereof.

In some embodiments, the composition comprises at least one tumor-specific promoter. In some embodiments, the tumor-specific promoter includes, but is not limited to, at least one of the following nucleotide sequences: Survivin promoter, human (SEQ ID NO: 176), hTert core promoter, human (SEQ ID NO: 177), CXCR4 promoter, human [GenBank ID: U81003.1](SEQ ID NO: 178), Hexokinase type II promoter, human [GenBank: AF148512.1] (SEQ ID NO: 179), Stromelysin 3 (MMP11) promoter, mouse [GenBank: AF297645.1] (SEQ ID NO: 180), Tyrosinase promoter, human, [GenBank: U03039.1] (SEQ ID NO: 181)Interleukin-10 promoter, human [GenBank: Z30175.1] (SEQ ID NO: 182), Epidermal growth factor receptor (EGFR) promoter, [GenBank: J03206.1](SEQ ID NO: 183), Mucin-like glycoprotein (DF3, MUC1) promoter, [GenBank: X69118.1] (SEQ ID NO: 184), Somatostatin receptor 2 (sst2)promoter, human [GenBank: AB260891.1] (SEQ ID NO: 185), c-erbB-2 promoters, human [GenBank ID: M16892.1] (SEQ ID NO: 186), c-erbB-3 promoter; human [GenBank ID: Z23134.1] (SEQ ID NO: 187), Thyroglobulin promoter, human [GenBank: X77275.1] (SEQ ID NO: 188), alpha-fetoprotein (AFP) promoter, human [GenBank: AB053572.1] (SEQ ID NO: 189), Villin 2 promoter, human [GenBank: EF184645.1] (SEQ ID NO: 190), or Albumin promoter (SEQ ID NO: 191).

In some embodiments, the tumor-specific promoter comprises at least one amino acid sequence that is at least about 70% identical to the amino acid sequence selected from Survivin promoter, human (SEQ ID NO: 176), hTert core promoter, human (SEQ ID NO: 177), CXCR4 promoter, human [GenBank ID: U81003.1](SEQ ID NO: 178), Hexokinase type II promoter, human [GenBank: AF148512.1] (SEQ ID NO: 179), Stromelysin 3 (MMP11) promoter, mouse [GenBank: AF297645.1] (SEQ ID NO: 180), Tyrosinase promoter, human, [GenBank: U03039.1] (SEQ ID NO: 181)Interleukin-10 promoter, human [GenBank: Z30175.1] (SEQ ID NO: 182), Epidermal growth factor receptor (EGFR) promoter, [GenBank: J03206.1](SEQ ID NO: 183), Mucin-like glycoprotein (DF3, MUC1) promoter, [GenBank: X69118.1] (SEQ ID NO: 184), Somatostatin receptor 2 (sst2)promoter, human [GenBank: AB260891.1] (SEQ ID NO: 185), c-erbB-2 promoters, human [GenBank ID: M16892.1] (SEQ ID NO: 186), c-erbB-3 promoter; human [GenBank ID: Z23134.1] (SEQ ID NO: 187), Thyroglobulin promoter, human [GenBank: X77275.1] (SEQ ID NO: 188), alpha-fetoprotein (AFP) promoter, human [GenBank: AB053572.1] (SEQ ID NO: 189), Villin 2 promoter, human [GenBank: EF184645.1] (SEQ ID NO: 190), or Albumin promoter (SEQ ID NO: 191).

In some embodiments, the nucleic acid molecule encoding an exogenous synthase is codon-optimized for mammalian cells.

In some embodiments, the nucleic acid molecule encoding an exogenous synthase is codon-optimized for human cells.

In various aspects, the present invention also provides a breath-based method of detecting cancer in a subject in need thereof, the method comprising the steps of: (a) administering to the subject at least one composition of the present invention; (b) capturing breath exhaled from the subject; (c) analyzing the exhaled breath for the volatile organic compound; (d) comparing the amount of the volatile organic compound in the exhaled breath to a comparator; and (e) determining the subject has cancer when the amount of the volatile organic compound in the exhaled breath is increased compared to a comparator.

For example, in some embodiments, the present invention provides a breath-based method of detecting cancer in a subject in need thereof, the method comprising the steps of: (a) administering to the subject at least one composition comprising a nucleic acid molecule encoding an enzyme limonene synthase, wherein the enzyme limonene synthase expresses preferentially in cancer cells compared to noncancerous cells and catalyzes production of limonene; (b) capturing breath exhaled from the subject; (c) analyzing the exhaled breath for the limonene; (d) comparing the amount of limonene in the exhaled breath to a comparator; and (e) determining the subject has cancer when the amount of limonene in the exhaled breath is increased compared to a comparator.

In other aspects, the present invention also provides a method of treating a cancer in a subject in need thereof, the method comprising the steps of: (a) administering to the subject at least one composition of the present invention; (b) capturing breath exhaled from the subject; (c) analyzing the exhaled breath for the volatile organic compound; (d) comparing the amount of the volatile organic compound in the exhaled breath to a comparator; (e) determining the subject has cancer when the amount of the volatile organic compound in the exhaled breath is increased compared to a comparator; and (f) administering a therapeutically effective amount of at least one anti-cancer agent to the subject having cancer.

In other aspects, the present invention also provides a method of evaluating the effectiveness of a cancer treatment in a subject in need thereof, the method comprising the steps of: (a) administering to the subject at least one composition of the present invention; (b) capturing breath exhaled from the subject; (c) analyzing the exhaled breath for the volatile organic compound; (d) comparing the amount of the volatile organic compound in the exhaled breath to a comparator; and (e) determining the cancer treatment as effective when the amount of the volatile organic compound in the exhaled breath is decreased compared to a comparator.

In various aspects, the present invention also provides a device for detecting cancer in a subject in need thereof, wherein the device comprises at least one composition of the present invention and at least one analyzer of the volatile organic compound. In some embodiments, the device is an electronic nose device, portable electronic nose device, or breath analyzer.

### BRIEF DESCRIPTION OF THE DRAWINGS

FIG. 1 shows according to an exemplary embodiment of a method of the invention.

FIG. 2 shows according to an exemplary embodiment a schematic representation of a cancer reporter strategy using an exogenous volatile organic compound. A cancer patient undergoing surveillance or a healthy subject undergoing cancer screening is administered a gene delivery vector (minicircle, liposome, or adenovirus) encoding an exogenous synthase (e.g. a terpene synthase, such as limonene synthase)—driven by a tumor-activatable promoter—which catalyzes production of an exogenous volatile organic compound (VOC)(e.g. a terpene, such as limonene) specifically in cancer cells that is not otherwise produced endogenously.

The VOC diffuses into the bloodstream and is transported to the lungs, where it is exhaled in the breath and detected by a breath analyzer (mass spectrometer or electronic nose sensor array), uniquely signaling the presence of cancer and overall tumor burden. In the case of lung cancer, the gene delivery vector could also be administered noninvasively; for example, using an inhalable formulation. While a lung tumor was shown above to illustrate the concept, this strategy is generalizable to many cancer types. Inset: Expressing a plant VOC in a human cell. Plants and humans share a conserved metabolic pathway for cholesterol production (blue arrows) but in plants, terpene synthases divert part of this metabolic stream towards production of volatile organic compounds that attract pollinators and protect from herbivorous insects, parasites, and pathogens. Selective expression of terpene synthases, such as limonene synthase (yellow arrow), in human cancer cells enable these cells to produce plant VOCs that are detectable in breath, serving as highly specific cancer reporters. Substrates in the cholesterol biosynthetic pathway: HMG-CoA, 3-hydroxy-3-methylglutaryl coenzyme A; DMAPP, dimethylallyl pyrophosphate; IPP, isopentenyl diphosphate; GPP, geranyl diphosphate; FPP, farnesyl pyrophosphate.

FIGS. 3A-G show according to exemplary embodiments schematic representations of vector design, transfection, and limonene production by HeLa cells. FIG. 3A shows a schematic representation of experimental methodology. (Top) Cultured HeLa cells were transfected with a vector containing LS and eGFP genes under the control of a CAG promoter. Antibiotic and FACS selection for stably transfected clones (sorting on eGFP-expressing cells) resulted in a HeLa cell line containing both LS and eGFP (HeLa-LS-eGFP cells, subsequently referred to as HeLa-LS cells). (Bottom) HeLa-LS cells were subsequently transfected with a vector containing the tHMGR and tRFP genes under the control of an EF1α promoter. Antibiotic and FACS selection (based on dual expression of eGFP and tRFP) resulted in a HeLa cell line containing LS, tHMGR, eGFP, and tRFP (HeLa-LS-tHMGR-eGFP-tRFP, subsequently referred to as HeLa-LS-tHMGR). Solid phase microextraction (SPME) fibers were used to sample the culture headspace of confluent stably transfected HeLa-LS and HeLa-LS-tHMGR cells for 30 minutes, and were then analyzed for limonene by GC-MS. FIG. 3B shows a schematic representation of (i) Piggybac transposon DNA vector containing truncated limonene synthase (LS) and enhanced green fluorescent protein (eGFP) driven by a CAG promoter, and puromycin resistance gene driven by a CMV promoter; and (ii) Piggybac transposon DNA vector containing truncated HMG CoA reductase (tHMGR) and turbo red fluorescent protein (tRFP) driven by an EF1α promoter, and hygromycin resistance gene driven by a CMV promoter as well as parental and minicircle plasmids. To create DNA minicircles, genes of interest (e.g. limonene synthase and firefly luciferase [Luc2]) and a promoter of interest (e.g. the survivin or hTert promoters) are cloned into a parental plasmid backbone (for example, the MN-100 PP backbone from System Biosciences, Palo Alto, CA) resulting in a parental plasmid containing the desired genes and promoter (iii). Minicircles are produced from the full-sized parental minicircle using PhiC31 Integrase, which mediates a recombination event between the PhiC321 attB and attP sites on the parental plasmid. This reaction results in two products—the minicircle, which is now free from any bacterial DNA sequences—and the parental plasmid. To get rid of the parental plasmid, the I-SceI endonuclease recognizes and acts on the I-SceI sites on the parental plasmid, resulting in degradation of the parental plasmid. The minicircle contains the limonene synthase gene and firefly luciferase (Luc2) gene, both driven by a tumor-specific promoter, such as the survivin or hTert promoter (iv). FIG. 3C shows representative bright-field and fluorescence images showing HeLa-LS and HeLa-LS-tHMGR cells after antibiotic selection and FACS sorting, compared with untransfected control HeLa cells. Scale bar=200 um for HeLa control and 400 μm for HeLa-LS and HeLa-LS-tHMGR. FIG. 3D shows a representative mass spectrum from an SPME fiber exposed to the headspace of confluent HeLa-LS cells (top) compared with the reference spectrum of limonene from a mass spectrum library (Mnova database) (bottom). Note the characteristic peaks at m/z=68, 93, and 136. FIG. 3E showss representative results demonstrating selected ion monitoring (SIM) mode chromatogram of an SPME headspace sample from HeLa-LS cells (left) and from a pure limonene standard (right), showing matching ion ratios and retention times. FIG. 3F shows representative results demonstrating calibration curve relating headspace limonene concentration as measured by SIFT-MS to the quantity of limonene spiked into culture media in a T75 flask (y=0.62x^{0.86}, R²=0.99). Over the range of limonene production by cultured cells (1 to 1000 ng, red bracket), the relationship is well-modeled by y=0.28×(R²=0.99). FIG. 3G shows representative results demonstrating headspace concentration of limonene as a function of cell number for HeLa-LS (y=[1.56×10⁻⁶]x+1.06, R²=0.99) and HeLa-LS-tHMGR cells (y=[3.21×10⁻⁶]x+2.70, R²=0.98) after incubation at 37° C. for 24 hours. Limonene measured from HeLa-LS-tHMGR cells was approximately double that from HeLa-LS cells over the cell density range examined.

FIGS. 4A-G show according to exemplary embodiments representative results demonstrating limonene detection from mice. FIG. 4A shows a schematic representation of intraperitoneal injection of limonene into a mouse, placement of the mouse in a sealed 0.5-L chamber, and SIFT-MS analysis of chamber air after 15 minutes. FIG. 4B shows representative results demonstrating limonene concentration in chamber headspace as a function of limonene dose injected intraperitoneally into mice (y=1.01x^{0.82}, R²=0.89) or spiked (i.e. pipetted) directly into a chamber containing 10 ml of water (y=83.83x^{0.84}, R²=0.99). Only ˜0.5% of limonene injected into mice was detected in chamber air at 15 minutes. Each data point represents mean±SD for n=3 mice (one mouse per chamber). FIG. 4C shows a schematic representation of ten-week-old athymic nude mice that were inoculated subcutaneously in both flanks with either HeLa-LS, HeLa-LS-tHMGR, or untransfected control HeLa cells. Tumor progression in the 3 groups was followed over a five-week period with weekly measurements of tumor size and collection of mouse VOCs using a specially-designed mouse chamber setup in which highly purified air was continuously flowed into 6 one-liter mouse chambers (4 mice per chamber) in parallel at 100 mL/min. Air exiting the chamber was flowed through a cold trap to eliminate moisture and then through a sorbent trap containing Tenax resin to capture VOCs from the mice. The sorbent traps were subsequently analyzed by GC-MS. FIG. 4D shows representative results demonstrating that limonene signal in HeLa-LS-tHMGR mice increases with sampling time, whereas limonene signal in control mice remains below the detection limit (<2.3 ng), demonstrating that signal-to-noise ratio and sensitivity can be increased by increasing the sampling time. FIG. 4E shows representative results demonstrating that five-week follow-up study of grouped mice implanted with HeLa-LS, HeLa-LS-tHMGR, and untransfected control HeLa cells. Limonene production increased with time post-implantation for HeLa-LS and HeLa-LS-tHMGR mice and was detectable above background at one-week post-implantation in HeLa-LS-tHMGR mice (p=0.049), but not in HeLa-LS mice (p=0.26). By the second week, evolved limonene was statistically higher in both HeLa-LS-tHMGR (p=0.025) and HeLa-LS mice (p=0.025) than in control mice. Peak limonene production in HeLa-LS-tHMGR mice was significantly greater than in HeLa-LS mice (94±14 ng vs. 60±16 ng, p=0.049).*(P<0.05), NS (P>0.05). FIG. 4F shows representative results demonstrating that limonene production by HeLa-LS and HeLa-LS-tHMGR mice increases approximately linearly with tumor volume over the first 4 weeks of the study. HeLa-LS: y=0.10x−3.2, R²=0.95. HeLa-LS-tHMGR: y=0.12x−1.76, R²=0.97. Limonene was undetectable in control mice with untransfected HeLa tumors. FIG. 4G shows representative results demonstrating that tumor growth rates for all three groups were modeled based on monoexponential growth. HeLa-LS-tHMGR: y=77.3e^{0.48}, R²=0.99. HeLa-LS: y=62.2e^{0.53t}, R²=0.96. HeLa controls: y=34.5e^{0.54t}, R²=0.98. Each bar or data point for limonene quantity represents mean±SD for 3 chambers of 4 mice each (n=12 mice). “Tumor volume” refers to the average tumor volume in a single mouse.

FIG. 5 shows according to an exemplary embodiment representative results demonstrating limonene signal from empty chambers and chambers containing HeLa control mice in 10-hour sorbent trap experiments by week. (Each bar represents mean±SD for 3 chambers of 4 mice each; n=12 mice).

FIG. 6 shows according to an exemplary embodiment representative mouse chamber/sorbent trap assembly. Six one-liter induction chambers were operated in parallel for simultaneous mouse limonene measurements. The outlet of each chamber was connected in series via tygon tubing to a glass condenser on ice (cold trap) and then to a sorbent tube containing Tenax TA resin that traps and concentrates the VOCs. The inlet of each chamber was connected in series to a sacrificial Tenax sorbent tube, which serves to purify inflowing air, and an upstream 0.25 inch stainless steel metering valve that individually controls air flow into each chamber. The metering valves to all six chambers were connected via reducing unions, union tees, and 0.125 inch copper tubing to a benchtop pressure regulator set to 5 psi, which was connected via a single copper line to a compressed gas cylinder containing highly pure air set to 20 psi. For ease of cleaning the induction chambers between experiments, the tygon connections to inlet and outlet components were interrupted by 0.25 inch snap-on/snap-off fasteners.

FIGS. 7A-E show according to exemplary embodiments representative results demonstrating transduction of adenoviral constructs containing the limonene synthase gene in cell culture and in vivo in a mouse tumor model. FIG. 7A shows representative image of human MeWo (melanoma) cell line cells were seeded at a density of ˜60,000 cells per cm² in cell culture media containing 10% FBS in T25 culture flask. FIG. 7B shows representative image of HCC827 (non-small cell lung cancer) cell line cells were seeded at a density of ˜60,000 cells per cm² in cell culture media containing 10% FBS in T75 culture flask. FIG. 7C shows representative results demonstrating limonene levels in parts-per-billion from MeWo cells in T25 flasks at day 4 after adenovirus transduction at MOIs of 200, 1000, or 5000, and from untransduced MeWo cells (no virus added). The dashed line represents background signal from untransduced cells. FIG. 7D shows representative images of nude mice that were implanted with 2.5 million MeWo cells in each flank. FIG. 7E shows representative images of nude mice that were implanted with HCC827 cells in each flank.

FIG. 8 shows according to an exemplary embodiment multisequence alignment of (+) limonene synthase amino acid sequences from 7 different citrus species (SEQ IDs 1-7). This multisequence alignment was used to determine the conserved amino acids within these sequences.

### DETAILED DESCRIPTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The term “about” will be understood by persons of ordinary skill in the art and will vary to some extent depending on the context in which it is used. As used herein when referring to a measurable value such as an amount, a temporal duration, and the like, the term “about” is meant to encompass variations of 20% or ±10%, more preferably ±5%, even more preferably ±1%, and still more preferably ±0.1% from the specified value, as such variations are appropriate to perform the disclosed methods.

The term “volatile” as used herein, refers to a material that is vaporizable at room temperature and atmospheric pressure without the need of an energy source. The volatile material may be a composition comprised entirely of a single volatile material. The volatile material may also be a composition comprised entirely of a volatile material mixture (i.e. the mixture has more than one volatile component). Further, it is not necessary for all of the component materials of the composition to be volatile. Any suitable volatile material in any amount or form, including a liquid or emulsion, may be used. Liquid suitable for use herein may, thus, also have non-volatile components, such as carrier materials (e.g., water, solvents, etc).

The volatile material can be a “volatile organic compound (VOC)”. Volatile organic compounds (VOCs) are low-molecular-weight (i.e. typically in the range of 50-300 Daltons) organic compounds that have a high vapor pressure (at least 0.01 kPa at a temperature of 293.15 K), low boiling point (i.e. less than 250° C. at a pressure of 1 bar or atmospheric pressure), low water solubility, and easily evaporate at room temperature. They encompass a wide variety of chemical substances with the common feature of being carbon compounds that are volatile at ambient temperature. Chemically, VOCs are compounds containing at least one carbon atom together with atoms of hydrogen, oxygen, nitrogen, sulfur, halogens (fluorine, chlorine, or bromine), phosphorous, excluding carbon monoxide, carbon dioxide, carbonic acid, metallic carbides or carbonates and ammonium carbonate. They can be categorized by structure (e.g., straight-chained, branched, ring structures), by the types of chemical bonds (alkanes, alkenes, alkynes, saturated, unsaturated), by the function of specific parts of the molecules (e.g., aldehydes, ketones, alcohols, etc.), or by specific elements included (e.g., chlorinated hydrocarbons that contain chlorine, hydrogen, and carbon). A non-exhaustive list of chemical classes includes isoprene, terpenes, aliphatic hydrocarbons, alkanes, alkenes, alkynes, alcohols, aldehydes, esters, ethers, carbonyls, carboxylic acids, aromatic hydrocarbons, amines, amides, thiols, and halogenated versions of these. They can arise by a variety of biosynthetic routes but principally from amino and fatty acids, and terpene biosynthetic pathways. Examples include, but are not limited to VOC from oil of bergamot, bitter orange, lemon, mandarin, caraway, cedar leaf, clove leaf, cedar wood, geranium, lavender, orange, origanum, petitgrain, white cedar, patchouli, neroili, rose absolute, vanillin, ethyl vanillin, coumarin, tonalid, calone, heliotropene, musk xylol, cedrol, musk ketone benzohenone, raspberry ketone, methyl naphthyl ketone beta, phenyl ethyl salicylate, veltol, maltol, maple lactone, proeugenol acetate, evemyl, and the like. Furthermore, the volatile material can be synthetically or naturally formed materials.

The term “derivative” refers to a small molecule that differs in structure from the reference molecule, but may retain or enhance the essential properties of the reference molecule and may have additional properties. A derivative may change its interaction with certain other molecules relative to the reference molecule. A derivative molecule may also include a salt, an adduct, tautomer, isomer, or other variant of the reference molecule.

The term “tautomers” are constitutional isomers of organic compounds that readily interconvert by a chemical process (tautomerization).

The term “isomers” or “stereoisomers” refers to compounds, which have identical chemical constitution, but differ with regard to the arrangement of the atoms or groups in space.

As used herein “endogenous” refers to any material from or produced inside an organism, cell, tissue or system.

As used herein, the term “exogenous” refers to any material introduced from or produced outside an organism, cell, tissue or system.

“Isolated” means altered or removed from the natural state. For example, a nucleic acid or a peptide naturally present in its normal context in a living subject is not “isolated,” but the same nucleic acid or peptide partially or completely separated from the coexisting materials of its natural context is “isolated.” An isolated nucleic acid or protein can exist in substantially purified form, or can exist in a non-native environment such as, for example, a host cell.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

The term “RNA” as used herein is defined as ribonucleic acid.

The term “DNA” as used herein is defined as deoxyribonucleic acid.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting there from. Thus, a gene encodes a protein if transcription of the gene to mRNA and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene. A “coding region” of a mRNA molecule also consists of the nucleotide residues of the mRNA molecule which are matched with an anti-codon region of a transfer RNA molecule during translation of the mRNA molecule or which encode a stop codon. The coding region may thus include nucleotide residues comprising codons for amino acid residues which are not present in the mature protein encoded by the mRNA molecule (e.g., amino acid residues in a protein export signal sequence).

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence.

Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least about 50%, and or at least about 75%, or at least about 90%, or at least about 95% of the nucleotide residues of the first portion are capable of base pairing with nucleotide residues in the second portion. In some embodiments, all nucleotide residues of the first portion are capable of base pairing with nucleotide residues in the second portion.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. Generally, a comparison is made when two sequences are aligned to give maximum homology.

“Variant” as the term is used herein, is a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations.

Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis. In various embodiments, the variant sequence is at least 99%, at least 98%, at least 97%, at least 96%, at least 95%, at least 94%, at least 93%, at least 92%, at least 91%, at least 90%, at least 89%, at least 88%, at least 87%, at least 86%, at least 85%, at least 80%, at least 75%, at least 70%, at least 65%, at least 60%, at least 65%, at least 50% identical to the reference sequence.

As used herein, the term “fragment,” as applied to a nucleic acid or a peptide, refers to a subsequence of a larger nucleic acid or a peptide sequence, respectively. A “fragment” of a nucleic acid can be at least about 15 nucleotides in length; for example, at least about 15 nucleotides to about 2500 nucleotides; at least about 50 nucleotides to about 100 nucleotides; at least about 100 to about 500 nucleotides, at least about 500 to about 1000 nucleotides, at least about 1000 nucleotides to about 1500 nucleotides; or about 1500 nucleotides to about 2500 nucleotides; or about 2500 nucleotides (and any integer value in between).

The term “promoter” as used herein is defined as a DNA sequence recognized by the synthetic machinery of the cell, or introduced synthetic machinery, required to initiate the specific transcription of a polynucleotide sequence.

The term “regulating” as used herein can mean any method of altering the level or activity of a substrate. Non-limiting examples of regulating with regard to a protein include affecting expression (including transcription and/or translation), affecting folding, affecting degradation or protein turnover, and affecting localization of a protein. Non-limiting examples of regulating with regard to an enzyme further include affecting the enzymatic activity. “Regulator” refers to a molecule whose activity includes affecting the level or activity of a substrate. A regulator can be direct or indirect. A regulator can function to activate or inhibit or otherwise modulate its substrate.

“Vector” as used herein may mean a nucleic acid sequence containing an origin of replication. A vector may be used as a vehicle to deliver or transfer a gene into a host cell. A vector may be a plasmid, virus, minicircle, liposome, bacteriophage, bacterial artificial chromosome or yeast artificial chromosome. A vector may be a DNA or RNA vector. A vector may be either a self-replicating extrachromosomal vector or a vector which integrates into a host genome.

A “minicircle” vector, as used herein, refers to a small, double stranded circular DNA molecule (e.g., ˜3-5 kpb) that provides for persistent, high level expression of a sequence of interest that is present on the vector, which sequence of interest may encode a polypeptide, an shRNA, an anti-sense RNA, an siRNA, and the like in a manner that is at least substantially expression cassette sequence and direction independent. The sequence of interest is operably linked to regulatory sequences present on the mini-circle vector, which regulatory sequences control its expression. Minicircles are non-replicative, episomal/non-integrating (minimizing the risk of insertional mutagenesis and carcinogenesis), and have low immunogenicity due to the lack of a prokaryotic backbone (e.g., antibiotic resistance marker, replication origin).

The term “liposome” as used herein refers to an artificially prepared vesicle composed of a lipid bilayer. A liposome may be classified as a unilamellar vesicle or a multilamellar vesicle. As used herein, the term “liposome” refers to phospholipid molecules assembled in a spherical configuration encapsulating an Interior aqueous volume that is segregated from ani aqueous exterior. The lipid molecules are not soluble in water but may be dissolved in a solvent.

The terms “effective amount” and “pharmaceutically effective amount” refer to a sufficient amount of an agent to provide the desired biological result. That result can be reduction and/or alleviation of a sign, symptom, or cause of a disease or disorder, or any other desired alteration of a biological system. An appropriate effective amount in any individual case may be determined by one of ordinary skill in the art using routine experimentation.

A “therapeutically effective amount” refers to that amount which provides a therapeutic effect for a given condition and administration regimen. In particular, “therapeutically effective amount” means an amount that is effective to prevent, alleviate or ameliorate symptoms of the disease or prolong the survival of the subject being treated, which may be a human or non-human animal. Determination of a therapeutically effective amount is within the skill of the person skilled in the art.

“Pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

As used herein, the term “pharmaceutical composition” refers to a mixture of at least one compound of the invention with other chemical components and entities, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

The term “pharmaceutically acceptable salt” refers to any pharmaceutically acceptable salt, which upon administration to the patient is capable of providing (directly or indirectly) a compound as described herein. Such salts preferably are acid addition salts with physiologically acceptable organic or inorganic acids. Examples of the acid addition salts include mineral acid addition salts such as, for example, hydrochloride, hydrobromide, hydroiodide, sulphate, nitrate, phosphate, and organic acid addition salts such as, for example, acetate, trifluoroacetate, maleate, fumarate, citrate, oxalate, succinate, tartrate, malate, mandelate, methane sulphonate and p-toluenesulphonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic amino acids salts. However, it will be appreciated that non-pharmaceutically acceptable salts also fall within the scope of the invention since those may be useful in the preparation of pharmaceutically acceptable salts. Procedures for salt formation are conventional in the art.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient.

Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art.

As used herein, the term “stabilizers” refers to either, or both, primary particle and/or secondary stabilizers, which may be polymers or other small molecules. Non-limiting examples of primary particle and/or secondary stabilizers for use with the present invention include, e.g., starch, modified starch, and starch derivatives, gums, including but not limited to polymers, polypeptides, albumin, amino acids, thiols, amines, carboxylic acid and combinations or derivatives thereof. Other examples include xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, potassium polymethacrylate, carrageenan (and derivatives), gum karaya and biosynthetic gum. Other examples of useful primary particle and/or secondary stabilizers include polymers such as: polycarbonates (linear polyesters of carbonic acid); microporous materials (bisphenol, a microporous poly(vinylchloride), micro-porous polyamides, microporous modacrylic copolymers, microporous styrene-acrylic and its copolymers); porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(mides), poly(benzimidazoles), collodion, regenerated proteins, semi-solid cross-linked poly(vinylpyrrolidone).

The terms “patient,” “subject,” “individual,” and the like are used interchangeably herein, and refer to any animal, or cells thereof whether in vitro or in situ, amenable to the methods described herein. In certain non-limiting embodiments, the patient, subject, or individual is a mammal, non-human mammal, primate, mouse, rat, pig, horse, ferret, dog, cat, cattle, or human.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

The term “inhibit,” as used herein, means to suppress or block an activity or function by at least about ten percent relative to a control value. Preferably, the activity is suppressed or blocked by 50% compared to a control value, more preferably by 75%, and even more preferably by 95%.

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease.

The term “treatment” as used herein covers any treatment of a disease in a subject and includes: (a) preventing a disease related to an undesired immune response from occurring in a subject which may be predisposed to the disease; (b) inhibiting the disease, i.e., arresting its development: or (c) relieving the disease, i.e., causing regression of the disease.

Throughout this description, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### End Definitions

### Compositions

In various aspects, the present invention relates, in part, to compositions comprising a nucleic acid molecule encoding an exogenous synthase. In some embodiments, the nucleic acid molecule is an RNA (e.g., rRNA, tRNA and mRNA) molecule, DNA molecule, or a combination thereof. Thus, in some embodiments, the composition comprises a DNA molecule encoding an exogenous synthase. In other embodiments, the composition comprises an RNA molecule encoding an exogenous synthase.

In other aspects, the present invention relates, in part, to compositions comprising an exogenous synthase. In some embodiments, the present invention relates, in part, to compositions comprising or encoding multiple exogenous synthases, each catalyzing production of a different volatile organic compound. In various embodiments, the exogenous synthase or exogenous synthases express preferentially in cancer cells compared to noncancerous cells.

In some embodiments, the exogenous synthase is any plant synthase. For example, in certain embodiments, the exogenous synthase is an enzyme limonene synthase. In some embodiments, the exogenous synthase contains at least one of the conserved amino acid motifs in limonene synthase. For example, in some embodiments, the exogenous synthase contains the amino acid sequence motif RRXsW (SEQ ID NOs: 51-70). In certain embodiments, the exogenous synthase contains the amino acid sequence motif RRXsW (SEQ ID NOs: 51-70) within the first 80 amino acids of the N-terminal region. In some embodiments, the exogenous synthase contains at least one of the amino acid sequences DDxxD (SEQ ID NOs: 71-90), NDxxD (SEQ ID NOs: 91-110), DDxxE (SEQ ID NOs: 111-130), DxDD (SEQ ID NOs: 131-150), DDIYD (SEQ ID NOs: 151), VxDDxx(D,E) (SEQ ID NOs: 152-153), (I,L,V)XDDX(D,E) (SEQ ID NOs: 154-159), or any combination thereof. In certain embodiments, the exogenous synthase contains at least one of the amino acid sequences DDxxD (SEQ ID NOs: 71-90), NDxxD (SEQ ID NOs: 91-110), DDxxE (SEQ ID NOs: 111-130), DxDD (SEQ ID NOs: 131-150), DDIYD (SEQ ID NOs: 151), VxDDxx(D,E) (SEQ ID NOs: 152-153), (I,L,V)XDDX(D,E) (SEQ ID NOs: 154-159), or any combination thereof, within the last 300 amino acids of the C-terminal region. Each of these sequences is involved in divalent metal ion binding (typically of Mg²⁺) within the catalytic domain of the active site. In some embodiments an RXR motif is located between 30 to 40 amino acid residues upstream of any of the sequences specified in SEQ ID NOs: 71-159. In some embodiments, the exogenous synthase contains at least one of the amino acid sequences (N,D)D(L,I,V)X(S,T)XXXE (SEQ ID NOs: 160-171) or (N,D)DXX(S,T)XXXE (SEQ ID NOs: 172-175). In certain embodiments, the exogenous synthase contains at least one of the amino acid sequences (N,D)D(L,I,V)X(S,T)XXXE (SEQ ID NOs: 160-171) or (N,D)DXX(S,T)XXXE (SEQ ID NOs: 172-175) between 130 to 180 amino acid residues downstream of one of the sequences specified in SEQ ID NOs: 71-130, 151-175. The (N,D)D(L,I,V)X(S,T)XXXE motif and (N,D)DXX(S,T)XXXE motif are also involved in divalent metal ion binding (typically of Mg²⁺) within the active site of the enzyme. In some embodiments, the exogenous synthase contains at least one of the amino acid sequences specified in SEQ ID NOs: 51-175, or any combination thereof.

In some embodiments, the exogenous plant synthase is a terpene synthase. A terpene synthase refers to any enzyme that enzymatically modifies isopentenyl pyrophosphate (IPP), dimethylallyl pyrophosphate (DMAPP), or a polyprenyl pyrophosphate, such that a terpene or a terpenoid precursor compound is produced. In plants, terpene synthases (TPSs) are responsible for the synthesis of the various terpene molecules from 5-carbon isoprene “building blocks” (C₅H₈), leading to 5-carbon hemiterpenes, 10-carbon monoterpenes, 15-carbon sesquiterpenes, 20-carbon diterpenes, 25 carbon sesterterpenes, and so on. In particular, one or more molecules of isopentenyl pyrophosphate (isopentenyl diphosphate or IPP) and its isomer dimethylallyl pyrophosphate (dimethylallyl diphosphate or DMAPP) undergo condensation to polyprenyl diphosphates, such as geranyl disphosphate (GPP), farnesyl diphosphate (FPP), or geranylgeranyl diphosphate (GGPP). The terpene synthase modifies the polyprenyl diphosphate substrate by cyclizing, rearranging, or coupling the substrate, yielding an isoprenoid or isoprenoid precursor. Modification of GPP to generate a monoterpene, FPP to generate a sesquiterpene, or geranylgeranyl diphosphate GGPP to generate a diterpene, is accomplished through the action of the prenyl disphosphate synthases: GPP synthase, FPP synthase, and GGPP synthase, respectively.

Examples of terpene synthases include, but are not limited to: amorphadiene synthase, bisabolene synthase, cadinene synthase, camphene synthase, caryophyllene synthase, cineole synthase, farnesene synthase, geraniol synthase, germacrene A synthase, germacrene D synthase, humulene synthase, limonene synthase, linanalool synthase, myrcene synthase, ocimene synthase, pinene synthase, sabinene synthase, selinene synthase, as well as synthases producing isomers and stereoisomers of the various terpenes.

In some embodiments, the exogenous synthase catalyzes production of a volatile organic compound. In some embodiments, the volatile organic compound is not endogenously produced. In some embodiments, the volatile organic compound is any plant volatile organic compound. For example, in some embodiments, the volatile organic compound is isoprene or an isoprenoid (“an isoprene derivative”). More specifically, in some embodiments, the volatile organic compound is a terpene. More specifically, in some embodiments, the volatile organic compound is a hemiterpene, monoterpene, diterpene, triterpene, sesquiterpene, sesterterpine, polyterpene, or any combination thereof. More specifically, in some embodiments, the volatile organic compound is the monoterpene limonene.

Examples of isoprenoids produced by terpene synthases include, but are not limited to: hemiterpenes, monoterpenes, diterpenes, triterpenes, and polyterpenes. I-leniterpenes consist of a single isoprene unit. Isoprene itself is considered the only hemiterpene and has the molecular formula C₅H₈.

Monoterpenes and monoterpenoids are made of two isoprene units, and have the molecular formula C₁₀H₁₆ Examples include: anethole, ascaridole, borneol, bornyl acetate, camphene, camphor, carene, carveol, carvone, carvacrol, 1,8-cineole, citral, citronellol, p-cymene geraniol, geranial, eucalyptol, eugenol, shinokitiol, limonene, linalool, menthol, myrcene, neral, nerol, ocimene, perillyl alcohol, phellandrene, a-pinene, P-pinene, pulegone, sabinene, terpineol, terpinene, terpinene-4-ol, terpinolene, thujene, thujone, thymol, umbellulone, and derivatives of these.

Diterpenes are made of four isoprene units, and have the molecular formula C₂₀H₃₂. Examples include: cafestol, cembrene, casbene, eleutherobin, ginkgolide, kahweol, paclitaxel, prostratin, and pseudopterosin, and taxadiene; triterpenes, including but not limited to, arbruside, bruceantin, testosterone, progesterone, cortisone, digitoxin. Isoprenoids also include, but are not limited to, carotenoids such as lycopene, α- and β-carotene, α- and β-cryptoxanthin, bixin, zeaxanthin, astaxanthin, and lutein, and derivatives of these. Isoprenoids also include, but are not limited to, triterpenes, steroid compounds, and compounds that are composed of isoprenoids modified by other chemical groups, such as mixed terpene-alkaloids, and coenzyme Q-10.

Triterpenes consist of six isoprene units, and have the molecular formula C₃₀H₄₈. Tetraterpenes contain eight isoprene units, and have the molecular formula C₄₀H₆₄.

Sesquiterpenes are composed of three isoprene units, and have the molecular formula C₁₅H₂₄. Examples include: aromadedndrane, alloaromadendrene, amorphadiene, amorphene, aristolochene, artemisinin, artemisinic acid, bergamotene, bisabolane, bisabolene, bourbonane, bourbonene, bulgarene, cacalol, cadinene, cadinol, calacorene, calamene, calarene, caryophyllene, cedrane, cedrene, cedrol, chamigrane, copaene, cubebene, cubenol, curcumene, cupranane, drimane, daucane, elemane, elemene, eremophilane, eudesmane, farnesene, farnesol, forskolin, germacrene, himalachane, humulane, humulene, gossypol, guaiene, gurjunene, himachalane, maaliene, muurolene, muurolol, nerolidol, nootkatone, patchoulane, patchoulol, periplanone, sanonin, santatol, scapanene, selinene, silphinene, valencene, viridiflorene, ylangene, zingiberene, and derivatives of these.

Sesterterpenes are made of five isoprene units, and have the molecular formula C₂₅H₄₀. An example of a sesterterenes is geranylfarnesol.

Other isoprenoids include abietadiene or geranylgeraniol.

The terpene skeletons can be further chemically modified (e.g., via oxidation or rearrangement of the carbon skeleton) by various enzymes, such as the cytochrome P450 oxygenases (CYPs), dehydrogenases, methyltransferases, acyltransferases, and glycosyltransferases to form more diverse compounds, known as terpenoids or isoprenoids.

In some embodiments, the enzyme limonene synthase comprises at least one amino acid sequence selected from SEQ ID NOs: 1, 3, 5, 7, 9, 11, 13, 15, 17, 19, 21, 23, 25, 27, 29, 31, 33, or 35-38, or fragments thereof. In some embodiments, the enzyme limonene synthase comprises at least one amino acid sequence that is substantially homologous to an amino acid sequence selected from SEQ ID NOs: 1, 3, 5, 7, 9, 11, 13, 15, 17, 19, 21, 23, 25, 27, 29, 31, 33, or 35-38, or fragments thereof. For example, in certain embodiments, the amino acid sequence has a degree of identity with respect to the original amino acid sequence of at least about 50%, at least about 55%, at least about 60%, of at least about 65%, of at least about 70%, of at least about 75%, of at least about 80%, of at least about 85%, of at least about 90%, of at least about 91%, of at least about 92%, of at least about 93%, of at least about 94%, of at least about 95%, of at least about 96%, of at least about 97%, of at least about 98%, of at least about 99%, or of at least about 99.5% to an amino acid sequence selected from SEQ ID NOs: 1, 3, 5, 7, 9, 11, 13, 15, 17, 19, 21, 23, 25, 27, 29, 31, 33, or 35-38, or fragments thereof.

In certain embodiments, the enzyme limonene synthase comprises an amino acid sequence that has one or more, two or more, three or more, four or more, five or more, six or more, seven or more, eight or more, nine or more, or ten or more mutations, such as point mutations, relative to an amino acid sequence selected from SEQ ID NOs: 1, 3, 5, 7, 9, 11, 13, 15, 17, 19, 21, 23, 25, 27, 29, 31, 33, or 35-38.

In some embodiments, the nucleotide sequence encoding the enzyme limonene synthase comprises at least one nucleotide sequence that encodes an amino acid sequence selected from SEQ ID NOs: 1, 3, 5, 7, 9, 11, 13, 15, 17, 19, 21, 23, 25, 27, 29, 31, 33, or 35-38, or fragments thereof. In some embodiments, the nucleotide sequence encoding the enzyme limonene synthase comprises at least one nucleotide sequence encoding an amino acid sequence that is substantially homologous to an amino acid sequence selected from SEQ ID NOs: 1, 3, 5, 7, 9, 11, 13, 15, 17, 19, 21, 23, 25, 27, 29, 31, 33, or 35-38, or fragments thereof. For example, in certain embodiments, the nucleotide sequence encoding the enzyme limonene synthase comprises at least one nucleotide sequence encoding the amino acid sequence having a degree of identity with respect to the original amino acid sequence of at least about 50%, at least about 55%, at least about 60%, of at least about 65%, of at least about 70%, of at least about 75%, of at least about 80%, of at least about 85%, of at least about 90%, of at least about 91%, of at least about 92%, of at least about 93%, of at least about 94%, of at least about 95%, of at least about 96%, of at least about 97%, of at least about 98%, of at least about 99%, or of at least about 99.5% to an amino acid sequence selected from SEQ ID NOs: 1, 3, 5, 7, 9, 11, 13, 15, 17, 19, 21, 23, 25, 27, 29, 31, 33, or 35-38, or fragments thereof.

In certain embodiments, the nucleotide sequence encoding the enzyme limonene synthase comprises at least one nucleotide sequence that encodes an amino acid sequence that has one or more, two or more, three or more, four or more, five or more, six or more, seven or more, eight or more, nine or more, or ten or more mutations, such as point mutations, substitutions, deletions, duplications, inversions, or insertions relative to an amino acid sequence selected from SEQ ID NOs: 1, 3, 5, 7, 9, 11, 13, 15, 17, 19, 21, 23, 25, 27, 29, 31, 33, or 35-38.

In some embodiments, the nucleotide sequence encoding an exogenous synthase comprises at least one nucleotide sequence that encodes at least one amino acid sequence selected from SEQ ID NOs: 51-175.

In various embodiments, the nucleic acid molecule encoding an exogenous synthase comprises at least one vector. For example, in some embodiments, the present invention also includes a vector in which the isolated nucleic acid of the present invention is inserted. The art is replete with suitable vectors that are useful in the present invention.

In some embodiments, the vector comprises at least one selected from any viral vector known in the art, including but not limited to adenovirus, retrovirus, adeno-associated virus, herpes virus, lentivirus, poxvirus, vaccina virus, or any combination thereof.

Thus, in some embodiments, the nucleic acid molecule encoding an exogenous synthase comprises at least one nucleotide sequence selected from SEQ ID NOs: 2, 4, 6, 8, 10, 12, 14, 16, 18, 20, 22, 24, 26, 28, 30, 32, 34, or 45-50, or fragments thereof. In some embodiments the nucleic acid molecule encoding an exogenous synthase comprises at least one nucleotide sequence that is substantially homologous to a nucleotide sequence selected from SEQ ID NOs: 2, 4, 6, 8, 10, 12, 14, 16, 18, 20, 22, 24, 26, 28, 30, 32, 34, or 45-50. For example, in certain embodiments, the nucleotide sequence has a degree of identity with respect to the original nucleotide sequence of at least about 50%, at least about 55%, at least about 60%, of at least about 65%, of at least about 70%, of at least about 75%, of at least about 80%, of at least about 85%, of at least about 90%, of at least about 91%, of at least about 92%, of at least about 93%, of at least about 94%, of at least about 95%, of at least about 96%, of at least about 97%, of at least about 98%, of at least about 99%, or of at least about 99.5% to a nucleotide sequence selected from SEQ ID NOs: 2, 4, 6, 8, 10, 12, 14, 16, 18, 20, 22, 24, 26, 28, 30, 32, 34, or 45-50, or fragments thereof.

In certain embodiments, the nucleic acid molecule encoding an exogenous synthase comprises a nucleotide sequence that has one or more, two or more, three or more, four or more, five or more, six or more, seven or more, eight or more, nine or more, or ten or more mutations, such as point mutations, base substitutions, deletions, duplications, inversions, or insertions relative to a nucleotide sequence selected from SEQ ID NOs: 2, 4, 6, 8, 10, 12, 14, 16, 18, 20, 22, 24, 26, 28, 30, 32, 34, or 45-50.

In brief summary, the expression of natural or synthetic nucleic acids encoding a peptide of the invention is typically achieved by operably linking a nucleic acid encoding the peptide or portions thereof to a promoter, and incorporating the construct into an expression vector. The vectors to be used are suitable for replication and, optionally, integration in eukaryotic cells. Typical vectors contain transcription and translation terminators, initiation sequences, and promoters useful for regulation of the expression of the desired nucleic acid sequence.

The vectors of the present invention may also be used gene therapy, using standard gene delivery protocols. Methods for gene delivery are known in the art. In another embodiment, the invention provides a gene therapy vector.

The isolated nucleic acid of the invention can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the vector may be provided to a cell in the form of a viral vector. Viruses, which are useful as vectors include, but are not limited to, retroviruses, adenoviruses, adeno-associated viruses, herpes viruses, and lentiviruses, poxviruses, and vaccinia viruses. In general, a suitable vector contains an origin of replication functional in at least one organism, a promoter sequence, convenient restriction endonuclease sites, and one or more selectable markers.

A number of viral based systems have been developed for gene transfer into mammalian cells.

For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In one embodiment, the composition includes a vector derived from an adeno-associated virus (AAV). Adeno-associated viral (AAV) vectors have become powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method.

In certain embodiments, the vector also includes conventional control elements which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α(EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Enhancer sequences found on a vector also regulates expression of the gene contained therein. Typically, enhancers are bound with protein factors to enhance the transcription of a gene. Enhancers may be located upstream or downstream of the gene it regulates. Enhancers may also be tissue-specific to enhance transcription in a specific cell or tissue type. In one embodiment, the vector of the present invention comprises one or more enhancers to boost transcription of the gene present within the vector.

In various embodiments, the nucleic acid molecule encoding an exogenous synthase is codon-optimized for mammalian cells, for example for human cells.

In some embodiments, the composition further comprises a gene delivery vector containing a nucleotide sequence encoding 3-hydroxy-3-methylglutaryl coenzyme-A (HMG-CoA) reductase (HMGR). In some embodiments, the composition comprises a gene delivery vector containing multiple copies of a nucleotide sequence encoding HMGR to increase its expression in cells.

In some embodiments, the composition comprises at least one gene delivery vector containing at least one nucleotide sequence encoding a truncated form of HMGR. In a preferred embodiment, the composition comprises at least one gene delivery vector containing at least one nucleotide sequence encoding HMGR with truncation or deletion of its regulatory domain so as to prevent feedback inhibition of the mevalonate biochemical pathway, thereby increasing production of precursors of VOCs of interest, such as limonene. In a preferred embodiment, the composition comprises at least one gene delivery vector containing at least one gene encoding only the catalytic portion of HMGR. In some embodiments, the composition comprises a gene delivery vector containing multiple copies of a nucleotide sequence encoding a truncated form HMGR to increase its expression in cells. In some embodiments, the gene delivery vector comprises at least one nucleotide sequence that is at least about 70% identical to a nucleotide sequence selected from SEQ ID NO: 39 or a fragment thereof, or SEQ ID NO: 41 or a fragment thereof. In some embodiments, the truncated HMGR comprises at least one amino acid sequence that is at least about 70% identical to an amino acid sequence selected from SEQ ID NO: 40 or a fragment thereof.

In some embodiments, the nucleic acid molecule encoding a truncated HMGR comprises at least one nucleotide sequence selected from SEQ ID NOs: 39 or 41, or fragments thereof. In some embodiments the nucleic acid molecule encoding a truncated HMGR comprises at least one nucleotide sequence comprises at least one nucleotide sequence that is substantially homologous to a nucleotide sequence selected from SEQ ID NOs: 39 or 41. For example, in certain embodiments, the nucleotide sequence has a degree of identity with respect to the original nucleotide sequence of at least about 50%, at least about 55%, at least about 60%, of at least about 65%, of at least about 70%, of at least about 75%, of at least about 80%, of at least about 85%, of at least about 90%, of at least about 91%, of at least about 92%, of at least about 93%, of at least about 94%, of at least about 95%, of at least about 96%, of at least about 97%, of at least about 98%, of at least about 99%, or of at least about 99.5% to the nucleotide sequence selected from SEQ ID NOs: 39 or 41, or fragments thereof.

In certain embodiments, the nucleic acid molecule encoding a truncated HMGR comprises a nucleotide sequence that has one or more, two or more, three or more, four or more, five or more, six or more, seven or more, eight or more, nine or more, or ten or more mutations, such as point mutations, base substitutions, deletions, duplications, inversions, or insertions relative to a nucleotide sequence selected from SEQ ID NOs: 39 or 41.

In some embodiments, the truncated HMGR comprises at least one amino acid sequence set forth in SEQ ID NO: 40, or fragments thereof. In some embodiments, the truncated HMGR comprises at least one amino acid sequence that is substantially homologous to the amino acid sequence set forth in SEQ ID NO: 40, or fragments thereof. For example, in certain embodiments, the amino acid sequence has a degree of identity with respect to the original amino acid sequence of at least about 50%, at least about 55%, at least about 60%, of at least about 65%, of at least about 70%, of at least about 75%, of at least about 80%, of at least about 85%, of at least about 90%, of at least about 91%, of at least about 92%, of at least about 93%, of at least about 94%, of at least about 95%, of at least about 96%, of at least about 97%, of at least about 98%, of at least about 99%, or of at least about 99.5% to the amino acid sequence set forth in SEQ ID NO: 40, or fragments thereof.

In certain embodiments, the truncated HMGR comprises an amino acid sequence that has one or more, two or more, three or more, four or more, five or more, six or more, seven or more, eight or more, nine or more, or ten or more mutations, such as amino acid substitutions, additions, or deletions relative to an amino acid sequence set forth in SEQ ID NO: 40.

In various embodiments, the composition comprises at least one tumor-specific promoter. For example, in one embodiment, the tumor-specific promoter is a lung tumor-specific promoter. In other embodiments, the tumor-specific promoter can be any suitable tumor-specific promoter known in the art including, but not limited to, Survivin promoter, a pan-tumor promoter (SEQ ID NO: 176); hTert promoter, a pan-tumor promoter (SEQ ID NO: 177); CXCR4 promoter tumor-specific in melanomas [GenBank ID: U81003.1] (SEQ ID NO: 178); Hexokinase type II promoter tumor-specific in lung cancer [GenBank: AF148512.1] (SEQ ID NO: 179); TRPM4 (Transient Receptor Potential-Melastatin 4) promoter is preferentially active in prostate cancer; stromelysin 3 promoter is specific for breast cancer cells [GenBank: AF297645.1] (SEQ ID NO: 180); surfactant protein A promoter specific for non-small cell lung cancer cells; secretory leukoprotease inhibitor (SLPI) promoter specific for SLPI-expressing carcinomas; tyrosinase promoter specific for melanoma cells [GenBank: U03039.1](SEQ ID NO: 181); stress-inducible grp78/BiP promoter specific for fibrosarcoma/tumorigenic cells; interleukin-10 promoter specific for glioblastoma multiform cells [GenBank: Z30175.1](SEQ ID NO: 182); α-B-crystallin/heat shock protein 27 promoter specific for brain tumor cells; epidermal growth factor receptor promoter specific for squamous cell carcinoma, glioma, and breast tumor cells [GenBank: J03206.1] (SEQ ID NO: 183); mucin-like glycoprotein (DF3, MUC1) promoter specific for breast carcinoma cells [GenBank: X69118.1] (SEQ ID NO: 184); mts 1 promoter specific for metastatic tumors; NSE promoter specific for small-cell lung cancer cells; somatostatin receptor promoter specific for small cell lung cancer cells [GenBank: AB260891.1] (SEQ ID NO: 185); c-erbB-2 [GenBank ID: M16892.1] (SEQ ID NO: 186), c-erbB-3 [GenBank ID: Z23134.1](SEQ ID NO: 187), and c-erbB-4 promoters are specific for breast cancer cells; cerbB4 promoter specific for breast and gastric cancer cells; thyroglobulin promoter specific for thyroid carcinoma cells [GenBank: X77275.1](SEQ ID NO: 188); α-fetoprotein promoter specific for hepatoma cells [GenBank: AB053572.1](SEQ ID NO: 189); villin promoter specific for gastric cancer cells [GenBank: EF184645.1]—SEQ ID NO: 190; and albumin promoter specific for hepatoma cells SEQ ID NO: 191. Additional examples of suitable promoters are an ATP binding cassette subfamily C member 4 (ABCC4) promoter, an anterior gradient 2, protein disulphide isomerase family member (AGR2) promoter, activation induced cytidine deaminase (AICDA) promoter, an UDP-GlcNAc:betaGal beta-1,3-N-acetylglucosaminyltransf erase 3 (B3GNT3) promoter, a cadherin 3 (CDH3) promoter, a CEA cell adhesion molecule 5 (CEACAM5) promoter, a centromere protein F (CENPF) promoter, a centrosomal protein 55 (CEP55) promoter, a claudin 3 (CLDN3) promoter, a claudin 4 (CLDN4) promoter, a collagen type XI alpha 1 chain (COL11 A1) promoter, a collagen type I alpha 1 chain (COL1 A1) promoter, a cystatin SN (CST1) promoter, a denticleless E3 ubiquitin protein ligase homolog (DTL) promoter, a family with sequence similarity 111 member B (FAM1 lIB) promoter, a forkhead box A1 (FOXA1) promoter, a kinesin family member 20 A (KIF20 A), a laminin subunit gamma 2 (LAMC2) promoter, a mitotic spindle positioning (MISP) promoter, a matrix metallopeptidase 1 (MMP1) promoter, a matrix metallopeptidase 12 (MMP12) promoter, a matrix metallopeptidase 13 (MMP13) promoter, a mesothelin (MSLN) promoter, a cell surface associated mucin 1 (MUC1) promoter, a phospholipase A2 group IID (PLA2G2D) promoter, a regulator of G protein signaling 13 (RGS13) promoter, a secretoglobin family 2 A member 1 (SCGB2 A1) promoter, topoisomerase II alpha (TOP2 A) promoter, a ubiquitin D (UBD) promoter, a ubiquitin conjugating enzyme E2 C (UBE2C), a USHl protein network component harmonin (USH1C), a V-set domain containing T cell activation inhibitor 1 (VTCN1) promoter, a ubiquitin conjugating enzyme E2 T (UBE2T) promoter, a checkpoint kinase 1 (CHEK1) promoter, an epithelial cell transforming 2 promoter (ECT2), a BCL2-like 12 (BCL2L12) promoter, a centromere protein I (CENPI) promoter, an E2F transcription factor 1 (E2F1) promoter, a flavin adenine dinucleotide synthetase 1 (FLAD1) promoter, a protein phosphatase, Mg2+/Mn2+ dependent 1G (PPM1G) promoter, an ubiquitin conjugating enzyme E2 S (EIBE2S) promoter, an aurora kinase A and ninein interacting protein (AUNIP) promoter, a cell division cycle 6 (CDC6) promoter, a centromere protein L (CENPL) promoter, a DNA replication helicase/nuclease 2 (DNA2) promoter, a DSN1 homolog, MIS 12 kinetochore complex component (DSN1) promoter, a deoxythymidylate kinase (DTYMK) promoter, a G protein regulated inducer of neurite outgrowth 1 (GPRIN1) promoter, a mitochondrial fission regulator 2 (MTFR2) promoter, a RAD51 associated protein 1 (RAD51AP1) promoter, a small nuclear ribonucleoprotein polypeptide A′ (SNRPA1) promoter, an ATPase family, AAA domain containing 2 (ATAD2) promoter, a BUB1 mitotic checkpoint serine/threonine kinase (BUB1) promoter, a calcyclin binding protein (CACYBP) promoter, a cell division cycle associated 3 (CDCA3) promoter, a centromere protein O (CENPO) promoter, a flap structure-specific endonuclease 1 (FEN1) promoter, a forkhead box Ml (FOXM1) promoter, a cell proliferation regulating inhibitor of protein phosphatase 2 A (KIAA1524) promoter, a kinesin family member 2C (KIF2C) promoter, a karyopherin subunit alpha 2 (KPNA2) promoter, a MYB protooncogene like 2 (MYBL2) promoter, a NIMA related kinase 2 (NEK2) promoter, a RAN binding protein 1 (RANBP1) promoter, a small nuclear ribonucleoprotein polypeptides B and B 1 (SNRPB) promoter, a SPC24/NDC80 kinetochore complex component (SPC24) promoter, a transforming acidic coiled-coil containing protein 3 (TACC3) promoter, a TBC1 domain family member 31 (TBC1D31) promoter, a thymidine kinase 1 (TK1) promoter, a zinc finger protein 695 (ZNF695) promoter, an aurora kinase A (AURKA) promoter, a BLM RecQ like helicase (BLM) promoter, a chromosome 17 open reading frame 53 (C17 or f53) promoter, a chromobox 3 (CBX30) promoter, a cyclin B 1 (CCNBl) promoter, a cyclin E1 (CCNEl) promoter, a cyclin F (CCNF), a cell division cycle 20 (CDC20) promoter, a cell division cycle 45 (CDC45) promoter, a cell division cycle associated 5 (CDCA5) promoter, a cyclin dependent kinase inhibitor 3 (CDKN3) promoter, a cadherin EGF LAG seven-pass G-type receptor 3 (CELSR3) promoter, a centromere protein A (CENPA) promoter, a centrosomal protein 72 (CEP72) promoter, a CDC28 protein kinase regulatory subunit 2 (CKS2) promoter, a collagen type X alpha 1 chain (COL1OA1) promoter, a chromosome segregation 1 like (CSE1L) promoter, a DBF4 zinc finger promoter, a GINS complex subunit 1 (GINS1) promoter, a G protein-coupled receptor 19 (GPR19) promoter, a kinesin family member 18 A (KIF18 A) promoter, a kinesin family member 4 A (KIF4 A) promoter, a kinesin family member Cl (KIFC1) promoter, a minichromosome maintenance 10 replication initiation factor (MCM10) promoter, a minichromosome maintenance complex component 2 (MCM2) promoter, a minichromosome maintenance complex component 7 (MCM7) promoter, a MRG domain binding protein (MRGBP) promoter, a methylenetetrahydrofolate dehydrogenase (NADP+ dependent) 2, methenyltetrahydrofolate cyclohydrolase (MTHFD2) promoter, a non-SMC condensin I complex subunit H (NCAPH) promoter, aNDC80, kinetochore complex component (NDC80) promoter, a nudix hydrolase 1 (NUDT1) promoter, a ribonuclease H2 subunit A (RNASEH2 A) promoter, a RuvB like AAA ATPase 1 (RUVBL1) promoter, a serologically defined breast cancer antigen NY-BR-85 (SGOL1) promoter, a SHC binding and spindle associated 1 (SHCBP1) promoter, a small nuclear ribonucleoprotein polypeptide G (SNRPG) promoter, a timeless circadian regulator promoter, a thyroid hormone receptor interactor 13 (TRIP 13) promoter, a trophinin associated protein (TROAP) promoter, a ubiquitin conjugating enzyme E2 C (UBE2C) promoter, aWD repeat and HMG-box DNA binding protein 1 (WDHD1) promoter, a functional fragment thereof, or any combination thereof.

In some embodiments, the tumor-specific promoter comprises at least one amino acid sequence that is at least about 70% identical to an amino acid sequence selected from Survivin promoter, human (SEQ ID NO: 176), hTert core promoter, human (SEQ ID NO: 177), CXCR4 promoter, human [GenBank ID: U81003.1](SEQ ID NO: 178), Hexokinase type II promoter, human [GenBank: AF148512.1] (SEQ ID NO: 179), Stromelysin 3 (MMP11) promoter, mouse [GenBank: AF297645.1] (SEQ ID NO: 180), Tyrosinase promoter, human, [GenBank: U03039.1] (SEQ ID NO: 181)Interleukin-10 promoter, human [GenBank: Z30175.1] (SEQ ID NO: 182), Epidermal growth factor receptor (EGFR) promoter, [GenBank: J03206.1](SEQ ID NO: 183), Mucin-like glycoprotein (DF3, MUC1) promoter, [GenBank: X69118.1] (SEQ ID NO: 184), Somatostatin receptor 2 (sst2)promoter, human [GenBank: AB260891.1] (SEQ ID NO: 185), c-erbB-2 promoters, human [GenBank ID: M16892.1] (SEQ ID NO: 186), c-erbB-3 promoter; human [GenBank ID: Z23134.1] (SEQ ID NO: 187), Thyroglobulin promoter, human [GenBank: X77275.1] (SEQ ID NO: 188), alpha-fetoprotein (AFP) promoter, human [GenBank: AB053572.1] (SEQ ID NO: 189), Villin 2 promoter, human [GenBank: EF184645.1] (SEQ ID NO: 190), or Albumin promoter (SEQ ID NO: 191).

In certain embodiments, the tumor-specific promoter comprises a nucleotide sequence that has one or more, two or more, three or more, four or more, five or more, six or more, seven or more, eight or more, nine or more, or ten or more mutations, such as point mutations, base substitutions, deletions, duplications, inversions, or insertions relative to a nucleotide sequence selected from Survivin promoter, human (SEQ ID NO: 176), hTert core promoter, human (SEQ ID NO: 177), CXCR4 promoter, human [GenBank ID: U81003.1](SEQ ID NO: 178), Hexokinase type promoter, human [GenBank: AF148512.1] (SEQ ID NO: 179), Stromelysin 3 (MMP11) promoter, mouse [GenBank: AF297645.1] (SEQ ID NO: 180), Tyrosinase promoter, human, [GenBank: U03039.1] (SEQ ID NO: 181)Interleukin-10 promoter, human [GenBank: Z30175.1] (SEQ ID NO: 182), Epidermal growth factor receptor 10 (EGFR) promoter, [GenBank: J03206.1](SEQ ID NO: 183), Mucin-like glycoprotein (DF3, MUC1) promoter, [GenBank: X69118.1] (SEQ ID NO: 184), Somatostatin receptor 2 (sst2)promoter, human [GenBank: AB260891.1] (SEQ ID NO: 185), c-erbB-2 promoters, human [GenBank ID: M16892.1] (SEQ ID NO: 186), c-erbB-3 promoter; human [GenBank ID: Z23134.1] (SEQ ID NO: 187), Thyroglobulin promoter, human [GenBank: X77275.1] (SEQ ID NO: 188), alpha-fetoprotein (AFP) promoter, human [GenBank: AB053572.1] (SEQ ID NO: 189), Villin 2 promoter, human [GenBank: EF184645.1] (SEQ ID NO: 190), or Albumin promoter (SEQ ID NO: 191).

In various embodiments, the composition comprises at least one agent that acts on the mevalonate pathway to increase production of a VOC of interest (e.g., limonene).

In various embodiments, the composition is a genetic delivery vector, minicircle, liposome, or any combination thereof.

### Pharmaceutical Composition

The present invention also provides pharmaceutical compositions comprising at least one exogenous synthase (e.g., limonene synthase, such as SEQ ID NOs: 1, 3, 5, 7, 9, 11, 13, 15, 17, 19, 21, 23, 25, 27, 29, 31, 33, or 35-38, or an exogenous synthase containing an amino acid sequence motif selected from SEQ ID Nos: 51-175 or any combination thereof) or nucleic acid molecule encoding thereof (e.g., vector comprising a nucleic acid sequence encoding limonene synthase, such as SEQ ID NOs: 2, 4, 6, 8, 10, 12, 14, 16, 18, 20, 22, 24, 26, 28, 30, 32, 34, or 45-50).

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

In exemplary embodiments, a pharmaceutical composition comprises a pharmaceutically acceptable excipient, such as a pharmaceutically acceptable carrier, and an exemplary compound described herein.

In certain exemplary embodiments, the pharmaceutical composition comprises, or is in the form of, a pharmaceutically acceptable salt, as generally described below.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, intraocular, oral, rectal, vaginal, parenteral, topical, pulmonary, intranasal, buccal, intravenous, intracerebral, intracerebroventricular, intradermal, transdermal, intramuscular, intrauterine, subcutaneous, sublingual, endotracheal, transungual, transmucosal, inhalational (nebulized form), intestinal, intramedullary, intrathecal, intravascular, intraperitoneal, direct intraventricular, intra-arterial, transcatheter, or another route of administration. Other contemplated formulations include nanoparticles, liposomal preparations, viral vector, exosome, extracellular vesicles, naked DNA (including naked plasmids or minicircles), resealed erythrocytes containing the active ingredient, and antibody-based or targeted formulations.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 99.99% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

In one embodiment, the pharmaceutical composition has increased bioavailability.

In one embodiment, the pharmaceutical composition has increased solubility. In some embodiments, the pharmaceutical composition comprises at least one pharmaceutical vehicle.

In one embodiment, the at least one nucleic acid molecule encoding at least one exogenous synthase (e.g., limonene synthase, such as SEQ ID NOs. 1, 3, 5, 7, 9, 11, 13, 15, 17, 19, 21, 23, 25, 27, 29, 31, 33, or 35-38, or an exogenous synthase containing an amino acid sequence motif selected from SEQ ID NOs: 51-175 or any combination thereof) or nucleic acid molecule encoding thereof (e.g., vector comprising a nucleic acid molecule encoding limonene synthase, such as SEQ ID NOs: 2, 4, 6, 8, 10, 12, 14, 16, 18, 20, 22, 24, 26, 28, 30, 32, 34, or 45-50) solubilized in a pharmaceutical vehicle has a solubility range of 0.001 mg/L-10.0 g/mL. For example, in one embodiment, the at least one exogenous synthase (e.g., limonene synthase, such as SEQ ID NOs: 1, 3, 5, 7, 9, 11, 13, 15, 17, 19, 21, 23, 25, 27, 29, 31, 33, or 35-38, or an exogenous synthase containing an amino acid sequence motif selected from SEQ ID NOs: 51-175 or any combination thereof) or nucleic acid molecule encoding thereof (e.g., vector comprising a nucleic acid molecule encoding limonene synthase, such as SEQ ID NOs: 2, 4, 6, 8, 10, 12, 14, 16, 18, 20, 22, 24, 26, 28, 30, 32, 34, or 45-50) has a solubility of 0.001 mg/mL. In one embodiment, the at least one exogenous synthase (e.g., limonene synthase, such as SEQ ID NOs: 1, 3, 5, 7, 9, 11, 13, 15, 17, 19, 21, 23, 25, 27, 29, 31, 33, or 35-38, or an exogenous synthase containing an amino acid sequence motif selected from SEQ ID NOs: 51-175 or any combination thereof) or nucleic acid molecule encoding thereof (e.g., vector comprising a nucleic acid molecule encoding limonene synthase, such as SEQ ID NOs: 2, 4, 6, 8, 10, 12, 14, 16, 18, 20, 22, 24, 26, 28, 30, 32, 34, or 45-50) has a solubility of 0.03 mg/mL. In one embodiment, the at least one exogenous synthase (e.g., limonene synthase, such as SEQ ID NOs: 1, 3, 5, 7, 9, 11, 13, 15, 17, 19, 21, 23, 25, 27, 29, 31, 33, or 35-38, or an exogenous synthase containing an amino acid sequence motif selected from SEQ ID NOs: 51-175 or any combination thereof) or nucleic acid molecule encoding thereof (e.g., vector comprising a nucleic acid molecule encoding limonene synthase, such as SEQ ID NOs: 2, 4, 6, 8, 10, 12, 14, 16, 18, 20, 22, 24, 26, 28, 30, 32, 34, or 45-50) has a solubility of 500.0 mg/mL. In one embodiment, the at least one exogenous synthase (e.g., limonene synthase, such as SEQ ID NOs: 1, 3, 5, 7, 9, 11, 13, 15, 17, 19, 21, 23, 25, 27, 29, 31, 33, or 35-38, or an exogenous synthase containing an amino acid sequence motif selected from SEQ ID NOs: 51-175 or any combination thereof) or nucleic acid molecule encoding thereof (e.g., vector comprising a nucleic acid molecule encoding limonene synthase, such as SEQ ID NOs: 2, 4, 6, 8, 10, 12, 14, 16, 18, 20, 22, 24, 26, 28, 30, 32, 34, or 45-50) has a solubility of 5.0 g/mL. In one embodiment, the at least one exogenous synthase (e.g., limonene synthase, such as SEQ ID NOs: 1, 3, 5, 7, 9, 11, 13, 15, 17, 19, 21, 23, 25, 27, 29, 31, 33, or 35-38, or an exogenous synthase containing an amino acid sequence motif selected from SEQ ID NOs: 51-175 or any combination thereof) or nucleic acid molecule encoding thereof (e.g., vector comprising a nucleic acid molecule encoding limonene synthase, such as SEQ ID NOs: 2, 4, 6, 8, 10, 12, 14, 16, 18, 20, 22, 24, 26, 28, 30, 32, 34, or 45-50) has a solubility of 10.0 g/mL. (Please note that, due to their length, SEQ ID NOs: 45-50 are only shown in the sequence listing).

In one embodiment, the pharmaceutical vehicle is selected from the group consisting of aqueous buffers, solvents, co-solvents, cyclodextrin complexes, lipid vehicles, and any combination thereof, and optionally further comprising at least one stabilizer, emulsifier, polymer, antioxidant, and any combination thereof.

In one embodiment, the aqueous buffer is selected from the group consisting of aqueous NaCl, aqueous HCl, aqueous citrate-HCl buffer, aqueous NaOH, aqueous citrate-NaOH buffer, aqueous phosphate buffer, aqueous KCl, aqueous borate-KCl—NaOH buffer, PBS buffer, and any combination thereof.

In one embodiment, the aqueous buffer has pH range of pH=0.5-10. In one embodiment, the aqueous buffer has pH range of pH=0.5. In one embodiment, the aqueous buffer has pH=1.0.

In one embodiment, the aqueous buffer has pH=2.0. In one embodiment, the aqueous buffer has pH=3.0. In one embodiment, the aqueous buffer has pH=4.0. In one embodiment, the aqueous buffer has pH=5.0. In one embodiment, the aqueous buffer has pH=5.5. In one embodiment, the aqueous buffer has pH=6.0. In one embodiment, the aqueous buffer has pH=7.0. In one embodiment, the aqueous buffer has pH=7.4. In one embodiment, the aqueous buffer has pH=8.0. In one embodiment, the aqueous buffer has pH=9.0. In one embodiment, the aqueous buffer has pH=9.5. In one embodiment, the aqueous buffer has pH=10.0.

In one embodiment, the aqueous buffer has a concentration range of 0.001 N—1.0 N. In one embodiment, the aqueous buffer has a concentration of 0.05 N. In one embodiment, the aqueous buffer has a concentration of 0.1 N. In one embodiment, the aqueous buffer has a concentration of 0.15 N. In one embodiment, the aqueous buffer has a concentration of 0.2 N. In one embodiment, the aqueous buffer has a concentration of 0.3 N. In one embodiment, the aqueous buffer has a concentration of 0.4 N. In one embodiment, the aqueous buffer has a concentration of 0.5 N. In one embodiment, the aqueous buffer has a concentration of 0.6 N. In one embodiment, the aqueous buffer has a concentration of 0.7 N. In one embodiment, the aqueous buffer has a concentration of 0.8 N. In one embodiment, the aqueous buffer has a concentration of 0.9 N. In one embodiment, the aqueous buffer has a concentration of 1.0 N.

In one embodiment, the solvent is selected from the group consisting of acetone, ethyl acetate, acetonitrile, pentane, hexane, heptane, methanol, ethanol, isopropyl alcohol, dimethyl sulfoxide (DMSO), water, chloroform, dichloromethane, diethyl ether, PEG400, Transcutol (diethylene glycomonoethyl ether), MCT 70, Labrasol (PEG-8 caprylic/capric glycerides), Labrafil M1944CS (PEG 5 Oleate), propylene glycol, Transcutol P, PEG400, propylene glycol, glycerol, Captex 300, Tween 85, Cremophor EL, Maisine 35-1, Maisine CC, Capmul MCM, maize oil, and any combination thereof.

In one embodiment, the co-solvent is selected from the group consisting of acetone, ethyl acetate, acetonitrile, pentane, hexane, heptane, methanol, ethanol, isopropyl alcohol, dimethyl sulfoxide (DMSO), water, chloroform, dichloromethane, diethyl ether, PEG400, Transcutol (diethylene glycomonoethyl ether), MCT 70, Labrasol (PEG-8 caprylic/capric glycerides), Labrafil M1944CS (PEG 5 Oleate), propylene glycol, Transcutol P, PEG400, propylene glycol, glycerol, Captex 300, Tween 85, Cremophor EL, Maisine 35-1, Maisine CC, Capmul MCM, maize oil, and any combination thereof.

In one embodiment, the cyclodextrin complexes is selected from the group consisting of methyl-β-cyclodextrin, methyl-γ-cyclodextrin, HP-β-cyclodextrin, HP-γ-cyclodextrin, SBE-β-cyclodextrin, α-cyclodextrin, γ-cyclodextrin,6-O-glucosyl-β-cyclodextrin, and any combination thereof.

In one embodiment, the lipid vehicle is selected from the group consisting of Captex 300, Tween 85, Cremophor EL, Maisine 35-1, Maisine CC, Capmul MCM, maize oil, and any combination thereof. In one embodiment, the lipid vehicle is an oil. In one embodiment, the lipid vehicle is an oil mixture. In one embodiment, the oil mixture comprises at least two oils. In one embodiment, the oil is selected from the group consisting of Captex 300, Tween 85, Cremophor EL, Maisine 35-1, Maisine CC, Capmul MCM, maize oil, and any combination thereof.

In one embodiment, the stabilizer is selected from the group consisting of Pharmacoat 603, SLS, Nisso HPC-SSL, Kolliphor, PVP K30, PVP VA 64, and any combination thereof. In one embodiment, the stabilizer is an aqueous solution.

In one embodiment, the polymer is selected from the group consisting of HPMC-AS-MG, HPMC-AS-LG, HPMC-AS-HG, HPMC, HPMC-P-55S, HPMC-P-50, methyl cellulose, HEC, HPC, Eudragit L100, Eudragit E100, PEO 100K, PEG 6000, PVP VA64, PVP K30, TPGS, Kollicoat IR, Carbopol 980NF, Povocoat MP, Soluplus, Sureteric, Pluronic F-68, and any combination thereof.

In one embodiment, the pharmaceutical composition is a suspension. In one embodiment, the pharmaceutical composition is a nanosuspension. In one embodiment, the pharmaceutical composition is an emulsion. In one embodiment, the pharmaceutical composition is a solution. In one embodiment, the pharmaceutical composition is a liquid formulation. In one embodiment, the pharmaceutical composition is a cream. In one embodiment, the pharmaceutical composition is a gel. In one embodiment, the pharmaceutical composition is a lotion. In one embodiment, the pharmaceutical composition is a paste. In one embodiment, the pharmaceutical composition is an ointment. In one embodiment, the pharmaceutical composition is an emollient. In one embodiment, the pharmaceutical composition is a liposome. In one embodiment, the pharmaceutical composition a nanosphere. In one embodiment, the pharmaceutical composition is a skin tonic. In one embodiment, the pharmaceutical composition is a mouth wash. In one embodiment, the pharmaceutical composition is an oral rinse. In one embodiment, the pharmaceutical composition is a mousse. In one embodiment, the pharmaceutical composition is a spray. In one embodiment, the pharmaceutical composition is a pack. In one embodiment, the pharmaceutical composition is a capsule. In one embodiment, the pharmaceutical composition is a tablet. In one embodiment, the pharmaceutical composition is a powder. In one embodiment, the pharmaceutical composition is a granule. In one embodiment, the pharmaceutical composition is a patch. In one embodiment, the pharmaceutical composition is a biodegradable, bioresorbable, or dissolving material. In one embodiment, the pharmaceutical composition is a microneedle or microneedle patch. In one embodiment, the pharmaceutical composition is an occlusive skin agent.

In one embodiment, the pharmaceutical composition is a dry powder formulation. In one embodiment, the pharmaceutical composition is a tablet, wherein the tablets, comprising the exogenous synthase (e.g., limonene synthase, such as SEQ ID NOs: 1, 3, 5, 7, 9, 11, 13, 15, 17, 19, 21, 23, 25, 27, 29, 31, 33, or 35-38, or an exogenous synthase containing an amino acid sequence motif selected from SEQ ID NOs: 51-175 or any combination thereof) or nucleic acid molecule encoding thereof (e.g., vector comprising a nucleic acid molecule encoding limonene synthase, such as SEQ ID NOs: 2, 4, 6, 8, 10, 12, 14, 16, 18, 20, 22, 24, 26, 28, 30, 32, 34, or 45-50), are prepared through two manufacturing steps: a granulation step and a tablet preparation step. In one embodiment, the granulation step is a preparation of the intermediate product (IP). In one embodiment, the granulation step comprises a granulating fluid containing excipients in ethanol that is added to primary powder particles and followed by solvent evaporation. In one 10 embodiment, the particle size of the resulting material is reduced by milling. In one embodiment, the tablet preparation step is a preparation of the Drug Product (DP). In one embodiment, an intermediate product (IP), wherein the intermediate product (IP) is obtained from the granulation step, is blended with excipients. In one embodiment, the Drug Product (DP) is tablet compressed by direct compression on a tablet press.

The pharmaceutical compositions and formulations described herein can be administered to a subject per se, or in pharmaceutical compositions where they are mixed with other active ingredients, as in combination therapy, or suitable carriers or excipient(s).

Alternatively, one may administer the compound in a local rather than systemic manner, for example, via injection of the compound directly into the area of pain, often in a depot or sustained release formulation. Furthermore, one may administer the drug in a targeted drug delivery system, for example, in a liposome coated with a tissue-specific antibody. The liposomes will be targeted to and taken up selectively by the organ.

The pharmaceutical compositions and formulations disclosed herein may be manufactured in a manner that is itself known, e.g., by means of conventional mixing, dissolving, granulating, dragee-making, levigating, emulsifying, encapsulating, entrapping or tabletting processes.

Pharmaceutical compositions and formulations for use in accordance with the present disclosure thus may be formulated in a conventional manner using one or more physiologically acceptable carriers comprising excipients and auxiliaries, which facilitate processing of the active compounds into preparations, which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

For injection, the agents disclosed herein may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art.

For oral administration, either solid or fluid unit dosage forms can be prepared. For preparing solid compositions such as tablets, the exogenous synthase (e.g., limonene synthase, such as SEQ ID NOs: 1, 3, 5, 7, 9, 11, 13, 15, 17, 19, 21, 23, 25, 27, 29, 31, 33, or 35-38, or an exogenous synthase containing an amino acid sequence motif selected from SEQ ID NOs: 51-175 or any combination thereof) or nucleic acid molecule encoding thereof (e.g., vector comprising a nucleic acid molecule encoding limonene synthase, such as SEQ ID NOs: 2, 4, 6, 8, 10, 12, 14, 16, 18, 20, 22, 24, 26, 28, 30, 32, 34, or 45-50), disclosed above herein, is mixed into formulations with conventional ingredients such as talc, magnesium stearate, dicalcium phosphate, magnesium aluminum silicate, calcium sulfate, starch, lactose, acacia, methylcellulose, and functionally similar materials as pharmaceutical diluents or carriers. For oral administration, the compounds can be also formulated readily by combining the active compounds with pharmaceutically acceptable carriers well known in the art. Such carriers enable the compounds disclosed herein to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination disclosed herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Capsules are prepared by mixing the compound with an inert pharmaceutical diluent, and filling the mixture into a hard gelatin capsule of appropriate size. Soft gelatin capsules are prepared by machine encapsulation of slurry of the compound with an acceptable vegetable oil, light liquid petrolatum or other inert oil. Fluid unit dosage forms for oral administration such as syrups, elixirs and suspensions can be prepared. The water-soluble forms can be dissolved in an aqueous vehicle together with sugar, aromatic flavoring agents and preservatives to form syrup. An elixir is prepared by using a hydro alcoholic (e.g., ethanol) vehicle with suitable sweeteners such as sugar and saccharin, together with an aromatic flavoring agent. Suspensions can be prepared with an aqueous vehicle with the aid of a suspending agent such as acacia, tragacanth, methylcellulose and the like.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Starch microspheres can be prepared by adding a warm aqueous starch solution, e.g., of potato starch, to a heated solution of polyethylene glycol in water with stirring to form an emulsion.

When the two-phase system has formed (with the starch solution as the inner phase) the mixture is then cooled to room temperature under continued stirring whereupon the inner phase is converted into gel particles. These particles are then filtered off at room temperature and slurred in a solvent such as ethanol, after which the particles are again filtered off and laid to dry in air.

The microspheres can be hardened by well-known cross-linking procedures such as heat treatment or by using chemical cross-linking agents. Suitable agents include dialdehydes, including glyoxal, malondialdehyde, succinic aldehyde, adipaldehyde, glutaraldehyde and phthalaldehyde, diketones such as butadione, epichlorohydrin, polyphosphate, and borate. Dialdehydes are used to crosslink proteins such as albumin by interaction with amino groups, and diketones form schiff bases with amino groups. Epichlorohydrin activates compounds with nucleophiles such as amino or hydroxyl to an epoxide derivative.

Pharmaceutical preparations, which can be used orally, include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers.

In soft capsules, the active compounds may be dissolved or suspended in suitable liquids, such as fatty oils, liquid paraffin, or liquid polyethylene glycols. In addition, stabilizers and/or antioxidants may be added. All formulations for oral administration should be in dosages suitable for such administration.

For buccal administration, the compositions may take the form of tablets or lozenges formulated in conventional manner.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Slow or extended-release delivery systems, including any of a number biopolymers (biological-based systems), systems employing liposomes, colloids, resins, and other polymeric delivery systems or compartmentalized reservoirs, can be utilized with the compositions described herein to provide a continuous or long term source of therapeutic compound. Such slow release systems are applicable to formulations for delivery via topical, intraocular, oral, and parenteral routes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances, which increase the viscosity of the suspension, such as sodium carboxymethyl cellulose, sorbitol, or dextran. Optionally, the suspension may also contain suitable stabilizers or agents, which increase the solubility of the compounds to allow for the preparation of highly, concentrated solutions.

Alternatively, the active ingredient may be in powder form for constitution with a suitable vehicle, e.g., sterile pyrogen-free water, before use.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Many of the compounds used in the pharmaceutical combinations disclosed herein may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acids or base forms.

Pharmaceutical compositions suitable for use in the methods disclosed herein include compositions where the active ingredients are contained in an amount effective to achieve its intended purpose.

The exact formulation, route of administration and dosage for the pharmaceutical compositions disclosed herein can be chosen by the individual physician in view of the patient's condition.

Typically, the dose about the composition administered to the patient can be from about 0.5 to 1000 mg/kg of the patient's body weight, or 1 to 500 mg/kg, or 10 to 500 mg/kg, or 50 to 100 mg/kg of the patient's body weight. The dosage may be a single one or a series of two or more given in the course of one or more days, as is needed by the patient. Note that for almost all of the specific compounds mentioned in the present disclosure, human dosages for treatment of at least some condition have been established. Thus, in most instances, the methods disclosed herein will use those same dosages, or dosages that are between about 0.1% and 500%, or between about 25% and 250%, or between 50% and 100% of the established human dosage. Where no human dosage is established, as will be the case for newly discovered pharmaceutical compounds, a suitable human dosage can be inferred from ED50 or ID50 values, or other appropriate values derived from in vitro or in vivo studies, as qualified by toxicity studies and efficacy studies in animals.

Although the exact dosage will be determined on a drug-by-drug basis, in most cases, some generalizations regarding the dosage can be made. The daily dosage regimen for an adult human patient may be, for example, an oral dose of between 0.1 mg and 2000 mg of each ingredient, preferably between 1 mg and 250 mg, e.g., 5 to 200 mg or an intravenous, subcutaneous, or intramuscular dose of each ingredient between 0.01 mg and 500 mg, preferably between 0.1 mg and 60 mg, e.g., 0.1 to 40 mg of each ingredient of the pharmaceutical compositions disclosed herein or a pharmaceutically acceptable salt thereof calculated as the free base, the composition being administered 1 to 4 times per day. Alternatively, the compositions disclosed herein may be administered by continuous intravenous infusion, preferably at a dose of each ingredient up to 400 mg per day. Thus, the total daily dosage by oral administration of each ingredient will typically be in the range 1 to 2000 mg and the total daily dosage by parenteral administration will typically be in the range 0.1 to 500 mg. Suitably the compounds will be administered for a period of continuous therapy, for example for a week or more, or for months or years.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The pharmaceutical compositions and formulations may be prepared with pharmaceutically acceptable excipients, which may be a carrier or a diluent, as a way of example. Such compositions can be in the form of a capsule, sachet, paper or other container. In making the compositions, conventional techniques for the preparation of pharmaceutical compositions may be used. For example, the exogenous synthase (e.g., limonene synthase, such as SEQ ID NOs: 1, 3, 5, 7, 9, 11, 13, 15, 17, 19, 21, 23, 25, 27, 29, 31, 33, or 35-38, or an exogenous synthase containing an amino acid sequence motif selected from SEQ ID NOs: 51-175 or any combination thereof) or nucleic acid molecule encoding thereof (e.g., vector comprising a nucleic acid molecule encoding limonene synthase, such as SEQ ID NOs: 2, 4, 6, 8, 10, 12, 14, 16, 18, 20, 22, 24, 26, 28, 30, 32, 34, or 45-50) disclosed above herein may be mixed with a carrier, or diluted by a carrier, or enclosed within a carrier that may be in the form of an ampoule, capsule, sachet, paper, or other container. When the carrier serves as a diluent, it may be solid, semi-solid, or liquid material that acts as a vehicle, excipient, or medium for the active compound. The exogenous synthase (e.g., limonene synthase, such as SEQ ID NOs: 1, 3, 5, 7, 9, 11, 13, 15, 17, 19, 21, 23, 25, 27, 29, 31, 33, or 35-38, or an exogenous synthase containing an amino acid sequence motif selected from SEQ ID NOs: 51-175 or any combination thereof) or nucleic acid molecule encoding thereof (e.g., vector comprising a nucleic acid molecule encoding limonene synthase, such as SEQ ID NOs: 2, 4, 6, 8, 10, 12, 14, 16, 18, 20, 22, 24, 26, 28, 30, 32, 34, or 45-50) and compositions comprising the same, for use as described above herein can be adsorbed on a granular solid container for example in a sachet. Some examples of suitable carriers are water, salt solutions, alcohols, polyethylene glycols, polyhydroxyethoxylated castor oil, peanut oil, olive oil, lactose, terra alba, sucrose, cyclodextrin, amylose, magnesium stearate, talc, gelatin, agar, pectin, acacia, stearic acid or lower alkyl ethers of cellulose, silicic acid, fatty acids, fatty acid amines, fatty acid mono glycerides and diglycerides, pentaerythritol fatty acid esters, polyoxyethylene, hydroxymethylcellulose, and polyvinylpyrrolidone. Similarly, the carrier or diluent may include any sustained release material known in the art, such as glyceryl monostearate or glyceryl distearate, alone or mixed with a wax. Said compositions may also include wetting agents, emulsifying and suspending agents, preserving agents, sweetening agents or flavoring agents. The compositions described in present invention may be formulated so as to provide quick, sustained, or delayed release of the exogenous synthase (e.g., limonene synthase, such as SEQ ID NOs: 1, 3, 5, 7, 9, 11, 13, 15, 17, 19, 21, 23, 25, 27, 29, 31, 33, or 35-38, or an exogenous synthase containing an amino acid sequence motif selected from SEQ ID NOs: 51-175, or any combination thereof) or nucleic acid molecule encoding thereof (e.g., vector comprising a nucleic acid molecule encoding limonene synthase, such as SEQ ID NOs: 2, 4, 6, 8, 10, 12, 14, 16, 18, 20, 22, 24, 26, 28, 30, 32, 34, or 45-50) disclosed herein after administration to the patient by employing procedures well known in the art.

The pharmaceutical compositions and formulations can be sterilized and mixed, if desired, with auxiliary agents, emulsifiers, salt for influencing osmotic pressure, buffers and/or coloring substances and the like, which do not deleteriously react with the compounds disclosed above herein.

The pharmaceutical compositions and formulations may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3 butane diol, for example. Other acceptable diluents and solvents include, but are not limited to, Ringer's solution, isotonic sodium chloride solution, and fixed oils such as synthetic mono or di-glycerides. Other parenterally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self propelling solvent/powder dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

In some embodiments, the compositions are formulated into a nano-sized droplets, micron-sized droplets, aerosols, or mist (for example by way of an inhaler or nebulizer). The compositions of the invention may, if desired, be presented in a pack or dispenser device, which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound disclosed herein formulated in a compatible pharmaceutical carrier may also be prepared, placed in an appropriate container, and labeled for treatment of an indicated condition.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, PA), which is incorporated herein by reference.

### Methods of Use

In various aspects, the present invention also provides breath-based methods of detecting cancer in a subject in need thereof using the compositions of the present invention (i.e., compositions comprising exogenous synthase (e.g., limonene synthase, such as SEQ ID NOs: 1, 3, 5, 7, 9, 11, 13, 15, 17, 19, 21, 23, 25, 27, 29, 31, 33, or 35-38, or an exogenous synthase containing an amino acid sequence motif selected from SEQ ID NOs: 51-175 or any combination thereof) or nucleic acid molecule encoding thereof (e.g., vector comprising a nucleic acid molecule encoding limonene synthase, such as SEQ ID NOs: 2, 4, 6, 8, 10, 12, 14, 16, 18, 20, 22, 24, 26, 28, 30, 32, 34, or 45-50). In some aspects, the present invention provides breath-based methods of monitoring a cancer or cancer treatment in a subject in need thereof using the compositions of the present invention.

In some embodiments, the method comprises (a) administering to the subject at least one composition of the present invention, wherein the exogenous synthase expresses preferentially in cancer cells compared to noncancerous cells and catalyzes production of a volatile organic compound, and wherein the volatile organic compound is not produced endogenously in the subject; (b) capturing breath exhaled from the subject; (c) analyzing the exhaled breath for the volatile organic compound; (d) comparing the amount of the volatile organic compound in the exhaled breath to a comparator; and (e) determining the subject has cancer when the amount of the volatile organic compound in the exhaled breath is increased compared to a comparator. In some embodiments, the comparator is an amount of the volatile organic compound in the exhaled breath from a subject not having cancer.

Exemplary cancers that can be detected using the compounds, compositions, and methods of the present invention include, but are not limited to, acute lymphoblastic leukemia, acute myeloid leukemia, adrenocortical carcinoma, appendix cancer, basal cell carcinoma, bile duct cancer, bladder cancer, bone cancer, brain and spinal cord tumors, brain stem glioma, brain tumor, breast cancer, bronchial tumors, Burkitt lymphoma, carcinoid tumor, central nervous system atypical teratoid/rhabdoid tumor, central nervous system embryonal tumors, central nervous system lymphoma, cerebellar astrocytoma, cerebral astrocytoma/malignant glioma, cerebral astrocytotna/malignant glioma, cervical cancer, childhood visual pathway tumor, chordoma, chronic lymphocytic leukemia, chronic myelogenous leukemia, chronic myeloproliferative disorders, colon cancer, colorectal cancer, craniopharyngioma, cutaneous cancer, cutaneous t-cell lymphoma, endometrial cancer, ependymoblastoma, ependymoma, esophageal cancer, Ewing family of tumors, extracranial cancer, extragonadal germ cell tumor, extrahepatic bile duct cancer, extrahepatic cancer, eye cancer, fungoides, gallbladder cancer, gastric (stomach) cancer, gastrointestinal cancer, gastrointestinal carcinoid tumor, gastrointestinal stromal tumor (gist), germ cell tumor, gestational cancer, gestational trophoblastic tumor, glioblastoma, glioma, hairy cell leukemia, head and neck cancer, hepatocellular (liver) cancer, histiocytosis, Hodgkin lymphoma, hypopharyngeal cancer, hypothalamic and visual pathway glioma, hypothalamic tumor, intraocular (eye) cancer, intraocular melanoma, islet cell tumors, Kaposi sarcoma, kidney (renal cell) cancer, langerhans cell cancer, langerhans cell histiocytosis, laryngeal cancer, leukemia, B-cell derived leukemia, T-cell derived leukemia, B-cell lymphoma, large B-cell diffuse lymphoma, lip and oral cavity cancer, liver cancer, lung cancer, lymphoma, macroglobulinemia, malignant fibrous histiocvtoma of bone and osteosarcoma, medulloblastoma, medulloepithelioma, melanoma, Merkel cell carcinoma, mesothelioma, metastatic squamous neck cancer with occult primary, mouth cancer, multiple endocrine neoplasia syndrome, multiple myeloma, mycosis, myelodysplastic syndromes, myelodysplastic/myeloproliferative diseases, myelogenous leukemia, myeloid leukemia, myeloma, myeloproliferative disorders, nasal cavity and paranasal sinus cancer, nasopharyngeal cancer, neuroblastoma, non-Hodgkin lymphoma, non-small cell lung cancer, oral cancer, oral cavity cancer, oropharyngeal cancer, osteosarcoma and malignant fibrous histiocytoma, osteosarcoma and malignant fibrous histiocytoma of bone, ovarian, ovarian cancer, ovarian epithelial cancer, ovarian germ cell tumor, ovarian low malignant potential tumor, pancreatic cancer, papillomatosis, paraganglioma, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm, plasma cell neoplasm/multiple myeloma, pleuropulmonary blastoma, primary central nervous system cancer, primary central nervous system lymphoma, prostate cancer, rectal cancer, renal cell (kidney) cancer, renal pelvis and ureter cancer, respiratory tract carcinoma involving the nut gene on chromosome 15, retinoblastoma, rhabdomyosarcoma, salivary gland cancer, sarcoma, sezary syndrome, skin cancer (melanoma), skin cancer (nonmelanoma), skin carcinoma, small cell lung cancer, small intestine cancer, soft tissue cancer, soft tissue sarcoma, squamous cell carcinoma, squamous neck cancer, stomach (gastric) cancer, supratentorial primitive neuroectodermal tumors, supratentorial primitive neuroectodermal tumors and pineoblastoma, T-cell lymphoma, testicular cancer, throat cancer, thymoma and thymic carcinoma, thyroid cancer, transitional cell cancer, transitional cell cancer of the renal pelvis and ureter, trophoblastic tumor, urethral cancer, uterine cancer, uterine sarcoma, vaginal cancer, visual pathway and hypothalamic glioma, vulvar cancer, Waldenstrom macroglobulinemia, and Wilms tumor.

In some aspects, the present invention also provides breath-based methods of evaluating the effectiveness of a cancer treatment in a subject in need thereof using the compositions of the present invention. For example, in some embodiments, the method comprises (a) administering to the subject at least one composition of the invention, wherein the exogenous synthase expresses preferentially in cancer cells compared to noncancerous cells and catalyzes production of a volatile organic compound, and wherein the volatile organic compound is not produced endogenously in the subject; (b) capturing breath exhaled from the subject; (c) analyzing the exhaled breath for the volatile organic compound; (d) comparing the amount of the volatile organic compound in the exhaled breath to a comparator; and (e) determining the cancer treatment as effective when the amount of the volatile organic compound in the exhaled breath is decreased compared to a comparator; or (e) determining the cancer treatment as ineffective when the amount of the volatile organic compound in the exhaled breath is increased compared to a comparator. In some embodiments, the comparator is an amount of the volatile organic compound in the exhaled breath from the subject having cancer before the cancer treatment.

In various embodiments of the methods of the invention, the level or amount of the volatile organic compound in the exhaled breath is determined to be increased when the level or amount of the volatile organic compound in the exhaled breath is increased by at least 0.1%, by at least 1%, by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator.

In various embodiments of the methods of the invention, the level or amount of the volatile organic compound in the exhaled breath is determined to be increased when the level or amount of the volatile organic compound in the exhaled breath is determined to be increased by at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, or at least 1000 fold, or at least 10000 fold, when compared with a comparator.

In one embodiment, the subject is determined to have cancer when the level or amount of the volatile organic compound in the exhaled breath is determined to be increased in the breath as compared to a comparator. For example, in one embodiment, the subject is determined to have cancer when the level or amount of the volatile organic compound in the exhaled breath is determined to be increased by at least 1 fold, at least 1.1 fold, at least 1.2 fold, at least 1.3 fold, at least 1.4 fold, or at least 1.5 fold.

In one embodiment, the cancer treatment is determined to be ineffective when the level or amount of the volatile organic compound in the exhaled breath is determined to be increased in the breath as compared to a comparator. For example, in one embodiment, the cancer treatment is determined to be ineffective when the level or amount of the volatile organic compound in the exhaled breath is determined to be increased by at least 1 fold, at least 1.1 fold, at least 1.2 fold, at least 1.3 fold, at least 1.4 fold, or at least 1.5 fold.

In various embodiments of the methods of the invention, the level or amount of the volatile organic compound in the exhaled breath is determined to be decreased when the level or amount of the volatile organic compound in the exhaled breath is decreased by at least 0.1%, by at least 1%, by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator.

In various embodiments of the methods of the invention, the level or amount of the volatile organic compound in the exhaled breath is determined to be decreased when the level or amount of the volatile organic compound in the exhaled breath is determined to be decreased by at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, or at least 1000 fold, or at least 10000 fold, when compared with a comparator.

In one embodiment, the cancer treatment is determined to be effective when the level or amount of the volatile organic compound in the exhaled breath is determined to be increased in the breath as compared to a comparator. For example, in one embodiment, the cancer treatment is determined to be effective when the level or amount of the volatile organic compound in the exhaled breath is determined to be increased by at least 1 fold, at least 1.1 fold, at least 1.2 fold, at least 1.3 fold, at least 1.4 fold, or at least 1.5 fold.

In one embodiment, the method comprises using a multi-dimensional non-linear algorithm to determine if the level or amount of the volatile organic compound in the exhaled breath is statistically different than the level in a comparator sample. In some embodiments, the algorithm is drawn from the group consisting essentially of: linear or nonlinear regression algorithms; linear or nonlinear classification algorithms; ANOVA; neural network algorithms; genetic algorithms; support vector machines algorithms; hierarchical analysis or clustering algorithms; hierarchical algorithms using decision trees; kernel based machine algorithms such as kernel partial least squares algorithms, kernel matching pursuit algorithms, kernel fisher discriminate analysis algorithms, or kernel principal components analysis algorithms; Bayesian probability function algorithms; Markov Blanket algorithms; a plurality of algorithms arranged in a committee network; and forward floating search or backward floating search algorithms.

Non-limiting examples of comparators include, but are not limited to, a negative control, a positive control, standard control, standard value, an expected normal background value of the subject, a historical normal background value of the subject, a reference standard, a reference level, an expected normal background value of a population that the subject is a member of, or a historical normal background value of a population that the subject is a member of.

In one embodiment, the comparator is a level or amount of the volatile organic compound in the exhaled breath in a sample obtained from a subject not having cancer. In one embodiment, the comparator is a level or amount of the volatile organic compound in the exhaled breath obtained from a subject known not to have cancer.

Breath exhaled by the subject can captured for subsequent analysis, or direct analysis of the breath in real-time. The exhaled breath is analyzed for volatile organic compound (e.g., limonene) released from cancer cells as a biomarker of cancer.

Various methods are known in the art for collecting and storing breath samples for offline analysis of a volatile organic compound in a gaseous phase. These include polymer sampling bags, cannisters (including passivated metal canisters), glass containers or bulbs, plastic containers, sorbent tubes, solid-phase microextraction (SPME) fibers, and rubber balloons. Sampling bags can be made of various polymers, including: Tedlar (polyvinyl fluoride), Nalophan, Mylar (polyethylene terephthalate), Kynar, ALTEF, (polyvinylidene difluoride), and Teflon (polytetrafluroethylene, perfluoroalkoxy polymer, tetrafluoroethylene hexafluoropropylene copolymer), and rubber balloons.

Various methods are known in the art for pre-concentrating (“pre-concentration” refers to obtaining a high concentration of trace analyte prior to analysis) breath samples for subsequent offline analysis of a volatile organic compound. These include solid-phase microextraction (SPME) fibers and sorbent tubes. In the SPME technique, a fused silica fiber coated with a polymeric stationary phase is contained in a specially designed syringe whose needle protects the fiber when septa are pierced. The fiber is directly exposed to a liquid or gaseous sample to extract and concentrate the analytes. After the absorption equilibration is attained, the fiber is withdrawn into the needle and introduced into an injector of a gas chromatograph, where the extracted compounds are thermally desorbed and analyzed. Types of adsorbent polymer films used in SPME fibers can include polydimethylsiloxane (PDMS), polyacrylate (PA), and polyethylene glycol (PEG). Types of adsorbent porous particles used in SPME include divinylbenzene (DVB), Carboxen® (CAR), or a combination of the two, usually with PDMS as the binder. Sorbent tubes are typically made of glass or stainless steel and contain various types of solid adsorbent material (sorbents). Commonly used sorbents include activated charcoal, silica gel, and organic porous polymers such as Tenax and Amberlite XAD resins. A breath sample can be placedAfter sample preconcentration, VOCs are extracted from the sorbent tube by thermal desorption (for example, by placing the sorbent tube in a thermal desorption unit attached to a GC-MS instrument) for analysis.

Various methods are known in the art for identifying a volatile organic compound in a gaseous phase. Individual components may be separated, analyzed, and characterized using methods known to those skilled in the art. In a non-limiting embodiment, the individual components may be partially or completely purified using, for example, chromatographic methods (such as, but not limited to, gas chromatography (GC). In another non-limiting embodiment, the partially or completely purified components of the library may be analyzed or characterized using methods such as, but not limited to, nuclear magnetic resonance (NMR), mass spectrometry (MS), gas chromatography-mass spectrometry (GC-MS), selected ion-flow tube mass spectrometry (SIFT-MS), proton transfer reaction mass spectrometry (PTR-MS), ion mobility spectrometry, ultraviolet-visible (UV-vis) spectroscopy, infrared (IR) spectroscopy, and electronic noses. SIFT-MS and PTR-MS allow for direct online analysis of the breath for VOCs of interest in real time. The information derived from these methods may be used to establish the structure of the specific components of the library.

Electronic nose sensors consist of a semi-selective sensor or an array of semi-selective sensors. Each sensor in the array may be sensitive to multiple volatile molecules. The combinatorial responses of the sensor components to a particular analyte or mixture yields a signal pattern or fingerprint that can identify a VOC or VOC class. Sensor elements in electronic noses can include colorimetric sensors, optical absorption (including surface plasmon resonance) and luminescence-based sensors, piezoelectric crystals, chemiresistors, field effect transistors, metal-oxide semiconductor sensors, conducting and non-conducting polymers, surface acoustic wave devices, thickness shear mode resonators (TSM), quartz crystal microbalances, and nanomaterial-based sensors.

In various embodiments, the limit of detection of the analyzer (e.g., GC-MS, MS, electronic nose device, etc.) is the limit of detection of the method of the present invention. For example, in some embodiments, the method detects at least about 2 parts per trillion (ppt) of the volatile organic compound of interest. In some embodiments, the method detects at least about 2 parts per billion (ppb) of the volatile organic compound of interest.

Thus, in some embodiments, the method detects at least one tumor having a diameter of at least about 4.6 mm.

In some embodiments, the method detects at least one tumor having a volume of at least about 0.10 cm³.

In some embodiments, the method detects at least one tumor having a volume of at least about 1 mm³.

In some embodiments, the method detects at least one tumor having a diameter of at least about 1.0 mm.

In some embodiments, the method detects at least 1 picogram of the volatile organic compound of interest.

In some embodiments, the method detects at least 1 nanogram of the volatile organic compound of interest.

In some embodiments, the method detects at least 1 microgram of the volatile organic compound of interest.

In various embodiments, the present invention also provides a method of administering at least one composition of the present invention (i.e., compositions comprising a gene encoding an exogenous synthase (e.g., limonene synthase, such as SEQ ID NOs: 1, 3, 5, 7, 9, 11, 13, 15, 17, 19, 21, 23, 25, 27, 29, 31, 33, or 35-38, or a gene encoding an exogenous synthase containing an amino acid sequence motif selected from SEQ ID NOs: 51-175 or any combination thereof) or nucleic acid molecule encoding thereof (e.g., vector comprising a nucleic acid molecule encoding limonene synthase, such as SEQ ID NOs: 2, 4, 6, 8, 10, 12, 14, 16, 18, 20, 22, 24, 26, 28, 30, 32, 34, or 45-50) to a subject in need thereof. For example, in some embodiments, the present invention provides a method of administering at least one composition of the present invention to a subject at risk of having a cancer. In some embodiments, the present invention provides a method of administering at least one composition of the present invention to a subject having a cancer. In some embodiments, the present invention provides a method of administering at least one composition of the present invention to a subject in remission.

The pharmaceutical compositions useful for practicing the invention may be administered to deliver a dose of from 0.001 ng/kg/day and 100 mg/kg/day. For example, in some embodiments, the pharmaceutical compositions useful for practicing the invention may be administered to deliver a dose of from 0.005 mg/kg/day and 5 mg/kg/day. In one embodiment, the invention envisions administration of a dose which results in a concentration of the synthase of interest from 10 nM and 10 μM in a mammal.

Typically, dosages which may be administered in a method of the invention to a mammal, preferably a human, range in amount from 0.01 μg to about 50 mg per kilogram of body weight of the mammal, while the precise dosage administered will vary depending upon any number of factors, including but not limited to, the type of mammal and type of disease state being treated, the age of the mammal and the route of administration. Preferably, the dosage of the compound will vary from about 0.1 μg to about 10 mg per kilogram of body weight of the mammal. More preferably, the dosage will vary from about 1 μg to about 5 mg per kilogram of body weight of the mammal. For example, in some embodiments, the dosage will vary from about 0.005 mg to about 5 mg per kilogram of body weight of the mammal.

The composition may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any 10 number of factors, such as, but not limited to, the type of disease being detected, the age or weight of the subject, etc.

In certain embodiments, administration of a composition of the present invention may be performed by single administration or multiple administrations.

### Devices

In various aspects, the present invention provides a device for detecting cancer in a subject in need thereof. In some aspects, the present invention provides a device for monitoring a cancer or cancer treatment in a subject in need thereof. In other aspects, the present invention provides a device for evaluating the effectiveness of a cancer treatment.

In various embodiments, the device comprises at least one composition of the present invention and at least one analyzer of the volatile organic compound. In some embodiments, the device is an electronic nose device, portable electronic nose device, breath analyzer, and/or breathalyzer.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1: Engineering Genetically-Encoded Synthetic Biomarkers for Breath-Based Cancer Detection

Engineered synthetic reporters provide an innovative solution to overcome the detection limitations of endogenous biomarkers. By effecting diseased cells to express an exogenous biomarker that is not naturally produced in human tissues, background signal from non-diseased tissues is minimized, thereby maximizing sensitivity and specificity. Moreover, exogenous reporters from biochemical classes that are orthogonal to the human metabolome can be distinguished from the complex milieu of endogenous molecules by mass spectrometry. Furthermore, detection of a single exogenous biomarker that uniquely signals disease presence avoids the statistical challenges associated with endogenous VOC analysis. Recent synthetic strategies include exogenous protein biomarkers encoded on in vivo-delivered DNA vectors and selectively secreted into the blood by cancer cells, as well as nanoparticles that release a volatile compound in the breath to signal lung infection or inflammation. Genetically-encoded synthetic biomarkers have practical and theoretical advantages, including: 1) integration with clinically established nonviral in vivo gene delivery methods, including those used in vaccines; 2) selective expression in many cancer types using tumor-activatable promoters and tumoritropic or tumor-targeted vectors; 3) continuous expression throughout the lifetime of the cancer, which can enable repeat monitoring after a single administration; and 4) modularity, in that the VOC reporter gene construct can be integrated with or swapped with an imaging reporter gene (PET, MR, or acoustic), enabling subsequent spatial localization with clinical imaging in the event of a positive test. However, there have been no reports thus far of strategies that genetically encode synthetic biomarkers for breath-based detection of cancer.

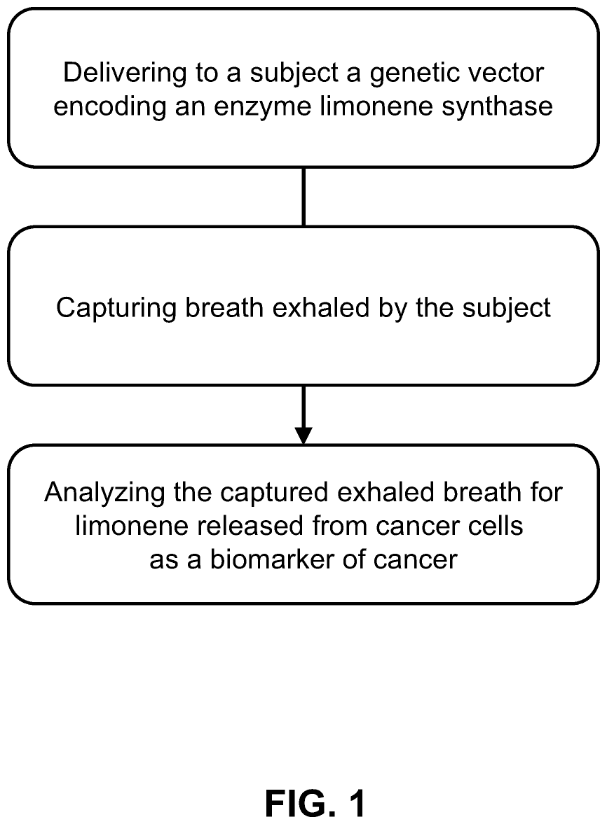

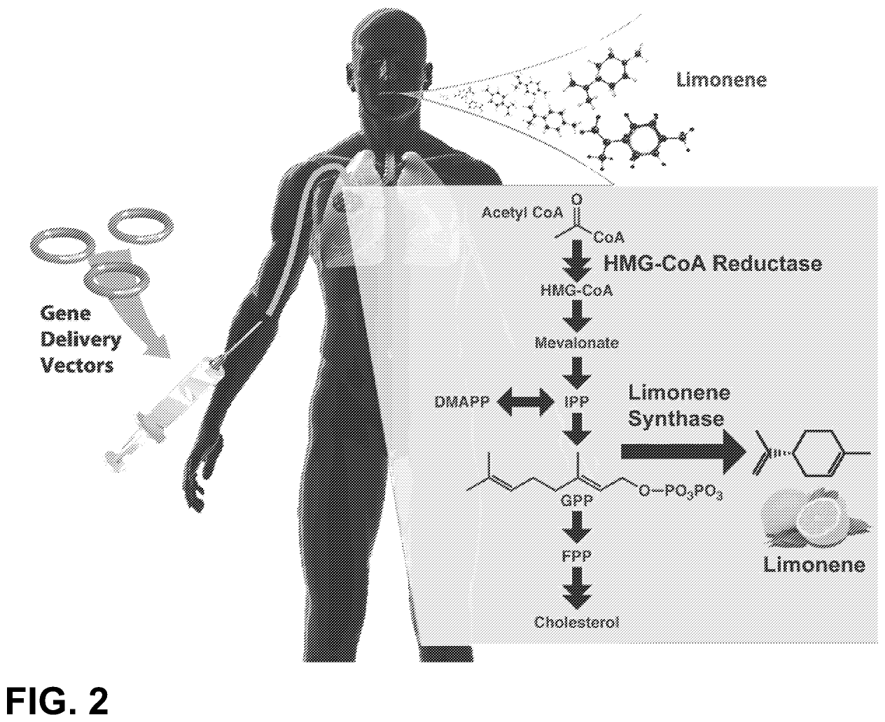

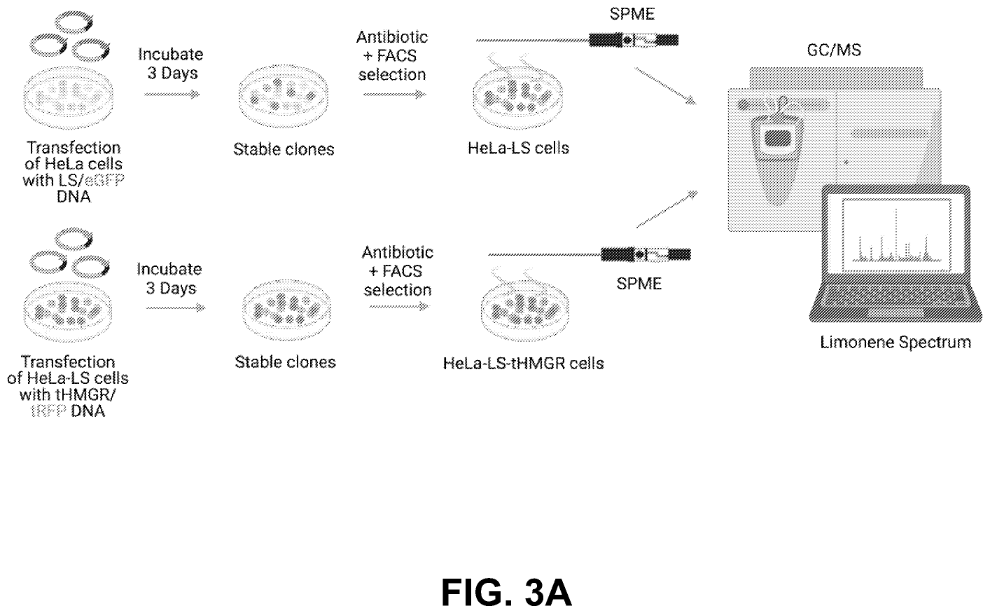

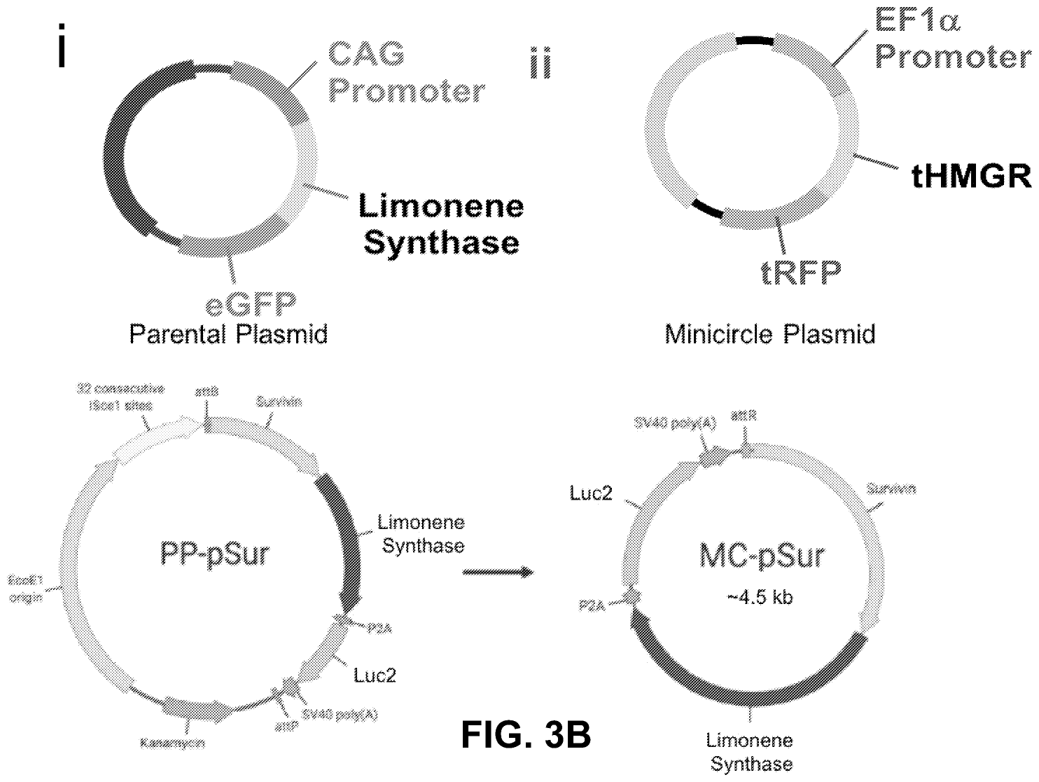

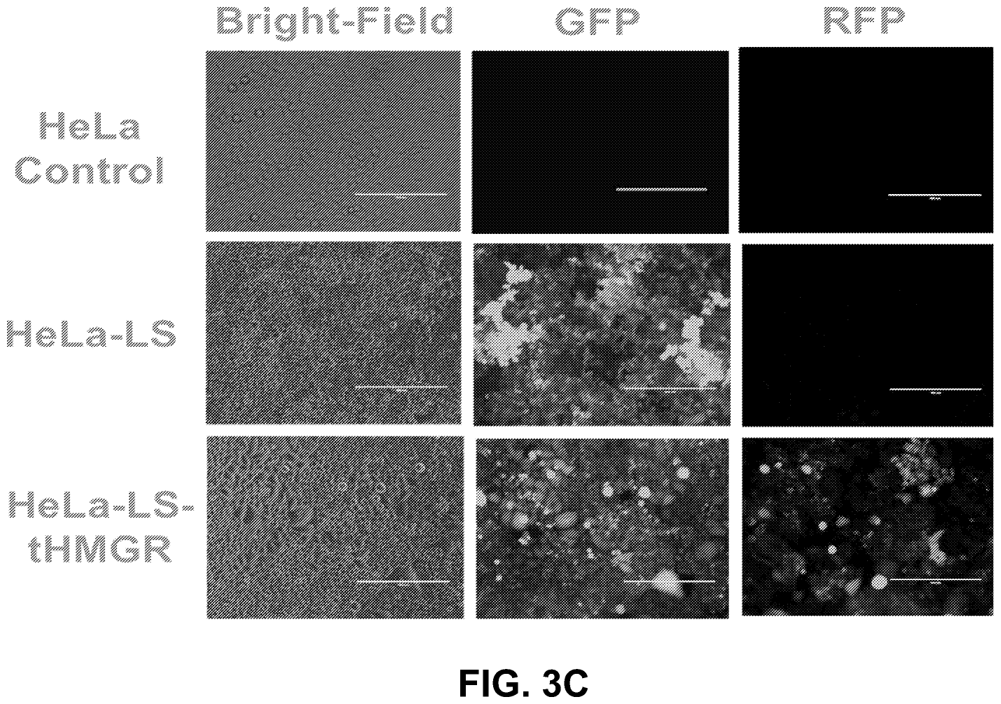

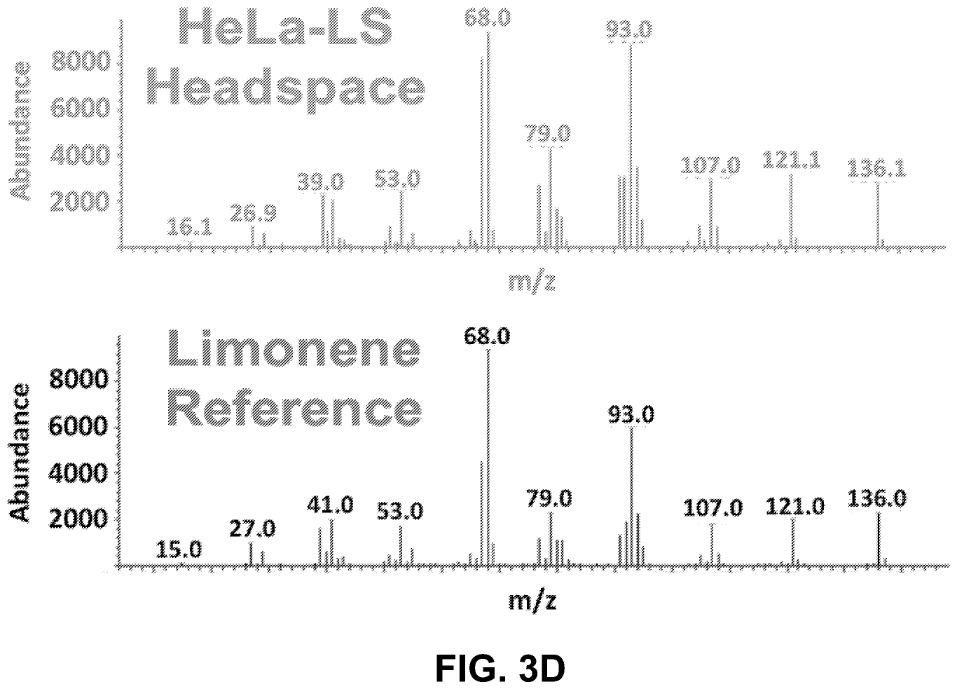

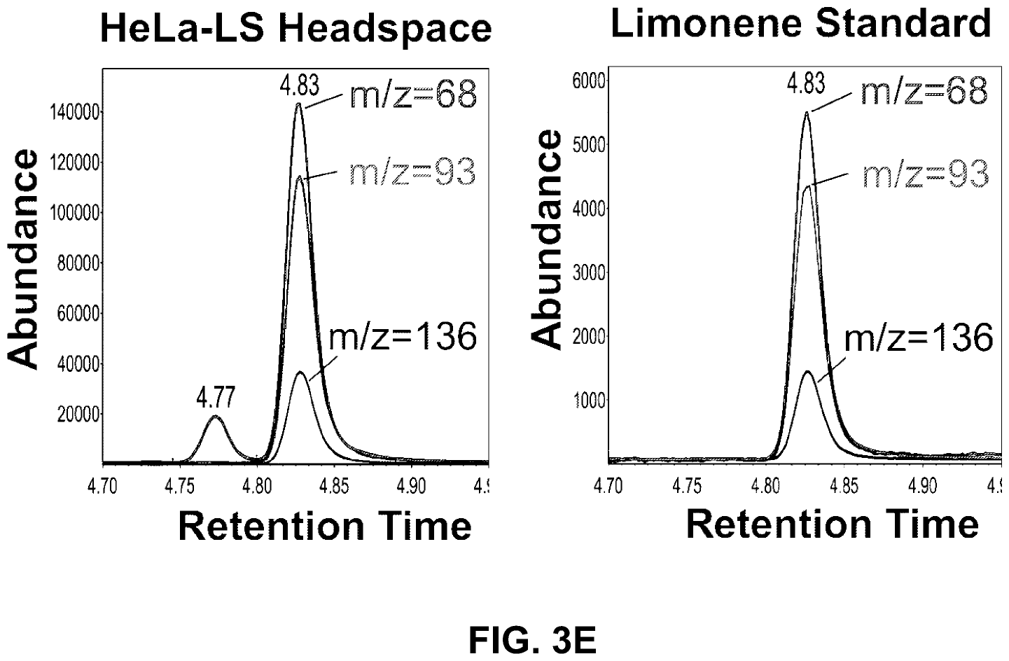

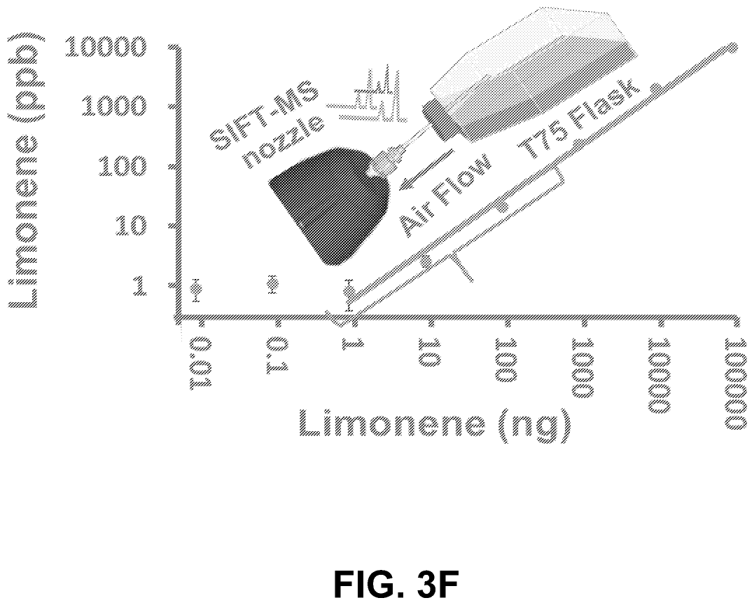

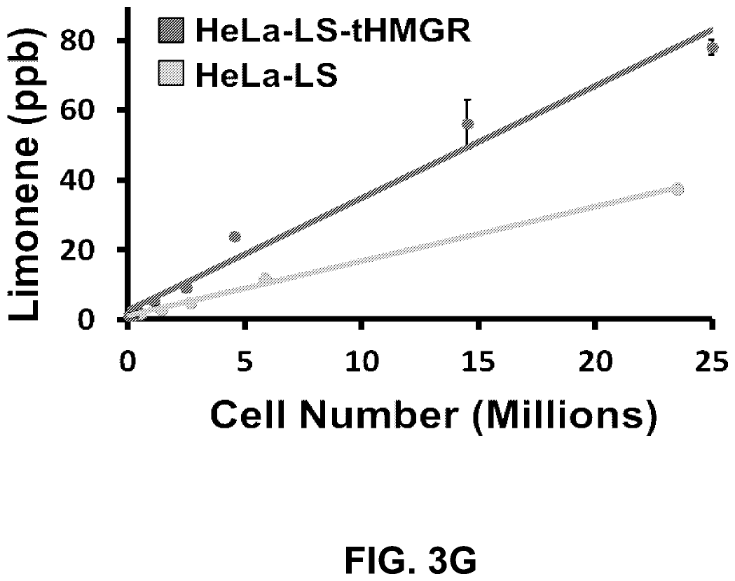

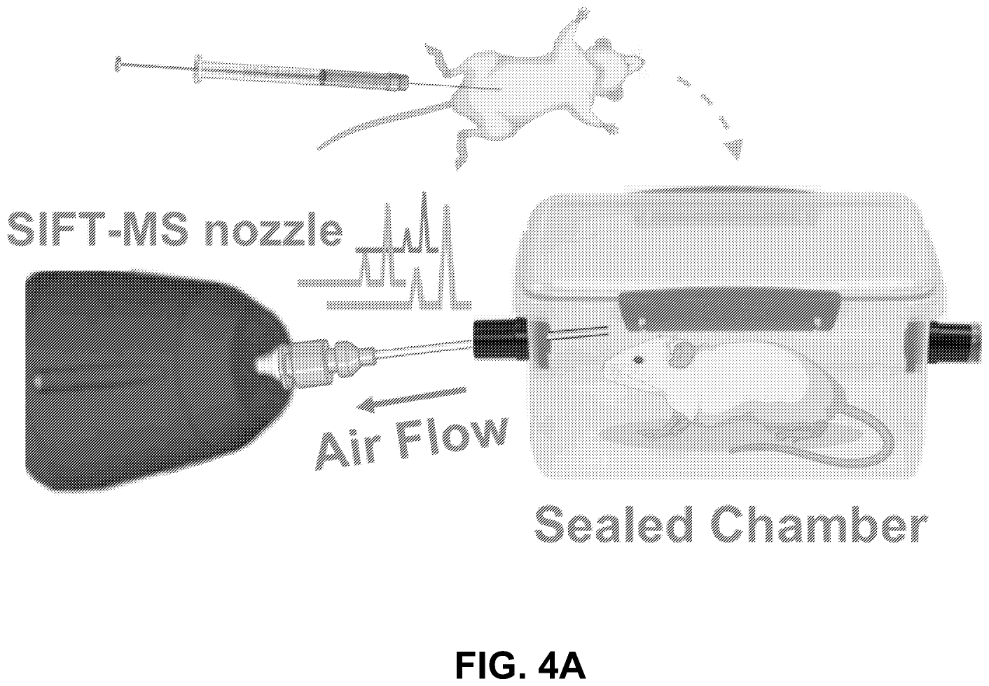

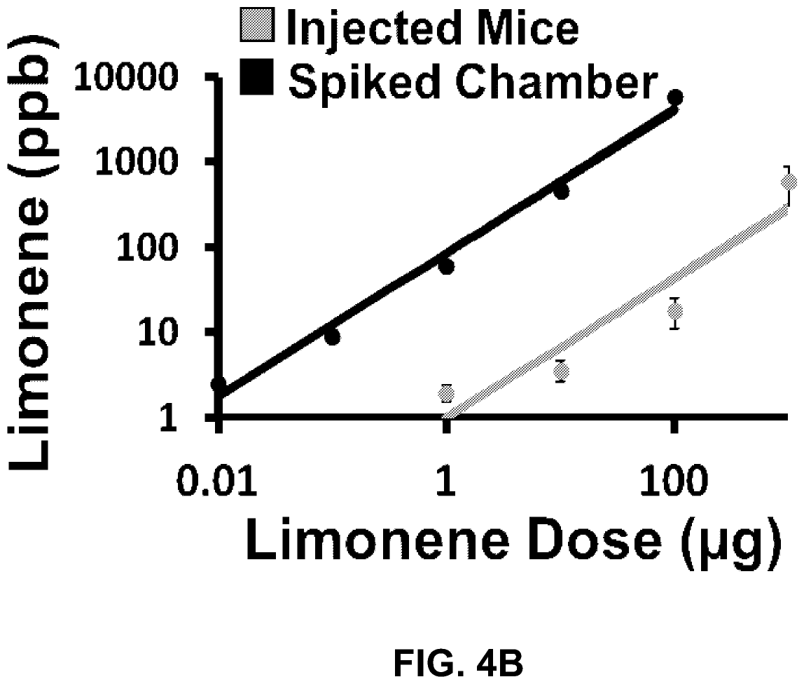

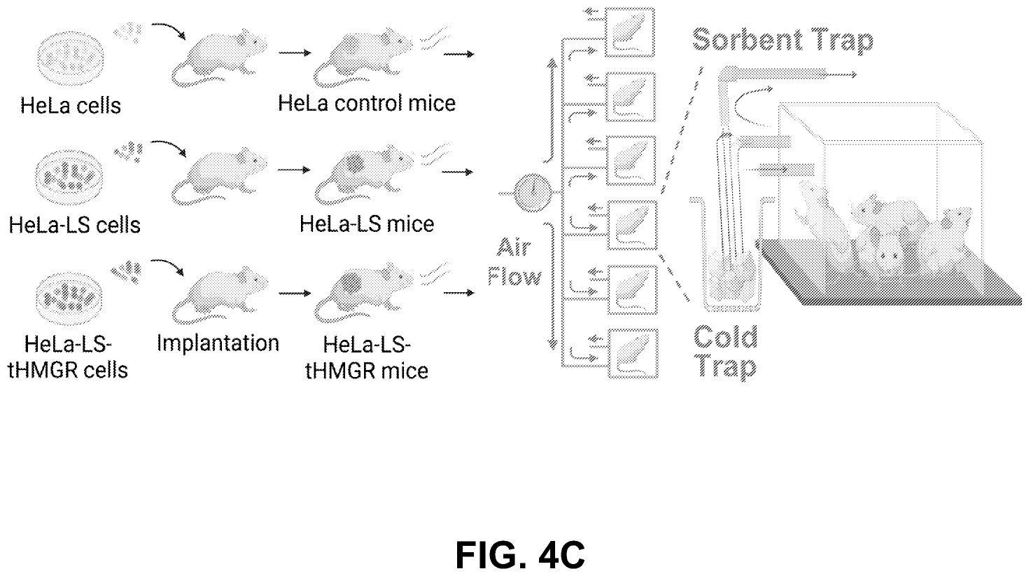

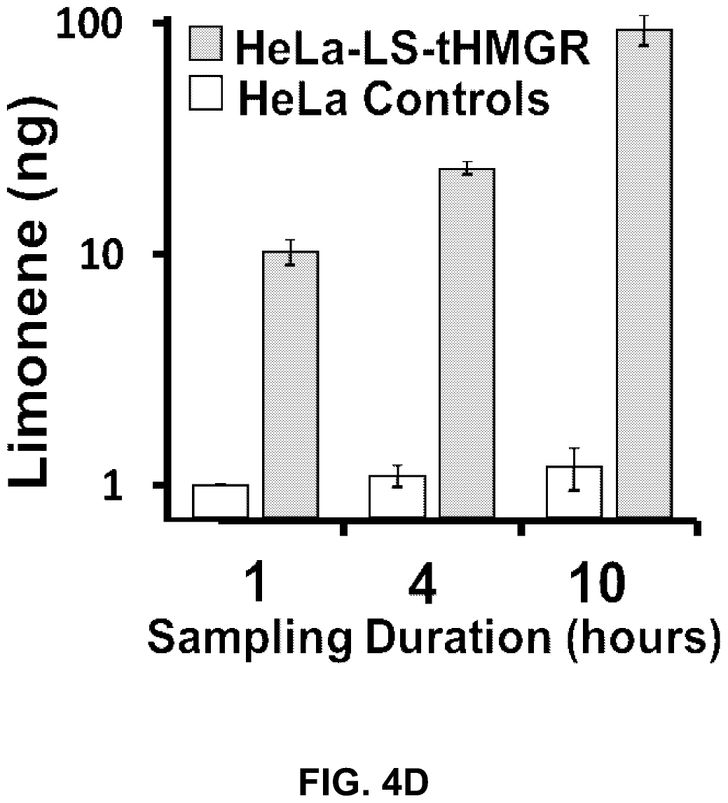

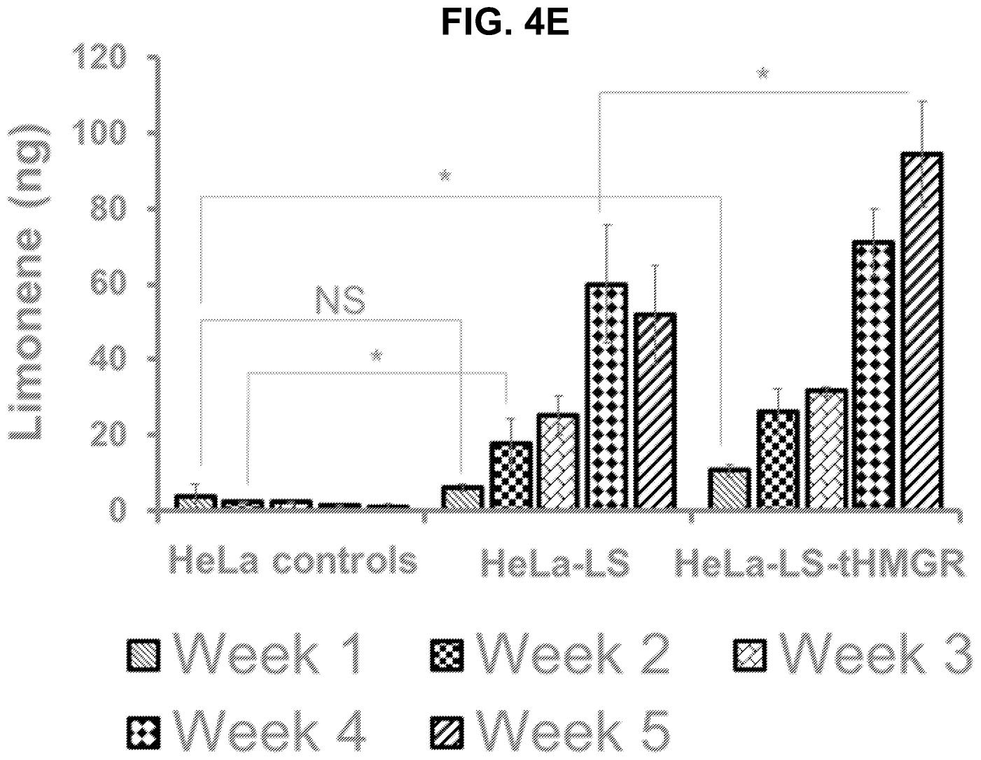

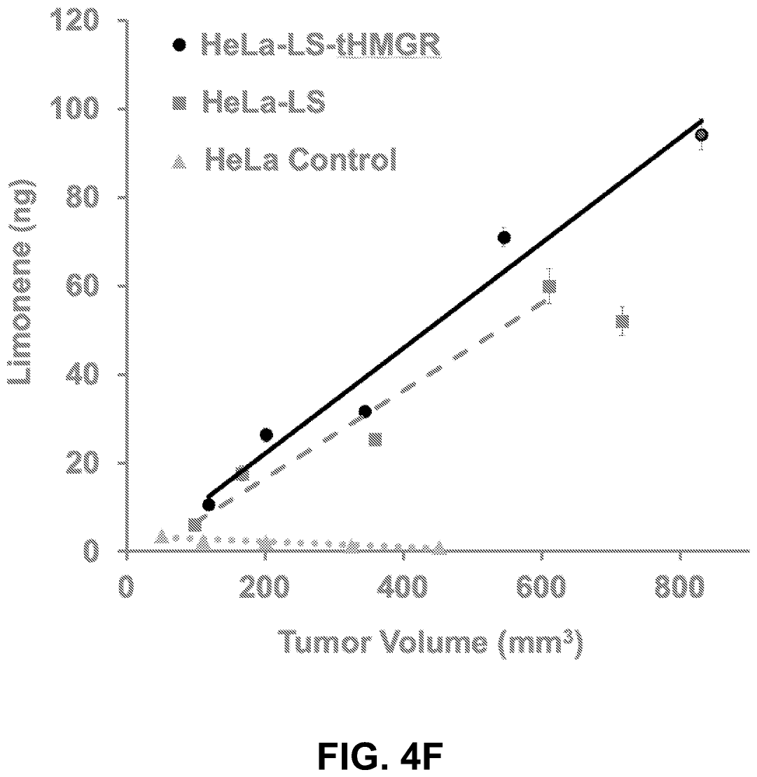

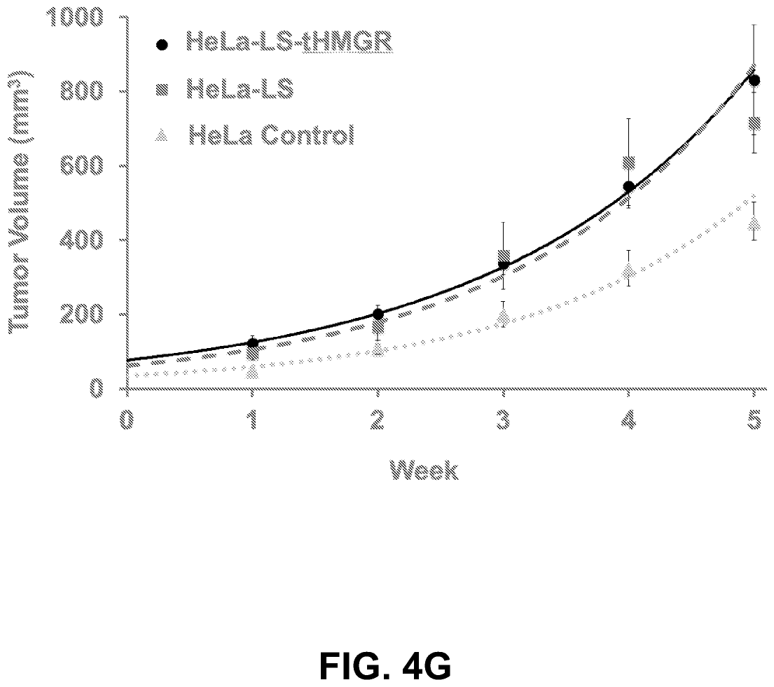

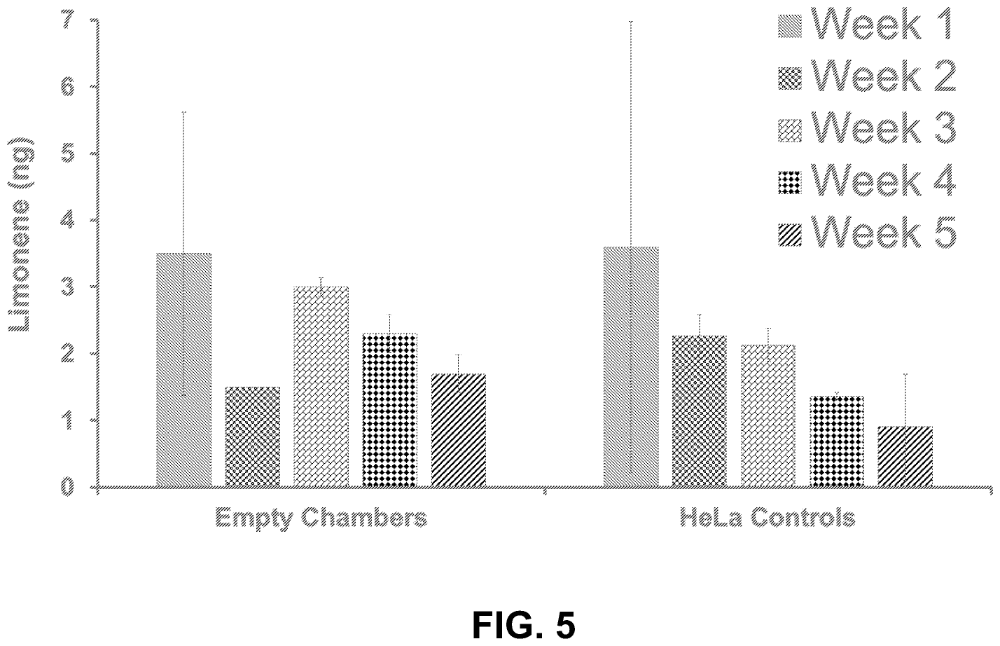

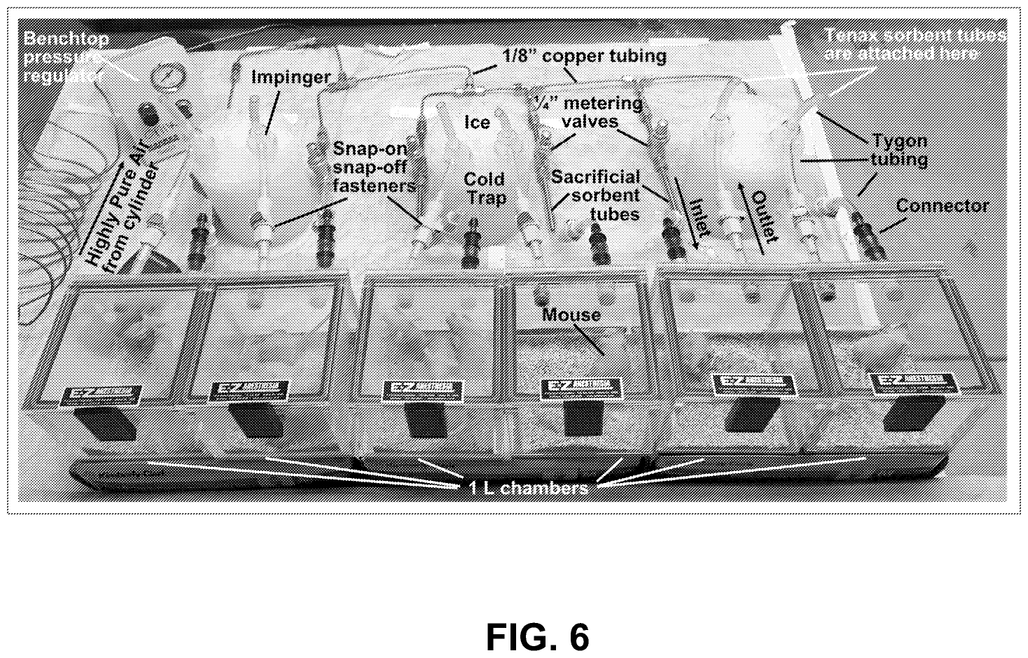

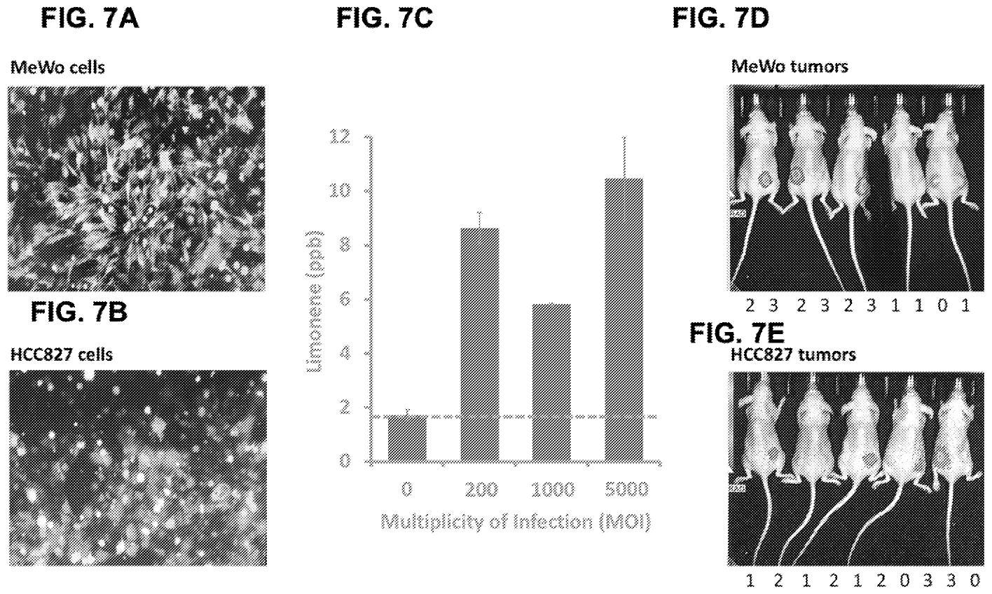

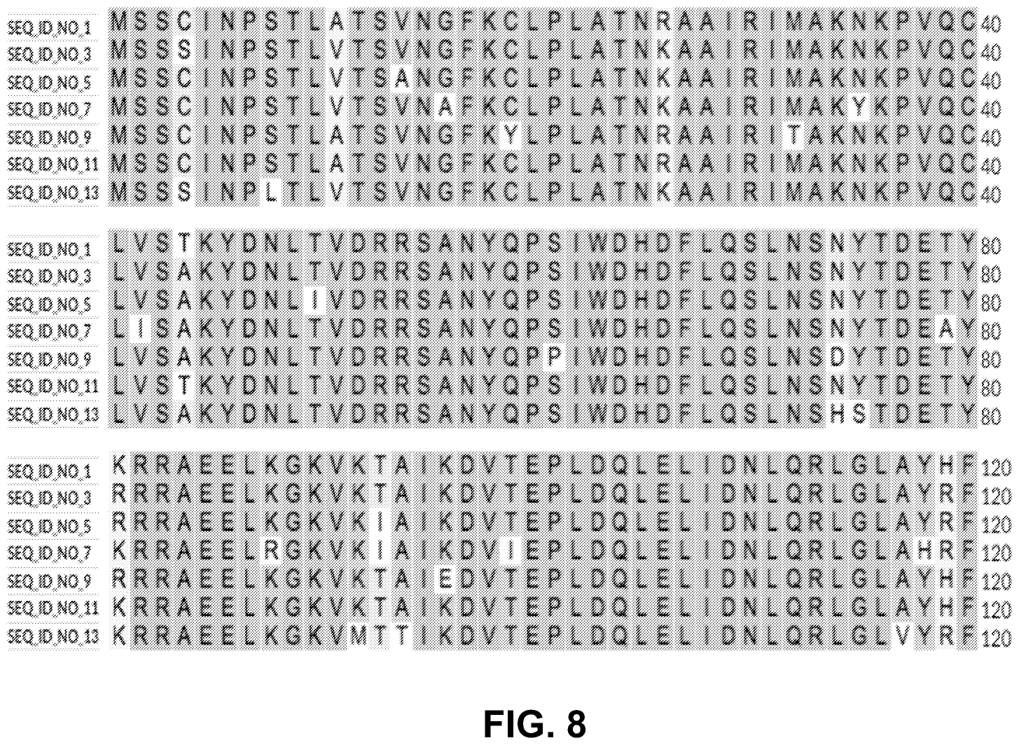

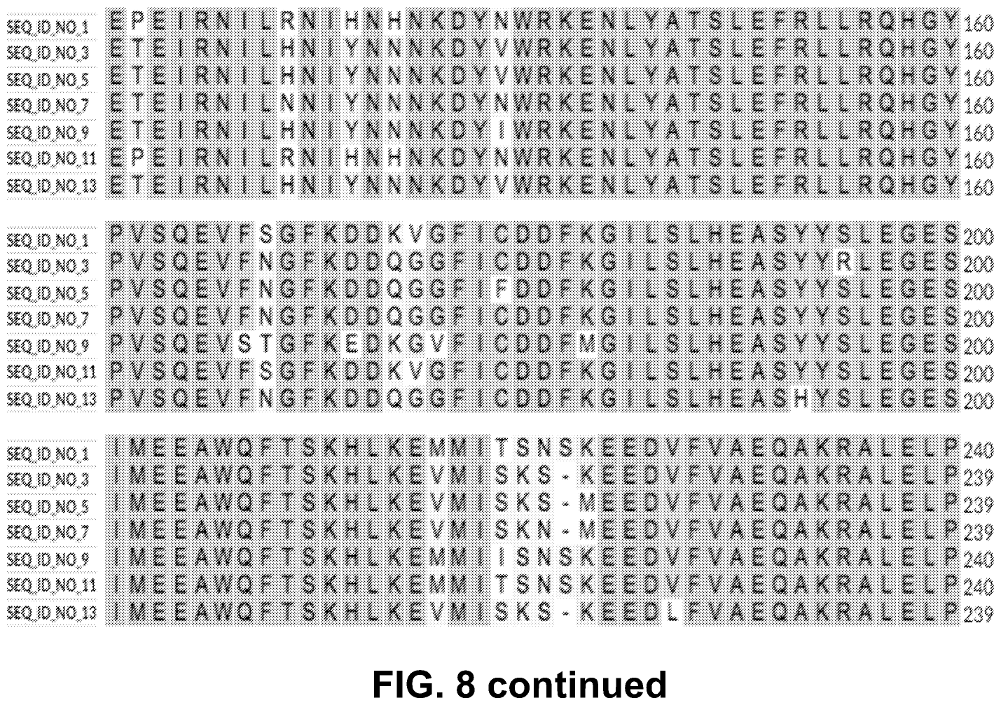

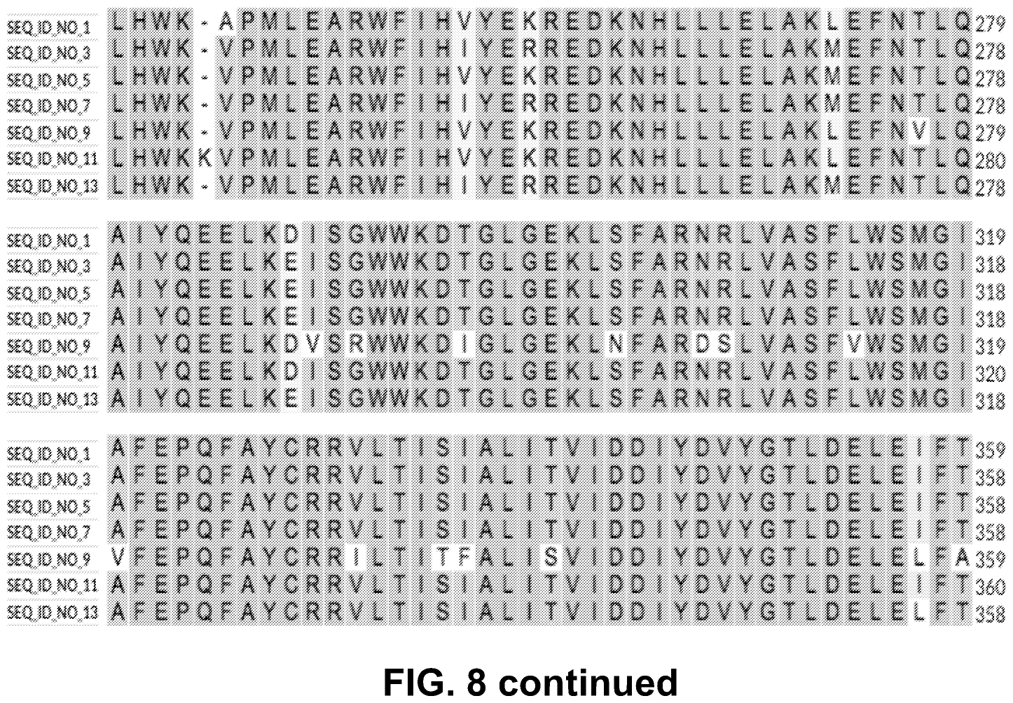

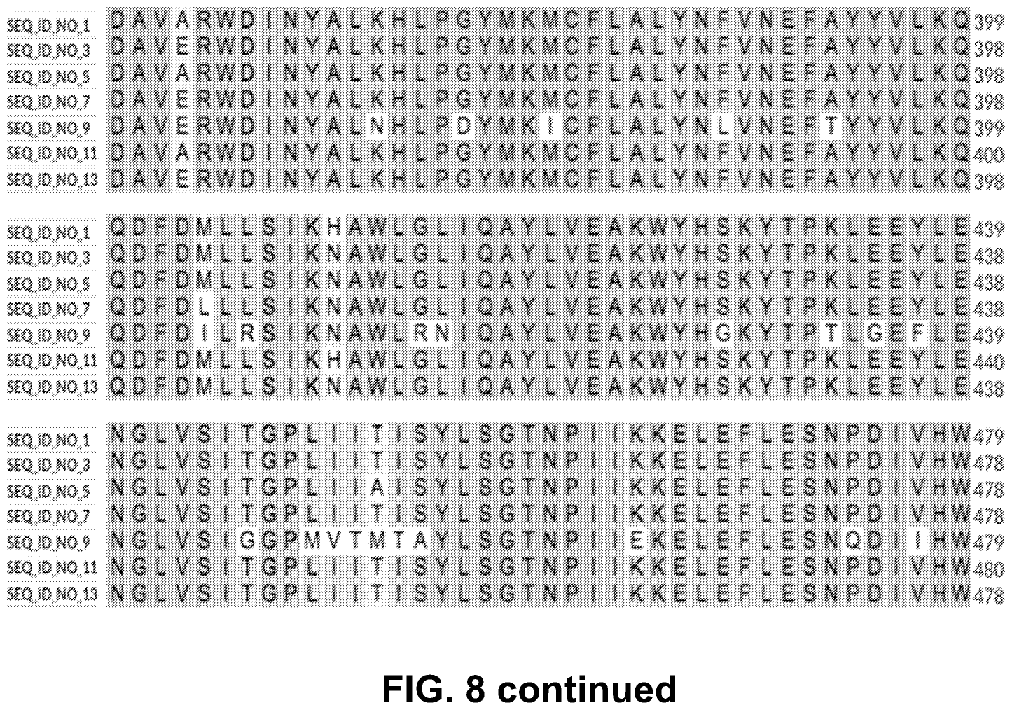

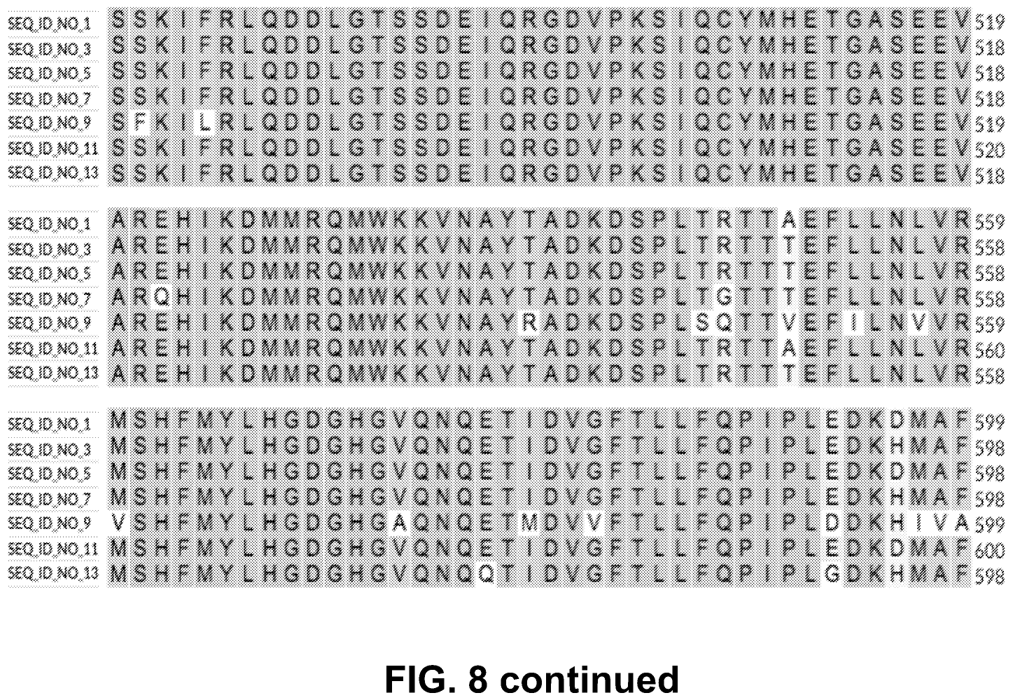

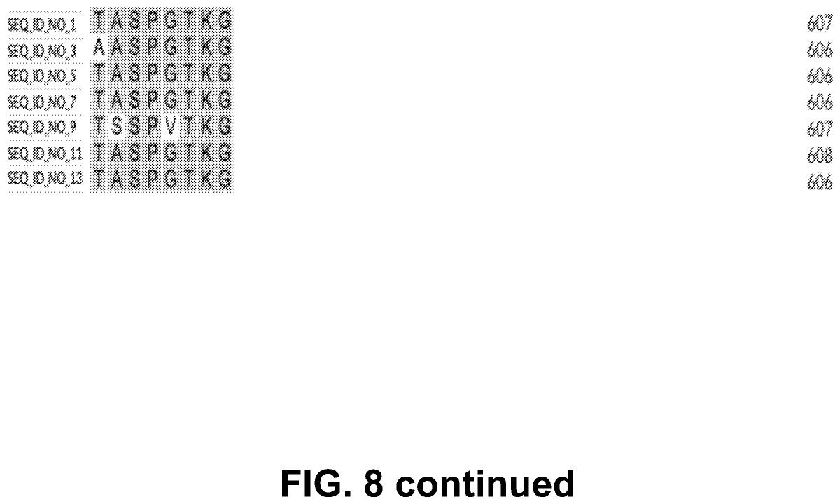

The present studies combined the high specificity and sensitivity of an exogenous cancer biomarker with the speed, simplicity, and non-invasive nature of breath VOC detection (FIG. 1). To genetically encode a VOC biomarker in cancer cells that is distinct from endogenous VOCs, plant volatiles were examined. Humans and plants share a common cholesterol biosynthesis pathway, but in plants this pathway also generates terpenes, the volatile compounds that attract pollinators and protect from herbivorous insects and pathogens. For this reason, the present study focused on the development of mammalian cell's cholesterol biosynthetic machinery that could be exploited to produce plant volatiles by genetically introducing the appropriate exogenous enzymes (FIG. 2).

While many plant volatiles require multiple biosynthetic steps, only a single enzyme, limonene synthase (LS), bridges the cholesterol biosynthesis pathway with production of limonene, the monoterpene that gives citrus fruits their characteristic scent. Limonene is already used clinically (for example, to treat gallstones and heartburn), has chemopreventive and chemotherapeutic effects in many types of cancers, and is safe at oral doses as high as 100 mg/kg (˜7 g for an average 70 kg adult). Due to its wide industrial use, metabolic engineering approaches for increasing limonene biosynthesis have been extensively studied in microbial systems and plants, and have the potential to be adapted to human cancer cells for breath-based diagnosis and eventually—at high expression levels—for therapy. The present studies demonstrated that limonene was genetically expressed in human cancer cells and reported on early tumor presence and growth in a xenograft mouse model. The present studies also extrapolated the VOC-based detection to humans using a whole-body physiologically-based pharmacokinetic (PBPK) model of VOC biodistribution, metabolism, and exhalation.

### Limonene Expression and Detection in Cultured Tumor Cells

HeLa cells were transfected with a vector containing LS and eGFP genes under the control of a single CAG promoter (FIG. 3A and FIG. 3B). Antibiotic selection and FACS sorting for high eGFP expressers yielded a stable cell line containing limonene synthase (HeLa-LS) (FIG. 3C). To maximize limonene production in cultured HeLa-LS cells, the present studies targeted a key regulatory enzyme of the mevalonate pathway, HMG-CoA reductase (HMGR). Truncation of HMGR by deletion of its N-terminal regulatory domain rendered it insensitive to feedback inhibition by downstream metabolites, augmenting flux through the mevalonate pathway and increasing the availability of limonene precursors. Previous studies in bacteria and yeast engineered to produce limonene have shown that expression of truncated HMGR (tHMGR) can markedly increase limonene production. HeLa-LS cells were transfected with a plasmid encoding human tHMGR and turbo red fluorescent protein (tRFP) under the control of an EF1α promoter (FIG. 3A and FIG. 3B). Antibiotic selection and FACS sorting for high expression of tRFP yielded a stable cell line expressing both eGFP and tRFP (FIG. 3C) and contained both LS and tHMGR (HeLa-LS-tHMGR). Solid phase microextraction (SPME) fibers (5, 43) were used to sample the culture headspace (i.e., the air above the cells) in flasks containing confluent stably transfected cells (FIG. 3A). Gas chromatography-mass spectrometry (GC-MS) analysis of the fibers showed a mass spectrum closely matching the limonene standard, with both exhibiting the characteristic ion peaks for limonene (m/z=68, 93, and 136) at the same relative ratios (FIG. 3D) and identical chromatogram retention times (FIG. 3E).

Quantification of Limonene from Transfected Cells

The present studies further confirmed the presence of headspace limonene using selected ion flow tube mass spectrometry (SIFT-MS), which affords continuous, real-time VOC detection with quantification down to the parts-per-billion level. To obtain quantitative measurements of headspace limonene, a calibration curve for limonene (10 pg to 100 pg) spiked into media within a 280 mL T75 flask was generated (FIG. 3F). Headspace concentrations increased as a function of x^{0.86} for limonene quantities within the range of 1 ng to 100 μg (R²=0.99) and demonstrated a nearly linear dependence with limonene quantities ranging from 1 ng to 1 μg (R²=0.99). The limit of detection (LOD) for limonene by SIFT-MS was 1.8 ng, corresponding to 0.5 ppb in the headspace. Next, the studies sought to quantify limonene generated by transfected HeLa cells over a 24-hour period. Limonene production increases linearly over a range of 45,000 to 25 million cells for both HeLa-LS(R²=0.99) and HeLa-LS-tHMGR (R²=0.99), with LODs of 360,000 cells and 107,000 cells, respectively, as compared to undetectable limonene levels in untransfected HeLa cells (FIG. 3G, Supplementary Calculations shown in Example 2, infra). For the largest number of HeLa-LS cells tested, a confluent culture of 23.5 million cells, the headspace limonene concentration was 38±2 ppb, corresponding to 131 ng of limonene or an average of ˜5.6 fg per cell per day. For the largest number of HeLa-LS-tHMGR cells tested, 25 million cells, the headspace limonene concentration was 78±2 ppb, corresponding to 277 ng of limonene or an average of ˜11 fg per cell per day. The slope of the best-fit line for HeLa-LS-tHMGR cells was twice that for HeLa-LS cells (3.2×10⁻⁶ vs. 1.6×10⁻⁶), demonstrating that HeLa-LS-tHMGR cells generated double the amount of limonene as HeLa-LS cells.

Quantification of Limonene Emitted from Limonene-Injected and Tumor-Bearing Mice

Having observed robust limonene expression in transfected HeLa cells in culture, the feasibility of detecting limonene in exhaled breath from rodents was then tested. A standard curve relating limonene concentration in chamber headspace to the quantity of limonene spiked into 0.5-L chambers was generated. To determine the fraction of limonene in mice that was emitted into the headspace, mice were injected intraperitoneally with different quantities of a limonene standard solution (from 0.01 μg to 1 mg) and individual mice were placed in a closed chamber for 15 minutes, at which point headspace limonene concentrations were measured by SIFT-MS (FIG. 4A and FIG. 4B).

Using the standard curve, the mass of limonene exhaled by mice at each quantity injected was determined and the fraction exhaled was calculated. At the LOD (0.5 ppb), limonene in the chamber headspace became detectable when 2.3 ng had been spiked into the chamber, whereas limonene evolving from mice only became detectable at an injected dose of 450 ng (FIG. 4B, Supplementary Calculations shown in Example 2, infra). A comparison of the graphs for these two conditions showed that only ˜0.5% of limonene at each injected dose was emitted into the chamber headspace within 15 minutes of injection. For this reason, mice bearing limonene-producing tumors were to emit a similar fraction into the chamber headspace over this time period.

Using the limonene production rate in cell culture to be an upper bound on the range of the cellular limonene production rate in tumor-bearing mice, it was calculated that large tumors with diameters of at least 3.4 cm (4 billion cells) are required in order to reach the detection limit of SIFT-MS within 15 minutes (Supplementary Calculations shown in Example 2, infra). To test this, one million HeLa-LS or HeLa-LS-tHMGR cells were implanted subcutaneously into each flank of immunocompromised nude mice and monitored them using SIFT-MS at 5 weeks post-implantation. Consistent with the calculations, it was found that no limonene was detected in the chamber headspace even when up to 4 mice with a combined tumor burden of ˜4 cm³ were contained in a single chamber.

To increase sensitivity for detecting limonene from tumor-bearing mice, a specially-designed experimental setup was built in which highly purified air was continuously flowed through a mouse chamber and exited through an air sampling tube containing a sorbent material (Tenax TA) that traped VOCs, thereby pre-concentrating them for subsequent GC-MS analysis. Compared to SPME fibers, sorbent traps contained significantly larger quantities of sorbent material and therefore had higher extraction capacities.

Six one-liter chambers were set up in parallel to allow for multiple simultaneous experiments (FIG. 4C and FIG. 5). Groups of HeLa-LS-tHMGR mice and control mice bearing untransfected HeLa tumors at 5 weeks post-implantation were placed into side-by-side chambers, with 4 mice per chamber (average tumor volume per mouse: 1.2±0.2 cm³), and sampled the chamber headspace (100 mL/min airflow) for 1, 4, or 10 hours. In the experimental group, limonene was detectable in chamber air at all sampling durations. Increasing the sampling duration from 1 hour to 4 hours enabled 2.3-fold greater limonene collection (10 ng to 23 ng), and an increase to 10 hours enabled 9.4-fold greater limonene collection (10 ng to 94 ng) (FIG. 4D). Limonene levels for control mice were below 1 ng at all sampling durations. Therefore, the present studies showed that increased signal-to-background was achievable simply by sampling the chamber headspace for a longer time. By integrating limonene signal over a number of hours, the sorbent trap method improved detection sensitivity 100-fold compared to direct SIFT-MS measurements in sealed unventilated chambers (Supplementary Calculations shown in Example 2), where measurements were limited to only a few minutes before mice become hypoxic. To maximize the sensitivity, 10-hour sampling times were chosen for all subsequent mouse experiments.

Additional studies focused on the determination of the minimum tumor size at which limonene was detectable and the evaluation of whether tumor growth could be monitored via exhaled limonene alone. HeLa-LS, HeLa-LS-tHMGR, and control mice (bearing untransfected HeLa tumors) were monitored over a 5-week period. Groups of four mice per chamber (n=3 chambers per cohort) were tested once a week for total limonene released into chamber air during a 10-hour period. At week one post-implantation of tumor cells, total evolved limonene from the HeLa-LS-tHMGR cohort (11±2 ng) was statistically higher compared to the HeLa-LS (6±1 ng, p=0.049) and control mouse groups (4±3 ng, p=0.025) (FIG. 4E and Table 1).

TABLE 1
Statistical significance (Mann Whitney p-values) of limonene expression
differences between HeLa-LS, HeLa-LS-tHMGR, and HeLa control mice
by week. P values < 0.05 are highlighted in yellow.

| Mann-Whitney P-values | Week 1 | Week 2 | Week 3 | Week 4 | Week 5 |
|---|---|---|---|---|---|
| HeLa-LS vs. Control | 0.256 | 0.025 | 0.025 | 0.023 | 0.025 |
| HeLa-LS-tHMGR vs. | 0.025 | 0.025 | 0.023 | 0.023 | 0.025 |
| Control | | | | | |
| HeLa-LS-tHMGR vs. | 0.049 | 0.184 | 0.105 | 0.376 | 0.049 |
| HeLa-LS | | | | | |

At this time, the average tumor volume per mouse was 0.12 cm³, 0.10 cm³, and 0.05 cm³, for HeLa-LS-tHMGR, HeLa-LS, and control mice, respectively (FIG. 4F and FIG. 4G). Average limonene per mouse in the HeLa-LS-tHMGR group (˜2.7 ng) at week one was very close to the calculated detection limit (2.3 ng), which indicated that the minimum detectable tumor size by VOC sampling is close to 0.1 cm³, or 4.6-mm diameter (corresponding to approximately 10 million HeLa cells, see Supplementary Calculations shown in Example 2, infra). Evolved limonene from HeLa-LS mice was not statistically different from controls (p=0.26) at week one.

Thus, the expression of tHMGR by limonene-producing cancer cells aided in detecting tumors earlier relative to mice with limonene-producing tumors that did not express tHMGR, as expected based on the higher production of limonene by HeLa-LS-tHMGR cells in culture. By the second week, evolved limonene was statistically higher in both HeLa-LS-tHMGR (26.3±6.0 ng, p=0.025) and HeLa-LS mice (17.6±6.9 ng, p=0.025) than in control mice (2.3±0.3 ng) (FIG. 4E and Table 1), at an average tumor volume per mouse of 0.2 cm³, 0.18 cm³, and 0.1 cm³, respectively (FIG. 4F and FIG. 4G).

Limonene emitted from HeLa-LS and HeLa-LS-tHMGR mice increased linearly with tumor volume over 4 and 5 weeks post-implantation, respectively (FIG. 4F). Limonene evolution was higher in HeLa-LS-tHMGR mice than in HeLa-LS mice throughout the study, though this difference was statistically significant only in weeks 1 and 5. Limonene evolution from HeLa-LS and HeLa-LS-tHMGR mice peaked in weeks 4 and 5 at 60±16 ng and 94±14 ng, respectively (when tumor burden per mouse was 0.6±0.1 cm³ and 0.8±0.2 cm³, respectively). This plateau in HeLa-LS mice corresponded with a leveling off in tumor growth (i.e. no statistical change) from weeks 4 to 5 (FIG. 4F). At week 5, mice were humanely euthanized due to tumor size.

Tumor growth rate, k, was slightly greater in control mice (k=0.54) than in HeLa-LS-tHMGR (k =0.48, p=0.049), whereas it was not statistically different between HeLa-LS-tHMGR and HeLa-LS mice (k=0.53, p=0.13) or between HeLa-LS and control mice (p=0.51) (FIG. 4G). Limonene quantities collected from HeLa control mice at each time point were very similar to blank chambers without mice, with a range of <1 ng to 4 ng (FIG. 5). These values represented ambient limonene that was degassing from the chamber walls, given that limonene levels both from control mice and blank chambers were below the detection limit by the end of the 5-week study. Moreover, limonene was not detected above background in chambers containing only mouse diet gel or bedding. Therefore, the studies demonstrated that the only sources of limonene in HeLa-LS-tHMGR and HeLa-LS mice were the tumors. The average percentage of tumor limonene exhaled in the breath over all weeks was calculated at 5.2%±1.5% and 7.6%±3.1% for HeLa-LS-tHMGR and HeLa-LS mice, respectively (Supplementary Calculations shown in Example 2 infra, Table 2 through 6).

TABLE 2
Calculated number of tumor cells (in millions of
cells) in HeLa-LS-tHMGR and HeLa-LS mice given an estimate of
10⁸ cells/cm³ of tumor tissue.

| Week | HeLa-LS-tHMGR | HeLa-LS |
|---|---|---|
| 1 | 49.2 | 20.6 |
| 2 | 80.6 | 44.3 |
| 3 | 134.8 | 80.5 |
| 4 | 218.0 | 129.9 |
| 5 | 332.4 | 180.8 |

TABLE 3
Calculated quantity of limonene (in ng) produced by
HeLa-LS-tHMGR and HeLa-LS tumors in mice based on limonene
production rates of 5.6 fg/cell/day for HeLa-LS cells
and 11.1 fg/cell/day for HeLa-LS-tHMGR cells.

| Week | HeLa-LS-tHMGR | HeLa-LS |
|---|---|---|
| 1 | 227.7 | 89.7 |
| 2 | 372.7 | 153.0 |
| 3 | 623.3 | 328.4 |
| 4 | 1008.3 | 559.9 |
| 5 | 1537.5 | 656.5 |

TABLE 4
Measured quantity of limonene (in ng) exhaled in the breath by
HeLa-LS-tHMGR and HeLa-LS mice over a ten hour period by week.

| Week | HeLa-LS-tHMGR | HeLa-LS |
|---|---|---|
| 1 | 7.1 | 2.6 |
| 2 | 24.8 | 16.1 |
| 3 | 28.7 | 22.3 |
| 4 | 68.7 | 57.7 |
| 5 | 92.6 | 50.3 |

TABLE 5
Percentage of tumor limonene that was exhaled in the
breath for HeLa-LS-tHMGR and HeLa-LS mice by week.

| Week | HeLa-LS-tHMGR | HeLa-LS |
|---|---|---|
| 1 | 3.1% | 2.9% |
| 2 | 6.7% | 10.5% |
| 3 | 4.6% | 6.8% |
| 4 | 6.8% | 10.3% |
| 5 | 6.0% | 7.7% |

TABLE 6
Percentage of tumor limonene exhaled (average over all weeks).

| HeLa-LS-tHMGR | HeLa-LS |
|---|---|
| 5.2% ± 1.5% | 7.6% ± 3.1% |

Thus, the present studies reported a novel strategy for sensitive and specific breath-based cancer detection that uses limonene, a plant terpene, as an exogenous VOC reporter. First, it was demonstrated that stable heterologous expression of limonene, as validated by mass spectrometry, was achieved in a cultured HeLa human cervical cancer cell line transfected with a plasmid encoding the plant enzyme limonene synthase. It was also demonstrated that genetically co-expressing a modified key mevalonate pathway enzyme, tHMGR, doubled limonene expression in HeLa cells, thereby improving detection sensitivity for these cells in culture and in vivo. Limonene was then validated as a sensitive and specific volatile reporter of tumor presence and growth in a xenograft mouse model after subcutaneous implantation of limonene-expressing HeLa cells. Moreover, limonene waws shown to be detected when tumors were as small as 120 mm³ (˜5 mm diameter). Using human whole-body PBPK modeling, tumor-derived limonene is also detectable in human breath from a tumor as small as 7 mm in diameter.

In the clinical scenario, human subjects are placed in a room with highly pure air or breathe through a one-way filter cartridge to prevent contamination of inhaled air by ambient limonene. Exhaled air would pass through an exhaust valve directly into a sorbent tube, which is subsequently analyzed offline by GC-MS. The small filter cartridge/sorbent tube assembly is worn portably to passively collect limonene over a few hours as the subject goes about their day or at night while sleeping. Subjects need to avoid wearing perfumes or consuming citrus prior to undergoing testing. The presence of limonene in the breath at screening or surveillance then prompts clinical imaging studies, such as PET or MRI, in an attempt to spatially localize the tumor. Monitoring of VOC reporter levels is also used to assess response to therapy inexpensively and more frequently than is practical or economical with in vivo imaging in patients with metastatic disease or large disease burden.

For cancer screening and early detection, targeting expression of the VOC reporter to cancer cells using clinically relevant in vivo gene delivery approaches, including nonviral vectors, can be performed. Nonviral vectors, such as minicircles and liposomes, are generally considered safer and less invasive than viral vectors because they are non-replicative, non-integrating (minimizing the risk of insertional mutagenesis and carcinogenesis), and have low immunogenicity, with proven safety and efficacy in a number of clinical trials. Moreover, because the nucleic acid constructs used in these approaches are episomal, genetic alterations to cells are transient and do not entail permanent changes to the genome.

Vector design (HeLa-LS and HeLa-LS-tHMGR)

The sequence for R-limonene synthase was codon-optimized for expression in human cells using the GenSmart Codon Optimization tool (GenSript, Pascataway, NJ). The plastid signaling peptide (PSP), which functions independently of enzyme activity to localize R-limonene synthase to plastids in plants, was excluded as it impairs proper folding in other expression systems. The truncated limonene synthase (LS) gene exhibited markedly higher limonene production in bacterial culture compared to the full-length gene (39), and was therefore used for the duration of the study. Mammalian PiggyBac transposon gene expression vectors coding for LS or a modified hydroxy-3-methylglutaryl-CoA reductase (tHMGR) were designed using VectorBuilder (en.vectorbuilder.com/design.html) and constructed by Cyagen Biosciences. The PiggyBac transposon system consists of a vector (the PiggyBac transposon gene expression plasmid) and a transposase enzyme which recognizes transposon-specific inverted terminal repeats (ITRs) and efficiently integrates the ITRs and intervening DNA into the genome at TTAA sites. The transposase is delivered to the cell via a transposase expression vector, which is co-transfected with the PiggyBac Vectors. The vector encoding LS also contained the gene for the fluorescent protein, enhanced green fluorescent protein (eGFP), linked by a P2 A ribosomal skip sequence, with both genes driven by the same CAG promoter. Ribosomal skip sequences allow multiple genes encoded on the same mRNA transcript to be translated into separate proteins. This vector also contained a puromycin resistance gene driven by a CMV promoter for antibiotic selection.

The vector encoding tHMGR also contained the gene for the fluorescent protein, turbo red fluorescent protein (tRFP), linked by a P2 A ribosomal skip sequence, with both genes driven by the same EFla promoter. This vector also contained a hygromycin resistance gene driven by a CMV promoter for antibiotic selection.

### Cell Culture

HeLa cells (American Type Culture Collection, Manassas, VA) were cultured in Dulbecco's Modified Eagle Medium (DMEM) media supplemented with penicillin-streptomycin and 10% fetal bovine serum (FBS) (ThermoFisher, Waltham, MA). Cells were verified to be free of mycoplasma contamination using the MycoAlert Mycoplasma Detection Kit (Lonza, Allendale, NJ) and passaged when reaching 80% confluence.

### HeLa Cell Transfection

HeLa cells were transfected with a LS-encoding vector using Lipofectamine 2000 (Invitrogen, Carlsbad, CA). The ratio of the LS vector to a helper plasmid containing the transposase gene was 1:1 (0.8 μg of each per well in a 12-well plate) in Gibco Opti-MEM Reduced Serum media (ThermoFisher, Waltham, MA). Stable transfection was assessed qualitatively under fluorescence microscopy by the visual presence of high GFP expression in cells at days 3-4 post-transfection. Cells subsequently underwent antibiotic selection and multiple rounds of fluorescence-activated cell sorting (FACS) to select for high-expressing GFP subclones and were tested for limonene production as described below. This cell line was named HeLa-LS. Transfection of limonene-producing cells with a tHMGR-encoding vector (HeLa-LS-tHMGR) was accomplished in a similar manner, with hygromycin B (ThermoFisher, Waltham, MA) used for antibiotic selection of stable cells, and with FACS selection performed by gating on RFP (FIG. 3A and FIG. 3B).

### Fluorescence-Activated Cell Sorting

Roughly 1-2 million confluent stably transfected cells were sorted on a FACS Aria II or Influx sorter (Becton Dickinson, San Jose, CA). The gating strategy included forward scatter (FSC) and side scatter (SSC) gating, doublets and dead cell exclusion, and selection for the top 1-2% highest expressers of eGFP for LS-expressing cells, or tRFP for pre-sorted LS-expressing cells transfected with the vector containing the tHMGR gene.

### Cell Culture Headspace Sampling (SPME)

Stably transfected HeLa-LS or HeLa-LS-tHMGR cells were grown to confluence in T75 flasks (MIDSCI, St. Louis, MO) at 37° C. The 24-gauge needle of a solid-phase microextraction (SPME) assembly (Sigma Aldrich, St. Louis, MO) was inserted through the screw cap septum of the T75 flask and the 65-μm PDMS/DVB fiber was deployed for 30 minutes to sample the cell culture headspace. The fiber was withdrawn and adsorbed VOCs were analyzed by gas chromatography/mass spectrometry (GC/MS).

### Gas Chromatography-Mass Spectrometry

Analysis of SPME fibers was performed on an Agilent 7890/5975 GC/MS instrument (Agilent Technologies, Santa Clara, CA) at the Stanford Mass Spectrometry Facility. One microliter of sample was injected through an SPME inlet guide (Supelco, Bellefonte, PA) into the GC injection port, equipped with a Thermogreen LB-2 pre-drilled septum (Supelco) and deactivated glass inlet liner (Supelco), and run in pulsed splitless mode. Helium was used as the carrier gas with a constant flow rate of 1.6 mL/min and velocity of 27.8 cm/s through an Agilent DB-WAX column (60 m×250 μm×0.25 μm). The initial oven temperature was held at 4° C. for 2 minutes, increased at a rate of 2° C./min up to 72° C., then ramped at 40° C./min to 220° C. Total run time was 21.7 minutes. Initial scans were run in full scan mode at m/z 10-400. Subsequently, samples were run in selected ion monitoring (SIM) mode, targeting the characteristic ion peaks for limonene: m/z 68, 93, and 136.

### Quantitation of Limonene Production in HeLa Cells

Prior to cell studies, a calibration curve was generated. Serial dilutions of pure limonene (Sigma Aldrich, St. Louis, MO) in ethanol were prepared in Eppendorf tubes and spiked into 10 mL of media (DMEM with 10% FBS) to final concentrations ranging from 0.01 ng to 100 μg in T75 flasks with screwcap septa (MIDSCI, St. Louis, MO). The flasks were manually agitated for 10 seconds and the screw cap septum was punctured by a needle. The flask headspace was sampled for 20 seconds at least 3 times per concentration using selected ion flow mass spectrometry (SIFT-MS, Syft Technologies, Christchurch, New Zealand) with a helium gas carrier. Limonene detection was performed by soft-ionization using H₃O⁺ (m/z, 137; branching ratio, 68%; reaction rate, 2.6×10⁻⁹ cm³/s), NO⁺ (m/z, 136; branching ratio, 88%; reaction rate, 2.2×10⁻⁹ cm³/s) and O₂⁺ (m/z, 93; branching ratio, 29%; reaction rate, 2.2×10⁻⁹ cm³/s) to calculate limonene concentration in real-time. After establishing the calibration curve, HeLa-LS and HeLa-LS-tHMGR cells were spiked into 10 mL media (DMEM with 10% FBS) in varying numbers ranging from 20,000 to 10 million cells in T75 flasks. The flasks were incubated at 37° C. for 24 hours, after which headspace limonene concentrations were measured using SIFT-MS. The cells were then harvested and counted with cell numbers at harvest ranging from −45,000 to 25 million.

Quantitation of Limonene Evolution from Limonene-Injected Mice

Prior to mouse studies, a calibration curve was generated. Known limonene quantities (10 μg to 100 μg) were added to 10 mL of water in 0.5-mL chambers (Kent Scientific, Torrington, CT). The chambers were capped, briefly agitated, and allowed to sit for 15 minutes to equilibrate. The chamber inlet was then uncapped and the headspace was sampled by SIFT-MS for limonene. After establishing the calibration curve, serial tenfold dilutions of limonene in ethanol were prepared and a twenty-microliter volume of each solution (1 to 1000 μg limonene) was injected intraperitoneally into immunocompromised nude mice. The injection site was rinsed thoroughly under warm water for 15 seconds to remove possible limonene residue from the skin. Each mouse was then placed in a closed 0.5-L chamber for 15 minutes, at which point the chamber inlet was uncapped and the headspace was sampled by SIFT-MS for 20 seconds.

### Xenograft Tumor Mouse Model

A “xenograft” refers to the transplant of an organ, tissue, or cells to an individual of another species. In this case, a “xenograft tumor mouse model” refers to implantation of human tumor cells into mice. Ten-week-old athymic nude (nu/nu) mice (Charles River Laboratories, Wilmington, MA) were inoculated subcutaneously in both flanks with either HeLa-LS, HeLa-LS-tHMGR, or untransfected control HeLa cells (1 million cells in 100 μL of Matrigel [ThermoFisher, Waltham, MA] into each flank). Prior to each experiment, mouse tumors on both flanks were measured via caliper and the tumor length (L), width (W), and depth (D) were

$$V = \frac{π}{6}$$

$$L \times W \times D .$$

### Mouse Chamber/Sorbent Trap Assembly

Six one-liter chambers (Braintree Scientific, Braintree, MA) were operated in parallel for simultaneous mouse limonene measurements (FIG. 6). The outlet of each chamber was connected in series via tygon tubing to a glass condenser (25 mL impinger, SKC Ltd., UK) on ice (cold trap) and then to a sorbent tube containing Tenax TA resin (Markes International Ltd., UK) that traps and concentrates VOCs. The cold trap prevents moisture from soaking the sorbent resin. The inlet of each chamber was connected in series to a sacrificial Tenax sorbent tube, which served to purify inflowing air, and an upstream 0.25 inch stainless steel metering valve (Swagelok Company, Solon, OH) that individually controlled air flow into each chamber. The metering valves to all six chambers were connected via reducing unions, union tees, and ⅛″ copper tubing to a benchtop pressure regulator (Markes International Ltd., UK, U-GAS03) set to 5 psi, which was connected via a single copper line to a compressed gas cylinder containing highly pure air (Vehicle Emission Grade Air, Airgas Inc., Radnor, PA) set to 20 psi. For ease of cleaning the induction chambers between experiments, the tygon connections to inlet and outlet components were interrupted by 0.25 inch snap-on/snap-off fasteners (Thermoplastic Quick Couplings, Omega Engineering Inc, Norwalk, CT).

Operation of Chamber/Sorbent Trap Assembly for VOC Sampling from Tumor Mice

Prior to initial mouse experiments, the induction chambers were flushed with highly pure air at 100 mL/min for 3 days. On the evening prior to experiments, 40 mL of mouse bedding and diet gel (CearH2O, Portland, ME) were placed in each chamber, and air flow was continued overnight (˜10 hours) with the Tenax tubes connected to measure the background limonene levels in empty chambers. On the day of experiments, mice were pre-hydrated with a subcutaneous injection of 0.5 mL sterile saline. Air flow was continued for 30 minutes after mice were placed in the induction chambers to remove any ambient limonene entering while the chambers were briefly open. Tenax tubes were then replaced. A flow meter (Ellutia 7000, Ellutia Ltd, UK) measured the air flow exiting each Tenax tube and the pin valves were tuned to achieve an air flow rate of 100 mL/min. When removing or replacing the screw caps on Tenax tubes, care was taken to keep the tube ends covered with a clean glove to prevent contamination from ambient air. Air was flowed continuously for the duration of the experiments (10 hours). After each experiment, mice were placed back in their cages. The chambers were then rinsed with water, 70% ethanol, and dried before highly pure air flow was resumed at 20 mL/min to maintain low background limonene levels in the chambers prior to subsequent experiments. Upon completion of mouse experiments, Tenax tubes were stored on ice and shipped to ALS Environmental (Simi Valley, CA) for thermal desorption and GC/MS analysis.

### Example 3: Transduction of Adenoviral Constructs Containing the Limonene Synthase Gene

Furthermore, studies also focused on transduction of adenoviral constructs containing the limonene synthase gene in cell culture and in vivo in a mouse tumor model. Human MeWo (melanoma) or HCC827 (non-small cell lung cancer) cell line cells were seeded at a density of ˜60,000 cells per cm² in cell culture media containing 10% FBS in T25 or T75 culture flasks, respectively (FIG. 7A and FIG. 7B). Twenty-four hours later, the culture media was replaced with serum-free media containing chimeric Ad5/F35 adenovirus at a multiplicity of infection (MOI) of 1000. The adenoviral DNA construct (named Ad5/F35-hTert-LS-HMGR-mKate) contains the genes encoding limonene synthase (LS), HMGR, and the red fluorescence reporter mKate, all driven by a human telomerase reverse transcriptase (hTert) promoter. After a 24 hour incubation at 37° C., the virus-containing media was replaced with media containing 10% FBS. Fluorescent images were taken using an EVOS cell imaging system with a red fluorescent protein (RFP) filter on day 4.

Limonene levels in parts-per-billion from MeWo cells in T25 flasks at day 4 after adenovirus transduction at MOIs of 200, 1000, or 5000, and from untransduced MeWo cells (no virus added) were also examined (FIG. 7C). The dashed line represents background signal from untransduced cells.

Additionally, nude mice were implanted with 2.5 million MeWo or HCC827 cells in each flank (FIG. 7D and FIG. 7E). Five days after implantation, adenovirus in 20 μL of saline was injected into each flank tumor. Bioluminescence images were taken within 10 minutes of retro-orbital intravenous d-Luciferin administration on day 4 after adenovirus injection. The numbers at the bottom of each image refer to the adenoviral construct injected into that tumor, as follows: 0. No virus injected; 1. Ad5/F35-hTert-LS-HMGR-mKate (10¹⁰ viral particles); 2. Ad5/F35-pSurv-LS-Luc2-mCherry: Ad5/F35 adenovirus encoding LS, Luc2, and the red fluorescence reporter mCherry, all driven by a human Survivin promoter (pSurv)(10⁸ viral particles); 3. Ad5/F35-hTert-LS-Luc2-mCherry: Ad5/F35 adenovirus encoding LS, Luc2, and mCherry, all driven by an hTert promoter (10⁸ viral particles). Note that construct 1 does not contain a bioluminescence reporter gene; therefore, tumors injected with this adenoviral construct do not bioluminesce after systemic injection of dLuc. As shown in FIG. 7D, the adenoviral construct injected into each flank tumor was also injected into the adjacent thigh muscle as a control. Note the absence of bioluminescence signal in thigh muscles. Not all tumors showed bioluminescence signal, likely attributable to injection technique.

### Example 5: Sequences

Enzyme (+)-limonene synthase from oranges (Citrus sinensis)-Genbank accession number
AOP12358.2-SEQ ID NO: 1

| | |
|---|---|
| 1 | MSSCINPSTL ATSVNGFKCL PLATNRAAIR IMAKNKPVQC LVSTKYDNLT VDRRSANYQP |
| 61 | SIWDHDFLQS LNSNYTDETY KRRAEELKGK VKTAIKDVTE PLDQLELIDN LQRLGLAYHF |
| 121 | EPEIRNILRN IHNHNKDYNW RKENLYATSL EFRLLRQHGY PVSQEVFSGF KDDKVGFICD |
| 181 | DFKGILSLHE ASYYSLEGES IMEEAWQFTS KHLKEMMITS NSKEEDVFVA EQAKRALELP |
| 241 | LHWKAPMLEA RWFIHVYEKR EDKNHLLLEL AKLEFNTLQA IYQEELKDIS GWWKDTGLGE |
| 301 | KLSFARNRLV ASFLWSMGIA FEPQFAYCRR VLTISIALIT VIDDIYDVYG TLDELEIFTD |
| 361 | AVARWDINYA LKHLPGYMKM CFLALYNFVN EFAYYVLKQQ DFDMLLSIKH AWLGLIQAYL |
| 421 | VEAKWYHSKY TPKLEEYLEN GLVSITGPLI ITISYLSGTN PIIKKELEFL ESNPDIVHWS |
| 481 | SKIFRLQDDL GTSSDEIQRG DVPKSIQCYM HETGASEEVA REHIKDMMRQ MWKKVNAYTA |
| 541 | DKDSPLTRTT AEFLLNLVRM SHFMYLHGDG HGVQNQETID VGFTLLFQPI PLEDKDMAFT |
| 601 | ASPGTKG |

A DNA sequence encoding enzyme (+)-limonene synthase from oranges (Citrus sinensis)-SEQ ID
NO: 2. The DNA sequence was codon optimized for expression in humans.
ATGAGCAGCTGCATCAATCCCAGCACCCTGGCAACATCCGTGAATGGCTTCAAATGCCTGCCTCTGGCAACAAACAGAGC
TGCTATCCGCATCATGGCCAAAAACAAGCCCGTGCAGTGCCTGGTGTCCACAAAATACGATAATCTGACAGTGGACCGGC
GGTCTGCCAACTACCAGCCATCTATCTGGGACCACGACTTCCTGCAGTCTCTGAATAGCAACTATACCGACGAGACCTAC
AAGAGGAGGGCCGAAGAGCTGAAAGGCAAGGTGAAGACCGCCATCAAGGACGTGACCGAGCCCCTGGATCAGCTGGAGCT
GATCGATAACCTGCAGCGCCTGGGACTGGCTTACCATTTTGAACCTGAGATTCGCAATATTCTGAGGAACATCCACAATC
ACAACAAGGATTATAACTGGAGAAAGGAGAACCTGTACGCTACCAGCCTCGAGTTTCGCCTGCTCAGGCAGCATGGGTAC
CCCGTGTCCCAGGAGGTGTTCAGCGGCTTCAAAGACGATAAAGTGGGCTTCATTTGTGACGATTTTAAGGGCATCCTGAG
TCTGCACGAGGCCTCTTACTATAGCCTGGAGGGAGAGAGCATCATGGAGGAGGCCTGGCAGTTTACCAGCAAACATCTCA
AAGAGATGATGATTACCTCCAATTCTAAGGAGGAGGACGTGTTCGTCGCTGAGCAGGCCAAAAGAGCCCTGGAGCTGCCC
CTGCACTGGAAAGCCCCCATGCTGGAAGCTCGGTGGTTCATCCACGTGTATGAGAAACGCGAGGATAAAAACCACCTGCT
GCTCGAGCTGGCCAAACTCGAGTTTAACACTCTCCAGGCCATCTACCAGGAGGAGCTGAAGGACATTTCCGGCTGGTGGA
AGGACACCGGACTGGGCGAAAAACTGAGCTTCGCCAGGAACCGGCTGGTGGCCTCCTTCCTGTGGTCCATGGGTATCGCC
TTCGAGCCACAGTTTGCCTACTGCAGGAGAGTGCTGACTATCAGCATCGCTCTGATCACCGTGATTGACGACATTTATGA
CGTGTACGGGACCCTGGATGAGCTGGAGATCTTTACTGACGCCGTGGCCCGGTGGGATATCAACTACGCCCTTAAGCACC
TGCCCGGCTACATGAAGATGTGCTTCCTGGCCCTGTACAACTTTGTGAATGAATTTGCCTACTACGTGCTGAAGCAGCAG
GACTTTGACATGCTCCTGTCCATTAAGCACGCATGGCTGGGACTGATCCAGGCCTATCTGGTGGAGGCCAAGTGGTACCA
CTCCAAGTACACACCTAAGCTGGAGGAGTACTTGGAGAACGGCCTGGTGAGCATCACCGGACCCCTGATCATCACCATCT
CCTATCTTTCTGGGACAAACCCTATTATCAAGAAGGAGCTGGAATTCCTGGAGTCTAATCCCGATATCGTTCACTGGAGC
TCCAAGATTTTCAGGCTGCAGGACGACCTGGGGACCAGTTCAGATGAGATCCAGAGAGGCGATGTGCCTAAGTCCATCCA
GTGTTACATGCACGAAACCGGCGCCTCCGAGGAGGTGGCCCGGGAACACATCAAGGACATGATGCGCCAGATGTGGAAGA
AAGTGAACGCCTACACCGCAGACAAGGACTCCCCCCTGACCCGCACCACAGCCGAGTTCCTGCTGAACCTGGTGAGAATG
AGCCACTTCATGTACCTGCACGGAGACGGCCACGGCGTGCAGAACCAGGAGACAATCGACGTGGGCTTCACTCTCCTGTT
CCAGCCCATCCCTCTGGAGGATAAAGATATGGCCTTCACAGCCAGTCCTGGAACCAAGGGATGA
Enzyme (+)-limonene synthase from kumquat (Citrus japonica)-Genbank accession number
QBK56496.1-SEQ ID NO: 3

| | |
|---|---|
| 1 | MSSSINPSTL VTSVNGFKCL PLATNKAAIR IMAKNKPVQC LVSAKYDNLT VDRRSANYQP |
| 61 | SIWDHDFLQS LNSNYTDETY RRRAEELKGK VKTAIKDVTE PLDQLELIDN LQRLGLAYRF |
| 121 | ETEIRNILHN IYNNNKDYVW RKENLYATSL EFRLLRQHGY PVSQEVENGF KDDQGGFICD |
| 181 | DFKGILSLHE ASYYRLEGES IMEEAWQFTS KHLKEVMISK SKEEDVFVAE QAKRALELPL |
| 241 | HWKVPMLEAR WFIHIYERRE DKNHLLLELA KMEFNTLQAI YQEELKEISG WWKDTGLGEK |
| 301 | LSFARNRLVA SFLWSMGIAF EPQFAYCRRV LTISIALITV IDDIYDVYGT LDELEIFTDA |
| 361 | VERWDINYAL KHLPGYMKMC FLALYNFVNE FAYYVLKQQD FDMLLSIKNA WLGLIQAYLV |
| 421 | EAKWYHSKYT PKLEEYLENG LVSITGPLII TISYLSGTNP IIKKELEFLE SNPDIVHWSS |
| 481 | KIFRLQDDLG TSSDEIQRGD VPKSIQCYMH ETGASEEVAR EHIKDMMRQM WKKVNAYTAD |
| 541 | KDSPLTRTTT EFLLNLVRMS HFMYLHGDGH GVQNQETIDV GFTLLFQPIP LEDKHMAFAA |
| 601 | SPGTKG |

A DNA sequence encoding enzyme (+)-limonene synthase from kumquat (Citrus japonica)-SEQ
ID NO: 4. The DNA sequence was codon optimized for expression in humans.
ATGAGCTCCAGCATTAACCCATCCACCCTTGTGACTAGCGTGAATGGCTTCAAGTGCCTGCCCCTGGCAACTAACAAGGC
CGCCATCCGGATCATGGCCAAGAACAAGCCAGTGCAGTGCCTGGTGTCTGCCAAGTATGACAATCTGACAGTGGACAGAC
GGAGCGCCAATTACCAGCCAAGCATCTGGGACCACGATTTCCTGCAGAGCCTGAACAGCAACTACACTGACGAGACCTAC
AGACGGCGCGCTGAGGAGCTGAAAGGGAAGGTGAAGACCGCCATCAAGGATGTGACCGAGCCACTGGACCAGCTGGAACT
GATTGATAACCTGCAGAGACTGGGCCTGGCCTACAGATTCGAAACCGAGATCAGGAACATTCTGCACAACATTTACAACA
ACAACAAGGACTACGTGTGGAGAAAAGAGAACCTGTATGCCACCAGCCTGGAGTTCAGACTGCTGCGCCAGCACGGATAC
CCAGTGAGCCAGGAGGTGTTCAATGGCTTCAAGGACGACCAGGGCGGATTCATCTGCGATGATTTTAAAGGGATCCTGAG
CCTGCACGAGGCCTCCTACTACCGCCTGGAGGGAGAATCTATTATGGAGGAGGCCTGGCAGTTCACCAGCAAGCACCTGA
AAGAGGTGATGATTTCCAAGAGCAAGGAGGAGGACGTGTTTGTCGCCGAACAGGCCAAGAGAGCTCTGGAACTGCCTCTG
CACTGGAAGGTGCCAATGCTGGAAGCCAGGTGGTTTATACACATTTACGAGAGAAGAGAGGACAAGAATCACCTGCTGCT
GGAGCTGGCTAAAATGGAGTTTAATACCTTGCAGGCCATTTATCAGGAGGAGCTGAAGGAAATCAGCGGCTGGTGGAAGG
ATACTGGATTGGGCGAGAAGCTCAGCTTTGCCCGGAACAGACTGGTGGCCAGCTTTCTGTGGTCTATGGGCATCGCCTTC
GAGCCCCAGTTTGCCTATTGTCGGAGAGTGCTGACAATTAGCATCGCCCTGATCACTGTGATCGACGACATCTACGACGT
GTACGGCACACTGGACGAGCTGGAAATCTTCACCGATGCCGTGGAGAGGTGGGACATCAACTACGCCCTGAAGCATCTGC
CAGGCTACATGAAGATGTGTTTTCTGGCCCTGTACAATTTCGTGAATGAGTTCGCCTATTACGTGCTCAAGCAGCAGGAC
TTTGACATGCTGCTGTCCATCAAGAACGCTTGGCTGGGGCTGATTCAGGCTTACCTGGTGGAGGCCAAATGGTACCACTC
TAAATACACTCCTAAACTGGAAGAGTACCTGGAAAACGGACTGGTGAGCATCACCGGCCCACTGATCATTACCATCAGCT
ACCTGTCCGGGACTAACCCCATCATCAAAAAGGAGCTCGAATTTCTGGAAAGTAATCCCGATATCGTGCACTGGAGCAGC
AAGATTTTCAGGCTTCAGGATGATCTGGGGACCTCCTCCGATGAGATCCAGAGAGGCGACGTGCCAAAAAGTATTCAGTG
CTACATGCACGAGACCGGGGCCTCTGAGGAGGTGGCCCGGGAACATATTAAAGATATGATGAGGCAGATGTGGAAAAAGG
TGAATGCCTATACAGCTGACAAGGACTCCCCCCTGACAAGGACAACAACAGAATTCTTGCTGAACCTGGTGAGAATGAGC
CATTTCATGTACCTGCACGGCGACGGCCATGGCGTGCAGAATCAGGAGACTATTGACGTGGGCTTCACACTGCTGTTCCA
GCCCATCCCCCTGGAGGACAAGCACATGGCCTTTGCAGCCAGCCCTGGCACTAAAGGCTAA
Enzyme (+)-limonene synthase from lemons (Citrus limon)-Genbank accession number AF514289-
SEQ ID NO: 5

| | |
|---|---|
| 0 | MSSCINPSTL VTSANGFKCL PLATNKAAIR IMAKNKPVQC LVSAKYDNLI VDRRSANYQP |
| 60 | SIWDHDFLQS LNSNYTDETY RRRAEELKGK VKIAIKDVTE PLDQLELIDN LQRLGLAYRF |
| 120 | ETEIRNILHN IYNNNKDYVW RKENLYATSL EFRLLRQHGY PVSQEVENGF KDDQGGFIFD |
| 180 | DFKGILSLHE ASYYSLEGES IMEEAWQFTS KHLKEVMISK SMEEDVFVAE QAKRALELPL |
| 240 | HWKVPMLEAR WFIHVYEKRE DKNHLLLELA KMEFNTLQAI YQEELKEISG WWKDTGLGEK |
| 300 | LSFARNRLVA SFLWSMGIAF EPQFAYCRRV LTISIALITV IDDIYDVYGT LDELEIFTDA |
| 360 | VARWDINYAL KHLPGYMKMC FLALYNFVNE FAYYVLKQQD FDMLLSIKNA WLGLIQAYLV |
| 420 | EAKWYHSKYT PKLEEYLENG LVSITGPLII AISYLSGTNP IIKKELEFLE SNPDIVHWSS |
| 480 | KIFRLQDDLG TSSDEIQRGD VPKSIQCYMH ETGASEEVAR EHIKDMMRQM WKKVNAYTAD |
| 540 | KDSPLTRTTT EFLLNLVRMS HFMYLHGDGH GVQNQETIDV GFTLLFQPIP LEDKDMAFTA |
| 600 | SPGTKG |

A DNA sequence encoding enzyme (+)-limonene synthase from lemons (Citrus limon)-SEQ ID
NO: 6. The DNA sequence was codon optimized for expression in humans.
ATGAGCTCCTGTATTAACCCATCCACCCTTGTGACTAGCGCCAATGGCTTCAAGTGCCTGCCCCTGGCAACTAACAAGGC
CGCCATCCGGATCATGGCCAAGAACAAGCCAGTGCAGTGCCTGGTGTCTGCCAAGTATGACAATCTGATTGTGGACAGAC
GGAGCGCCAATTACCAGCCAAGCATCTGGGACCACGATTTCCTGCAGAGCCTGAACAGCAACTACACTGACGAGACCTAC
AGACGGCGCGCTGAGGAGCTGAAAGGGAAGGTGAAGATCGCCATCAAGGATGTGACCGAGCCACTGGACCAGCTGGAACT
GATTGATAACCTGCAGAGACTGGGCCTGGCCTACAGATTCGAAACCGAGATCAGGAACATTCTGCACAACATTTACAACA
ACAACAAGGACTACGTGTGGAGAAAAGAGAACCTGTATGCCACCAGCCTGGAGTTCAGACTGCTGCGCCAGCACGGATAC
CCAGTGAGCCAGGAGGTGTTCAATGGCTTCAAGGACGACCAGGGCGGATTCATCTTCGATGATTTTAAAGGGATCCTGAG
CCTGCACGAGGCCTCCTACTACTCCCTGGAGGGAGAATCTATTATGGAGGAGGCCTGGCAGTTCACCAGCAAGCACCTGA
AAGAGGTGATGATTTCCAAGAGCATGGAGGAGGACGTGTTTGTCGCCGAACAGGCCAAGAGAGCTCTGGAACTGCCTCTG
CACTGGAAGGTGCCAATGCTGGAAGCCAGGTGGTTTATACACGTGTACGAGAAGAGAGAGGACAAGAATCACCTGCTGCT
GGAGCTGGCTAAAATGGAGTTTAATACCTTGCAGGCCATTTATCAGGAGGAGCTGAAGGAAATCAGCGGCTGGTGGAAGG
ATACTGGATTGGGCGAGAAGCTCAGCTTTGCCCGGAACAGACTGGTGGCCAGCTTTCTGTGGTCTATGGGCATCGCCTTC
GAGCCCCAGTTTGCCTATTGTCGGAGAGTGCTGACAATTAGCATCGCCCTGATCACTGTGATCGACGACATCTACGACGT
GTACGGCACACTGGACGAGCTGGAAATCTTCACCGATGCCGTGGCAAGGTGGGACATCAACTACGCCCTGAAGCATCTGC
CAGGCTACATGAAGATGTGTTTTCTGGCCCTGTACAATTTCGTGAATGAGTTCGCCTATTACGTGCTCAAGCAGCAGGAC
TTTGACATGCTGCTGTCCATCAAGAACGCTTGGCTGGGGCTGATTCAGGCTTACCTGGTGGAGGCCAAATGGTACCACTC
TAAATACACTCCTAAACTGGAAGAGTACCTGGAAAACGGACTGGTGAGCATCACCGGCCCACTGATCATTGCCATCAGCT
ACCTGTCCGGGACTAACCCCATCATCAAAAAGGAGCTCGAATTTCTGGAAAGTAATCCCGATATCGTGCACTGGAGCAGC
AAGATTTTCAGGCTTCAGGATGATCTGGGGACCTCCTCCGATGAGATCCAGAGAGGCGACGTGCCAAAAAGTATTCAGTG
CTACATGCACGAGACCGGGGCCTCTGAGGAGGTGGCCCGGGAACATATTAAAGATATGATGAGGCAGATGTGGAAAAAGG
TGAATGCCTATACAGCTGACAAGGACTCCCCCCTGACAAGGACAACAACAGAATTCTTGCTGAACCTGGTGAGAATGAGC
CATTTCATGTACCTGCACGGCGACGGCCATGGCGTGCAGAATCAGGAGACTATTGACGTGGGCTTCACACTGCTGTTCCA
GCCCATCCCCCTGGAGGACAAGGACATGGCCTTTACAGCCAGCCCTGGCACTAAAGGCTAA
Enzyme (+)-limonene synthase from rough lemon (Citrus jambhiri)-Genbank accession numbers
AF514287 and BAF73932-SEQ ID NO: 7

| | |
|---|---|
| 0 | MSSCINPSTL VTSVNAFKCL PLATNKAAIR IMAKYKPVQC LISAKYDNLT VDRRSANYQP |
| 60 | SIWDHDFLQS LNSNYTDEAY KRRAEELRGK VKIAIKDVIE PLDQLELIDN LQRLGLAHRF |
| 120 | ETEIRNILNN IYNNNKDYNW RKENLYATSL EFRLLRQHGY PVSQEVFNGF KDDQGGFICD |
| 180 | DFKGILSLHE ASYYSLEGES IMEEAWQFTS KHLKEVMISK NMEEDVFVAE QAKRALELPL |
| 240 | HWKVPMLEAR WFIHIYERRE DKNHLLLELA KMEFNTLQAI YQEELKEISG WWKDTGLGEK |
| 300 | LSFARNRLVA SFLWSMGIAF EPQFAYCRRV LTISIALITV IDDIYDVYGT LDELEIFTDA |
| 360 | VERWDINYAL KHLPGYMKMC FLALYNFVNE FAYYVLKQQD FDLLLSIKNA WLGLIQAYLV |
| 420 | EAKWYHSKYT PKLEEYLENG LVSITGPLII TISYLSGTNP IIKKELEFLE SNPDIVHWSS |
| 480 | KIFRLQDDLG TSSDEIQRGD VPKSIQCYMH ETGASEEVAR QHIKDMMRQM WKKVNAYTAD |
| 540 | KDSPLTGTTT EFLLNLVRMS HFMYLHGDGH GVQNQETIDV GFTLLFQPIP LEDKHMAFTA |
| 600 | SPGTKG |

A DNA sequence encoding enzyme (+)-limonene synthase from rough lemon (Citrus jambhiri)-
SEQ ID NO: 8. The DNA sequence was codon optimized for expression in humans.
ATGAGCTCCTGTATTAACCCATCCACCCTTGTGACTAGCGTGAATGCCTTCAAGTGCCTGCCCCTGGCAACTAACAAGGC
CGCCATCCGGATCATGGCCAAGTACAAGCCAGTGCAGTGCCTGATCTCTGCCAAGTATGACAATCTGACAGTGGACAGAC
GGAGCGCCAATTACCAGCCAAGCATCTGGGACCACGATTTCCTGCAGAGCCTGAACAGCAACTACACTGACGAGGCCTAC
AAGCGGCGCGCTGAGGAGCTGCGCGGGAAGGTGAAGATCGCCATCAAGGATGTGATCGAGCCACTGGACCAGCTGGAACT
GATTGATAACCTGCAGAGACTGGGCCTGGCCCACAGATTCGAAACCGAGATCAGGAACATTCTGAATAACATTTACAACA
ACAACAAGGACTACAATTGGAGAAAAGAGAACCTGTATGCCACCAGCCTGGAGTTCAGACTGCTGCGCCAGCACGGATAC
CCAGTGAGCCAGGAGGTGTTCAATGGCTTCAAGGACGACCAGGGCGGATTCATCTGCGATGATTTTAAAGGGATCCTGAG
CCTGCACGAGGCCTCCTACTACTCCCTGGAGGGAGAATCTATTATGGAGGAGGCCTGGCAGTTCACCAGCAAGCACCTGA
AAGAGGTGATGATTTCCAAGAATATGGAGGAGGACGTGTTTGTCGCCGAACAGGCCAAGAGAGCTCTGGAACTGCCTCTG
CACTGGAAGGTGCCAATGCTGGAAGCCAGGTGGTTTATACACATTTACGAGAGAAGAGAGGACAAGAATCACCTGCTGCT
GGAGCTGGCTAAAATGGAGTTTAATACCTTGCAGGCCATTTATCAGGAGGAGCTGAAGGAAATCAGCGGCTGGTGGAAGG
ATACTGGATTGGGCGAGAAGCTCAGCTTTGCCCGGAACAGACTGGTGGCCAGCTTTCTGTGGTCTATGGGCATCGCCTTC
GAGCCCCAGTTTGCCTATTGTCGGAGAGTGCTGACAATTAGCATCGCCCTGATCACTGTGATCGACGACATCTACGACGT
GTACGGCACACTGGACGAGCTGGAAATCTTCACCGATGCCGTGGAGAGGTGGGACATCAACTACGCCCTGAAGCATCTGC
CAGGCTACATGAAGATGTGTTTTCTGGCCCTGTACAATTTCGTGAATGAGTTCGCCTATTACGTGCTCAAGCAGCAGGAC
TTTGACCTCCTGCTGTCCATCAAGAACGCTTGGCTGGGGCTGATTCAGGCTTACCTGGTGGAGGCCAAATGGTACCACTC
TAAATACACTCCTAAACTGGAAGAGTACCTGGAAAACGGACTGGTGAGCATCACCGGCCCACTGATCATTACCATCAGCT
ACCTGTCCGGGACTAACCCCATCATCAAAAAGGAGCTCGAATTTCTGGAAAGTAATCCCGATATCGTGCACTGGAGCAGC
AAGATTTTCAGGCTTCAGGATGATCTGGGGACCTCCTCCGATGAGATCCAGAGAGGCGACGTGCCAAAAAGTATTCAGTG
CTACATGCACGAGACCGGGGCCTCTGAGGAGGTGGCCCGGCAGCATATTAAAGATATGATGAGGCAGATGTGGAAAAAGG
TGAATGCCTATACAGCTGACAAGGACTCCCCCCTGACAGGGACAACAACAGAATTCTTGCTGAACCTGGTGAGAATGAGC
CATTTCATGTACCTGCACGGCGACGGCCATGGCGTGCAGAATCAGGAGACTATTGACGTGGGCTTCACACTGCTGTTCCA
GCCCATCCCCCTGGAGGACAAGCACATGGCCTTTACAGCCAGCCCTGGCACTAAAGGCTAA
Enzyme (+)-limonene synthase from trifoliate orange (Citrus_trifoliata)-Genbank accession
number BAG74774.1-SEQ ID NO: 9.
MSSCINPSTL ATSVNGFKYL PLATNRAAIR ITAKNKPVQC LVSAKYDNLT VDRRSANYQP
PIWDHDFLQS LNSDYTDETY RRRAEELKGK VKTAIEDVTE PLDQLELIDN LQRLGLAYHF
ETEIRNILHN IYNNNKDYIW RKENLYATSL EFRLLRQHGY PVSQEVSTGF KEDKGVFICD
DEMGILSLHE ASYYSLEGES IMEEAWQFTS KHLKEMMIIS NSKEEDVEVA EQAKRALELP
LHWKVPMLEA RWFIHVYEKR EDKNHLLLEL AKLEFNVLQA IYQEELKDVS RWWKDIGLGE
KLNFARDSLV ASFVWSMGIV FEPQFAYCRR ILTITFALIS VIDDIYDVYG TLDELELFAD
AVERWDINYA LNHLPDYMKI CFLALYNLVN EFTYYVLKQQ DEDILRSIKN AWLRNIQAYL
VEAKWYHGKY TPTLGEFLEN GLVSIGGPMV TMTAYLSGTN PIIEKELEFL ESNQDIIHWS
FKILRLQDDL GTSSDEIQRG DVPKSIQCYM HETGASEEVA REHIKDMMRQ MWKKVNAYRA
DKDSPLSQTT VEFILNVVRV SHFMYLHGDG HGAQNQETMD VVFTLLFQPI PLDDKHIVAT
SSPVTKG
A DNA sequence encoding enzyme (+)-limonene synthase from trifoliate orange (Citrus_trifoliata)
-SEQ ID NO: 10. The DNA sequence was codon optimized for expression in humans.
ATGTCCAGCTGCATTAACCCTTCCACACTGGCCACATCCGTGAACGGCTTCAAGTACCTGCCTCTGGCCACCAATCGGGC
CGCCATCAGAATCACCGCCAAAAACAAGCCAGTGCAGTGTCTGGTGTCCGCCAAGTACGACAATCTGACTGTGGACAGAC
GCTCCGCCAATTACCAGCCCCCTATCTGGGACCACGATTTTCTGCAGAGCCTGAATTCCGATTATACCGACGAGACCTAC
AGGAGAAGGGCCGAAGAACTGAAGGGAAAAGTCAAGACCGCCATCGAAGACGTGACCGAGCCCCTTGATCAGCTGGAACT
GATCGATAATCTGCAGAGGCTGGGGCTGGCCTACCACTTTGAGACAGAGATCAGGAACATCCTGCACAATATTTACAACA
ACAACAAGGACTATATTTGGCGCAAGGAGAACCTGTACGCCACCAGCCTGGAGTTCAGGCTGCTGAGGCAGCACGGATAC
CCTGTGAGCCAGGAGGTGAGCACAGGCTTTAAGGAGGACAAAGGCGTCTTTATCTGTGACGATTTCATGGGAATCCTGTC
CCTGCATGAGGCCTCATACTACAGCCTGGAGGGCGAGTCCATCATGGAAGAGGCTTGGCAGTTCACCTCCAAACACCTGA
AGGAGATGATGATCATCTCCAACTCTAAGGAGGAGGACGTCTTCGTGGCCGAGCAGGCCAAGAGAGCTCTGGAGCTGCCA
CTGCACTGGAAGGTGCCCATGCTGGAGGCCCGGTGGTTCATCCACGTGTACGAGAAGCGCGAGGATAAGAACCACCTGCT
GCTGGAACTCGCCAAACTTGAGTTTAATGTGCTGCAGGCCATCTACCAGGAGGAGCTGAAAGATGTGAGCAGATGGTGGA
AGGATATTGGCCTGGGAGAGAAACTGAATTTCGCCCGAGACAGCCTGGTCGCTTCCTTCGTCTGGTCTATGGGCATCGTG
TTCGAGCCACAGTTCGCCTATTGCAGACGGATCCTGACTATTACATTCGCCCTGATTAGTGTGATCGACGACATCTATGA
TGTGTACGGTACACTGGACGAGCTGGAGCTGTTCGCCGACGCCGTGGAGAGGTGGGACATCAACTACGCCCTGAACCACC
TGCCCGACTATATGAAGATCTGCTTCCTGGCTTTGTACAACCTGGTGAACGAGTTTACCTACTACGTGCTGAAGCAGCAG
GACTTCGACATCCTGAGGAGCATCAAGAATGCCTGGCTGCGAAATATTCAGGCCTACCTGGTGGAAGCTAAGTGGTACCA
CGGCAAATATACACCGACCTTGGGCGAGTTCCTGGAGAACGGCCTGGTGTCCATCGGAGGGCCTATGGTGACTATGACCG
CCTACTTGAGCGGCACCAATCCTATCATTGAGAAAGAGCTGGAGTTTCTGGAGAGCAATCAGGACATCATTCACTGGTCT
TTCAAGATCCTGAGGCTGCAGGATGATCTGGGCACTAGCAGCGACGAGATCCAGAGGGGCGACGTTCCTAAAAGCATCCA
GTGCTACATGCATGAGACTGGCGCCAGCGAAGAGGTGGCCCGCGAGCATATCAAAGACATGATGAGGCAGATGTGGAAAA
AGGTGAACGCCTACAGAGCCGACAAAGATAGCCCTCTGTCCCAGACCACCGTGGAGTTCATTCTGAATGTGGTGAGAGTG
TCTCACTTCATGTACCTCCACGGAGACGGACACGGCGCCCAGAACCAGGAGACCATGGATGTGGTGTTTACCCTGCTGTT
CCAGCCTATCCCACTGGATGACAAGCACATTGTGGCTACAAGCAGCCCCGTGACCAAAGGCTGA
Enzyme (+)-limonene synthase from satsuma mandarin (Citrus_unshiu)-Genbank accession
number BAD27257.1. SEQ ID NO: 11.
MSSCINPSTL ATSVNGFKCL PLATNRAAIR IMAKNKPVQC LVSTKYDNLT VDRRSANYQP
SIWDHDFLQS LNSNYTDETY KRRAEELKGK VKTAIKDVTE PLDQLELIDN LQRLGLAYHE
EPEIRNILRN IHNHNKDYNW RKENLYATSL EFRLLRQHGY PVSQEVFSGF KDDKVGFICD
DFKGILSLHE ASYYSLEGES IMEEAWQFTS KHLKEMMITS NSKEEDVFVA EQAKRALELP
LHWKKVPMLE ARWFIHVYEK REDKNHLLLE LAKLEFNTLQ AIYQEELKDI SGWWKDTGLG
EKLSFARNRL VASFLWSMGI AFEPQFAYCR RVLTISIALI TVIDDIYDVY GTLDELEIFT
DAVARWDINY ALKHLPGYMK MCFLALYNFV NEFAYYVLKQ QDFDMLLSIK HAWLGLIQAY
LVEAKWYHSK YTPKLEEYLE NGLVSITGPL IITISYLSGT NPIIKKELEF LESNPDIVHW
SSKIFRLQDD LGTSSDEIQR GDVPKSIQCY MHETGASEEV AREHIKDMMR QMWKKVNAYT
ADKDSPLTRT TAEFLLNLVR MSHFMYLHGD GHGVQNQETI DVGFTLLFQP IPLEDKDMAF
TASPGTKG
A DNA sequence encoding enzyme (+)-limonene synthase from satsuma mandarin (Citrus_unshiu)-
SEQ ID NO: 12. The DNA sequence was codon optimized for expression in humans.
ATGTCCTCCTGCATCAATCCGAGCACTCTGGCAACAAGCGTGAACGGCTTCAAGTGCCTGCCACTGGCCACCAACCGCGC
CGCCATCAGGATTATGGCCAAGAATAAGCCCGTGCAGTGTCTGGTGTCTACTAAATATGACAATCTGACCGTGGACAGGC
GGTCCGCCAACTACCAGCCCTCCATCTGGGATCACGACTTTCTGCAGTCCCTCAACTCCAATTACACCGACGAGACCTAC
AAAAGGCGAGCCGAGGAGCTGAAGGGCAAGGTGAAAACCGCCATTAAGGACGTGACAGAACCTCTGGACCAGCTGGAGCT
GATCGACAATCTCCAGAGGCTGGGCCTGGCTTATCACTTCGAACCCGAGATCCGCAATATCCTGCGGAACATTCACAATC
ATAACAAGGACTACAATTGGAGGAAGGAAAACCTGTATGCCACCTCTCTGGAGTTTAGACTGCTCAGACAGCACGGCTAT
CCCGTCAGCCAGGAGGTGTTCTCCGGCTTTAAGGATGACAAGGTGGGCTTTATTTGCGATGACTTCAAAGGCATCCTGTC
TCTGCACGAGGCCTCCTACTACAGTCTGGAGGGAGAGTCCATCATGGAAGAGGCATGGCAGTTCACCTCAAAGCACCTGA
AGGAGATGATGATCACCAGCAATAGCAAGGAGGAGGACGTGTTCGTGGCTGAGCAGGCTAAGCGCGCCCTCGAACTGCCA
CTGCACTGGAAAAAAGTGCCAATGCTGGAGGCTCGCTGGTTCATCCATGTGTACGAGAAGCGCGAAGACAAGAACCACCT
GCTGTTGGAACTCGCCAAGCTGGAGTTCAACACACTGCAGGCCATCTACCAGGAAGAGCTGAAGGATATTAGTGGCTGGT
GGAAAGACACCGGACTGGGGGAGAAGCTGAGCTTCGCCCGGAACAGACTGGTGGCCTCCTTCCTGTGGAGCATGGGAATC
GCCTTTGAACCTCAGTTTGCCTATTGTCGGAGAGTGCTGACAATCAGCATCGCCCTGATCACCGTGATCGACGACATTTA
CGACGTCTATGGAACCCTGGACGAGCTGGAAATCTTTACAGACGCCGTGGCTCGCTGGGATATTAACTACGCCCTGAAGC
ACCTGCCTGGCTATATGAAGATGTGCTTCCTCGCCCTGTACAACTTTGTGAACGAGTTCGCCTATTATGTGCTGAAGCAG
CAGGATTTTGACATGCTGCTGAGCATTAAGCACGCCTGGCTGGGCCTGATTCAGGCCTACCTGGTAGAGGCCAAGTGGTA
CCACAGCAAGTACACTCCTAAACTGGAGGAGTATCTGGAGAACGGCCTGGTGTCCATCACTGGGCCCCTGATCATTACCA
TCTCCTACCTGTCCGGCACCAACCCGATCATCAAGAAGGAGCTGGAGTTCCTGGAGAGCAATCCTGACATCGTGCATTGG
AGTTCCAAGATTTTCAGGCTGCAGGATGACCTGGGCACAAGCTCAGACGAGATTCAGAGGGGCGATGTGCCTAAGTCCAT
CCAGTGCTATATGCACGAGACAGGAGCATCCGAAGAAGTGGCCCGCGAGCACATTAAGGACATGATGCGCCAGATGTGGA
AGAAAGTGAATGCCTACACCGCCGACAAGGACTCTCCTCTGACACGCACCACCGCCGAGTTCCTGCTGAACCTGGTGAGA
ATGTCCCACTTTATGTATCTGCACGGCGACGGCCACGGCGTGCAGAACCAGGAGACTATCGACGTGGGATTTACCCTGCT
GTTCCAGCCAATCCCCCTGGAAGACAAGGACATGGCATTCACTGCCTCTCCCGGCACCAAGGGCTAA
Enzyme (+)-limonene synthase from clementines (Citrus_clementina)-Genbank accession
number XP_024040294.1. SEQ ID NO: 13.
MSSSINPLTLVTSVNGFKCLPLATNKAAIRIMAKNKPVQCLVSAKYDNLTVDRRSANYQPSIWDHDFLQSLNSHSTDETY
KRRAEELKGKVMTTIKDVTEPLDQLELIDNLQRLGLVYRFETEIRNILHNIYNNNKDYVWRKENLYATSLEFRLLRQHGY
PVSQEVENGFKDDQGGFICDDFKGILSLHEASHYSLEGESIMEEAWQFTSKHLKEVMISKSKEEDLFVAEQAKRALELPL
HWKVPMLEARWFIHIYERREDKNHLLLELAKMEFNTLQAIYQEELKEISGWWKDTGLGEKLSFARNRLVASFLWSMGIAF
EPQFAYCRRVLTISIALITVIDDIYDVYGTLDELELFTDAVERWDINYALKHLPGYMKMCFLALYNFVNEFAYYVLKQQD
FDMLLSIKNAWLGLIQAYLVEAKWYHSKYTPKLEEYLENGLVSITGPLIITISYLSGTNPIIKKELEFLESNPDIVHWSS
KIFRLQDDLGTSSDEIQRGDVPKSIQCYMHETGASEEVAREHIKDMMRQMWKKVNAYTADKDSPLTRTTTEFLLNLVRMS
HFMYLHGDGHGVQNQQTIDVGFTLLFQPIPLGDKHMAFTASPGTKG
A DNA sequence encoding enzyme (+)-limonene synthase from clementines (Citrus_clementina)-
SEQ ID NO: 14. The DNA sequence was codon optimized for expression in humans.
ATGTCCTCTAGCATCAACCCTCTGACCCTGGTGACAAGCGTGAACGGCTTTAAGTGTCTGCCACTGGCCACAAACAAGGC
CGCCATTCGGATCATGGCCAAAAACAAGCCCGTGCAGTGCCTGGTGTCCGCCAAGTATGACAACCTGACAGTGGATCGGA
GGAGCGCAAATTACCAGCCCTCCATCTGGGACCACGATTTTCTGCAGTCACTGAATTCTCATTCCACCGACGAGACCTAC
AAGAGACGGGCCGAGGAACTGAAGGGCAAGGTCATGACCACCATCAAGGACGTGACTGAGCCTCTGGACCAGCTGGAACT
GATCGACAATCTGCAGCGGCTCGGCCTGGTGTACAGGTTTGAGACCGAGATCAGGAACATCCTGCACAATATTTACAATA
ACAACAAGGACTATGTGTGGAGAAAGGAGAATCTGTACGCCACAAGCCTGGAGTTCCGACTGCTGCGACAGCATGGGTAT
CCTGTCAGCCAGGAGGTGTTTAACGGCTTCAAAGACGACCAGGGCGGATTCATCTGCGACGATTTCAAGGGCATTCTGAG
CCTGCACGAGGCCAGCCACTACTCACTCGAAGGGGAATCCATTATGGAGGAGGCCTGGCAGTTCACAAGCAAGCACCTTA
AGGAAGTTATGATTAGCAAGAGCAAAGAGGAAGACCTGTTTGTGGCCGAGCAGGCCAAGAGAGCCCTGGAGCTTCCTCTC
CACTGGAAGGTGCCCATGCTGGAGGCCCGATGGTTCATTCACATCTACGAAAGAAGAGAGGACAAAAACCACCTGCTGCT
GGAGCTGGCCAAAATGGAATTCAATACCCTGCAGGCCATCTACCAGGAGGAGCTGAAGGAGATCAGCGGCTGGTGGAAGG
ATACCGGCCTGGGCGAGAAGCTGTCCTTCGCCCGGAATAGGCTCGTTGCCAGTTTCCTGTGGTCTATGGGCATCGCCTTC
GAGCCACAGTTCGCCTACTGTAGAAGAGTGCTGACCATCAGCATCGCACTGATTACCGTGATCGACGACATCTACGATGT
GTACGGCACACTGGACGAACTGGAGCTGTTTACAGACGCCGTGGAGAGATGGGATATCAACTACGCCCTGAAGCACCTGC
CCGGGTATATGAAGATGTGTTTCCTGGCCCTCTACAACTTCGTCAACGAGTTCGCCTACTATGTGCTGAAGCAGCAGGAC
TTCGACATGTTGCTGTCCATCAAGAACGCCTGGCTGGGCCTGATTCAGGCATATCTGGTGGAGGCCAAGTGGTACCACTC
TAAGTACACTCCAAAGCTGGAGGAATACTTGGAGAACGGACTGGTGAGCATCACTGGGCCTCTGATCATCACTATTAGCT
ACCTGAGCGGCACCAACCCCATTATTAAAAAGGAGCTGGAGTTCCTGGAGAGTAATCCCGATATCGTGCACTGGTCAAGT
AAGATTTTCAGACTGCAGGATGACCTGGGAACCTCAAGCGATGAGATACAGCGCGGAGACGTGCCAAAGTCCATTCAGTG
TTATATGCACGAGACCGGCGCCTCAGAGGAGGTGGCCCGCGAGCACATTAAGGACATGATGCGGCAGATGTGGAAGAAGG
TGAACGCCTACACCGCCGACAAGGACTCCCCCCTGACAAGGACTACAACCGAGTTTCTGCTGAATCTGGTGAGAATGTCC
CACTTCATGTACCTGCATGGCGACGGCCACGGCGTGCAGAACCAGCAGACCATCGACGTGGGATTCACCCTGCTCTTCCA
GCCCATTCCACTGGGCGACAAGCACATGGCCTTCACCGCCAGCCCTGGCACCAAGGGCTGA
Enzyme (+)-limonene synthase set forth in SEQ ID NO: 1 is truncated to exclude the plastid
signaling peptide-SEQ ID NO: 15
MDRRSANYQP SIWDHDFLQS LNSNYTDETY KRRAEELKGK VKTAIKDVTE PLDQLELIDN LQRLGLAYHF
EPEIRNILRN IHNHNKDYNW RKENLYATSL EFRLLRQHGY PVSQEVFSGF KDDKVGFICD DFKGILSLHE
ASYYSLEGES IMEEAWQFTS KHLKEMMITS NSKEEDVFVA EQAKRALELP LHWKAPMLEA RWFIHVYEKR
EDKNHLLLEL AKLEFNTLQA IYQEELKDIS GWWKDTGLGE KLSFARNRLV ASFLWSMGIA FEPQFAYCRR
VLTISIALIT VIDDIYDVYG TLDELEIFTD AVARWDINYA LKHLPGYMKM CFLALYNFVN EFAYYVLKQQ
DFDMLLSIKH AWLGLIQAYL VEAKWYHSKY TPKLEEYLEN GLVSITGPLI ITISYLSGTN PIIKKELEFL
ESNPDIVHWS SKIFRLQDDL GTSSDEIQRG DVPKSIQCYM HETGASEEVA REHIKDMMRQ MWKKVNAYTA
DKDSPLTRTT AEFLLNLVRM SHFMYLHGDG HGVQNQETID VGFTLLFQPI PLEDKDMAFT ASPGTKG
A DNA sequence encoding enzyme (+)-limonene synthase set forth in SEQ ID NO: 1 that is
truncated to exclude the plastid signaling peptide-SEQ ID NO: 16. The DNA sequence was codon
optimized for expression in humans.
ATGGATAGACGGTCCGCCAACTACCAGCCCTCAATCTGGGATCACGACTTCCTGCAGAGCCTGAATAGCAACTACACCGA
CGAGACTTATAAGCGGAGGGCCGAAGAGCTGAAAGGGAAGGTGAAGACTGCCATAAAGGATGTGACTGAGCCCCTCGATC
AGCTGGAACTGATTGACAACTTGCAGAGGCTGGGCCTGGCCTATCACTTTGAGCCAGAGATCCGCAACATCCTCCGCAAT
ATCCACAACCATAATAAAGATTACAACTGGAGGAAGGAAAATCTGTACGCCACCTCCCTGGAATTCCGGCTGCTGAGACA
GCACGGGTACCCCGTTAGTCAGGAAGTGTTTAGCGGCTTCAAGGACGACAAAGTGGGGTTCATCTGCGATGATTTCAAGG
GCATCCTGTCCCTGCACGAAGCCAGCTACTACTCCCTGGAGGGGGAGAGCATCATGGAAGAAGCCTGGCAGTTCACCTCT
AAGCACCTGAAGGAGATGATGATTACATCCAATTCCAAGGAAGAGGATGTGTTCGTTGCCGAGCAGGCCAAGAGAGCCCT
GGAGCTGCCCCTGCACTGGAAGGCACCCATGCTGGAGGCCCGCTGGTTCATCCACGTGTACGAGAAGAGAGAGGACAAGA
ACCACCTGCTGCTGGAGCTGGCCAAGCTGGAGTTTAACACACTGCAGGCCATATACCAGGAGGAGCTGAAGGATATCTCA
GGATGGTGGAAAGACACCGGCCTTGGCGAGAAGCTGTCCTTCGCCAGGAATCGGCTCGTGGCCTCTTTTCTGTGGAGCAT
GGGCATTGCTTTCGAACCCCAGTTCGCTTACTGCAGACGGGTGCTGACCATCAGCATCGCCCTGATCACCGTGATTGACG
ACATTTACGACGTGTACGGCACCCTGGACGAGCTGGAGATTTTCACCGACGCTGTGGCCAGGTGGGATATCAACTACGCC
CTGAAGCACCTGCCTGGCTATATGAAGATGTGTTTCCTGGCCCTGTACAATTTCGTGAACGAGTTCGCATACTACGTGCT
GAAGCAGCAGGACTTTGACATGCTGCTGTCCATCAAGCATGCCTGGCTGGGACTGATCCAGGCATACCTGGTGGAGGCAA
AGTGGTACCACAGCAAATATACACCCAAGCTGGAGGAGTATCTGGAGAATGGCCTGGTGAGCATCACCGGCCCCCTGATT
ATTACCATTTCCTACCTGAGTGGCACAAACCCAATCATCAAAAAGGAGCTGGAGTTCCTCGAGAGCAATCCAGATATCGT
GCACTGGAGCAGCAAAATTTTCCGCCTGCAGGACGACCTCGGCACCAGCAGCGACGAAATTCAGAGAGGCGACGTGCCAA
AGAGCATCCAGTGCTATATGCACGAGACCGGCGCCTCCGAGGAGGTGGCCAGGGAGCACATCAAGGATATGATGCGCCAG
ATGTGGAAGAAGGTGAATGCCTACACAGCTGACAAGGACTCCCCACTGACCAGAACCACCGCTGAGTTCCTGCTGAATCT
GGTGCGGATGAGTCACTTCATGTATCTGCACGGCGATGGCCATGGGGTGCAGAATCAGGAGACAATTGATGTGGGGTTCA
CACTGCTCTTTCAGCCCATCCCCCTGGAGGACAAGGACATGGCCTTTACTGCCAGCCCCGGCACCAAGGGCTAA
Enzyme (+)-limonene synthase set forth in SEQ ID NO: 3 is truncated to exclude the plastid
signaling peptide-SEQ ID NO: 17
MDRRSANYQP SIWDHDFLQS LNSNYTDETY RRRAEELKGK VKTAIKDVTE PLDQLELIDN LQRLGLAYRF
ETEIRNILHN IYNNNKDYVW RKENLYATSL EFRLLRQHGY PVSQEVFNGF KDDQGGFICD DFKGILSLHE
ASYYRLEGES IMEEAWQFTS KHLKEVMISK SKEEDVFVAE QAKRALELPL HWKVPMLEAR WFIHIYERRE
DKNHLLLELA KMEFNTLQAI YQEELKEISG WWKDTGLGEK LSFARNRLVA SFLWSMGIAF EPQFAYCRRV
LTISIALITV IDDIYDVYGT LDELEIFTDA VERWDINYAL KHLPGYMKMC FLALYNFVNE FAYYVLKQQD
FDMLLSIKNA WLGLIQAYLV EAKWYHSKYT PKLEEYLENG LVSITGPLII TISYLSGTNP IIKKELEFLE
SNPDIVHWSS KIFRLQDDLG TSSDEIQRGD VPKSIQCYMH ETGASEEVAR EHIKDMMRQM WKKVNAYTAD
KDSPLTRTTT EFLLNLVRMS HFMYLHGDGH GVQNQETIDV GFTLLFQPIP LEDKHMAFAA SPGTKG
A DNA sequence encoding enzyme (+)-limonene synthase set forth in SEQ ID NO: 3 that is
truncated to exclude the plastid signaling peptide-SEQ ID NO: 18. The DNA sequence was codon
optimized for expression in humans.
ATGGACCGGCGGAGCGCCAATTATCAGCCATCCATCTGGGACCACGACTTTCTGCAGTCCCTGAACTCCAACTACACTGA
CGAAACCTACAGAAGACGGGCCGAAGAGCTGAAGGGCAAAGTGAAGACAGCCATCAAGGATGTGACCGAACCTCTGGACC
AGCTGGAGCTGATCGATAACCTGCAGAGGCTGGGCCTGGCTTACCGGTTCGAAACAGAGATCCGGAACATTCTGCATAAC
ATTTACAACAACAACAAAGACTACGTCTGGAGAAAGGAAAATCTGTACGCCACCTCCCTGGAGTTCAGACTGCTGAGGCA
GCACGGCTACCCCGTGTCCCAGGAAGTTTTCAACGGCTTCAAGGATGACCAGGGGGGATTCATCTGTGACGACTTCAAAG
GCATCCTGTCTCTGCACGAAGCTTCCTACTATAGACTGGAGGGCGAGTCCATCATGGAGGAGGCCTGGCAGTTCACATCC
AAGCACCTGAAGGAGGTGATGATCTCCAAGTCAAAAGAGGAGGACGTGTTTGTGGCCGAACAGGCAAAGAGAGCCCTGGA
GCTGCCCTTGCATTGGAAGGTGCCCATGCTGGAGGCACGCTGGTTTATTCACATTTATGAGCGCAGAGAGGATAAAAATC
ACCTGCTGCTGGAGCTGGCGAAAATGGAGTTCAATACCCTCCAGGCCATCTACCAGGAGGAGCTGAAAGAAATCAGCGGG
TGGTGGAAAGACACTGGCCTGGGCGAGAAGCTGTCATTTGCCAGGAATCGGCTGGTGGCCTCCTTCCTGTGGAGCATGGG
CATCGCCTTCGAGCCCCAGTTCGCTTACTGCCGGAGAGTGCTTACAATCTCTATTGCCCTCATCACAGTGATCGATGATA
TCTACGACGTGTACGGCACGCTGGATGAGCTGGAGATTTTTACCGATGCCGTGGAGAGGTGGGACATCAACTACGCCCTG
AAACACCTGCCAGGATACATGAAGATGTGTTTCCTGGCTCTGTATAACTTCGTGAATGAGTTTGCCTATTATGTGCTGAA
GCAGCAGGACTTCGATATGCTGCTGTCTATCAAGAACGCCTGGCTCGGCCTGATTCAGGCTTACCTGGTGGAAGCCAAAT
GGTACCACTCTAAGTACACTCCCAAGCTGGAGGAGTACCTGGAGAACGGGTTGGTGAGCATCACCGGCCCTCTGATTATC
ACCATCAGCTACCTGTCCGGCACCAACCCAATCATTAAGAAGGAGCTGGAGTTTCTGGAGTCCAACCCCGACATTGTGCA
CTGGTCATCTAAGATCTTCCGCCTGCAGGATGACCTGGGCACCTCTAGCGATGAAATTCAGAGAGGGGACGTGCCTAAGT
CCATCCAATGTTACATGCACGAGACCGGAGCCAGTGAGGAGGTGGCCCGCGAACACATTAAGGACATGATGAGGCAGATG
TGGAAGAAGGTGAACGCCTACACCGCCGATAAGGACTCCCCCCTGACACGGACCACCACAGAGTTTCTGCTGAATCTGGT
GCGGATGTCCCACTTCATGTACCTGCATGGGGACGGACACGGAGTGCAGAATCAGGAAACAATCGATGTGGGCTTTACAC
TGCTGTTCCAGCCTATCCCCCTGGAGGATAAGCACATGGCCTTCGCCGCCTCCCCTGGCACAAAGGGCTGA
Enzyme (+)-limonene synthase set forth in SEQ ID NO: 5 is truncated to exclude the plastid
signaling peptide-SEQ ID NO: 19
MDRRSANYQP SIWDHDFLQS LNSNYTDETY RRRAEELKGK VKIAIKDVTE PLDQLELIDN LQRLGLAYRF
ETEIRNILHN IYNNNKDYVW RKENLYATSL EFRLLRQHGY PVSQEVFNGF KDDQGGFIFD DFKGILSLHE
ASYYSLEGES IMEEAWQFTS KHLKEVMISK SMEEDVFVAE QAKRALELPL HWKVPMLEAR WFIHVYEKRE
DKNHLLLELA KMEFNTLQAI YQEELKEISG WWKDTGLGEK LSFARNRLVA SFLWSMGIAF EPQFAYCRRV
LTISIALITV IDDIYDVYGT LDELEIFTDA VARWDINYAL KHLPGYMKMC FLALYNFVNE FAYYVLKQQD
FDMLLSIKNA WLGLIQAYLV EAKWYHSKYT PKLEEYLENG LVSITGPLII AISYLSGTNP IIKKELEFLE
SNPDIVHWSS KIFRLQDDLG TSSDEIQRGD VPKSIQCYMH ETGASEEVAR EHIKDMMRQM WKKVNAYTAD
KDSPLTRTTT EFLLNLVRMS HFMYLHGDGH GVQNQETIDV GFTLLFQPIP LEDKDMAFTA SPGTKG
A DNA sequence encoding enzyme (+)-limonene synthase set forth in SEQ ID NO: 5 that is
truncated to exclude the plastid signaling peptide-SEQ ID NO: 20. The DNA sequence was codon
optimized for expression in humans.
ATGGATAGGCGGAGTGCTAATTACCAGCCAAGCATCTGGGATCACGATTTCCTGCAGTCCCTGAACTCCAACTATACCGA
CGAAACATACCGGAGGAGAGCCGAGGAGCTGAAGGGGAAAGTGAAGATCGCCATTAAGGACGTGACCGAGCCCCTGGACC
AGCTGGAGCTGATTGATAACCTGCAGCGCCTGGGCCTGGCCTATCGGTTTGAGACGGAAATCCGGAATATCCTGCACAAC
ATCTATAATAATAACAAGGATTACGTGTGGAGAAAGGAAAATCTGTACGCCACCTCCCTGGAGTTTAGACTGCTGAGGCA
GCACGGATACCCCGTGTCCCAGGAAGTGTTCAACGGCTTCAAGGATGACCAGGGCGGCTTTATCTTCGATGACTTCAAGG
GAATTCTGTCCCTGCACGAGGCCAGTTACTACTCTCTGGAGGGCGAGTCCATCATGGAGGAGGCTTGGCAGTTCACCTCC
AAGCACCTGAAAGAGGTGATGATTAGCAAATCCATGGAAGAGGACGTGTTTGTGGCCGAGCAGGCTAAGAGAGCCCTGGA
GCTGCCTCTGCACTGGAAGGTGCCAATGCTGGAGGCAAGGTGGTTTATCCACGTGTATGAGAAGCGCGAGGATAAGAATC
ACCTGCTGCTGGAGCTGGCCAAAATGGAGTTCAACACTCTGCAGGCAATCTACCAGGAAGAGCTGAAAGAGATCAGCGGC
TGGTGGAAAGATACCGGGCTGGGGGAGAAGCTGAGCTTTGCCCGAAATAGGCTGGTGGCCAGCTTTCTGTGGAGCATGGG
GATTGCTTTCGAGCCTCAGTTCGCCTACTGCCGGAGAGTGCTCACCATCAGTATCGCCCTGATCACCGTGATCGACGACA
TCTACGACGTGTACGGCACCCTGGACGAACTGGAGATCTTCACTGATGCAGTGGCCAGGTGGGATATCAACTATGCACTG
AAACACCTGCCCGGATACATGAAAATGTGCTTTCTGGCCCTGTATAACTTCGTGAACGAGTTCGCTTATTACGTGCTGAA
GCAGCAGGATTTCGACATGCTGCTCAGCATCAAGAACGCCTGGCTGGGCCTGATCCAGGCCTACCTGGTGGAGGCCAAAT
GGTACCATAGCAAGTACACCCCCAAGCTGGAAGAGTACCTTGAGAACGGCCTGGTGTCTATTACTGGCCCTCTGATCATC
GCCATCAGCTACCTCTCTGGCACCAACCCAATCATTAAGAAGGAGCTGGAGTTTCTGGAGTCAAACCCAGATATCGTGCA
TTGGTCCAGCAAAATCTTCCGGCTGCAGGATGACCTGGGGACCTCCAGCGACGAGATCCAAAGAGGAGACGTGCCAAAAT
CCATCCAGTGCTATATGCACGAAACCGGAGCCAGCGAAGAGGTGGCCAGAGAGCATATCAAGGACATGATGAGGCAGATG
TGGAAGAAAGTAAACGCCTACACCGCAGATAAGGACAGCCCCCTCACCCGCACCACAACCGAATTCCTGCTGAATCTGGT
GCGGATGTCCCATTTCATGTACCTGCATGGCGATGGCCATGGTGTCCAGAACCAGGAAACCATCGATGTGGGCTTCACCC
TGCTGTTTCAGCCTATCCCTCTGGAGGACAAGGACATGGCCTTTACCGCAAGTCCCGGCACAAAGGGCTGA
Enzyme (+)-limonene synthase set forth in SEQ ID NO: 7 is truncated to exclude the plastid
signaling peptide-SEQ ID NO: 21

| | |
|---|---|
| 0 | MDRRSANYQP SIWDHDFLQS LNSNYTDEAY KRRAEELRGK VKIAIKDVIE PLDQLELIDN |
| 60 | LQRLGLAHRF ETEIRNILNN IYNNNKDYNW RKENLYATSL EFRLLRQHGY PVSQEVENGF |
| 120 | KDDQGGFICD DFKGILSLHE ASYYSLEGES IMEEAWQFTS KHLKEVMISK NMEEDVFVAE |
| 180 | QAKRALELPL HWKVPMLEAR WFIHIYERRE DKNHLLLELA KMEFNTLQAI YQEELKEISG |
| 240 | WWKDTGLGEK LSFARNRLVA SFLWSMGIAF EPQFAYCRRV LTISIALITV IDDIYDVYGT |
| 300 | LDELEIFTDA VERWDINYAL KHLPGYMKMC FLALYNFVNE FAYYVLKQQD FDLLLSIKNA |
| 360 | WLGLIQAYLV EAKWYHSKYT PKLEEYLENG LVSITGPLII TISYLSGTNP IIKKELEFLE |
| 420 | SNPDIVHWSS KIFRLQDDLG TSSDEIQRGD VPKSIQCYMH ETGASEEVAR QHIKDMMRQM |
| 480 | WKKVNAYTAD KDSPLTGTTT EFLLNLVRMS HFMYLHGDGH GVQNQETIDV GFTLLFQPIP |
| 540 | LEDKHMAFTA SPGTKG |

A DNA sequence encoding enzyme (+)-limonene synthase set forth in SEQ ID NO: 7 that is
truncated to exclude the plastid signaling peptide-SEQ ID NO: 22. The DNA sequence was codon
optimized for expression in humans.
ATGGACCGGCGGAGCGCCAATTATCAGCCATCCATCTGGGACCACGACTTTCTGCAGTCCCTGAACTCCAACTACACTGA
CGAAGCCTACAAGAGACGGGCCGAAGAGCTGCGGGGCAAAGTGAAGATTGCCATCAAGGATGTGATCGAACCTCTGGACC
AGCTGGAGCTGATCGATAACCTGCAGAGGCTGGGCCTGGCTCACCGGTTCGAAACAGAGATCCGGAACATTCTGAATAAC
ATTTACAACAACAACAAAGACTACAACTGGAGAAAGGAAAATCTGTACGCCACCTCCCTGGAGTTCAGACTGCTGAGGCA
GCACGGCTACCCCGTGTCCCAGGAAGTTTTCAACGGCTTCAAGGATGACCAGGGGGGATTCATCTGTGACGACTTCAAAG
GCATCCTGTCTCTGCACGAAGCTTCCTACTATTCACTGGAGGGCGAGTCCATCATGGAGGAGGCCTGGCAGTTCACATCC
AAGCACCTGAAGGAGGTGATGATCTCCAAGAACATGGAGGAGGACGTGTTTGTGGCCGAACAGGCAAAGAGAGCCCTGGA
GCTGCCCTTGCATTGGAAGGTGCCCATGCTGGAGGCACGCTGGTTTATTCACATTTATGAGCGCAGAGAGGATAAAAATC
ACCTGCTGCTGGAGCTGGCGAAAATGGAGTTCAATACCCTCCAGGCCATCTACCAGGAGGAGCTGAAAGAAATCAGCGGG
TGGTGGAAAGACACTGGCCTGGGCGAGAAGCTGTCATTTGCCAGGAATCGGCTGGTGGCCTCCTTCCTGTGGAGCATGGG
CATCGCCTTCGAGCCCCAGTTCGCTTACTGCCGGAGAGTGCTTACAATCTCTATTGCCCTCATCACAGTGATCGATGATA
TCTACGACGTGTACGGCACGCTGGATGAGCTGGAGATTTTTACCGATGCCGTGGAGAGGTGGGACATCAACTACGCCCTG
AAACACCTGCCAGGATACATGAAGATGTGTTTCCTGGCTCTGTATAACTTCGTGAATGAGTTTGCCTATTATGTGCTGAA
GCAGCAGGACTTCGATCTGCTGCTGTCTATCAAGAACGCCTGGCTCGGCCTGATTCAGGCTTACCTGGTGGAAGCCAAAT
GGTACCACTCTAAGTACACTCCCAAGCTGGAGGAGTACCTGGAGAACGGGTTGGTGAGCATCACCGGCCCTCTGATTATC
ACCATCAGCTACCTGTCCGGCACCAACCCAATCATTAAGAAGGAGCTGGAGTTTCTGGAGTCCAACCCCGACATTGTGCA
CTGGTCATCTAAGATCTTCCGCCTGCAGGATGACCTGGGCACCTCTAGCGATGAAATTCAGAGAGGGGACGTGCCTAAGT
CCATCCAATGTTACATGCACGAGACCGGAGCCAGTGAGGAGGTGGCCCGCCAGCACATTAAGGACATGATGAGGCAGATG
TGGAAGAAGGTGAACGCCTACACCGCCGATAAGGACTCCCCCCTGACAGGCACCACCACAGAGTTTCTGCTGAATCTGGT
GCGGATGTCCCACTTCATGTACCTGCATGGGGACGGACACGGAGTGCAGAATCAGGAAACAATCGATGTGGGCTTTACAC
TGCTGTTCCAGCCTATCCCCCTGGAGGATAAGCACATGGCCTTCACCGCCTCCCCTGGCACAAAGGGCTGA
Enzyme (+)-limonene synthase set forth in SEQ ID NO: 9 is truncated to exclude the plastid
signaling peptide-SEQ ID NO: 23.
MDRRSANYQP
PIWDHDFLQS LNSDYTDETY RRRAEELKGK VKTAIEDVTE PLDQLELIDN LQRLGLAYHF
ETEIRNILHN IYNNNKDYIW RKENLYATSL EFRLLROHGY PVSQEVSTGF KEDKGVFICD
DFMGILSLHE ASYYSLEGES IMEEAWQFTS KHLKEMMIIS NSKEEDVFVA EQAKRALELP
LHWKVPMLEA RWFIHVYEKR EDKNHLLLEL AKLEFNVLQA IYQEELKDVS RWWKDIGLGE
KLNFARDSLV ASFVWSMGIV FEPQFAYCRR ILTITFALIS VIDDIYDVYG TLDELELFAD
AVERWDINYA LNHLPDYMKI CFLALYNLVN EFTYYVLKQQ DFDILRSIKN AWLRNIQAYL
VEAKWYHGKY TPTLGEFLEN GLVSIGGPMV TMTAYLSGTN PIIEKELEFL ESNQDIIHWS
FKILRLODDL GTSSDEIQRG DVPKSIQCYM HETGASEEVA REHIKDMMRQ MWKKVNAYRA
DKDSPLSQTT VEFILNVVRV SHFMYLHGDG HGAQNQETMD VVFTLLFQPI PLDDKHIVAT
SSPVTKG
A DNA sequence encoding enzyme (+)-limonene synthase set forth in SEQ ID NO: 9 that is
truncated to exclude the plastid signaling peptide-SEQ ID NO: 24. The DNA sequence was codor
optimized for expression in humans.
ATGGATAGACGGTCCGCCAACTACCAGCCCCCTATCTGGGATCACGACTTCCTGCAGAGCCTGAATAGCGACTACAC
CGACGAGACTTATAGACGGAGGGCCGAAGAGCTGAAAGGGAAGGTGAAGACTGCCATAGAGGATGTGACTGAGCCCC
TCGATCAGCTGGAACTGATTGACAACTTGCAGAGGCTGGGCCTGGCCTATCACTTTGAGACAGAGATCCGCAACATC
CTCCACAATATCTACAACAATAATAAAGATTACATCTGGAGGAAGGAAAATCTGTACGCCACCTCCCTGGAATTCCG
GCTGCTGAGACAGCACGGGTACCCCGTTAGTCAGGAAGTGAGTACAGGCTTCAAGGAGGACAAAGGAGTGTTCATCT
GCGATGATTTCATGGGCATCCTGTCCCTGCACGAAGCCAGCTACTACTCCCTGGAGGGGGAGAGCATCATGGAAGAA
GCCTGGCAGTTCACCTCTAAGCACCTGAAGGAGATGATGATTATTTCCAATTCCAAGGAAGAGGATGTGTTCGTTGC
CGAGCAGGCCAAGAGAGCCCTGGAGCTGCCCCTGCACTGGAAGGTGCCCATGCTGGAGGCCCGCTGGTTCATCCACG
TGTACGAGAAGAGAGAGGACAAGAACCACCTGCTGCTGGAGCTGGCCAAGCTGGAGTTTAACGTGCTGCAGGCCATA
TACCAGGAGGAGCTGAAGGATGTCTCAAGATGGTGGAAAGACATCGGCCTTGGCGAGAAGCTGAACTTCGCCAGGGA
TTCCCTCGTGGCCTCTTTTGTGTGGAGCATGGGCATTGTGTTCGAACCCCAGTTCGCTTACTGCAGACGGATCCTGA
CCATCACATTCGCCCTGATCTCCGTGATTGACGACATTTACGACGTGTACGGCACCCTGGACGAGCTGGAGCTGTTC
GCCGACGCTGTGGAGAGGTGGGATATCAACTACGCCCTGAACCACCTGCCTGACTATATGAAGATCTGTTTCCTGGC
CCTGTACAATCTGGTGAACGAGTTCACATACTACGTGCTGAAGCAGCAGGACTTTGACATCCTGAGATCCATCAAGA
ATGCCTGGCTGAGGAATATCCAGGCATACCTGGTGGAGGCAAAGTGGTACCACGGAAAATATACACCCACACTGGGG
GAGTTTCTGGAGAATGGCCTGGTGAGCATCGGCGGCCCCATGGTGACTATGACTGCCTACCTGAGTGGCACAAACCC
AATCATCGAGAAGGAGCTGGAGTTCCTCGAGAGCAATCAGGATATCATTCACTGGAGCTTTAAAATTCTGCGCCTGC
AGGACGACCTCGGCACCAGCAGCGACGAAATTCAGAGAGGCGACGTGCCAAAGAGCATCCAGTGCTATATGCACGAG
ACCGGCGCCTCCGAGGAGGTGGCCAGGGAGCACATCAAGGATATGATGCGCCAGATGTGGAAGAAGGTGAATGCCTA
CAGGGCTGACAAGGACTCCCCACTGTCCCAGACCACCGTGGAGTTCATCCTGAATGTGGTGCGGGTGAGTCACTTCA
TGTATCTGCACGGCGATGGCCATGGGGCCCAGAATCAGGAGACAATGGATGTGGTGTTCACACTGCTCTTTCAGCCC
ATCCCCCTGGACGACAAGCACATCGTGGCCACTTCTAGCCCCGTGACCAAGGGCTAA
Enzyme (+)-limonene synthase set forth in SEQ ID NO: 11 is truncated to exclude the plastid
signaling peptide-SEQ ID NO: 25.
MDRRSANYQP
SIWDHDFLQS LNSNYTDETY KRRAEELKGK VKTAIKDVTE PLDQLELIDN LQRLGLAYHF
EPEIRNILRN IHNHNKDYNW RKENLYATSL EFRLLRQHGY PVSQEVFSGF KDDKVGFICD
DFKGILSLHE ASYYSLEGES IMEEAWQFTS KHLKEMMITS NSKEEDVFVA EQAKRALELP
LHWKKVPMLE ARWFIHVYEK REDKNHLLLE LAKLEFNTLQ AIYQEELKDI SGWWKDTGLG
EKLSFARNRL VASFLWSMGI AFEPQFAYCR RVLTISIALI TVIDDIYDVY GTLDELEIFT
DAVARWDINY ALKHLPGYMK MCFLALYNFV NEFAYYVLKQ QDFDMLLSIK HAWLGLIQAY
LVEAKWYHSK YTPKLEEYLE NGLVSITGPL IITISYLSGT NPIIKKELEF LESNPDIVHW
SSKIFRLQDD LGTSSDEIQR GDVPKSIQCY MHETGASEEV AREHIKDMMR QMWKKVNAYT
ADKDSPLTRT TAEFLLNLVR MSHFMYLHGD GHGVQNQETI DVGFTLLFQP IPLEDKDMAF
TASPGTKG
A DNA sequence encoding enzyme (+)-limonene synthase set forth in SEQ ID NO: 11 that is
truncated to exclude the plastid signaling peptide-SEQ ID NO: 26. The DNA sequence was codon
optimized for expression in humans.
ATGGATCGCAGATCTGCCAATTATCAGCCTTCCATTTGGGACCATGATTTCCTGCAGTCCCTGAATAGCAACTACAC
AGACGAGACCTACAAGCGTCGGGCCGAGGAGCTTAAGGGAAAGGTGAAGACCGCGATCAAGGACGTGACTGAGCCAC
TGGACCAGCTGGAGCTGATTGACAACCTGCAGAGGCTGGGACTGGCCTACCACTTCGAGCCAGAAATCCGCAATATC
CTGCGCAATATTCATAACCATAACAAGGACTACAACTGGAGGAAGGAGAATCTGTACGCCACATCCCTGGAATTCAG
GCTTCTGAGACAGCACGGATACCCAGTGAGCCAGGAGGTGTTCAGCGGCTTCAAGGACGACAAAGTGGGCTTCATTT
GCGATGACTTCAAGGGAATCCTGAGTCTGCACGAAGCTAGCTATTACTCACTGGAAGGCGAGAGCATCATGGAAGAG
GCCTGGCAGTTTACCAGCAAGCACCTGAAGGAGATGATGATCACTTCTAATTCTAAGGAGGAAGACGTGTTCGTGGC
CGAGCAGGCCAAACGCGCCCTTGAGCTGCCCCTGCACTGGAAAAAGGTCCCTATGCTGGAAGCCAGATGGTTTATCC
ATGTGTATGAGAAAAGGGAGGACAAGAACCACCTGCTGCTGGAGCTGGCCAAGCTGGAGTTCAACACTCTGCAGGCC
ATTTACCAGGAGGAGCTGAAGGATATCAGCGGCTGGTGGAAGGACACCGGCCTGGGCGAAAAACTGTCTTTCGCCAG
AAACAGACTGGTGGCATCCTTTCTGTGGAGCATGGGAATCGCCTTTGAACCTCAGTTCGCCTACTGCAGGAGAGTGC
TGACCATTTCCATCGCCCTGATTACAGTGATCGATGATATCTACGACGTCTACGGCACCCTGGACGAGCTGGAGATT
TTTACAGACGCCGTGGCTAGGTGGGATATTAATTACGCCCTGAAGCACCTGCCTGGATATATGAAGATGTGCTTCCT
GGCCCTGTACAACTTTGTGAACGAGTTTGCCTACTACGTGCTGAAACAGCAGGACTTCGACATGCTGCTGTCTATCA
AGCATGCTTGGCTGGGACTGATCCAGGCCTACCTGGTGGAAGCCAAGTGGTATCACAGCAAGTATACACCCAAGCTG
GAGGAGTACCTGGAGAACGGCCTGGTGAGCATTACAGGCCCCCTGATCATCACAATCTCATATCTCTCCGGGACCAA
CCCAATCATTAAAAAGGAACTGGAATTCCTGGAATCCAACCCTGACATTGTGCACTGGTCTAGCAAGATCTTTAGGC
TGCAGGACGACCTGGGAACCAGCTCTGATGAGATTCAGCGCGGCGATGTGCCCAAGTCCATCCAGTGTTACATGCAC
GAGACCGGCGCCTCTGAGGAAGTGGCCAGGGAGCACATCAAGGATATGATGAGGCAGATGTGGAAAAAAGTTAATGC
CTACACCGCCGACAAGGACTCACCTCTGACTAGGACAACCGCAGAATTCCTGCTGAATCTGGTGCGGATGTCTCACT
TTATGTACCTGCATGGGGACGGGCACGGCGTGCAGAACCAGGAGACAATCGATGTGGGCTTCACCCTGCTGTTTCAG
CCCATTCCCCTGGAGGACAAAGACATGGCCTTCACAGCCTCTCCCGGCACAAAAGGCTGA
Enzyme (+)-limonene synthase set forth in SEQ ID NO: 13 is truncated to exclude the plastid
signaling peptide-SEQ ID NO: 27.
MDRRSANYQPSIWDHDFLQSLNSHSTDETY
KRRAEELKGKVMTTIKDVTEPLDQLELIDNLQRLGLVYRFETEIRNILHNIYNNNKDYVWRKENLYATSLEFRLLRQHGY
PVSQEVENGFKDDQGGFICDDFKGILSLHEASHYSLEGESIMEEAWQFTSKHLKEVMISKSKEEDLFVAEQAKRALELPL
HWKVPMLEARWFIHIYERREDKNHLLLELAKMEFNTLQAIYQEELKEISGWWKDTGLGEKLSFARNRLVASFLWSMGIAF
EPQFAYCRRVLTISIALITVIDDIYDVYGTLDELELFTDAVERWDINYALKHLPGYMKMCFLALYNFVNEFAYYVLKQQD
FDMLLSIKNAWLGLIQAYLVEAKWYHSKYTPKLEEYLENGLVSITGPLIITISYLSGTNPIIKKELEFLESNPDIVHWSS
KIFRLQDDLGTSSDEIQRGDVPKSIQCYMHETGASEEVAREHIKDMMRQMWKKVNAYTADKDSPLTRTTTEFLLNLVRMS
HFMYLHGDGHGVQNQQTIDVGFTLLFQPIPLGDKHMAFTASPGTKG
A DNA sequence encoding enzyme (+)-limonene synthase set forth in SEQ ID NO: 13 that is
truncated to exclude the plastid signaling peptide-SEQ ID NO: 28. The DNA sequence was codon
optimized for expression in humans.
ATGGACCGGCGGAGCGCCAATTATCAGCCATCCATCTGGGACCACGACTTTCTGCAGTCCCTGAACTCCCACTCCAC
TGACGAAACCTACAAGAGACGGGCCGAAGAGCTGAAGGGCAAAGTGATGACAACCATCAAGGATGTGACCGAACCTC
TGGACCAGCTGGAGCTGATCGATAACCTGCAGAGGCTGGGCCTGGTGTACCGGTTCGAAACAGAGATCCGGAACATT
CTGCATAACATTTACAACAACAACAAAGACTACGTCTGGAGAAAGGAAAATCTGTACGCCACCTCCCTGGAGTTCAG
ACTGCTGAGGCAGCACGGCTACCCCGTGTCCCAGGAAGTTTTCAACGGCTTCAAGGATGACCAGGGGGGATTCATCT
GTGACGACTTCAAAGGCATCCTGTCTCTGCACGAAGCTTCCCACTATTCACTGGAGGGCGAGTCCATCATGGAGGAG
GCCTGGCAGTTCACATCCAAGCACCTGAAGGAGGTGATGATCTCCAAGTCAAAAGAGGAGGACCTGTTTGTGGCCGA
ACAGGCAAAGAGAGCCCTGGAGCTGCCCTTGCATTGGAAGGTGCCCATGCTGGAGGCACGCTGGTTTATTCACATTT
ATGAGCGCAGAGAGGATAAAAATCACCTGCTGCTGGAGCTGGCGAAAATGGAGTTCAATACCCTCCAGGCCATCTAC
CAGGAGGAGCTGAAAGAAATCAGCGGGTGGTGGAAAGACACTGGCCTGGGCGAGAAGCTGTCATTTGCCAGGAATCG
GCTGGTGGCCTCCTTCCTGTGGAGCATGGGCATCGCCTTCGAGCCCCAGTTCGCTTACTGCCGGAGAGTGCTTACAA
TCTCTATTGCCCTCATCACAGTGATCGATGATATCTACGACGTGTACGGCACGCTGGATGAGCTGGAGCTGTTTACC
GATGCCGTGGAGAGGTGGGACATCAACTACGCCCTGAAACACCTGCCAGGATACATGAAGATGTGTTTCCTGGCTCT
GTATAACTTCGTGAATGAGTTTGCCTATTATGTGCTGAAGCAGCAGGACTTCGATATGCTGCTGTCTATCAAGAACG
CCTGGCTCGGCCTGATTCAGGCTTACCTGGTGGAAGCCAAATGGTACCACTCTAAGTACACTCCCAAGCTGGAGGAG
TACCTGGAGAACGGGTTGGTGAGCATCACCGGCCCTCTGATTATCACCATCAGCTACCTGTCCGGCACCAACCCAAT
CATTAAGAAGGAGCTGGAGTTTCTGGAGTCCAACCCCGACATTGTGCACTGGTCATCTAAGATCTTCCGCCTGCAGG
ATGACCTGGGCACCTCTAGCGATGAAATTCAGAGAGGGGACGTGCCTAAGTCCATCCAATGTTACATGCACGAGACC
GGAGCCAGTGAGGAGGTGGCCCGCGAACACATTAAGGACATGATGAGGCAGATGTGGAAGAAGGTGAACGCCTACAC
CGCCGATAAGGACTCCCCCCTGACACGGACCACCACAGAGTTTCTGCTGAATCTGGTGCGGATGTCCCACTTCATGT
ACCTGCATGGGGACGGACACGGAGTGCAGAATCAGCAGACAATCGATGTGGGCTTTACACTGCTGTTCCAGCCTATC
CCCCTGGGCGATAAGCACATGGCCTTCACCGCCTCCCCTGGCACAAAGGGCTGA
6-Histidine tag is added to the N-terminus of SEQ ID NO: 21-SEQ ID NO: 29

| | |
|---|---|
| 0 | MHHHHHHDRR SANYQPSIWD HDFLQSLNSN YTDEAYKRRA EELRGKVKIA IKDVIEPLDQ |
| 60 | LELIDNLQRL GLAHRFETEI RNILNNIYNN NKDYNWRKEN LYATSLEFRL LRQHGYPVSQ |
| 120 | EVENGFKDDQ GGFICDDFKG ILSLHEASYY SLEGESIMEE AWQFTSKHLK EVMISKNMEE |
| 180 | DVFVAEQAKR ALELPLHWKV PMLEARWFIH IYERREDKNH LLLELAKMEF NTLQAIYQEE |
| 240 | LKEISGWWKD TGLGEKLSFA RNRLVASFLW SMGIAFEPQF AYCRRVLTIS IALITVIDDI |
| 300 | YDVYGTLDEL EIFTDAVERW DINYALKHLP GYMKMCFLAL YNFVNEFAYY VLKQQDFDLL |
| 360 | LSIKNAWLGL IQAYLVEAKW YHSKYTPKLE EYLENGLVSI TGPLIITISY LSGTNPIIKK |
| 420 | ELEFLESNPD IVHWSSKIFR LQDDLGTSSD EIQRGDVPKS IQCYMHETGA SEEVARQHIK |
| 480 | DMMRQMWKKV NAYTADKDSP LTGTTTEFLL NLVRMSHFMY LHGDGHGVQN QETIDVGFTL |
| 540 | LFQPIPLEDK HMAFTASPGT KG |

Genetic delivery vector containing a DNA sequence for the limonene synthase set forth in SEQ
ID 29 that is codon-optimized for mammalian cells-SEQ ID NO: 30
(Start) A
TGCATCACCA TCATCACCAC GACAGAAGAA GTGCTAACTA CCAGCCATCC ATTTGGGACC
ACGATTTCCT GCAGAGCCTG AACAGCAATT ACACAGATGA GGCCTATAAG AGGAGAGCAG
AGGAGCTGCG CGGCAAGGTG AAGATCGCCA TCAAGGACGT GATCGAGCCC CTGGATCAGC
TGGAGCTGAT CGACAACCTC CAGCGGCTGG GCCTGGCCCA CCGCTTCGAG ACAGAGATCC
GGAACATCCT GAACAACATC TACAACAACA ACAAGGACTA CAACTGGCGG AAGGAGAACC
TGTACGCCAC CAGCCTGGAG TTTCGGCTGC TGAGACAGCA CGGCTACCCC GTGAGCCAGG
AGGTGTTCAA TGGCTTTAAG GACGATCAGG GCGGCTTCAT CTGCGACGAC TTCAAGGGCA
TCCTGTCTCT GCACGAGGCC TCCTACTATT CTCTGGAGGG CGAGAGCATC ATGGAGGAGG
CCTGGCAGTT CACCTCCAAG CACCTGAAGG AAGTGATGAT CAGCAAGAAC ATGGAGGAGG
ACGTGTTTGT GGCCGAGCAG GCCAAGAGAG CCCTGGAGCT GCCCCTGCAC TGGAAGGTGC
CTATGCTGGA GGCCAGGTGG TTCATCCACA TCTATGAGAG GCGCGAGGAT AAGAATCACC
TGCTGCTGGA GCTGGCCAAG ATGGAGTTTA ACACACTCCA GGCCATCTAC CAGGAGGAGC
TGAAGGAGAT CAGCGGATGG TGGAAGGACA CCGGCCTGGG AGAGAAGCTG TCTTTCGCCA
GGAATCGCCT GGTGGCCTCT TTTCTGTGGA GCATGGGCAT CGCCTTCGAG CCTCAGTTTG
CCTATTGCCG GAGAGTGCTG ACAATCAGCA TCGCCCTGAT CACCGTGATC GACGACATCT
ACGACGTGTA CGGCACACTG GACGAGCTGG AGATTTTCAC CGATGCCGTG GAGCGGTGGG
ACATCAACTA CGCCCTGAAG CACCTGCCAG GCTATATGAA GATGTGCTTC CTGGCCCTGT
ACAATTTCGT GAACGAGTTT GCCTACTATG TGCTGAAGCA GCAGGACTTT GATCTGCTGC
TGAGCATCAA GAATGCCTGG CTGGGCCTGA TCCAGGCCTA CCTGGTGGAG GCCAAGTGGT
ATCACTCTAA GTATACACCC AAGCTGGAGG AGTATCTGGA GAACGGCCTG GTGAGCATCA
CAGGCCCACT GATCATCACC ATCAGCTACC TGTCCGGCAC CAATCCCATC ATCAAGAAGG
AGCTGGAGTT CCTGGAGTCC AACCCTGACA TCGTGCACTG GAGCAGCAAG ATTTTCCGGC
TCCAGGACGA TCTGGGCACA TCTAGCGATG AGATCCAGCG GGGCGACGTG CCAAAGAGCA
TCCAGTGTTA CATGCACGAG ACAGGAGCCT CCGAGGAGGT GGCAAGACAG CACATCAAGG
ACATGATGAG GCAGATGTGG AAGAAGGTGA ACGCCTATAC AGCCGACAAG GATTCCCCCC
TGACCGGCAC CACAACCGAG TTCCTGCTGA ATCTGGTGAG AATGTCTCAC TTTATGTACC
TGCACGGCGA TGGCCACGGC GTGCAGAACC AGGAGACAAT CGACGTGGGC TTCACCCTGC
TGTTTCAGCC TATCCCCCTG GAGGACAAGC ACATGGCATT CACCGCAAGC CCTGGCACTA
AAGGATGA (Stop)
Exemplary Limonene synthase consensus sequence 1-SEQ ID NO: 31
This sequence was derived based on the most common amino acid at each position in SEQ ID
NOs 1-7 as determined from multisequence alignment of these seven sequences (FIG. 8).
MSSCINPSTLVTSVNGFKCLPLATNKAAIRIMAKNKPVQCLVSAKYDNLTVDRRSANYQP
SIWDHDFLQSLNSNYTDETYKRRAEELKGKVKTAIKDVTEPLDQLELIDNLQRLGLAYRF
ETEIRNILHNIYNNNKDYNWRKENLYATSLEFRLLRQHGYPVSQEVENGFKDDQGGFICD
DFKGILSLHEASYYSLEGESIMEEAWQFTSKHLKEVMISKNKEEDVFVAEQAKRALELPL
HWKVPMLEARWFIHVYEKREDKNHLLLELAKMEFNTLQAIYQEELKEISGWWKDTGLGEK
LSFARNRLVASFLWSMGIAFEPQFAYCRRVLTISIALITVIDDIYDVYGTLDELEIFTDA
VERWDINYALKHLPGYMKMCFLALYNFVNEFAYYVLKQQDFDMLLSIKNAWLGLIQAYLV
EAKWYHSKYTPKLEEYLENGLVSITGPLIITISYLSGTNPIIKKELEFLESNPDIVHWSS
KIFRLQDDLGTSSDEIQRGDVPKSIQCYMHETGASEEVAREHIKDMMRQMWKKVNAYTAD
KDSPLTRTTTEFLLNLVRMSHFMYLHGDGHGVQNQETIDVGFTLLFQPIPLEDKHMAFTA
SPGTKG
A DNA sequence encoding limonene synthase consensus sequence 1 set forth in SEQ ID NO: 31
that is truncated to exclude the plastid signaling peptide-SEQ ID NO: 32. The DNA sequence was
codon optimized for expression in humans.
ATGAGCTCCTGTATTAACCCATCCACCCTTGTGACTAGCGTGAATGGCTTCAAGTGCCTGCCCCTGGCAACTAACAA
GGCCGCCATCCGGATCATGGCCAAGAACAAGCCAGTGCAGTGCCTGGTGTCTGCCAAGTATGACAATCTGACAGTGG
ACAGACGGAGCGCCAATTACCAGCCAAGCATCTGGGACCACGATTTCCTGCAGAGCCTGAACAGCAACTACACTGAC
GAGACCTACAAGCGGCGCGCTGAGGAGCTGAAAGGGAAGGTGAAGACCGCCATCAAGGATGTGACCGAGCCACTGGA
CCAGCTGGAACTGATTGATAACCTGCAGAGACTGGGCCTGGCCTACAGATTCGAAACCGAGATCAGGAACATTCTGC
ACAACATTTACAACAACAACAAGGACTACAATTGGAGAAAAGAGAACCTGTATGCCACCAGCCTGGAGTTCAGACTG
CTGCGCCAGCACGGATACCCAGTGAGCCAGGAGGTGTTCAATGGCTTCAAGGACGACCAGGGCGGATTCATCTGCGA
TGATTTTAAAGGGATCCTGAGCCTGCACGAGGCCTCCTACTACTCCCTGGAGGGAGAATCTATTATGGAGGAGGCCT
GGCAGTTCACCAGCAAGCACCTGAAAGAGGTGATGATTTCCAAGAATAAGGAGGAGGACGTGTTTGTCGCCGAACAG
GCCAAGAGAGCTCTGGAACTGCCTCTGCACTGGAAGGTGCCAATGCTGGAAGCCAGGTGGTTTATACACGTGTACGA
GAAGAGAGAGGACAAGAATCACCTGCTGCTGGAGCTGGCTAAAATGGAGTTTAATACCTTGCAGGCCATTTATCAGG
AGGAGCTGAAGGAAATCAGCGGCTGGTGGAAGGATACTGGATTGGGCGAGAAGCTCAGCTTTGCCCGGAACAGACTG
GTGGCCAGCTTTCTGTGGTCTATGGGCATCGCCTTCGAGCCCCAGTTTGCCTATTGTCGGAGAGTGCTGACAATTAG
CATCGCCCTGATCACTGTGATCGACGACATCTACGACGTGTACGGCACACTGGACGAGCTGGAAATCTTCACCGATG
CCGTGGAGAGGTGGGACATCAACTACGCCCTGAAGCATCTGCCAGGCTACATGAAGATGTGTTTTCTGGCCCTGTAC
AATTTCGTGAATGAGTTCGCCTATTACGTGCTCAAGCAGCAGGACTTTGACATGCTGCTGTCCATCAAGAACGCTTG
GCTGGGGCTGATTCAGGCTTACCTGGTGGAGGCCAAATGGTACCACTCTAAATACACTCCTAAACTGGAAGAGTACC
TGGAAAACGGACTGGTGAGCATCACCGGCCCACTGATCATTACCATCAGCTACCTGTCCGGGACTAACCCCATCATC
AAAAAGGAGCTCGAATTTCTGGAAAGTAATCCCGATATCGTGCACTGGAGCAGCAAGATTTTCAGGCTTCAGGATGA
TCTGGGGACCTCCTCCGATGAGATCCAGAGAGGCGACGTGCCAAAAAGTATTCAGTGCTACATGCACGAGACCGGGG
CCTCTGAGGAGGTGGCCCGGGAACATATTAAAGATATGATGAGGCAGATGTGGAAAAAGGTGAATGCCTATACAGCT
GACAAGGACTCCCCCCTGACAAGGACAACAACAGAATTCTTGCTGAACCTGGTGAGAATGAGCCATTTCATGTACCT
GCACGGCGACGGCCATGGCGTGCAGAATCAGGAGACTATTGACGTGGGCTTCACACTGCTGTTCCAGCCCATCCCCC
TGGAGGACAAGCACATGGCCTTTACAGCCAGCCCTGGCACTAAAGGCTAA
Enzyme (+)-limonene synthase set forth in SEQ ID NO: 31 is truncated to exclude the plastid
signaling peptide-SEQ ID NO: 33.
MDRRSANYQPSIWDHDFLQSLNSNYTDETYKRRAEELKGKVKTAIKDVTEPLDQLELIDNLQRLGLAYRFETEIRNILHNIYN
NNKDYNWRKENLYATSLEFRLLRQHGYPVSQEVENGFKDDQGGFICDDFKGILSLHEASYYSLEGESIMEEAWQFTSKHLKEV
MISKNKEEDVFVAEQAKRALELPLHWKVPMLEARWFIHVYEKREDKNHLLLELAKMEFNTLQAIYQEELKEISGWWKDTGLGE
KLSFARNRLVASFLWSMGIAFEPQFAYCRRVLTISIALITVIDDIYDVYGTLDELEIFTDAVERWDINYALKHLPGYMKMCEL
ALYNFVNEFAYYVLKQQDFDMLLSIKNAWLGLIQAYLVEAKWYHSKYTPKLEEYLENGLVSITGPLIITISYLSGTNPIIKKE
LEFLESNPDIVHWSSKIFRLQDDLGTSSDEIQRGDVPKSIQCYMHETGASEEVAREHIKDMMRQMWKKVNAYTADKDSPLTRT
TTEFLLNLVRMSHFMYLHGDGHGVQNQETIDVGFTLLFQPIPLEDKHMAFTASPGTKG
A DNA sequence encoding enzyme (+)-limonene synthase set forth in SEQ ID NO: 31 that is
truncated to exclude the plastid signaling peptide-SEQ ID NO: 34. The DNA sequence was codon
optimized for expression in humans.
ATGGACCGGCGGAGCGCCAATTATCAGCCATCCATCTGGGACCACGACTTTCTGCAGTCCCTGAACTCCAACTACAC
TGACGAAACCTACAAGAGACGGGCCGAAGAGCTGAAGGGCAAAGTGAAGACAGCCATCAAGGATGTGACCGAACCTC
TGGACCAGCTGGAGCTGATCGATAACCTGCAGAGGCTGGGCCTGGCTTACCGGTTCGAAACAGAGATCCGGAACATT
CTGCATAACATTTACAACAACAACAAAGACTACAACTGGAGAAAGGAAAATCTGTACGCCACCTCCCTGGAGTTCAG
ACTGCTGAGGCAGCACGGCTACCCCGTGTCCCAGGAAGTTTTCAACGGCTTCAAGGATGACCAGGGGGGATTCATCT
GTGACGACTTCAAAGGCATCCTGTCTCTGCACGAAGCTTCCTACTATTCACTGGAGGGCGAGTCCATCATGGAGGAG
GCCTGGCAGTTCACATCCAAGCACCTGAAGGAGGTGATGATCTCCAAGAACAAAGAGGAGGACGTGTTTGTGGCCGA
ACAGGCAAAGAGAGCCCTGGAGCTGCCCTTGCATTGGAAGGTGCCCATGCTGGAGGCACGCTGGTTTATTCACGTGT
ATGAGAAAAGAGAGGATAAAAATCACCTGCTGCTGGAGCTGGCGAAAATGGAGTTCAATACCCTCCAGGCCATCTAC
CAGGAGGAGCTGAAAGAAATCAGCGGGTGGTGGAAAGACACTGGCCTGGGCGAGAAGCTGTCATTTGCCAGGAATCG
GCTGGTGGCCTCCTTCCTGTGGAGCATGGGCATCGCCTTCGAGCCCCAGTTCGCTTACTGCCGGAGAGTGCTTACAA
TCTCTATTGCCCTCATCACAGTGATCGATGATATCTACGACGTGTACGGCACGCTGGATGAGCTGGAGATTTTTACC
GATGCCGTGGAGAGGTGGGACATCAACTACGCCCTGAAACACCTGCCAGGATACATGAAGATGTGTTTCCTGGCTCT
GTATAACTTCGTGAATGAGTTTGCCTATTATGTGCTGAAGCAGCAGGACTTCGATATGCTGCTGTCTATCAAGAACG
CCTGGCTCGGCCTGATTCAGGCTTACCTGGTGGAAGCCAAATGGTACCACTCTAAGTACACTCCCAAGCTGGAGGAG
TACCTGGAGAACGGGTTGGTGAGCATCACCGGCCCTCTGATTATCACCATCAGCTACCTGTCCGGCACCAACCCAAT
CATTAAGAAGGAGCTGGAGTTTCTGGAGTCCAACCCCGACATTGTGCACTGGTCATCTAAGATCTTCCGCCTGCAGG
ATGACCTGGGCACCTCTAGCGATGAAATTCAGAGAGGGGACGTGCCTAAGTCCATCCAATGTTACATGCACGAGACC
GGAGCCAGTGAGGAGGTGGCCCGCGAACACATTAAGGACATGATGAGGCAGATGTGGAAGAAGGTGAACGCCTACAC
CGCCGATAAGGACTCCCCCCTGACACGGACCACCACAGAGTTTCTGCTGAATCTGGTGCGGATGTCCCACTTCATGT
ACCTGCATGGGGACGGACACGGAGTGCAGAATCAGGAAACAATCGATGTGGGCTTTACACTGCTGTTCCAGCCTATC
CCCCTGGAGGATAAGCACATGGCCTTCACCGCCTCCCCTGGCACAAAGGGCTGA
Exemplary Limonene synthase consensus sequence 2, which shows the base pairs in common
(conserved regions)-SEQ ID NO: 35. Positions at which there are amino acid variations between
the different sequences are denoted by Xᵢ, with i = 1, 2, 3...30. The table below shows the
two most common amino acids for each Xᵢ from X₁ to X₃₀.
MSSX₁INPSTLX₂TSVNGFKCLPLATNX₃AAIRIMAKNKPVQCLVSX₄KYDNLTVDRRSANYQPSIWDHDFLQSLNSNYT
DETYX₅RRAEELKGKVKX₆AIKDVTEPLDQLELIDNLQRLGLAYX₇FEX₈EIRNILX₉NIX₁₀NX₁₁NKDYX₁₂WRKENLYA
TSLEFRLLRQHGYPVSQEVFX₁₃GFKDDX₁₄X₁₅GFICDDEKGILSLHEASYYSLEGESIMEEAWQFTSKHLKEX₁₆MIX₁₇
X₁₈X₁₉X₂₀X₂₁EEDVFVAEQAKRALELPLHWKVPMLEARWFIHX₂₂YEX₂₃REDKNHLLLELAKX₂₄EFNTLQAIYQEELKX₂₅
ISGWWKDTGLGEKLSFARNRLVASFLWSMGIAFEPQFAYCRRVLTISIALITVIDDIYDVYGTLDELEX₂₆FTDAVX₂₇
RWDINYALKHLPGYMKMCFLALYNFVNEFAYYVLKQQDFDMLLSIKX₂₈AWLGLIQAYLVEAKWYHSKYTPKLEEYLEN
GLVSITGPLIITISYLSGTNPIIKKELEFLESNPDIVHWSSKIFRLQDDLGTSSDEIQRGDVPKSIQCYMHETGASEE
VAREHIKDMMRQMWKKVNAYTADKDSPLTRTTX₂₉EFLLNLVRMSHFMYLHGDGHGVQNQETIDVGFTLLFQPIPLEDK
X₃₀MAFTASPGTKG

| | | |
|---|---|---|
| X₁ = C or S | X₁₁ = N or H | X₂₁ = K or M |
| X₂ = V or A | X₁₂ = V or N | X₂₂ = V or I |
| X₃ = K or R | X₁₃ = N or S | X₂₃ = K or R |
| X₄ = A or T | X₁₄ = Q or K | X₂₄ = M or L |
| X₅ = K or R | X₁₅ = G or V | X₂₅ = D or E |
| X₆ = T or I | X₁₆ = M or V | X₂₆ = I or L |
| X₇ = R or H | X₁₇ = S or T | X₂₇ = E or A |
| X₈ = T or P | X₁₈ = S or K | X₂₈ = N or H |
| X₉ = H or R | X₁₉ = S or N | X₂₉ = T or A |
| X₁₀ = Y or H | X₂₀ = S or skip | X₃₀ = H or D |

Enzyme (+)-limonene synthase set forth in SEQ ID NO: 35 is truncated to exclude the plastid
signaling peptide-SEQ ID NO: 36. Positions at which there are amino acid variations between
the different sequences are denoted by Xᵢ, with i = 1, 2, 3...30. The table below shows the
two most common amino acids for each Xᵢ from X₁ to X₃₀.
MDRRSANYQPSIWDHDFLQSLNSNYTDETYX₅RRAEELKGKVKX₆AIKDVTEPLDQLELIDNLQRLGLAYX₇FEX₈EI
RNILX₉NIX₁₀NX₁₁NKDYX₁₂WRKENLYATSLEFRLLRQHGYPVSQEVFX₁₃GFKDDX₁₄X₁₅GFICDDFKGILSLHEASY
YSLEGESIMEEAWQFTSKHLKEX₁₆MIX₁₇X₁₈X₁₉X₂₀X₂₁EEDVFVAEQAKRALELPLHWKVPMLEARWFIHX₂₂YEX₂₃R
EDKNHLLLELAKX₂₄EFNTLQAIYQEELKX₂₅ISGWWKDTGLGEKLSFARNRLVASFLWSMGIAFEPQFAYCRRVLTI
SIALITVIDDIYDVYGTLDELEX₂₆FTDAVX₂₇RWDINYALKHLPGYMKMCFLALYNFVNEFAYYVLKQQDFDMLLSI
KX₂₈AWLGLIQAYLVEAKWYHSKYTPKLEEYLENGLVSITGPLIITISYLSGTNPIIKKELEFLESNPDIVHWSSKIF
RLQDDLGTSSDEIQRGDVPKSIQCYMHETGASEEVAREHIKDMMRQMWKKVNAYTADKDSPLTRTTX₂₉EFLLNLVRM
SHFMYLHGDGHGVQNQETIDVGFTLLFQPIPLEDKX₃₀MAFTASPGTKG

| | | |
|---|---|---|
| X₅ = K or R | X₁₁ = N or H | X₂₁ = K or M |
| X₆ = T or I | X₁₂ = V or N | X₂₂ = V or I |
| X₇ = R or H | X₁₃ = N or S | X₂₃ = K or R |
| X₈ = T or P | X₁₄ = Q or K | X₂₄ = M or L |
| X₉ = H or R | X₁₅ = G or V | X₂₅ = D or E |
| X₁₀ = Y or H | X₁₆ = M or V | X₂₆ = I or L |
| | X₁₇ = S or T | X₂₇ = E or A |
| | X₁₈ = S or K | X₂₈ = N or H |
| | X₁₉ = S or N | X₂₉ = T or A |
| | X₂₀ = S or skip | X₃₀ = H or D |

Exemplary Limonene synthase consensus sequence 3, which shows the base pairs in common
(conserved regions)-SEQ ID NO: 37. Positions at which there are amino acid variations
between the different sequences are denoted by Xᵢ, with i = 1, 2, 3...116. The table below
shows the two most common amino acids for each Xᵢ from X₁ to X₁₁₆.
MSSX₁INPX₂TLX₃TSX₄NX₅FKX₆LPLATNX₇AAIRIX₈AKX₉KPVQCLX₁₀SX₁₁KYDNLX₁₂VDRRSANYQPX₁₃IWDHDF
LQSLNSX₁₄X₁₅TDEX₁₆YX₁₇RRAEELX₁₈GKVX₁₉X₂₀X₂₁IX₂₂DVX₂₃EPLDQLELIDNLQRLGLX₂₄X₂₅X₂₆FEX₂₇EIRNI
LX₂₈NIX₂₉NX₃₀NKDYX₃₁WRKENLYATSLEFRLLRQHGYPVSQEVX₃₂X₃₃GFKX₃₄DX₃₅X₃₆X₃₇FIX₃₈DDFX₃₉GILSLHE
ASX₄₀YX₄₁LEGESIMEEAWQFTSKHLKEX₄₂MIX₄₃X₄₄X₄₅X₄₆X₄₇EEDX₄₈FVAEQAKRALELPLHWKX₄₉X₅₀PMLEARWF
IHX₅₁YEX₅₂REDKNHLLLELAKX₅₃EFNX₅₄LQAIYQEELKX₅₅X₅₆SX₅₇WWKDX₅₈GLGEKLX₅₉FARX₆₀X₆₁LVASFX₆₂WS
MGIX₆₃FEPQFAYCRRX₆₄LTIX₆₅X₆₆ALIX₆₇VIDDIYDVYGTLDELEX₆₈FX₆₉DAVX₇₀RWDINYALX₇₁HLPX₇₂YMKX₇₃
CFLALYNX₇₄VNEFX₇₅YYVLKQQDFDX₇₆LX₇₇SIKX₇₈AWLX₇₉X₈₀IQAYLVEAKWYHX₈₁KYTPX₈₂LX₈₃EX₈₄LENGLVS
IX₈₅GPX₈₆X₈₇X₈₈X₈₉X₉₀X₉₁YLSGTNPIIX₉₂KELEFLESNX₉₃DIX₉₄HWSX₉₅KIX₉₆RLQDDLGTSSDEIQRGDVPKSIQ
CYMHETGASEEVARX₉₇HIKDMMRQMWKKVNAYX₉₈ADKDSPLX₉₉X₁₀₀TTX₁₀₁EFX₁₀₂LNX₁₀₃VRX₁₀₄SHFMYLHGDGHG
X₁₀₅QNQX₁₀₆TX₁₀₇DVX₁₀₈FTLLFQPIPLX₁₀₉DKX₁₁₀X₁₁₁X₁₁₂X₁₁₃X₁₁₄X₁₁₅SPX₁₁₆TKG

| | | | |
|---|---|---|---|
| X₁ = C or S | X₃₁ = V or N | X₆₁ = R or S | X₉₁ = S or A |
| X₂ = S or L | X₃₂ = F or S | X₆₂ = L or V | X₉₂ = K or E |
| X₃ = A or V | X₃₃ = N or S | X₆₃ = A or V | X₉₃ = P or Q |
| X₄ = A or V | X₃₄ = D or E | X₆₄ = V or I | X₉₄ = V or I |
| X₅ = A or G | X₃₅ = Q or K | X₆₅ = S or T | X₉₅ = S or F |
| X₆ = C or Y | X₃₆ = G or V | X₆₆ = I or F | X₉₆ = F or L |
| X₇ = R or K | X₃₇ = G or V | X₆₇ = T or S | X₉₇ = E or Q |
| X₈ = M or T | X₃₈ = C or F | X₆₈ = I or L | X₉₈ = T or R |
| X₉ = N or Y | X₃₉ = K or M | X₆₉ = T or A | X₉₉ = T or S |
| X₁₀ = V or I | X₄₀ = Y or H | X₇₀ = A or E | X₁₀₀ = R or Q |
| X₁₁ = A or T | X₄₁ = S or R | X₇₁ = K or N | X₁₀₁ = T or A |
| X₁₂ = T or I | X₄₂ = V or M | X₇₂ = G or D | X₁₀₂ = L or I |
| X₁₃ = S or P | X₄₃ = S or T | X₇₃ = M or I | X₁₀₃ = L or V |
| X₁₄ = N or D | X₄₄ = K or S | X₇₄ = F or L | X₁₀₄ = M or V |
| X₁₅ = T or A | X₄₅ = N or S | X₇₅ = A or T | X₁₀₅ = V or A |
| X₁₆ = Y or S | X₄₆ = S or skip | X₇₆ = M or I | X₁₀₆ = E or Q |
| X₁₇ = K or R | X₄₇ = K or M | X₇₇ = L or R | X₁₀₇ = I or M |
| X₁₈ = K or R | X₄₈ = V or L | X₇₈ = N or H | X₁₀₈ = G or V |
| X₁₉ = K or M | X₄₉ = K or skip | X₇₉ = G or R | X₁₀₉ = E or D |
| X₂₀ = T or I | X₅₀ = V or A | X₈₀ = L or N | X₁₁₀ = H or D |
| X₂₁ = A or T | X₅₁ = V or I | X₈₁ = S or G | X₁₁₁ = M or I |
| X₂₂ = K or E | X₅₂ = K or R | X₈₂ = K or T | X₁₁₂ = A or V |
| X₂₃ = T or I | X₅₃ = M or L | X₈₃ = E or G | X₁₁₃ = F or A |
| X₂₄ = A or V | X₅₄ = T or V | X₈₄ = Y or F | X₁₁₄ = T or A |
| X₂₅ = H or Y | X₅₅ = E or D | X₈₅ = T or G | X₁₁₅ = A or S |
| X₂₆ = H or R | X₅₆ = I or V | X₈₆ = L or M | X₁₁₆ = G or V |
| X₂₇ = T or P | X₅₇ = G or R | X₈₇ = I or V | |
| X₂₈ = H or R | X₅₈ = T or I | X₈₈ = I or T | |
| X₂₉ = Y or H | X₅₉ = S or N | X₈₉ = T or M | |
| X₃₀ = N or H | X₆₀ = N or D | X₉₀ = I or T | |

Enzyme (+)-limonene synthase set forth in SEQ ID NO: 37 is truncated to exclude the plastid
signaling peptide-SEQ ID NO: 38. Positions at which there are amino acid variations between
the different sequences are denoted by Xᵢ, with i = 1, 2, 3...116. The table below shows the
two most common amino acids for each Xᵢ from X₁ to X₁₁₆.
MDRRSANYQPX₁₃IWDHDFLQSLNSX₁₄X₁₅TDEX₁₆YX₁₇RRAEELX₁₈GKVX₁₉X₂₀X₂₁IX₂₂DVX₂₃EPLDQLELIDNLQR
LGLX₂₄X₂₅X₂₆FEX₂₇EIRNILX₂₈NIX₂₉NX₃₀NKDYX₃₁WRKENLYATSLEFRLLRQHGYPVSQEVX₃₂X₃₃GFKX₃₄DX₃₅
X₃₆X₃₇FIX₃₈DDFX₃₉GILSLHEASX₄₀YX₄₁LEGESIMEEAWQFTSKHLKEX₄₂MIX₄₃X₄₄X₄₅X₄₆X₄₇EEDX₄₈FVAEQAK
RALELPLHWKX₄₉X₅₀PMLEARWFIHX₅₁YEX₅₂REDKNHLLLELAKX₅₃EFNX₅₄LQAIYQEELKX₅₅X₅₆SX₅₇WWKDX₅₈G
LGEKLX₅₉FARX₆₀X₆₁LVASFX₆₂WSMGIX₆₃FEPQFAYCRRX₆₄LTIX₆₅X₆₆ALIX₆₇VIDDIYDVYGTLDELEX₆₈FX₆₉D
AVX₇₀RWDINYALX₇₁HLPX₇₂YMKX₇₃CFLALYNX₇₄VNEFX₇₅YYVLKQQDFDX₇₆LX₇₇SIKX₇₈AWLX₇₉X₈₀IQAYLVEA
KWYHX₈₁KYTPX₈₂LX₈₃EX₈₄LENGLVSIX₈₅GPX₈₆X₈₇X₈₈X₈₉X₉₀X₉₁YLSGTNPIIX₉₂KELEFLESNX₉₃DIX₉₄HWSX₉₅
KIX₉₆RLQDDLGTSSDEIQRGDVPKSIQCYMHETGASEEVARX₉₇HIKDMMRQMWKKVNAYX₉₈ADKDSPLX₉₉X₁₀₀TTX₁₀₁
EFX₁₀₂LNX₁₀₃VRX₁₀₄SHFMYLHGDGHGX₁₀₅QNQX₁₀₆TX₁₀₇DVX₁₀₈FTLLFQPIPLX₁₀₉DKX₁₁₀X₁₁₁X₁₁₂X₁₁₃X₁₁₄X₁₁₅
SPX₁₁₆TKG

| | | | |
|---|---|---|---|
| X₁₃ = S or P | X₃₁ = V or N | X₆₁ = R or S | X₉₁ = S or A |
| X₁₄ = N or D | X₃₂ = F or S | X₆₂ = L or V | X₉₂ = K or E |
| X₁₅ = T or A | X₃₃ = N or S | X₆₃ = A or V | X₉₃ = P or Q |
| X₁₆ = Y or S | X₃₄ = D or E | X₆₄ = V or I | X₉₄ = V or I |
| X₁₇ = K or R | X₃₅ = Q or K | X₆₅ = S or T | X₉₅ = S or F |
| X₁₈ = K or R | X₃₆ = G or V | X₆₆ = I or F | X₉₆ = F or L |
| X₁₉ = K or M | X₃₇ = G or V | X₆₇ = T or S | X₉₇ = E or Q |
| X₂₀ = T or I | X₃₈ = C or F | X₆₈ = I or L | X₉₈ = T or R |
| X₂₁ = A or T | X₃₉ = K or M | X₆₉ = T or A | X₉₉ = T or S |
| X₂₂ = K or E | X₄₀ = Y or H | X₇₀ = A or E | X₁₀₀ = R or Q |
| X₂₃ = T or I | X₄₁ = S or R | X₇₁ = K or N | X₁₀₁ = T or A |
| X₂₄ = A or V | X₄₂ = V or M | X₇₂ = G or D | X₁₀₂ = L or I |
| X₂₅ = H or Y | X₄₃ = S or T | X₇₃ = M or I | X₁₀₃ = L or V |
| X₂₆ = H or R | X₄₄ = K or S | X₇₄ = F or L | X₁₀₄ = M or V |
| X₂₇ = T or P | X₄₅ = N or S | X₇₅ = A or T | X₁₀₅ = V or A |
| X₂₈ = H or R | X₄₆ = S or skip | X₇₆ = M or I | X₁₀₆ = E or Q |
| X₂₉ = Y or H | X₄₇ = K or M | X₇₇ = L or R | X₁₀₇ = I or M |
| X₃₀ = N or H | X₄₈ = V or L | X₇₈ = N or H | X₁₀₈ = G or V |
| | X₄₉ = K or skip | X₇₉ = G or R | X₁₀₉ = E or D |
| | X₅₀ = V or A | X₈₀ = L or N | X₁₁₀ = H or D |
| | X₅₁ = V or I | X₈₁ = S or G | X₁₁₁ = M or I |
| | X₅₂ = K or R | X₈₂ = K or T | X₁₁₂ = A or V |
| | X₅₃ = M or L | X₈₃ = E or G | X₁₁₃ = F or A |
| | X₅₄ = T or V | X₈₄ = Y or F | X₁₁₄ = T or A |
| | X₅₅ = E or D | X₈₅ = T or G | X₁₁₅ = A or S |
| | X₅₆ = I or V | X₈₆ = L or M | X₁₁₆ = G or V |
| | X₅₇ = G or R | X₈₇ = I or V | |
| | X₅₈ = T or I | X₈₈ = I or T | |
| | X₅₉ = S or N | X₈₉ = T or M | |
| | X₆₀ = N or D | X₉₀ = I or T | |

HMGCR[NM_00859.2]: Full Genbank DNA sequence-SEQ ID NO: 39
ORIGIN

| | |
|---|---|
| 1 | ctcttattgg tcgaaggctc gtccagctcc gagcgtgcgt aaggtgaggg ctccttccgc |
| 61 | tccgcgactg cgttaactgg agccaggctg agcgtcggcg ccggggttcg gtggcctcta |
| 121 | gtgagatctg gaggatccaa ggattctgta gctacaatgt tgtcaagact ttttcgaatg |
| 181 | catggcctct ttgtggcctc ccatccctgg gaagtcatag tggggacagt gacactgacc |
| 241 | atctgcatga tgtccatgaa catgtttact ggtaacaata agatctgtgg ttggaattat |
| 301 | gaatgtccaa agtttgaaga ggatgttttg agcagtgaca ttataattct gacaataaca |
| 361 | cgatgcatag ccatcctgta tatttacttc cagttccaga atttacgtca acttggatca |
| 421 | aaatatattt tgggtattgc tggccttttc acaattttct caagttttgt attcagtaca |
| 481 | gttgtcattc acttcttaga caaagaattg acaggcttga atgaagcttt gccctttttc |
| 541 | ctacttttga ttgacctttc cagagcaagc acattagcaa agtttgccct cagttccaac |
| 601 | tcacaggatg aagtaaggga aaatattgct cgtggaatgg caattttagg tcctacgttt |
| 661 | accctcgatg ctcttgttga atgtcttgtg attggagttg gtaccatgtc aggggtacgt |
| 721 | cagcttgaaa ttatgtgctg ctttggctgc atgtcagttc ttgccaacta cttcgtgttc |
| 781 | atgactttct tcccagcttg tgtgtccttg gtattagagc tttctcggga aagccgcgag |
| 841 | ggtcgtccaa tttggcagct cagccatttt gcccgagttt tagaagaaga agaaaataag |
| 901 | ccgaatcctg taactcagag ggtcaagatg attatgtctc taggcttggt tcttgttcat |
| 961 | gctcacagtc gctggatagc tgatccttct cctcaaaaca gtacagcaga tacttctaag |
| 1021 | gtttcattag gactggatga aaatgtgtcc aagagaattg aaccaagtgt ttccctctgg |
| 1081 | cagttttatc tctctaaaat gatcagcatg gatattgaac aagttattac cctaagttta |
| 1141 | gctctccttc tggctgtcaa gtacatcttc tttgaacaaa cagagacaga atctacactc |
| 1201 | tcattaaaaa accctatcac atctcctgta gtgacacaaa agaaagtccc agacaattgt |
| 1261 | tgtagacgtg aacctatgct ggtcagaaat aaccagaaat gtgattcagt agaggaagag |
| 1321 | acagggataa accgagaaag aaaagttgag gttataaaac ccttagtggc tgaaacagat |
| 1381 | accccaaaca gagctacatt tgtggttggt aactcctcct tactcgatac ttcatcagta |
| 1441 | ctggtgacac aggaacctga aattgaactt cccagggaac ctcggcctaa tgaagaatgt |
| 1501 | ctacagatac ttgggaatgc agagaaaggt gcaaaattcc ttagtgatgc tgagatcatc |
| 1561 | cagttagtca atgctaagca tatcccagcc tacaagttgg aaactctgat ggaaactcat |
| 1621 | gagcgtggtg tatctattcg ccgacagtta ctttccaaga agctttcaga accttcttct |
| 1681 | ctccagtacc taccttacag ggattataat tactccttgg tgatgggagc ttgttgtgag |
| 1741 | aatgttattg gatatatgcc catccctgtt ggagtggcag gacccctttg cttagatgaa |
| 1801 | aaagaatttc aggttccaat ggcaacaaca gaaggttgtc ttgtggccag caccaataga |
| 1861 | ggctgcagag caataggtct tggtggaggt gccagcagcc gagtccttgc agatgggatg |
| 1921 | actcgtggcc cagttgtgcg tcttccacgt gcttgtgact ctgcagaagt gaaagcctgg |
| 1981 | ctcgaaacat ctgaagggtt cgcagtgata aaggaggcat ttgacagcac tagcagattt |
| 2041 | gcacgtctac agaaacttca tacaagtata gctggacgca acctttatat ccgtttccag |
| 2101 | tccaggtcag gggatgccat ggggatgaac atgatttcaa agggtacaga gaaagcactt |
| 2161 | tcaaaacttc acgagtattt ccctgaaatg cagattctag ccgttagtgg taactattgt |
| 2221 | actgacaaga aacctgctgc tataaattgg atagagggaa gaggaaaatc tgttgtttgt |
| 2281 | gaagctgtca ttccagccaa ggttgtcaga gaagtattaa agactaccac agaggctatg |
| 2341 | attgaggtca acattaacaa gaatttagtg ggctctgcca tggctgggag cataggaggc |
| 2401 | tacaacgccc atgcagcaaa cattgtcacc gccatctaca ttgcctgtgg acaggatgca |
| 2461 | gcacagaatg ttggtagttc aaactgtatt actttaatgg aagcaagtgg tcccacaaat |
| 2521 | gaagatttat atatcagctg caccatgcca tctatagaga taggaacggt gggtggtggg |
| 2581 | accaacctac tacctcagca agcctgtttg cagatgctag gtgttcaagg agcatgcaaa |
| 2641 | gataatcctg gggaaaatgc ccggcagctt gcccgaattg tgtgtgggac cgtaatggct |
| 2701 | ggggaattgt cacttatggc agcattggca gcaggacatc ttgtcaaaag tcacatgatt |
| 2761 | cacaacaggt cgaagatcaa tttacaagac ctccaaggag cttgcaccaa gaagacagcc |
| 2821 | tgaatagccc gacagttctg aactggaaca tgggcattgg gttctaaagg actaacataa |
| 2881 | aatctgtgaa ttaaaaaagc tcaatgcatt gtcttgtgga ggatgaatag atgtgatcac |
| 2941 | tgagacagcc acttggtttt tggctctttc agagaggtct caggttcttt ccatgcagac |
| 3001 | tcctcagatc tgaacacagt ttagtgcttt acatgctgtg ctctttgaag agatttcaac |
| 3061 | aagaatattg tatgttaaag catcagagat ggtaatctac agctcacctc tgaaggcaaa |
| 3121 | tataagctgg gaaaaaagtt ttgatgaaat tcttgaagtt catggtgatc agtgcaattg |
| 3181 | accttctccc tcactcctgc cagttgaaaa tggattttta aattatactg tagctgatga |
| 3241 | aactcctgat tttgtagtta atttattaag tctgggatgt agaacttcaa gaagtaagag |
| 3301 | ctaagttcta agttcatgtt tgtaaattaa tacttcattt ggtgctggtc tattttgatt |
| 3361 | ttggggggta atcagcatta ttcttcagaa ggggacctgt tttcttcaag ggaagaaaca |
| 3421 | ctcttattcc caaactacag aataatgtgt taaacatgct aaatagttct atcaggaaaa |
| 3481 | caaatcactg tatttatctc cgcaggctat ttgttcagag aggccttttg tttaaatata |
| 3541 | aatgtttaaa tataaatgtt tgtctggatt ggctataaca tgtctttcag cattaggctt |
| 3601 | ttaagaaaca cagggttttg tattctttac taaagatatc agagctctta atgttgctta |
| 3661 | gatgagggtg actgtcaagt acaagcaaga ctgggacctt agaaatcatt gtagaaacac |
| 3721 | agttttgaaa gaaaaatacc atgtctctaa gccaacttta attgcttaaa agacattttt |
| 3781 | atttagttga aaaatctagt tttttttgta aactgtatca aatctgtata tgttgtaata |
| 3841 | aaacttatgc tagtttattg gaagtgttca agaaataaaa atcaacttgt gtactgataa |
| 3901 | aatactctag cctgggccag agaagataat gttctttaat gttgtccagg aaaccctggc |
| 3961 | ttgcttgccg agcctaatga aagggaaagt cagctttcag agccagtgaa ggagccacgt |
| 4021 | gaatggccct agaactgtgc ctagttcctg tggccaggag gttggtgact gaaacattca |
| 4081 | cacagggctc tttgatggac ccacgaacgc tcttagcttt ctcagggggt cagcagagtt |
| 4141 | attgaatctt aatttttttt aatgtacaag ttttgtataa ataataaaga actccttatt |
| 4201 | ttgtattaca tctaatgctt caagtgttgc tcttggaaag ctgatgatgt ctcttgtaga |
| 4261 | agatggactc tgaaaaacat tccaggaaac catggcagca tggagagcct cttagtgatt |
| 4321 | gtgtctgcat tgttattgtg gaagatttac cttttctgtt gtacgtaaag cttaaattgc |
| 4381 | ttttgttgtg actttttagc cagtgacttt ttctgagctt ttcatggaag tggcagtgaa |
| 4441 | aaatatgttg agtgttcatt ttagtgactg taattaatat cttgctggat taatgttttg |
| 4501 | tacaattact aaattgtata cattttgtta tagaatactt ttttctagtt tcagtaaata |
| 4561 | atgaaaagga agttaatacc aaaaaaaaa |

Truncated HMGR (tHMGR) Sequence (truncated to include only the catalytic domain and
exclude the transmembrane regulatory domain of HMGR); (aa 426-aa 888) catalytic portion of
enzyme (From: “Crystal structure of the catalytic portion of human HMG-COA reductase: insights
into regulation of activity and catalysis.”)-SEQ ID NO: 40
MSSVLVTQEPEIELPREPRPNEECLQILGNAEKGAKFLSDAEIIQLVNAKHIPAYKLETLMETHERGVSIRRQLLSK
KLSEPSSLQYLPYRDYNYSLVMGACCENVIGYMPIPVGVAGPLCLDEKEFQVPMATTEGCLVASTNRGCRAIGLGGG
ASSRVLADGMTRGPVVRLPRACDSAEVKAWLETSEGFAVIKEAFDSTSRFARLQKLHTSIAGRNLYIRFQSRSGDAM
GMNMISKGTEKALSKLHEYFPEMQILAVSGNYCTDKKPAAINWIEGRGKSVVCEAVIPAKVVREVLKTTTEAMIEVN
INKNLVGSAMAGSIGGYNAHAANIVTAIYIACGQDAAQNVGSSNCITLMEASGPTNEDLYISCTMPSIEIGTVGGGT
NLLPQQACLQMLGVQGACKDNPGENARQLARIVCGTVMAGELSLMAALAAGHLVKSHMIHNRSKINLQDLQGACTKK
TA
tHMGR Nucleotide Sequence-SEQ ID NO: 41 (nt 1431-nt 2820)
Start (atg)

| | |
|---|---|
| 1432 | tcatcagta |
| 1441 | ctggtgacac aggaacctga aattgaactt cccagggaac ctcggcctaa tgaagaatgt |
| 1501 | ctacagatac ttgggaatgc agagaaaggt gcaaaattcc ttagtgatgc tgagatcatc |
| 1561 | cagttagtca atgctaagca tatcccagcc tacaagttgg aaactctgat ggaaactcat |
| 1621 | gagcgtggtg tatctattcg ccgacagtta ctttccaaga agctttcaga accttcttct |
| 1681 | ctccagtacc taccttacag ggattataat tactccttgg tgatgggagc ttgttgtgag |
| 1741 | aatgttattg gatatatgcc catccctgtt ggagtggcag gacccctttg cttagatgaa |
| 1801 | aaagaatttc aggttccaat ggcaacaaca gaaggttgtc ttgtggccag caccaataga |
| 1861 | ggctgcagag caataggtct tggtggaggt gccagcagcc gagtccttgc agatgggatg |
| 1921 | actcgtggcc cagttgtgcg tcttccacgt gcttgtgact ctgcagaagt gaaagcctgg |
| 1981 | ctcgaaacat ctgaagggtt cgcagtgata aaggaggcat ttgacagcac tagcagattt |
| 2041 | gcacgtctac agaaacttca tacaagtata gctggacgca acctttatat ccgtttccag |
| 2101 | tccaggtcag gggatgccat ggggatgaac atgatttcaa agggtacaga gaaagcactt |
| 2161 | tcaaaacttc acgagtattt ccctgaaatg cagattctag ccgttagtgg taactattgt |
| 2221 | actgacaaga aacctgctgc tataaattgg atagagggaa gaggaaaatc tgttgtttgt |
| 2281 | gaagctgtca ttccagccaa ggttgtcaga gaagtattaa agactaccac agaggctatg |
| 2341 | attgaggtca acattaacaa gaatttagtg ggctctgcca tggctgggag cataggaggc |
| 2401 | tacaacgccc atgcagcaaa cattgtcacc gccatctaca ttgcctgtgg acaggatgca |
| 2461 | gcacagaatg ttggtagttc aaactgtatt actttaatgg aagcaagtgg tcccacaaat |
| 2521 | gaagatttat atatcagctg caccatgcca tctatagaga taggaacggt gggtggtggg |
| 2581 | accaacctac tacctcagca agcctgtttg cagatgctag gtgttcaagg agcatgcaaa |
| 2641 | gataatcctg gggaaaatgc ccggcagctt gcccgaattg tgtgtgggac cgtaatggct |
| 2701 | ggggaattgt cacttatggc agcattggca gcaggacatc ttgtcaaaag tcacatgatt |
| 2761 | cacaacaggt cgaagatcaa tttacaagac ctccaaggag cttgcaccaa gaagacagcc |
| 2820 | |

Plastid-signaling peptide consensus amino acid sequence 1-SEQ ID NO: 42
SSCINPSTLVTSVNGFKCLPLATNKAAIRIMAKNKPVQCLVSAKYDNLTVD
Plastid-signaling peptide consensus amino acid sequence 2-SEQ ID NO: 43
SSX₁INPSTLX₂TSVNGFKCLPLATNX₃AAIRIMAKNKPVQCLVSX₄KYDNLTVD
X₁ = C or S
X₂ = V or A
X₃ = K or R
X₄ = A or T
Plastid-signaling peptide consensus amino acid sequence 3-SEQ ID NO: 44
SSX₁INPX₂TLX₃TSX₄NX₅FKX₆LPLATNX₇AAIRIX₈AKX₉KPVQCLX₁₀SX₁₁KYDNLX₁₂VD
X₁ = C or S
X₂ = S or L
X₃ = A or V
X₄ = A or V
X₅ = A or G
X₆ = C or Y
X₇ = R or K
X₈ = M or T
X₉ = N or Y
X₁₀ = V or I
X₁₁ = A or T
X₁₂ = T or I
SEQ IDs NO: 45-50 are long sequences and are only referred to in the accompanied sequence
listing and hereby incorporated to the description in their entirety.
Specific examples of the RRX8W motif include the following amino acid sequences (SEQ ID
NOs: 51-70):
RRX8W motif1₋SEQ ID NO: 51
RRXXXXXXXAW
RRX8W motif2₋SEQ ID NO: 52
RRXXXXXXXRW
RRX8W motif3₋SEQ ID NO: 53
RRXXXXXXXNW
RRX8W motif4₋SEQ ID NO: 54
RRXXXXXXXDW
RRX8W motif5₋SEQ ID NO: 55
RRXXXXXXXCW
RRX8W motif6₋SEQ ID NO: 56
RRXXXXXXXQW
RRX8W motif7₋SEQ ID NO: 57
RRXXXXXXXEW
RRX8W motif8₋SEQ ID NO: 58
RRXXXXXXXGW
RRX8W motif9₋SEQ ID NO: 59
RRXXXXXXXHW
RRX8W motif10₋SEQ ID NO: 60
RRXXXXXXXIW
RRX8W motif11₋SEQ ID NO: 61
RRXXXXXXXLW
RRX8W motif12₋SEQ ID NO: 62
RRXXXXXXXKW
RRX8W motif13₋SEQ ID NO: 63
RRXXXXXXXMW
RRX8W motif14₋SEQ ID NO: 64
RRXXXXXXXFW
RRX8W motif15₋SEQ ID NO: 65
RRXXXXXXXPW
RRX8W motif16₋SEQ ID NO: 66
RRXXXXXXXSW
RRX8W motif17₋SEQ ID NO: 67
RRXXXXXXXTW
RRX8W motif18₋SEQ ID NO: 68
RRXXXXXXXWW
RRX8W motif19₋SEQ ID NO: 69
RRXXXXXXXYW
RRX8W motif 20- SEQ ID NO: 70
RRXXXXXXXVW
Specific examples of the DDXXD motif include the following amino acid sequences (SEQ ID
NOs: 71-90):
DDXXD motif1₋SEQ ID NO: 71
DDXAD
DDXXD motif2₋SEQ ID NO: 72
DDXRD
DDXXD motif3₋SEQ ID NO: 73
DDXND
DDXXD motif4₋SEQ ID NO: 74
DDXDD
DDXXD motif5₋SEQ ID NO: 75
DDXCD
DDXXD motif6₋SEQ ID NO: 76
DDXQD
DDXXD motif7₋SEQ ID NO: 77
DDXED
DDXXD motif8₋SEQ ID NO: 78
DDXGD
DDXXD motif9₋SEQ ID NO: 79
DDXHD
DDXXD motif10₋SEQ ID NO: 80
DDXID
DDXXD motif11₋SEQ ID NO: 81
DDXLD
DDXXD motif12₋SEQ ID NO: 82
DDXKD
DDXXD motif13₋SEQ ID NO: 83
DDXMD
DDXXD motif14₋SEQ ID NO: 84
DDXFD
DDXXD motif15₋SEQ ID NO: 85
DDXPD
DDXXD motif16₋SEQ ID NO: 86
DDXSD
DDXXD motif17₋SEQ ID NO: 87
DDXTD
DDXXD motif18₋SEQ ID NO: 88
DDXWD
DDXXD motif19₋SEQ ID NO: 89
DDXYD
DDXXD motif20₋SEQ ID NO: 90
DDXVD
Specific examples of the NDXXD motif include the following amino acid sequences (SEQ ID
NOs: 91-110):
NDXXD motif1₋SEQID NO: 91
NDXAD
NDXXD motif2₋SEQID NO: 92
NDXRD
NDXXD motif3₋SEQID NO: 93
NDXND
NDXXD motif4₋SEQID NO: 94
NDXDD
NDXXD motif5₋SEQID NO: 95
NDXCD
NDXXD motif6₋SEQID NO: 96
NDXQD
NDXXD motif7₋SEQID NO: 97
NDXED
NDXXD motif8₋SEQID NO: 98
NDXGD
NDXXD motif9₋SEQID NO: 99
NDXHD
NDXXD motif10₋SEQID NO: 100
NDXID
NDXXD motif11₋SEQID NO: 101
NDXLD
NDXXD motif12₋SEQID NO: 102
NDXKD
NDXXD motif13₋SEQID NO: 103
NDXMD
NDXXD motif14₋SEQID NO: 104
NDXFD
NDXXD motif15-SEQID NO: 105
NDXPD
NDXXD motif16₋SEQID NO: 106
NDXSD
NDXXD motif17-SEQID NO: 107
NDXTD
NDXXD motif18₋SEQID NO: 108
NDXWD
NDXXD motif19₋SEQID NO: 109
NDXYD
NDXXD motif20₋SEQID NO: 110
NDXVD
Specific examples of the DDXXE motif include the following amino acid sequences (SEQ ID NOs:
111-130):
DDXXE motif1₋SEQ ID NO: 111
DDXAE
DDXXE motif2₋SEQ ID NO: 112
DDXRE
DDXXE motif3₋SEQ ID NO: 113
DDXNE
DDXXE motif4₋SEQ ID NO: 114
DDXDE
DDXXE motif5₋SEQ ID NO: 115
DDXCE
DDXXE motif6₋SEQ ID NO: 116
DDXQE
DDXXE motif7₋SEQ ID NO: 117
DDXEE
DDXXE motif8₋SEQ ID NO: 118
DDXGE
DDXXE motif9₋SEQ ID NO: 119
DDXHE
DDXXE motif10₋SEQ ID NO: 120
DDXIE
DDXXE motif11₋SEQ ID NO: 121
DDXLE
DDXXE motif12₋SEQ ID NO: 122
DDXKE
DDXXE motif13₋SEQ ID NO: 123
DDXME
DDXXE motif14₋SEQ ID NO: 124
DDXFE
DDXXE motif15₋SEQ ID NO: 125
DDXPE
DDXXE motif16₋SEQ ID NO: 126
DDXSE
DDXXE motif17₋SEQ ID NO: 127
DDXTE
DDXXE motif18₋SEQ ID NO: 128
DDXWE
DDXXE motif19₋SEQ ID NO: 129
DDXYE
DDXXE motif20₋SEQ ID NO: 130
DDXVE
Specific examples of the DXDD motif include the following amino acid sequences (SEQ ID
NOs: 131-150):
DXDD motif1₋SEQ ID NO: 131
DADD
DXDD motif2₋SEQ ID NO: 132
DRDD
DXDD motif3₋SEQ ID NO: 133
DNDD
DXDD motif4₋SEQ ID NO: 134
DDDD
DXDD motif5₋SEQ ID NO: 135
DCDD
DXDD motif6₋SEQ ID NO: 136
DQDD
DXDD motif7₋SEQ ID NO: 137
DEDD
DXDD motif8₋SEQ ID NO: 138
DGDD
DXDD motif9₋SEQ ID NO: 139
DHDD
DXDD motif10₋SEQ ID NO: 140
DIDD
DXDD motif11₋SEQ ID NO: 141
DLDD
DXDD motif12₋SEQ ID NO: 142
DKDD
DXDD motif13₋SEQ ID NO: 143
DMDD
DXDD motif14₋SEQ ID NO: 144
DEDD
DXDD motif15₋SEQ ID NO: 145
DPDD
DXDD motif16₋SEQ ID NO: 146
DSDD
DXDD motif17₋SEQ ID NO: 147
DTDD
DXDD motif18₋SEQ ID NO: 148
DWDD
DXDD motif19₋SEQ ID NO: 149
DYDD
DXDD motif20₋SEQ ID NO: 150
DVDD
DDIYD motif₋SEQID NO: 151
Specific examples of the VXDDXX(D, E) motif include the following amino acid sequences
(SEQ ID NO: 152 and 153):
VXDDXXD motif₋SEQ ID NO: 152
VXDDXXD
VXDDXXE motif₋SEQ ID NO: 153
VXDDXXE
Specific examples of the (I,L,V)XDDX(D,E) motif include the following amino acid sequences
(SEQ ID NOs: 154-159):
(I,L,V)XDDX(D,E) motif1₋SEQ ID NO: 154
IXDDXD
(I,L,V)XDDX(D,E) motif2₋SEQ ID NO: 155
LXDDXD
(I,L,V)XDDX(D,E) motif3-SEQ ID NO: 156
VXDDXD
(I,L,V)XDDX(D,E) motif4₋SEQ ID NO: 157
IXDDXE
(I,L,V)XDDX(D,E) motif5₋SEQ ID NO: 158
LXDDXE
(I,L,V)XDDX(D,E) motif6₋SEQ ID NO: 159
VXDDXE
Specific examples of the (N,D)D(L,I,V)X(S,T)XXXE motif include the following amino acid
sequences (SEQ ID NOs: 160-171):
(N,D)D(L,I,V)X(S,T)XXXE motif1-SEQ ID NO: 160
NDLXSXXXE
(N,D)D(L,I,V)X(S,T)XXXE motif2₋SEQ ID NO: 161
NDIXSXXXE
(N,D)D(L,I,V)X(S,T)XXXE motif3₋SEQ ID NO: 162
NDVXSXXXE
(N,D)D(L,I,V)X(S,T)XXXE motif4₋SEQ ID NO: 163
NDLXTXXXE
(N,D)D(L,I,V)X(S,T)XXXE motif5₋SEQ ID NO: 164
NDIXTXXXE
(N,D)D(L,I,V)X(S,T)XXXE motif6₋SEQ ID NO: 165
NDVXTXXXE
(N,D)D(L,I,V)X(S,T)XXXE motif7₋SEQ ID NO: 166
DDLXSXXXE
(N,D)D(L,I,V)X(S,T)XXXE motif8₋SEQ ID NO: 167
DDIXSXXXE
(N,D)D(L,I,V)X(S,T)XXXE motif9₋SEQ ID NO: 168
DDVXSXXXE
(N,D)D(L,I,V)X(S,T)XXXE motif10₋SEQ ID NO: 169
DDLXTXXXE
(N,D)D(L,I,V)X(S,T)XXXE motif11₋SEQ ID NO: 170
DDIXTXXXE
(N,D)D(L,I,V)X(S,T)XXXE motif12₋SEQ ID NO: 171
DDVXTXXX
Specific examples of the (N,D)DXX(S,T)XXXE motif include the following amino acid
sequences (SEQ ID NOs: 172-175):
(N,D)DXX(S,T)XXXE motif1₋SEQ ID NO: 172
NDXXSXXXE
(N,D)DXX(S,T)XXXE motif2₋SEQ ID NO: 173
NDXXTXXXE
(N,D)DXX(S,T)XXXE motif3₋SEQ ID NO: 174
DDXXSXXXE
(N,D)DXX(S,T)XXXE motif4₋SEQ ID NO: 175
DDXXTXXXE
Examples of suitable tumor-specific promoters include, but are not limited to:
Survivin promoter; human₋SEQ ID NO: 176
gccatagaaccagagaagtgagtggatgtgatgcccagctccagaagtgactccagaacaccctgtt
ccaaagcagaggacacactgattttttttttaataggctgcaggacttactgttggtgggacgccct
gctttgcgaagggaaaggaggagtttgccctgagcacaggcccccaccctccactgggctttcccca
gctcccttgtcttcttatcacggtagtggcccagtccctggcccctgactccagaaggtggccctcc
tggaaacccaggtcgtgcagtcaacgatgtactcgccgggacagcgatgtctgctgcactccatccc
tcccctgttcatttgtccttcatgcccgtctggagtagatgctttttgcagaggtggcaccctgtaa
agctctcctgtctgactttttttttttttttagactgagttttgctcttgttgcctaggctggagtg
caatggcacaatctcagctcactgcaccctctgcctcccgggttcaagcgattctcctgcctcagcc
tcccgagtagttgggattacaggcatgcaccaccacgcccagctaatttttgtatttttagtagaga
caaggtttcaccgtgatggccaggctggtcttgaactccaggactcaagtgatgctcctgcctaggc
ctctcaaagtgttgggattacaggcgtgagccactgcacccggcctgcacgcgttctttgaaagcag
tcgagggggcgctaggtgtgggcagggacgagctggcgcggcgtcgctgggtgcaccgcgaccacgg
gcagagccacgcggcgggaggactacaactcccggcacaccccgcgccgccccgcctctactcccag
aaggccgcggggggtggaccgcctaagagggcgtgcgctcccgacatgccccgcggcgcgccattaa
ccgccagatttgaatcgcgggacccgttggcagaggtggcggcggcggc
hTert core promoter; human₋SEQ ID NO: 177
ccagacccccgggtccgcccggagcagctgcgctgtcggggccaggccgggct
cccagtggattcgcgggcacagacgcccaggaccgcgcttcccacgtggcgga
gggactggggacccgggcacccgtcctgccccttcaccttccagctccgcctc
ctccgcgcggaccccgccccgtcccgacccctcccgggtccccggcccagccc
cctccgggccctcccagcccctccccttcctttccgcggccccgccctctcct
cgcggcgcgagtttcaggcagcgctgcgtcctgctgcgcacgtgggaagccct
ggccccggccacccccgcg
CXCR4 promoter, human [GenBank ID: U81003.1]₋SEQ ID NO: 178

| | |
|---|---|
| 1 | aaacgtctga cccccacccc cactccgccc cgcccagttc ttcaacctaa tttctgattc |
| 61 | gtgccaaagc ttgtcctctg ctcaaaatcg tggaagacgc cgagtatggg gaccgaagac |
| 121 | ctgggttcaa gcccggcttg gaatccctgc ccatccctgg catttcatct ctccgggctt |
| 181 | atttgctggt ttctccgaat gcgggccttg tctggttcac gctggatccc caacgcctag |
| 241 | aacagtgcgt ggcacgcagt tcgtccttct ataaatatcg gactaaatgc atctctgtga |
| 301 | tggtaatacc cacacggtgt tgtgagaatg aatgagtgat tctgtgcaag ttcctagtga |
| 361 | tctgttacaa aaagtactgg tcgctaaatt actcttataa taaagcatac ttttaggata |
| 421 | ataaagcact attcgcgaat tggttaccgc tattatgaaa ttactgagca atacatatct |
| 481 | acatctgatc agtctccaga attatgccaa atcgtacctt cttctgaaag tatgtcctaa |
| 541 | ttatctgcac ctgaccctag tgatgctgtg aatgtgcaag tatagataca tcctccgaag |
| 601 | gaaggatctt tactcctttt acctcctgaa tgggctgcgt ctgctgaaag cgcggggaat |
| 661 | ggcgttggaa gcttggccct acttccagca ttgccgccta ctggttgggt tactccagca |
| 721 | agtcactccc cttccctggg cctcagtgtc tctactgtag cattcccagg tctggaattc |
| 781 | catccacttt agcaaggatg gacgcgccac agagagacgc gttcctagcc cgcgcttccc |
| 841 | acctgtcttc aggcgcatcc cgcttccctc aaacttagga aatgcctctg ggaggtcctg |
| 901 | tccggctccg gactcactac cgaccacccg caaacagcag ggtcccctgg gcttcccaag |
| 961 | ccgcgcacct ctccgccccg cccctgcgcc ctccttcctc gcgtctgccc ctctccccca |
| 1021 | ccccgccttc tccctccccg ccccagcggc gcatgcgccg cgctcggagc gtgtttttat |
| 1081 | aaaagtccgg ccgcggccag aaacttcagt ttgttggctg cggcagcagg tagcaaagtg |
| 1141 | acgccgaggg cctgag |

Hexokinase type II promoter, human [GenBank: AF148512.1]₋SEQ ID NO: 179

| | |
|---|---|
| 1 | gatcacttga ggttaggagt ttgagaccag cctggccaac atgtcaaaac cctgtctcta |
| 61 | ctaaaaatat aaaaattagc tgggcatggt ggtgagtgcc tataatttca gctatttggg |
| 121 | aggctgaggc aggagaatcg cttgaaccca ggaggcggag gtggcagtga gccgagattg |
| 181 | tgccactgca ccccagcctg ggcgactaga gcaagaccct atctaaaaaa aacaaaaaac |
| 241 | aaacaacaaa caaacaaaga atctttgtta aatatctaag tctatatatt tatgggtgtc |
| 301 | tatatctgaa gagggaaagc ccagttatga ggctgttcca gtcaggtgag agataactgg |
| 361 | gcatatgatc tagggtagac agagaaatgg gaaaagattt gggaaataat atataaaact |
| 421 | ataaactcta tgtgtgtgtg tattgttaca caacatgtga acagtagtca tctctaagat |
| 481 | tcttctatga attcattcaa taaacgttta ttgcatgtct gccatgcgtc aggcaccatt |
| 541 | ttaggcactg caaacttgaa gggatgacag acacagaccc tgctgtcttt tagcttatca |
| 601 | tctattgagg agagggagaa catagcgaaa ataaatagga atcaactagg gccaagtgat |
| 661 | agtgacttgg ggaactattt gagataaact ggtcaaggaa agcctgatga ggtagaaggt |
| 721 | ggggacttga ctctggaggt gggggctaag actcgggacc agactctaga ttagagttcc |
| 781 | agatttaaca cctagaagtc actgcccctt tccatggcaa tgactcaaca acccgttacc |
| 841 | aacctttttc tagaaatttc tgtataatct gccccttaat ttgcatgtta actaaaagtg |
| 901 | ggtagaaata tgagtgcaga gctgcctctg agctgctact ctgggcacac ggccttatgg |
| 961 | ggtagccctg ctctgcaaag accagtgcct ctgctcctga tgtacactgc cacttcaata |
| 1021 | taagctgctg tctaatgcca cctgcttgcc cttgaatttt tttttttttt ttgaaatgga |
| 1081 | gtctctttct gttgcccagg ctggagtcag tggcgcgatc tcggctcact gcagctccgc |
| 1141 | ctcccgggtt cacgccattc tcctgcctca gcctcccgag tagctgggac tacaggagcc |
| 1201 | cgccaccacg cctaattttt ttgtattttt tttttttttg tagagatggg gtttcaccgt |
| 1261 | gttagctagg atggtctcga tctcctgacc tcgtgatccg tccacctcag cctcccaaag |
| 1321 | tgctgggatt acaggtgtga gccaccgcgc ccggcatccc ttgaattctt tactgggtga |
| 1381 | agccaagaat cttcccaggc taagtccaaa ttttggggcc tgcctgccct gcatcatgag |
| 1441 | gaggtatctg agtggaacgt caatgaggag gaagaatgag ttggagacag ccctggagaa |
| 1501 | gaatattcta gatagaagga aaaggaagag caaagaccct tgggtgagaa agagtttgta |
| 1561 | tttttgagga aagcatgcta gtgtgaatgc caagcagtat tctgtgggaa gatctcagga |
| 1621 | ggtgtctaag ggcatggaga taagtggtca gatgcacggt ctgttttata ggtggaatta |
| 1681 | actgcttgct gatggattga ctggctgtga gggtgagtgg caagaaggaa tcgaagatga |
| 1741 | gttagggtgg tggcgatgcc atttgctgag acaactggga aagaaaaaga tttgggaaaa |
| 1801 | aagttgagtt cagctttgga catgttaagt gtgatatgct agtcacttca gtggagatga |
| 1861 | caaatggcaa gctggagaat aagcctgaac tccagggagg acctcctgta gatttactat |
| 1921 | ggtgagtcat cagcatgcat atgatataac agtcatgggc tagaagttag tttctcctca |
| 1981 | gggagtttga aactgtaact agttcagaga agagggtgga gggcagcccc gataccccag |
| 2041 | catttaccaa tagagcaaac agggactcag gagcctgggg agtgaggtta gccggaaacc |
| 2101 | ctcagagtgg agcactggtg ctcttactga gagaggaagg tgtgtccaga tggaggatgt |
| 2161 | gattaactgt cctcaacatc cctgagagaa ggagtaagac aagggcaggg aagagaagag |
| 2221 | aatgcaagat ttggcaacat gtaggtcatt atgatgacta tgacaaaagc agtttgagct |
| 2281 | caattctgtg tggagtatag ggaaggaggg ttgaggacgt gcatttagaa gggtacatag |
| 2341 | ttctcaagaa gttttgctga gcacatctgt aatcccagct atttgggacg ctgaagtggg |
| 2401 | aggactgctt gagcccagga gttcaagacc agcctgggca acatatcgag tccctgctta |
| 2461 | aaaaaaaaaa aaaggaagtt ttgctgagag gctagatgga ttatgatttt tgtttatttt |
| 2521 | tcctgtttat ccatatatta tttttcaaca atgagtattg attacttata taataatttt |
| 2581 | aaggctgtac acattgcaga cagcacccca ctgtttgaaa aactcctcct cagtagaaca |
| 2641 | tggcagacct tcatcttcct tccctgaacc ttttccaacc ttaggcttgc cattctccac |
| 2701 | cagtgctaat gtcatgtctc ttgaaatctg tattgaagtc agtatttcat tcttgccagt |
| 2761 | ttccactgtg tgtttaaatt tggagtctgg tgtctagcat tagctggggt tggggcttcc |
| 2821 | actcctctca gcattggtaa gcctcctcac ccaccccatc ccatgtccaa gatcacccag |
| 2881 | ttacacactt accatctacc cagttcattc acatcatcag tcccagagct gcagagatgc |
| 2941 | tctttttcta cctcctactt ctctggctct tagagaggca gcatgggata atggggcaag |
| 3001 | cgaatagggc cttaaagtag agggacaagg gttctcttcc ctatctgcca cttattagct |
| 3061 | atgtgacctc gtgtaagtct cttttctttt tgagacaggg tctccctctg tcacctaggc |
| 3121 | tggagtacag tggtatgatc atagctcact gcagcctcga actcctgggc tcaagctatc |
| 3181 | cttccacctt agccttctga gcagcaggga ctacaggcac atgccaccat gtccggctga |
| 3241 | tttatttatt tttatttggg aagatggggg tctcactatg tcgcccaggc tggtcatgaa |
| 3301 | ctcctggtct caagcaaccc tccaaccttg gactcccaaa gtgctgggat tacaggtgtg |
| 3361 | agccctggcc ttgccacaat ttcctcatct gtaaaacggg gttagtgaaa ctcacatcct |
| 3421 | atcagtggtt ttgaggatgg gccgactctt gtattgcctg ctctagtaca atcagcagct |
| 3481 | aaggcggctc actttccggc cgtgctacaa taggtaagaa ctaggatgct ttagacgtgt |
| 3541 | gactgggcag tgggagcccc tcacatgatc ccgagatgcc agacagtgtc tctccgcaca |
| 3601 | gggcgtgtgc tggtccagag gcccgttttt ccagtcgccc cacaccccgg gtccgcgatc |
| 3661 | acgctccccc cacccatagc cgagcctgac gcggcggtgg ctcatgcgcc tttccgtccc |
| 3721 | agcctttagc cacggaccac acgtcccatc tcaggcgccc cgcccctccc ccgccccccg |
| 3781 | cccccggcgc gcctccccag gctgccggct ccggtgtctg agcggccgcg cccgcgagcc |
| 3841 | gtgagcgatg attggctgcg ccacggcggc gggcggtccg tgggcgcaca caccctcccc |
| 3901 | gcgcagccaa tgggcgtgcg cacgtcactg atccggagcc cgcgggccgg cagcccctca |
| 3961 | ataagccaca ttgttgcatg aaactccggc gcaggagtcc cgggctgccg ctggcaacat |
| 4021 | cgtgtcaccc agctaagaaa atccgcgggc ccgagccacg cgcctgtgaa tcggagaggt |
| 4081 | cccactgccc gagtggagcc gggctgagat tcttctcaag ttgagcctca gtgatcctgt |
| 4141 | ggccgaagtt agcgccttga cgtgggacaa ccggacacgt cgccaggaga gaactgaggc |
| 4201 | gccttctagc agttgtgacg ccaaaatcac gtctccggag acccgcgccc tccgccagcc |
| 4261 | gggcgcaccc tcgccggtag ccttctttgt gcgccgtccg gactcccagc tcccggcccg |
| 4321 | gcagccgagc cccagcacaa agcagtcgga ccgcgccgcc cgcctcccct ctcgcgtctc |
| 4381 | cgcctcggtt tcccaactct gcgccgtcgg gccgcggcag g |

Stromelysin 3 (MMP11) promoter, mouse [GenBank: AF297645.1]₋SEQ ID NO: 180

| | |
|---|---|
| 1 | ggcggccgct gagggtggtg ggtgcgcaag aggcggggct gggcggctgc aggaagcaag |
| 61 | aggagagaaa caaggttaat gctccgggaa taaacccctc tactccaggg tccagtggga |
| 121 | ccctcgttta gactcacgct tctgcgtccc cgtcctccca ccccaccccc cagccaacat |
| 181 | ggcgcagcag actccaaggt cattctgcgg acgcccttgg gagagcaccc acgtttccct |
| 241 | cacccgaccc cacggggtcc ctgtcgctct ctctctcacc tgaccggcct ggcagccgca |
| 301 | ctgcggcttc cccgaggcat gactgcggtg ggatcaagtt ggagctgagt aagaagcgtg |
| 361 | gaccgtagca gccgctcgct cagtgccggg caactaacac ggcagcgtcc ttagagtcag |
| 421 | gtgaaatggg cgggatctgg ggcggggcct ctgatccacg ccctccaaat gggaggggcc |
| 481 | aaagctcgcc cttccattaa cctcctggat ttagggctcc ggagctatcc cagtgcaggg |
| 541 | cggagctacg cgagtcctgg ggacaccggt cagctttgga aagcccaagg cttagttagg |
| 601 | cacggggcag cgagggcagg tctttctgtc agaactcaag caatgcaata ggggtttgcc |
| 661 | acgagcccag gaggaaagaa agagacacat agaccgccag cggagaagcg aatggagact |
| 721 | gcaggccagg ttgtgttctc tgagacccat cacaagacag agtttgaaaa taaccatccc |
| 781 | aggatcacca aaggcccttc cctgtcctct ggaactcggt tttcacagac ttttcctcag |
| 841 | agaccttggc tgggatcatg ccataacctc tggagagaga aaaaaaaaaa aaaggttaaa |
| 901 | agagcacaca cctgtaaccc cagaacttgg gggggggcag gtataggtag gtgcatcttg |
| 961 | tgtgttcgag gccagcctgg tctacagact gagttccagg gctacacaga actgtctccc |
| 1021 | acaaaacaaa acaaaataaa acaaataata ataacctttg gccagagtag aaagggcaca |
| 1081 | gggggccttg agttctttct cctcttcttt cagtgtttat tttactgtga caacagagat |
| 1141 | cacttggcac aaacaattca aatgcctcag caacaggggt caaaactatt caggaccatg |
| 1201 | cattgcccat tcaggtgtcc caaacctggt ttctttaagc agcctctgta gcaggcctct |
| 1261 | ttcattaaga cttcagcttt cctcccaagt ggaatcacca cccatctgca ggatagactt |
| 1321 | tctggtgagg tgagtaggta aaagacaagc cactctttcc tttaaaaaaa tgccagactc |
| 1381 | aggctagaga ggtggcttaa ctgttaagag cactgactgg ggctggagag agatggttca |
| 1441 | gtggttatga gagctgtctg ctcttccaga ggtcctgagt tcaattccca gcaactacat |
| 1501 | ggtggctcac aactgtctat aatgagatct gatgccctct tctagtgtgt ctgaaggcag |
| 1561 | cgacaatgta cccacataca cgaaataaat aaatacatct tttaaaaaag ggggggcagg |
| 1621 | gggctggcga gatggctcag tggttaagag tgccgactgc tcttctgaag gtcccgagtt |
| 1681 | caaatcccag caaccacatg gtggctcaca accatccata acgaaatctg atgccctctt |
| 1741 | cttgagtgtc tgaagacagc tacagtgtac ttacatataa taaataaata aatcttttaa |
| 1801 | aaaaatgtgc tgttgtagaa aattaaaaaa aaaaaggggg gggcaggctt gagcagaccc |
| 1861 | cactggctta tctatttggc ctctgcttac cttgtatcca gtaggcaagt ggtaacattc |
| 1921 | ttccagcttc aaccccttct gtgggcctcc gtggctagcc caccttccag atcctctact |
| 1981 | aagtgtggta atgtggggta atggggcagg ttggggggta agggggtggg aagtggaggg |
| 2041 | tggggggggt ggggggtctg gctgataagc tgcaagttcc tcagaaaata gtcgtgcatc |
| 2101 | cctggcaaac actgaaggct gtttaggttg cacaaataaa tgttttaggg tttgggggtt |
| 2161 | cttttgttga gacaggatct tcatatagcc tggctcactc tgtagagcag gttggtctca |
| 2221 | aacccacaga aatccacttg cttctgtctc ccaaatgtca ggattaaagg catgcatcca |
| 2281 | atgaagagtt tatttttaaa atgctatgca tggtggtgca tgcctttaat agaggcagat |
| 2341 | ctctgagttc aaggccagcc tactctacag agtcccagga ttgccagggc tacacagaga |
| 2401 | aacccactct tgggggtgga gggtaggact atgaatgcct catccatttt atgattcttt |
| 2461 | gaccaacact gctgagatga gtctgaacca gacctggaaa ttctagctat gatgatacat |
| 2521 | gcttgcagtc ctatcagtca gaataggcaa agacaggaat cttgagttgg aggcctgcct |
| 2581 | gggctacatg tacacatact agactctgtc aaaaaaaaga gagagagaga gagagagaga |
| 2641 | gagagagaga gagagagaga gagactctgt cataaaaaaa agaaaagagg ggtgggtggg |
| 2701 | aagggaggaa ggacgacggg aaggaatggc agcctttaaa aggtgaggct ttttaaaaga |
| 2761 | ttgcagatgg ccaagtaaaa cttaccactc tctgccccta ctttgcagca gctcagggcc |
| 2821 | ccactggccc accagaattc agaagagagc tgaaggcctg gtggaagagg cctgcagtgc |
| 2881 | cttgtagagc cattgtcatc caagagggaa cactgcacag ttggacactc gctgcagaga |
| 2941 | ttagagtagt tgaactgttt tcagcacgta gacctccctc tcagatgtga ttctgtccct |
| 3001 | gtctcagatg gctgagcctg actggtcagg aaaagcctgt tggctagtgt gccccccggc |
| 3061 | cagggaacaa cctgagattc cagtgtccaa ctcaaacatc cctgaccctt tcctcccagc |
| 3121 | caactgaccc agttgcctgc tagtagagaa aaaatctggt ccctccctcc aagatcctcg |
| 3181 | ctgactgcct ctggtctgaa attgtttaag tgtgcgcatt tgcatcagcc atttgcatca |
| 3241 | gcgtgtgcca agtgtcagta gaggtcagaa gaaggcatca gttcctagat ggccaccctg |
| 3301 | tgggtgctag gaacgggagc caggtttctc tgcaggagca acaagtgctc ctaaccactg |
| 3361 | atccatcttt ccagacccgt ctctgttttg ttttaaggtg tggggactgg ccaacttccg |
| 3421 | ccatatgcct cagtttcccc tgaggtcaca tttcaatagt ccgctccttg caagagctat |
| 3481 | tgtaccactt tcctgttagc tagggctgtt tagattgggg atctgaccac ctgccacagg |
| 3541 | ctgaaacaag tcaagccacc atggagagac ctggcgaagg atgagacttc tgaagtgggg |
| 3601 | ctggagagca gaattgagct ctccccggct ctcactagtt ctaagggacc cagcccctcc |
| 3661 | gggcactcct ccctaactca gaccgctgct gcaggccggt tgagtttaga acaaaaggca |
| 3721 | gggggaggcg gggcggtggg gggtgcggtc ccggcgccgg cgggggcggg gcgaatgcta |
| 3781 | taaggggcgg cggcccggcc tggcccagca ggcccaacag ccccggggcg gatg |

Tyrosinase promoter, human, [GenBank: U03039.1]₋SEQ ID NO: 181

| | |
|---|---|
| 1 | tagactgttg agtacaacac gtgtaggcca gaggagacag tggcctatac ttgggacaaa |
| 61 | taaagaggtc tgtcctattt aagaaaatca accctgtaaa ggaaattaat aggactaagt |
| 121 | acattttagt aaggcctcta agcaggctct aaagattatg aaaaatacac gggacagcag |
| 181 | acacaaaagc ccttaaagag catgaagact ttctaagtta tttcactgga agcctgatag |
| 241 | tggggcaagt gtaaggcaaa attcttaatt aaattgaaaa tgataagttg aattctgtct |
| 301 | tcgagaacat agaaaagaat tatgaaatgc caacatgtgg ttacaagtaa tgcagaccca |
| 361 | aggctcccca gggacaagaa gtcttgtgtt aactctttgt ggctctgaaa gaaagagaga |
| 421 | gagaaaagat taagcctcct tgtggagatc atgtgatgac ttcctgattc cagccagagc |
| 481 | gagcatttcc atggaaactt ctcttcctct tcacccacac actgctccat gtacctgcaa |
| 541 | agcctgttct gtctcaaaaa agttgtttgg atgagccgtg actttttttt ttcttaaata |
| 601 | atgagacaaa ctccagaaaa agagaaaaaa gcagagcagt ctgacattcc ggcatcatcg |
| 661 | aaatagtgat ggcttttcct agaatgcttc agctaaggac ccaaaatact aatgatctcc |
| 721 | tcaaagcttc agaggggcaa ctttgatttg actactcttt ttgtcactct tcagctcaca |
| 781 | aaagagctca ctttagttca aaacacaaag ctttaagccc ctccatagat tggtccaggt |
| 841 | ttaattttct atgatgagtg gaggcctcag tttaatgctc caacttgata gatgaaacac |
| 901 | agttccctcc tctacacatt tcccctgact caggagtttg tatatattct cagttgtctg |
| 961 | tccaacttat gcccactctt tgagatatta atcaaggcac tcccttgata acacttgcat |
| 1021 | attattatca aaattatgca attctttcta atatcagccc acaaatacat ctcttccatt |
| 1081 | aaaagtttga ctaattatct atactactca tttgaaaact aacatagtta agttgtattt |
| 1141 | ttagccatga atttcagttt ccctagctca ctatacacag agaaggaaac ttttgaaata |
| 1201 | attgagatga tcaaaaatat ttgctgaagt aaatatattt ctccttttca ttcactcact |
| 1261 | aattgagaat gtctttgcac aaaacacatt gcaaaaacat tttcaaaaaa attcctaatt |
| 1321 | tctagaattg ataggaaaaa caatatggct acagcattgg agagagagag aaaggagaga |
| 1381 | ggagaaagga gagagagaga aaggagagag gagagagaca gaggagagag agagaggata |
| 1441 | gagggggaga gagagagagg agagagacag aggagagaga gagaggatag aggggagaga |
| 1501 | gagggagagg gagagagagg gagagagagg gagagagaga gagagagagg gagagagaga |
| 1561 | gagaaagaga gagagaggga gagagagaga gagagctctt taacgtgaga tatcccacaa |
| 1621 | tgaacaaatc tgcccagtta tcaaagtgca gctatcctta ggagttgtca gaaaatgcat |
| 1681 | caggattatc agagaaaagt atcagaaaga tttttttttc tgatacgttg tataaaataa |
| 1741 | acaaactgaa attcaataac atataaggaa ttctgtctgg gctctgaaga caatctctct |
| 1801 | ctgcatattg agttcttcaa acattgtagc ctctttatgg tctctgagaa ataactacct |
| 1861 | taaacccata atctttaata cttcctaaac tttcttaata agagaagctc tattcctgac |
| 1921 | actacctctc atttgcaagg tcaaatcatc attagttttg tagtctatta actgggtttg |
| 1981 | cttaggtcag gcattattat tactaacctt attgttaata ttctaaccat aagaattaaa |
| 2041 | ctattaatgg tgaatagagt ttttcacttt aacataggcc tatcccactg gtgggatacg |
| 2101 | agccaattcg aaagaaaaag tcagtcatgt gcttttcaga ggatgaaagc ttaagataaa |
| 2161 | gactaaaagt gtttgatgct ggaggtggga gtggtattat ataggtctca gccaagacat |
| 2221 | gtgataatca ctgtagtagt agctggaaag agaaatctgt gactccaatt agccagttcc |
| 2281 | tgcagacctt gtgaggacta gaggaagaat g |

Interleukin-10 promoter, human [GenBank: Z30175.1]₋SEQ ID NO: 182

| | |
|---|---|
| 1 | gatccccaga gactttccag atatctgaag aagtcctgat gtcactgccc cggtccttcc |
| 61 | ccaggtagag caacactcct cgtcgcaacc caactggctc cccttacctt ctacacacac |
| 121 | acacacacac acacacacac acacacacac acacacaaat ccaagacaac actactaagg |
| 181 | cttctttggg agggggaagt agggataggt aagaggaaag taagggacct cctatccagc |
| 241 | ctccatggaa tcctgacttc ttttccttgt tatttcaact tcttccaccc catcttttaa |
| 301 | actttagact ccagccacag aagcttacaa ctaaaagaaa ctctaaggcc aatttaatcc |
| 361 | aaggtttcat tctatgtgct ggagatggtg tacagtaggg tgaggaaacc aaattctcag |
| 421 | ttggcactgg tgtacccttg tacaggtgat gtaacatctc tgtgcctcag tttgctcact |
| 481 | ataaaataga gacggtaggg gtcatggtga gcactacctg actagcatat aagaagcttt |
| 541 | cagcaagtgc agactactct tacccacttc ccccaagcac agttggggtg ggggacagct |
| 601 | gaagaggtgg aaacatgtgc ctgagaatcc taatgaaatc ggggtaaagg agcctggaac |
| 661 | acatcctgtg accccgcctg tcctgtagga agccagtctc tggaaagtaa aatggaaggg |
| 721 | ctgcttggga actttgagga tatttagccc accccctcat ttttacttgg ggaaactaag |
| 781 | gcccagagac ctaaggtgac tgcctaagtt agcaaggaga agtcttgggt attcatccca |
| 841 | ggttgggggg acccaattat ttctcaatcc cattgtattc tggaatgggc aatttgtcca |
| 901 | cgtcactgtg acctaggaac acgcgaatga gaacccacag ctgagggcct ctgcgcacag |
| 961 | aacagctgtt ctccccagga aatcaacttt ttttaattga gaagctaaaa aattattcta |
| 1021 | agagaggtag cccatcctaa aaatagctgt aatgcagaag ttcatgttca accaatcatt |
| 1081 | tttgcttacg atgcaaaaat tgaaaactaa gtttattaga gaggttagag aaggaggagc |
| 1141 | tctaagcaga aaaaatcctg tgccgggaaa ccttgattgt ggctttttaa tgaatgaaga |
| 1201 | ggcctccctg agcttacaat ataaaagggg gacagagagg tgaaggtcta cacatcaggg |
| 1261 | gcttgctctt gcaaaaccaa accacaagac agacttgcaa aagaaggcat gcacagctca |
| 1321 | gcactgc |

Epidermal growth factor receptor (EGFR) promoter, human [GenBank: J03206.1]₋SEQ ID NO: 183

| | |
|---|---|
| 1 | ctcctcctcc cgccctgcct cccgcgcctc ggcccgcgcg agctagacgt tcgggcagcc |
| 61 | cccggcgcag cgcggccgca gcgcctccgc cccccgcacg gtgtgagcgc ccgcccgccg |
| 121 | aggcggccgg agtcccgage tagccccggc ggcgccgccg cccagaccgg acgacaggcc |
| 181 | acctcgtcgc gtccgcccga gtccccgcct cgccgccaac gccacaacca ccgcgcacgg |
| 241 | ccccctgact ccgtccagta ttgatcggga gagccggagc gagctcttcg gggagcagcg |

Mucin-like glycoprotein (DF3, MUC1) promoter, human [GenBank: X69118.1]₋SEQ ID NO: 184

| | |
|---|---|
| 1 | gaattcagaa ttttagaccc tttggccttg gggtccatcc tggagaccct gaggtctaag |
| 61 | ctacagcccc tcagccaacc acagaccctt ctctggctcc caaaaggagt tcagtcccag |
| 121 | agggtggtca cccacccttc agggatgaga agttttcaag gggtattact caggcactaa |
| 181 | ccccaggaaa gatgacagca cattgccata aagttttggt tgttttctaa gccagtgcaa |
| 241 | ctgcttattt tagggatttt ccgggatagg gtggggaagt ggaaggaatc ggcgagtaga |
| 301 | agagaaagcc tgggagggtg gaagttaggg atctagggga agtttggctg atttggggat |
| 361 | gcgggtgggg gaggtgctgg atggagttaa gtgaaggata gggtgcctga gggaggatgc |
| 421 | ccgaagtcct cccagaccca cttactcacg gtggcagcgg cgacactcca gtctatcaaa |
| 481 | gatccgccgg gatggagagc caggaggcgg gggctgcccc tgaggtagcg gggaggccgg |
| 541 | ggggccgggg ggcggacggg acgagtgcaa tattggcggg ggaaaaaaca acactgcacc |
| 601 | gcgtcccgtc cctcccgccc gcccgggccc ggatcccgct ccccaccgcc tgaagccggc |
| 661 | ccgacccgga acccgggccg ctggggagtt gggttcacct tggaggccag agagacttgg |
| 721 | cgcccggaag caaagggaat ggcaaggggg aggggggagg gagaacggga gtttgcggag |
| 781 | tccagaaggc cgctttccga cgcccgggcg ttgcgcgcgc ttgctcttta agtactcaga |
| 841 | ctgcgcggcg cgagccgtcc gcatggtgac gcgtgtccca gcaaccgaac tgaatggctg |
| 901 | ttgcttggca atgccgggag ttgaggtttg gggccgccca cctagctact cgtgttttct |
| 961 | ccggcctgcg agttgggggg ctcccgcctc cccggcccgc tcctgggcgc gctgacgtca |
| 1021 | gatgtcccca ccccgcccag cgcctgcccc aagggtctcg ccgcacacaa agctcggcct |
| 1081 | cgggcgccgg cgcgcgggcg agagcggtgg tctctcgcct gctgatctga tgcgctccaa |
| 1141 | tcccgtgcct cgccgaagtg tttttaaagt gttctttcca acctgtgtct ttggggctga |
| 1201 | gaactgtttt ctgaatacag gcggaactgc ttccgtcggc ctagaggcac gctgcgactg |
| 1261 | cgggacccaa gttccacgtg ctgccgcggc ctgggatagc ttcctcccct cgtgcactgc |
| 1321 | tgccgcacac acctcttggc tgtcgcgcat tacgcacctc acgtgtgctt ttgccccccg |
| 1381 | ctacgtgcct acctgtcccc aataccactc tgctccccaa aggatagttc tgtgtccgta |
| 1441 | aatcccattc tgtcacccca cctactctct gcccccccct tttttgtttt gagacggagc |
| 1501 | tttgctctgt cgcccaggct ggagtgcaat ggcgcgatct cggctcactg caacctccgc |
| 1561 | ctcccgggtt caagcgattc tcctgcctca gcctcctgag tagctggggt tacagcgccc |
| 1621 | gccaccacgc tcggctaatt tttgtagttt ttagtagaga cgaggtttca ccatcttggc |
| 1681 | caggctggtc ttgaacccct gaccttgtga tccactcgcc tcggccttcc aaagtgttgg |
| 1741 | gattacgggc gtgacgaccg tgccacgcat ctgcctctta agtacataac ggcccacaca |
| 1801 | gaacgtgtcc aactcccccg cccacgttcc aacgtcctct cccacatacc tcggtgcccc |
| 1861 | ttccacatac ctcaggaccc cacccgctta gctccatttc ctccagacgc caccaccacg |
| 1921 | cgtcccggag tgccccctcc taaagctccc agccgtccac catgctgtgc gttcctccct |
| 1981 | ccctggccac ggcagtgacc cttctctccc gggccctgct tccctctcgc gggctctgct |
| 2041 | gcctcactta ggcagcgctg cccttactcc tctccgcccg gtccgagcgg cccctcagct |
| 2101 | tcggcgccca gccccgcaag gctcccggtg accactagag ggcgggagga gctcctggcc |
| 2161 | agtggtggag agtggcaagg aaggacccta gggttcatcg gagcccaggt ttactccctt |
| 2221 | aagtggaaat ttcttccccc actcctcctt ggctttctcc aaggagggaa cccaggctgc |
| 2281 | tggaaagtcc ggctggggcg gggactgtgg gttcagggga gaacggggtg tggaacggga |
| 2341 | cagggagcgg ttagaagggt ggggctattc cgggaagtgg tggggggagg gagcccaaaa |
| 2401 | ctagcaccta gtccactcat tatccagccc tcttatttct cggccgctct gcttcagtgg |
| 2461 | acccggggag ggcggggaag tggagtggga gacctagggg tgggcttccc gaccttgctg |
| 2521 | tacaggacct cgacctagct ggctttgttc cccatcccca cgttagttgt tgccctgagg |
| 2581 | ctaaaactag agcccagggg ccccaagttc cagactgccc ctcccccctc ccccggagcc |
| 2641 | agggagtggt tggtgaaagg gggaggccag ctggagaaca aacgggtagt cagggggttg |
| 2701 | agcgattaga gcccttgtac cctacccagg aatggttggg gaggaggagg aagaggtagg |
| 2761 | aggtagggga gggggcgggg ttttgtcacc tgtcacctgc tcgctgtgcc tagggcgggc |
| 2821 | gggcggggag tggggggacc ggtataaagc ggtaggcgcc tgtgcccgct ccacctctca |
| 2881 | agcagccagc gcctgcctga atctgttctg ccccctcccc acccatttca ccaccaccat |
| 2941 | g |

Somatostatin receptor 2 (sst2)promoter, human [GenBank: AB260891.1]₋SEQ ID NO: 185

| | |
|---|---|
| | caacgggtac ccttgcctga gtaagggggc tgtgggtaga gtgtgctgga acggacgtgt |
| 4261 | cctcgcagcc tcatgcccgt gtgcgtggcg tgtgcccttt agcccgagat ttcaggtagc |
| 4321 | tgcgacgggt gacaacttct ctcccagccc cctacaaaag agacctggcg cgaggggagc |
| 4381 | gaggccgtga gatgccagct ggggctcctg cgggagcgca cccggagatc cgagcctgcc |
| 4441 | agaggcaggc ggcgggcgca gagcggagaa agaggggctt ctctccctag acgctgaacg |
| 4501 | atctaggatc cgtccccgtc ccccacctcg ggacagaaag gacagtttgt ctaggtttgg |
| 4561 | agagaaaaaa ccactgcata ggccgtgccc aaaagccgct ggccaagtcc cccaagcgac |
| 4621 | tgtcttctgc gccccgatgt ctctgtcctc agcgcccccc ccccacaccc ggcacccctg |
| 4681 | ctgtgcgttt cgatactggg cgtgctggcg ccacaatctc cgctcttgcc tcgtcttcct |
| 4741 | ggaaatggca cagagtcctt tgggaaaccc ttgctctgag gatcagcgag ttggatggcc |
| 4801 | aggaggagga ctttctgtgc cagccgggag caaccggctc cgcggtcctg acactcgccc |
| 4861 | ctccatttct caaccccgta ggccagcacc gccccggctt ttcccaggcg ctcacgcgcc |
| 4921 | gcggtggccc tcaggggctt ttgtcaccct gccagtgggg gctctcgctc tagccgcaca |
| 4981 | gagaccaagc cgggttctgc aggccctgag ggaggtgggg ggtgggaagt gaatgcggga |
| 5041 | aacatgatgg ggagaggaga aactgaagct gagtaggatt taggacctcc cctgatgtcg |
| 5101 | ggtcgccatc ccaacactca tttcttgggc tggtaatcac agcccctatg taaaaggggg |
| 5161 | gcgggggggg caggtgcgta agaccattct caccctcctc tctacagagc ctggacatgg |
| 5221 | ttcagaggaa accgaccact agccatttcc agcatctaac aattcttggg ctggaaaaac |
| 5281 | aaagaatgca gaaaacgaaa cttccttgta catttaattt aaccacaatt catctagaat |
| 5341 | tgtctgcctg gcattggaat attctttctc tgaaacaaaa atgaaacaga agtctctgga |
| 5401 | agaccttaag cggctgactt ctttgttaaa taagactccc catgatttaa gctcatttct |
| 5461 | tgcttagagg agccttccca ctctcagccg gctccccagc ctcccacctc caccaccttc |
| 5521 | accaagactc tgaaccctgt ctgttgctac cattaagcaa ttctgtcctg ttgactcaaa |
| 5581 | ctccagttaa aatgaccgag ttagggctgg aaagcaacac tcaaccctct ctcatactcc |
| 5641 | ctgcaccatc atcgttccta gcccaaaagc tcttagacag gggctctgcc aacccagggg |
| 5701 | gattccgtgt tactcagaca ttggagtgtg accattcatg ttatatagat gggcccctgg |
| 5761 | aaatccccat gataaggtac actctgattg caggcagctt gaataggatt ctggctctgt |
| 5821 | agaattaaac caactgacca gatggttaga agtgataacg aaactaccca agttaatcca |
| 5881 | gggatactaa ccacagtttc tgtacagctt ctgttttaat tgctgccagt ctatgctttt |
| 5941 | ttacgcaatg cagacatgaa attccaggtg cctcaaatac ttcacaaaat ggtcagccac |
| 6001 | aaagcccaga tctcacttca cagacagttg tgtggtaggg aaatgagcac agaaggaacg |
| 6061 | agcaatgcac ctggcagttc agaatcaatc agaagcaaag gtgagcaagg atcctcaagt |
| 6121 | acttgttgct ggccaagtct cctttaactg atctgcagtc tttccaagga ttaagaagta |
| 6181 | atcttccatc tacacccagg caccaggaaa aggacctagc tcaggggaaa tgtgtcagcc |
| 6241 | aagtgaatta gtcccactct gctgaacaca ccaccctttg aacatctcgc ctcttcctag |
| 6301 | attggcctct ttgctgtcct cctgcttcac tcttcatata cccaagaccc agctcaaaca |
| 6361 | attctctttg gaagcctcct ctgagtcccc caggaaagga aggcattctt aagtccttca |
| 6421 | tttatctctc gtgcaatgcc caccctatat gagctggctt cctttcctat ctcccctttt |
| 6481 | aaattatcac ctcctagagg gcactggcca agtttgttca tttctacatc cctgctgtca |
| 6541 | gcacaaagaa gcctcctctc caggccccca acccccgtga tattttttga atggctgtat |
| 6601 | atcaatcatt taattatggg atgaactatt gttttagatc ttaagccaag ccaatagtgc |
| 6661 | tccaattatt ttctcagcaa ggaagtaaca caggagtcag ttgcttcaaa ccaaagccca |
| 6721 | gttatcagcc gttcggtctc taggccactg aggagcagag gggatgcctt gagacgtgca |
| 6781 | aaagacttgg ggccaggtgg cctgtgttca catcccagct ccaccaatta tgtgcaagag |
| 6841 | aatggggtga gctccttaaa ctctcttaag cctcagtttc cacatctcta aaatgggggt |
| 6901 | aattatccct accacctagg acagttgggg agatcaaggg actcgtgaat gtgaatgaat |
| 6961 | tatatcagta ctggaagcct tctgcttact tctgtgaaag agcttgtgtc ccacacctgc |
| 7021 | ttcccgtttt tgtccgtaat tagaaaacgg caggcaaatt ctctggagtg ttacagcact |
| 7081 | tgggagcagc atccccttag ggactttggg aaagagctct tgaggaagtc aagcattagg |
| 7141 | tattggaaaa caaaaataga agaaaaacaa aaaataaact gaagcctaca tttcaaaaat |
| 7201 | gaaagcaaac cagactttta tttttaatac tgaagactat aaattgtttc accacgtagg |
| 7261 | tagatttcaa taaatcaggg ataatgagat ggtagaggaa aacatggggg gaaacaactt |
| 7321 | acgaggttcc cattatgagc ccaacgcaag gctaggcatt ttcacatata ttccatcatt |
| 7381 | taaccttcat gacgccccca tgtgaagaaa taagagtcag aaccattaag gaccaggcat |
| 7441 | gtggtcacac gggctcagca gtggaacccg gtttgttctg cctctagagt ctgggttttt |
| 7501 | tccactatgg cattttcaga atggaaagac tccaaggcag tcagcaagtc agcatagatt |
| 7561 | tcctggtagg gaagaggcca ggaatgtcag tgtcagaccc ttctgaggtc aggcgctgaa |
| 7621 | cttctccaag ctctgccttt ctgcagttta gatcagtcaa cttcttaggg gtcaaagtat |
| 7681 | gtgctttttg aagccacagc cctccccgac atgtgcgtca gcagatgatg gctgaaccca |
| 7741 | aacccttccc tactattgga aaaacaactc aaaaagtctg cacactgatg aggaactcta |
| 7801 | gagcttaatg ttgatgtgga aagataatac atttttcaat ttaagagtat gtctgagagg |
| 7861 | ctaaaccaga aatgtgtaaa tttggtgaga ctttaaacag cctgtgaccg acgggccaat |
| 7921 | cttcctcttt tccttccaga tgtcacactg gatccttggc ctccagggtc cattaaggtg |
| 7981 | agaataagat ctctgggctg gctggaacta gcctaagact gaaaagcagc c |

c-erbB-2 promoters, human [GenBank ID: M16892.1]₋SEQ ID NO: 186

| | |
|---|---|
| 1 | cccgggggtc ctggaagcca caaggtaaac acaacacatc cccctccttg actatcaatt |
| 61 | ttactagagg atgtggtggg aaaaccatta tttgatatta aaacaaatag gcttgggatg |
| 121 | gagtaggatg caagctccca ggaaagttta agataaaacc tgagacttaa aagggtgtta |
| 181 | agagtggcag cctagggaat ttatcccgga ctccggggga gggggcagag tcaccagcct |
| 241 | ctgcatttag ggattctccg aggaaaagtg tgagaacggc tgcaggcaac ccagcttccc |
| 301 | ggcgctagga gggacgcacc caggcctgcg cgaagagagg gagaaagtga agctgggagt |
| 361 | tgccactccc agacttgttg gaatgcagtt ggagggggcg agctgggagc gcgcttgctc |
| 421 | ccaatcacag gagaaggagg aggtggagga ggagggctgc ttgaggaagt ataagaatga |
| 481 | agttgtgaag ctgagattcc cctccattgg gaccggagaa accagggagc ccccccggg |

c-erbB-3 promoter; human [GenBank ID: Z23134.1]₋SEQ ID NO: 187

| | |
|---|---|
| 1 | ggatccgtcc cgggactagc agggctttgg gcagcaaccc gcaggagccc gaccgcctct |
| 61 | ggccaggtcc gggcagctgg tgggggaggt tccagaggtc cacgccattc gtggacgcag |
| 121 | tctctagtgt cctctccgcg tcccacttca ctgccccatc ccctttcctg cgagagcctg |
| 181 | gacttggaag gcacctggga gggtgtaagc gccttggtgt gtgcccatct gggtccccag |
| 241 | aagagcggcg ggaactgcgg ccgcccggac ggtgcggcca gactccagtg tggaagggga |
| 301 | ggcagctgtt ctcccaggcg gccgtggggg gcagcagagg ggacggcgac aggtgcggga |
| 361 | gcccctcccg gggtagaagt ggaaaggcgg gctccggggt ctgttcccag gctggaaacc |
| 421 | acccccgccc cccatccaaa tccccgggag aggcccggcc ggcgccgggt ctggaggagg |
| 481 | aagcggccag agacagtgca atttcacgcg gtctctgtgg ctcgggttcc tgggctgggt |
| 541 | ggatgaatta tggggtttcg agtctgggag aaactgaggt ggcctggacg tgaggcaaaa |
| 601 | aacaccctcc ccctcaaaaa cacacagaga gaaatattca cattctgaga gaaaatccac |
| 661 | caagtgaacc aaccggctag gggagttgag tgatttggtt aatgggcgag gccaactttc |
| 721 | agggggcagg gctttggaga gctttccact ccctcattca ttacccttcc ctggatctgg |
| 781 | gggctttcgg aatctcgacc tccccttggc ctatctcctg cagaaaaatt agggtgagcc |
| 841 | ccatcctcga tctgctccgc caagttgcgg gaccgcgggg cgtggcacgc tcaggggcag |
| 901 | gcggtccgag gctccgcaat ccccactcca gcctcgcgcg ggagggggcg cggcccgtgt |
| 961 | gactcacccc cttccctctg cgttcctccc tccctctctc tctctctctc acacacacac |
| 1021 | acccctcccc tgccatccct ccccggactc cggctccggc tccgattgca atttgcaacc |
| 1081 | tccgctgccg tcgccgcagc agccaccaat tcgccagcgg ttcaggtggc tcttgcctcg |
| 1141 | atgtcctagc ctaggggccc ccgggccgga cttggctggg ctcccttcac cctctgcgga |
| 1201 | gtcatgaggg cgaacgacgc tctgcaggtg ctgggcttgc ttttcagcct ggcccggggc |
| 1261 | tccgaggtgg gcaactctca ggcaggtaag tgcccagaga gcacc |

Thyroglobulin promoter, human [GenBank: X77275.1]₋SEQ ID NO: 188

| | |
|---|---|
| 1 | ggatccagca atatggtggc aggctggact aaaggagaga tgactgggaa gcaatttcct |
| 61 | gtggtgcatg acagctgatg gatggatgtc agaaacagtg gtgtctgatg atccatttga |
| 121 | agccatttcc tcctctatat tgctattact gtccatctcc ccctaaattt tcagtaagca |
| 181 | cctattatat aaagcacctt agtattaaaa aatgaaggag atgaaagaga aggttgtgca |
| 241 | gttgtatttt gggccaagaa gagtgggaga ggtggcaggg ccagcgatga agagcctgcc |
| 301 | agagtgatgg aggcctgagc aaggagcaag ttggtgaaga aagattagga cattgccatg |
| 361 | tggagtcgct gtggaagcct gtttgttctc acgagctcag tggagaagag gtaaaagtag |
| 421 | ggaccagtag ctgagtcatt atgagaaaga gggtttcatg gtggtggaag tgacacattg |
| 481 | cctcgattct cttgaagctt tctgctttgt tgcttgagtg gagagaagca cctctgctat |
| 541 | tgcgtatgga gggaagctct ttgcatggat tttgaaggcg gcctctgcat ttcggactac |
| 601 | tgggtgctcc cccacaggct cctaacacct tgctgcttct ccaggtgggg tctgacgtgg |
| 661 | agtcagctca cagacctgcc attcctctct catagtactc ctcattccag tgatatcttg |
| 721 | gcctgcttca tgaaccctga gcccagagtt cctaaagcac caaacccagt gaagcagaga |
| 781 | cacttctggc atgggtctgt gggttgcttc tcaggggcca ggccagcaag aatgattcag |
| 841 | cacacaggcc aacctgtgca agctttatgc atgcatttta gggcaatggg aagagtggtg |
| 901 | agtgaggttt atggtaaatc tttaaccaca ttcaattttt tctaagactt ttctgcttta |
| 961 | gaacatgtag aaatggagaa atgaccaggg gctgcacaat gctgtgctta ttatattgct |
| 1021 | gtagagagaa ggatgctgcc agctctccat agcctggggt gaacttggcc tatgtaatga |
| 1081 | ggtagcaggg agtcaggcag gtgagttctt cctcttgtat tgccttttcc agtaaatgcc |
| 1141 | aatacactcc ccagctcacc tttacctaac atctaggtct taatccaagt tgtcctccca |
| 1201 | ctccccaggg tgaattgatc ttcctgccac ggcacctcct gagcatctgt tggctgagtc |
| 1261 | tccatcccca cccgtgaaaa cagccttgtg atgtgctgtt taatatcaca gaatggaaac |
| 1321 | agtgttttga ttcaccagga tcc |

Alpha-fetoprotein (AFP) promoter, human [GenBank: AB053572.1]₋SEQ ID NO: 189

| | |
|---|---|
| 1 | caaagagctc tgtgtccttg aacataaaat acaaataacc gctatgctgt taattattgg |
| 61 | caaatgtccc attttcaacc taaggaaata ccataaagta acagatatac caacaaaagg |
| 121 | ttactagtta acaggcattg cctgaaaaga gtataaaaga atttcagcat gattttccat |
| 181 | attgtgcttc caccactgcc aataacaaaa taactagcaa cca |

Villin 2 promoter, human [GenBank: EF184645.1]₋SEQ ID NO: 190

| | |
|---|---|
| 1 | agtgaatgct gttgctgctc gtctggaagc cagacgttga gaaccccttc tagagtgagc |
| 61 | tctcccgcag caaattctac tggcccccaa agtatgtgtt ttgtgtgtct taaaaatttg |
| 121 | ttgagaacca ttagcaaaaa aacaaacaaa aaaacttaat tcctagaatt ccagagaaat |
| 181 | cccatggagc tttttgccag tcacgtcaag agaggccaca aacgtgccac ttaaccagag |
| 241 | cttcggaaag gcggcggctg ggccggccac gtgcaccgag actcggggcc aggtgcagcc |
| 301 | gccccagggc cgaggcctcg gaactggccc ccggtcccgg ccccaagcgg tccagcgatt |
| 361 | cccccaagcc gtccgcccct ccagatttat ttacgttttc ctgacttccc cctgcccgct |
| 421 | gtgggacaaa cagcctcccc acttgcatct gcgaggggag tagcgcgcac ttccgccaag |
| 481 | ttccgccccc acccagcccg aggcccggct gccgccatct tgcggggggc gcacctcaca |
| 541 | ggtcgggagc tgggcgggaa ggggcgtggt cccgggaccc gccccgccgg ggcttttggg |
| 601 | agcgcgggca gcgagcgcac tcggcggacg caagggcggc ggggagcaca cggagcactg |
| 661 | caggcgccgg gtgaggcgtg cggcggccgg ggtcgggacg ggggttctgg gcggggggtt |
| 721 | cctggtggag ggcccgggcg ggcggcgggg ttcggcggca ggtgcggcgg gcagcctagg |
| 781 | gggcgcggcg cggggttctc gcccggcacc cccggggcag gtggagctga gccggcccgc |
| 841 | ggccccgcga ccttcccctc ggcgccgggt cccctcaggt ctctcccgaa ggaaacgcgg |
| 901 | agcctgggtg cctgggcgcc gtccctcggc ggctcccgag cggttgcagt ttttgaaaga |
| 961 | gtttctcaaa ggcttgacgg ttgtgactgc agccgcgggg caacggttgc tacacaaagt |
| 1021 | gaaacttgcc gagtgctcgg cttctcacgg gcttcctggc agccccggga agttcctcgg |
| 1081 | cggaccccga gcccgcgccc cctctccacg gatccctccc cagcgagtgc ccccccgccc |
| 1141 | gccctgtgcc ccctctcccc tgacccctcc ctgtcgggtg ccccgcgggc tcgcgctggc |
| 1201 | tgtcctggga ctccttcctc ctaggtgttc ctcctgcccc tcgccctctc tctcccaggc |
| 1261 | gcgcgctccc tctccccggg cctttccccg ccgggtatcc ctgggcccgc gccccgtctt |
| 1321 | ctccgcctct ctccgctggg tgcacctcga gtgtccccca gacccctccc cgcccggccg |
| 1381 | gcgctctctc ccctgaccct cctggccgag tgttccccgg ggcccgcgcc ccctcccccc |
| 1441 | gatcctcccc actgagtgtt ccccctgccc tctctctccc gggcctgcgc cccccaccag |
| 1501 | ccccttcatg ctgggggtcc cctgggtgcg caccccctct cctcggaccc acccccaact |
| 1561 | ggggggcacc tccagtgccc gccggctgcc ccttgggcgc gcgcccccgc tctcgggcgc |
| 1621 | ctcctcgccg ggggcccggc ccggccccgc cccgcccgtg ccccctcccc atgcccgcag |
| 1681 | tgctgggcgg ggcgctgact cacccgggcc cgggctggcc ggttcttaag cggcagcgcg |
| 1741 | ctgcgggcgc cgagtgtcgg gcgcggcagg aggacgaggc agggcgggcg ggcgctctaa |
| 1801 | gggttctgct ctgactccag gttgggacag cgtcttcgct gctgctggat agtcgtgttt |
| 1861 | tcggggatcg aggatactca ccagaaaccg aaa |

Albumin promoter, human₋SEQ ID NO: 191
ttaaactcttatgtaaaatttgataagatgttttacacaactttaatacattgacaaggtcttg
tggagaaaacagttccagatggtaaatatacacaagggatttagtcaaacaattttttggcaag
aatattatgaattttgtaatcggttggcagccaatgaaatacaaagatgagtctagttaacacg
tatattaatctacaattattggttaaagaatagtgctaatttccctccgtttgtcctagctttt
ctcttctgtcaaccccacacgcctttgg

What is claimed is:

1. A composition, comprising: a nucleic acid molecule encoding an exogenous synthase, wherein the exogenous synthase expresses preferentially in cancer cells compared to noncancerous cells and catalyzes production of a volatile organic compound, and wherein the volatile organic compound is not endogenously produced.

2. The composition as set forth in claim 1, wherein the volatile organic compound is a plant volatile organic compound, a terpene, a terpenoid, a monoterpene, or limonene.

3. The composition as set forth in claim 1, wherein the exogenous synthase is an enzyme limonene synthase.

4. The composition as set forth in claim 3, wherein the enzyme limonene synthase comprises at least one amino acid sequence that is at least about 70% identical to the amino acid sequence selected from SEQ ID NOs: 1, 3, 5, 7, 9, 11, 13, 15, 17, 19, 21, 23, 25, 27, 29, 31, 33, or 35-38, or a fragment thereof.

5. The composition as set forth in claim 1, wherein the exogenous synthase comprises at least one amino acid sequence selected from SEQ ID NOs: 51-175 or any combination thereof.

6. The composition as set forth in claim 1, wherein the nucleic acid molecule encoding an exogenous synthase comprises at least one vector.

7. The composition as set forth in claim 8, wherein the vector comprises at least an adenovirus, a retrovirus, an adeno-associated virus, a herpes virus, a poxvirus, a vaccinia virus, a lentivirus, or any combination thereof.

8. The composition as set forth in claim 1, wherein the composition comprises at least one nucleotide sequence that is at least about 70% identical to the nucleotide sequence selected from SEQ ID NOs: 2, 4, 6, 8, 10, 12, 14, 16, 18, 20, 22, 24, 26, 28, 30, 32, 34, or 45-50 or a fragment thereof.

9. The composition as set forth in claim 1, wherein the composition comprises at least one selected from a genetic delivery vector, a minicircle, a liposome, a plasmid, a viral vector, or any combination thereof.

10. The composition as set forth in claim 1, wherein the composition further comprises a nucleic acid molecule encoding 3-hydroxy-3-methylglutaryl coenzyme-A (HMG-CoA) reductase (HMGR) or a truncated form of HMGR.

11. The composition as set forth in claim 10, wherein the nucleic acid molecule comprises at least one nucleotide sequence that is at least about 70% identical to the nucleotide sequence selected from SEQ ID NO: 39 or a fragment thereof, or from SEQ ID NO: 41 or a fragment thereof.

12. The composition as set forth in claim 10, wherein the truncated HMGR comprises at least one amino acid sequence that is at least about 70% identical to the amino acid sequence selected from SEQ ID NO: 40 or a fragment thereof.

13. The composition as set forth in claim 1, wherein the composition comprises at least one tumor-specific promoter.

14. The composition as set forth in claim 13, wherein the tumor-specific promoter comprises one of the following nucleotide sequences: Survivin promoter, human (SEQ ID NO: 176), hTert core promoter, human (SEQ ID NO: 177), CXCR4 promoter, human [GenBank ID: U81003.1](SEQ ID NO: 178), Hexokinase type promoter, human [GenBank: AF148512.1] (SEQ ID NO: 179), Stromelysin 3 (MMP11) promoter, mouse [GenBank: AF297645.1] (SEQ ID NO: 180), Tyrosinase promoter, human, [GenBank: U03039.1] (SEQ ID NO: 181)Interleukin-10 promoter, human [GenBank: Z30175.1] (SEQ ID NO: 182), Epidermal growth factor receptor (EGFR) promoter, [GenBank: J03206.1](SEQ ID NO: 183), Mucin-like glycoprotein (DF3, MUC1) promoter, [GenBank: X69118.1] (SEQ ID NO: 184), Somatostatin receptor 2 (sst2)promoter, human [GenBank: AB260891.1] (SEQ ID NO: 185), c-erbB-2 promoters, human [GenBank ID: M16892.1] (SEQ ID NO: 186), c-erbB-3 promoter; human [GenBank ID: Z23134.1] (SEQ ID NO: 187), Thyroglobulin promoter, human [GenBank: X77275.1] (SEQ ID NO: 188), alpha-fetoprotein (AFP) promoter, human [GenBank: AB053572.1] (SEQ ID NO: 189), Villin 2 promoter, human [GenBank: EF184645.1] (SEQ ID NO: 190), or Albumin promoter (SEQ ID NO: 191).

15. The composition as set forth in claim 13, wherein the tumor-specific promoter comprises at least one amino acid sequence that is at least about 70% identical to the amino acid sequence selected from Survivin promoter, human (SEQ ID NO: 176), hTert core promoter, human (SEQ ID NO: 177), CXCR4 promoter, human [GenBankID: U81003.1](SEQ ID NO: 178), Hexokinase type promoter, human [GenBank: AF148512.1] (SEQ ID NO: 179), Stromelysin 3 (MMP11) promoter, mouse [GenBank: AF297645.1] (SEQ ID NO: 180), Tyrosinase promoter, human, [GenBank: U03039.1] (SEQ ID NO: 181)Interleukin-10 promoter, human [GenBank: Z30175.1] (SEQ ID NO: 182), Epidermal growth factor receptor (EGFR) promoter, [GenBank: J03206.1](SEQ ID NO: 183), Mucin-like glycoprotein (DF3, MUC1) promoter, [GenBank: X69118.1] (SEQ ID NO: 184), Somatostatin receptor 2 (sst2)promoter, human [GenBank: AB260891.1] (SEQ ID NO: 185), c-erbB-2 promoters, human [GenBank ID: M16892.1] (SEQ ID NO: 186), c-erbB-3 promoter; human [GenBank ID: Z23134.1] (SEQ ID NO: 187), Thyroglobulin promoter, human [GenBank: X77275.1] (SEQ ID NO: 188), alpha-fetoprotein (AFP) promoter, human [GenBank: AB053572.1] (SEQ ID NO: 189), Villin 2 promoter, human [GenBank: EF184645.1] (SEQ ID NO: 190), or Albumin promoter (SEQ ID NO: 191).

16. The composition as set forth in claim 1, wherein the nucleic acid molecule encoding an exogenous synthase is codon-optimized for mammalian cells.

17. The composition as set forth in claim 1, wherein the nucleic acid molecule encoding an exogenous synthase is codon-optimized for human cells.

18. A breath-based method of detecting cancer in a subject in need thereof, comprising:
(a) administering to the subject at least one composition, wherein the at least one composition comprises a nucleic acid molecule encoding an exogenous synthase, wherein the exogenous synthase expresses preferentially in cancer cells compared to noncancerous cells and catalyzes production of a volatile organic compound, and wherein the volatile organic compound is not produced endogenously in the subject;
(b) capturing breath exhaled from the subject;
(c) analyzing the exhaled breath for the volatile organic compound;
(d) comparing the amount of the volatile organic compound in the exhaled breath to a comparator; and
(e) determining the subject has cancer when the amount of the volatile organic compound in the exhaled breath is increased compared to a comparator.
